(19) United States
(12) Patent Application Publication
(10) Pub. No.: US 2023/0279351 A1
(43) Pub. Date: Sep. 7, 2023

(54) ARTIFICIAL ANTIGEN-SPECIFIC IMMUNOREGULATORY T (AIRT) CELLS

(71) Applicants: Seattle Children's Hospital (dba Seattle Children's Research Institute), Seattle, WA (US); Benaroya Research Institute at Virginia Mason, Seattle, WA (US)

(72) Inventors: Jane Buckner, Seattle, WA (US); David J. Rawlings, Seattle, WA (US); Karen Sommer, Seattle, WA (US); Yuchi Chiang Honaker, Seattle, WA (US); Peter Cook, Seattle, WA (US); Akhilesh Kumar Singh, Seattle, WA (US); Soo Jung Yang, Seattle, WA (US)

(21) Appl. No.: 17/596,493

(22) PCT Filed: Jun. 24, 2020
(86) PCT No.: PCT/US2020/039445

§ 371 Date: Dec. 10, 2021

Related U.S. Application Data
(60) Provisional application No. 62/867,670, filed on Jun. 27, 2019.
(60) Provisional application No. 62/987,810, filed on Mar. 10, 2020.

(51) Int. Cl. C12N 5/0783; C07K 14/47; C07K 14/725

(52) U.S. Cl. CPC C12N 5/0637; C07K 14/4702; C07K 14/7051; C12N 2310/20; C12N 2510/00

(57) ABSTRACT

Some embodiments of the compositions and methods disclosed herein include gene-edited, artificial immunoregulatory T cells (airT cells) comprising a constitutively expressed FoxP3 gene product expressed at a level equal to or greater than the level of FoxP3 expression in natural T regulatory (Treg or suppressor T) cells, and a transduced (e.g., artificially engineered by gene editing, viral vector transduction, transfection or other genetic engineering methodologies) T cell receptor (TCR). In some embodiments, the TCR is preferably specific for an antigen associated with an autoimmune, allergic, or other inflammatory condition. Some embodiments include methods for the preparation and/or use of airT cells. Some such embodiments include use of airT cells for the treatment and/or amelioration of a disorder, in which antigen-specific immunosuppression may be beneficial, such as an autoimmune, allergic, or other inflammatory disorder.

### CROSS-REFERENCE TO RELATED APPLICATIONS

This application is a U.S. National Phase Application of PCT International Application Number PCT/US2020/039445, filed on Jun. 24, 2020, designating the United States of America and published in the English language, which is an International Application of and claims the benefit of priority to U.S. Provisional Application No. 62/867670, filed on Jun. 27, 2019 and U.S. Provisional Application No. 62/987810, filed on Mar. 10, 2020. The disclosures of the abovereferenced applications are hereby expressly incorporated by reference in their entireties.

### REFERENCE TO SEQUENCE LISTING

The present application is being filed along with a Sequence Listing in electronic format. The Sequence Listing is provided as a file entitled SEQLISTSCRI252NP, created Aug. 25, 2022, which is approximately 564113 bytes in size. The information in the electronic format of the Sequence Listing is incorporated herein by reference in its entirety.

### FIELD OF THE INVENTION

Some embodiments provided herein include artificial antigen-specific immunoregulatory T (airT) cells. AirT cells include artificially engineered immune system T lymphocytes stably reprogrammed by gene editing to exhibit certain regulatory T cell (Treg) properties and are also artificially engineered by gene editing, viral vector transduction, transfection or other genetic engineering methodologies to express desired functional T cell antigen receptors (TCR) or other antigen receptors such as chimeric antigen receptors (CAR). In some embodiments, the airT cells are capable of immunosuppressive activity in response to specific antigen recognition by TCR.

### BACKGROUND OF THE INVENTION

Autoimmune diseases, such as type 1 diabetes mellitus, multiple sclerosis, myocarditis, rheumatoid arthritis (RA), and systemic lupus erythematosus (SLE, or “lupus”), are chronic, often life-threatening conditions that result from alterations in immunological self-tolerance, leading to aberrant immune activity and end-organ pathology. Inappropriate and deleterious dysregulation of immune tolerance can also contribute undesirably to pathologies associated with allergy, asthma, transplant rejection, and/or graft-versus-host disease (GVHD). The role of specialized antigen-recognizing thymic-derived T lymphocytes known as regulatory T cells (Treg, also referred to as suppressor T cells) in the maintenance of immune tolerance and prevention of autoimmunity is well established, and multiple autoimmune conditions are characterized by dysfunctional or dysregulated Treg compartments.

As a potential therapy for autoimmune disease, adoptive transfer to an afflicted subject of functional Treg selected for their immunosuppressive ability has been explored in mouse models and early phase clinical trials. However, a lack of autoantigen specificity of such Treg cells, and uncontrolled cell plasticity (e.g., conversion from immunosuppressive negative regulator of immunity to pro-inflammatory effector-like phenotype) resulting in the loss of immunosuppressive Treg activity, comprise two major limitations for the effective and sustained therapeutic benefit of such Treg adoptive transfer. It is believed that the use of immunosuppressive Treg selected to respond antigen-specifically to disease-associated autoantigens would lead to a safer, more effective adoptive transfer strategy than simple transfer of polyspecific Treg. In this respect, following infusion into a subject, the autoantigen-specific Treg cells would be expected to home specifically to tissue sites where autoimmune activity is manifest and, importantly, would mediate immune suppression specifically in response to the autoantigens that drive autoimmune disease pathogenesis. In support of this concept, studies in mice have shown that antigen-specific Tregs are more efficacious than polyclonal Tregs in murine models of autoimmune disease. (Duggleby et al., 2018 Front. Immunol. 9:252; Tang et al 2004 J Exp Med. 199(11):1455-1465; Tarbell et al 2004 J Exp Med 199:1467-1477.)

Therapeutic applications of adoptively transferred antigen-specific Treg or even of polyclonal Treg to treat autoimmune disease have, however, been limited, inter alia, by difficulties encountered in the course of isolating sufficient quantities of rare, antigen-specific Treg cells from a natural source, such as blood, and lymph, and by the overall scarcity of natural Treg in the peripheral blood, for example, approximately 1-4% of peripheral blood mononuclear cells include natural Treg. Development of Treg adoptive transfer therapies has also been hindered by challenges associated with expanding Treg populations to therapeutic numbers ex vivo while maintaining their immunosuppressive function, with the poor ability of adoptively transferred Treg cells to persist in an adoptive host and to proliferate after re-infusion. Also problematic has been Treg plasticity, such as conversion from immunosuppressive negative regulator of immunity to pro-inflammatory effector-like phenotype, in inflammatory settings in vivo. (Singer et al., 2014 Front. Immunol. 5:Art. 46; Trzonkowski et al., 2015 Sci. Translat. Med. 7(304):ps18 Romano et al., 2016 Transplant. Internatl. 30:745, McGovern et al., 2017 Front. Immunol. 8:Art. 1517).

No prior approaches provide bulk populations of stable Treg cells with suppressive activity having a desired antigen specificity, such as specificity for an antigen involved in the pathogenesis of a condition where antigen-specific immunosuppression would be beneficial, for instance, autoimmune disease, allergy, and/or other inflammatory conditions.

Accordingly, there remains a need for stable, antigen-specific immunoregulatory cells that maintain antigen-specific immunosuppressive capability in vitro and in vivo without exhibiting plasticity, as may be usefully administered to subjects in need of antigen-specific immunosuppression by adoptive transfer immunotherapy. Embodiments provided herein address this need, and offers other related advantages.

### SUMMARY OF THE INVENTION

Some embodiments of the methods and compositions provided herein include an artificial CD4+CD25+ antigen-specific immunoregulatory T (airT) cell, comprising: (a) an artificial modification of a forkhead box protein 3/winged helix transcription factor (FOXP3) gene, wherein the modified gene constitutively expresses a FOXP3 gene product at a FOXP3 expression level that is equal to or greater than the FOXP3 expression level of a naturally occurring regulatory T (Treg) cell; and (b) at least one transduced polynucleotide encoding an antigen-specific T cell receptor (TCR) polypeptide.

In some embodiments there is provided an artificial CD4+CD25+ antigen-specific immunoregulatory T (airT) cell obtained by artificial modification of a forkhead box protein 3/winged helix transcription factor (FOXP3) gene in a CD4+CD25⁻ T cell, wherein the artificial modification causes the airT cell to constitutively express a FOXP3 gene product at a FOXP3 expression level that is equal to or greater than the FOXP3 expression level of a naturally occurring regulatory T (Treg) cell; and at least one transduced polynucleotide encoding an antigen-specific T cell receptor (TCR) polypeptide.

In some embodiments the FOXP3 gene is present in a FOXP3 gene locus comprising an intronic regulatory T cell (Treg)-specific demethylation region (TSDR) having a plurality of cytosine-guanine (CG) dinucleotides, wherein each CG dinucleotide comprises a methylated cytosine (C) nucleotide at a nucleotide position that comprises a demethylated C nucleotide in a naturally occurring Treg cell. In some embodiments at least 80%, 85%, 90%, 95%, 96%, 97%, 98%, or 99% of the TSDR C nucleotides at nucleotide positions that comprise a demethylated C nucleotide in a naturally occurring Treg cell are methylated. In some embodiments the FOXP3 gene product is expressed at a level sufficient for the airT cell to maintain a CD4+CD25+ phenotype for at least 21 days in vitro. In some embodiments the FOXP3 gene product is expressed at a level sufficient for the airT cell to maintain a CD4+CD25+ phenotype for at least 60 days in vivo following adoptive transfer to an immunocompatible mammalian host in need of antigen-specific immunosuppression. In some embodiments the cell comprises a phenotype selected from one or more of: (i) HeliosLo, (ii) CD152+, (iii) CD127⁻, or (iv) ICOS+. In some embodiments the artificial modification comprises a knockout of a native FOXP3 gene locus in the cell.

In some embodiments the artificial modification comprises an inserted nucleic acid molecule comprising a constitutively active promoter at a native FOXP3 gene locus of the cell, wherein the promoter is positioned in the FOXP3 gene so as to be capable of promoting transcription of an endogenous FOXP3-encoding nucleotide sequence of the FOXP3 gene locus. In some embodiments the inserted nucleic acid molecule further comprises a nucleic acid sequence encoding a first chemically inducible signaling complex (CISC) component capable of specifically binding to a CISC inducer molecule. In some embodiments the transduced polynucleotide encoding the TCR polypeptide further comprises a nucleic acid sequence encoding a second chemically inducible signaling complex (CISC) component that is different from the first CISC component and is capable of specifically binding to the CISC inducer molecule. In some embodiments the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments the nucleic acid molecule comprising the constitutively active promoter is inserted downstream of an intronic regulatory T cell (Treg)-specific demethylation region (TSDR) in the native FOXP3 gene locus. In some embodiments the constitutively active promoter is an MND promoter. In some embodiments the artificial modification comprises an inserted nucleic acid molecule comprising an exogenous FOXP3-encoding polynucleotide operably linked to a constitutively active promoter at a native FOXP3 gene locus of the cell. In some embodiments the inserted nucleic acid molecule comprising the exogenous FOXP3-encoding polynucleotide operably linked to the constitutively active promoter further comprises a nucleic acid sequence encoding a first chemically inducible signaling complex (CISC) component capable of specifically binding to a CISC inducer molecule. In some embodiments the transduced polynucleotide encoding the TCR polypeptide further comprises a nucleic acid sequence encoding a second chemically inducible signaling complex (CISC) component that is different from the first CISC component and is capable of specifically binding to the CISC inducer molecule. In some embodiments at least one of the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule.

In some embodiments the nucleic acid molecule comprising the exogenous FOXP3-encoding polynucleotide operably linked to the constitutively active promoter is inserted downstream of an intronic regulatory T cell (Treg)-specific demethylation region (TSDR) in the native FOXP3 gene locus. In some embodiments the constitutively active promoter is an MND promoter.

In some embodiments the artificial modification comprises an insertion of a nucleic acid molecule comprising an exogenous FOXP3-encoding polynucleotide operably linked to a constitutively active promoter at a chromosomal site other than a native FOXP3 gene locus of the cell. In some embodiments at least one native T cell receptor (TCR) gene locus of the airT cell is knocked out or inactivated and replaced with the at least one transduced polynucleotide encoding an antigen-specific T cell receptor (TCR) polypeptide. In some embodiments the at least one native TCR gene locus that is knocked out or inactivated is a native TCR alpha chain (TRAC) locus. In some embodiments the inserted nucleic acid molecule comprising the exogenous FOXP3-encoding polynucleotide operably linked to the constitutively active promoter further comprises a nucleic acid sequence encoding a first chemically inducible signaling complex (CISC) component capable of specifically binding to a CISC inducer molecule. In some embodiments the transduced polynucleotide encoding the TCR polypeptide further comprises a nucleic acid sequence encoding a second chemically inducible signaling complex (CISC) component that is different from the first CISC component and is capable of specifically binding to the CISC inducer molecule. In some embodiments at least one of the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments the constitutively active promoter is an MND promoter. In some embodiments the chromosomal site that is other than a native FOXP3 gene locus, and at which is inserted the nucleic acid molecule comprising the exogenous FOXP3-encoding polynucleotide operably linked to the constitutively active promoter, is within a T cell receptor alpha chain (TRAC) locus of the cell.

In some embodiments in the airT cell at least one native T cell receptor (TCR) gene locus is knocked out or inactivated and replaced with the at least one transduced polynucleotide encoding an antigen-specific T cell receptor (TCR) polypeptide. In some embodiments the at least one native TCR gene locus that is knocked out is a native TCR alpha chain (TRAC) locus.

In some embodiments there is provided an artificial CD4+CD25+ antigen-specific immunoregulatory T (airT) cell, comprising: (a) a transduced nucleic acid sequence encoding an exogenous forkhead box protein 3/winged helix transcription factor (FOXP3) gene product, wherein the cell constitutively expresses the FOXP3 gene product at a FOXP3 expression level that is equal to or greater than the FOXP3 expression level of a naturally occurring regulatory T (Treg) cell; and (b) at least one transduced polynucleotide encoding an exogenous antigen-specific T cell receptor (TCR) polypeptide; wherein the transduced nucleic acid sequence encoding the exogenous FOXP3 gene product further comprises a nucleic acid sequence encoding a first chemically inducible signaling complex (CISC) component capable of specifically binding to a CISC inducer molecule; and wherein the transduced nucleic acid sequence encoding the exogenous TCR gene product further comprises a nucleic acid sequence encoding a second chemically inducible signaling complex (CISC) component that is different from the first CISC component and is capable of specifically binding to the CISC inducer molecule.

In some embodiments there is provided an artificial CD4+CD25+antigen-specific immunoregulatory T (airT) cell, comprising: (a) a native FOXP3 gene locus that has been knocked out or inactivated, and into which FOXP3 locus has been inserted, by homology-directed repair, either: (i) a nucleic acid molecule comprising a constitutively active promoter that is capable of promoting transcription of an endogenous FOXP3-encoding nucleotide sequence of the FOXP3 gene, or (ii) a nucleic acid molecule comprising a constitutively active promoter operably linked to a nucleotide sequence encoding an exogenous FOXP3 protein or a functional derivative thereof, and which constitutively expresses the FOXP3 gene product at a FOXP3 expression level that is equal to or greater than the FOXP3 expression level of a naturally occurring regulatory T (Treg) cell, wherein the inserted nucleic acid molecule encoding the constitutively active promoter or encoding the constitutively active promoter operably linked to the nucleotide sequence encoding exogenous FoxP3 protein or functional derivative thereof further comprises a nucleic acid sequence encoding a first chemically inducible signaling complex (CISC) component capable of specifically binding to a CISC inducer molecule; and (b) a native T-cell receptor alpha (TRAC) locus that has been knocked out and into which TRAC locus has been inserted, by homology-directed repair, at least one transduced polynucleotide encoding an exogenous antigen-specific T cell receptor (TCR) polypeptide, wherein the transduced nucleic acid sequence encoding the exogenous TCR polypeptide further comprises a nucleic acid sequence encoding a second chemically inducible signaling complex (CISC) component that is different from the first CISC component and is capable of specifically binding to the CISC inducer molecule.

In some embodiments at least one of the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments the airT cell comprises at least a first and a second transduced polynucleotide each encoding an antigen-specific TCR polypeptide, wherein said first transduced polynucleotide encodes a TCR V-alpha polypeptide and said second transduced polynucleotide encodes a TCR V-beta polypeptide, wherein said V-alpha polypeptide and said V-beta polypeptide comprise a functional TCR capable of specific antigen recognition. In some embodiments the airT cell expresses an antigen-specific T cell receptor (TCR) comprising the antigen-specific TCR polypeptide encoded by the at least one transduced polynucleotide encoding said TCR polypeptide and which is capable of antigen-specifically induced immunosuppression in response to HLA-restricted stimulation by an antigen that is specifically recognized by said TCR polypeptide. In some embodiments the antigen-specifically induced immunosuppression comprises one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines, perforin/ granzyme, or anti-inflammatory products by the airT cell or induction in the airT cell of at least one of indoleamine 2,3-dioxygenase (IDO), competition for IL2 or adenosine, catabolism of tryptophan, and expression of inhibitory receptors, and (iv) inhibition of either or both of activation and proliferation of effector T cells that do not recognize the antigen that is specifically recognized by the airT TCR comprising the TCR polypeptide that is encoded by the at least one transduced polynucleotide.

In some embodiments the TCR specifically recognizes an antigen associated with pathogenesis of an autoimmune condition, an allergic condition, or an inflammatory condition. In some embodiments (i) the autoimmune condition is selected from type 1 diabetes mellitus, multiple sclerosis, systemic lupus erythematosus, myasthenia gravis, rheumatoid arthritis, Crohn’s disease, bullous pemphigoid, pemphigus vulgaris, autoimmune hepatitis, psoriasis, Sjogren’s syndrome, or celiac disease; (ii) the allergic condition is selected from allergic asthma, pollen allergy, food allergy, drug hypersensitivity, or contact dermatitis; and (iii) the inflammatory condition is selected from pancreatic islet cell transplantation, asthma, hepatitis, inflammatory bowel disease (IBD), ulcerative colitis, graft-versus-host disease (GVHD), tolerance induction for transplantation, transplant rejection, or sepsis. In some embodiments (i) the antigen associated with pathogenesis of the autoimmune condition is selected from an autoantigen set forth in any one or more of FIGS. 141-144, (ii) the antigen associated with pathogenesis of the allergic condition is selected from an allergenic antigen set forth in any one or more of FIGS. 141-144, and (iii) the antigen associated with pathogenesis of the inflammatory condition is selected from an inflammation-associated antigen set forth in any one or more of FIGS. 141-144.

In some embodiments the airT cell comprises at least one transduced polynucleotide sequence encoding a TCR polypeptide that specifically binds in a human HLA-restricted manner to an antigenic polypeptide epitope of no more than 35, 34, 33, 32, 31, 30, 29, 28, 27, 26, 25, 24, 23, 22, 21, 20, 19, 18, 17, 16, 15, 14, 13, 12, 11, 10, 9, 8, or 7 consecutive amino acids of an amino acid sequence selected from any one of the antigenic polypeptide sequences set forth in any one or more of FIGS. 141-144, or that is encoded by a nucleotide sequence set forth in any one or more of FIGS. 139-140. In some embodiments the airT cell comprises at least a first and a second transduced polynucleotide sequence encoding, respectively, a TCR V-alpha polypeptide and a TCR V-beta polypeptide of a TCR that specifically binds in a human HLA-restricted manner to an antigenic polypeptide epitope of no more than 35, 34, 33, 32, 31, 30, 29, 28, 27, 26, 25, 24, 23, 22, 21, 20, 19, 18, 17, 16, 15, 14, 13, 12, 11, 10, 9, 8, or 7 consecutive amino acids of an amino acid sequence selected from any one of the antigenic polypeptide sequences set forth in any one or more of FIGS. 141-144, or that comprises any one TCR-alpha polypeptide sequence set forth in any one or more of FIGS. 136-140 or encoded by a nucleotide sequence set forth in any one or more of FIGS. 139-140. In some embodiments the airT cell comprises at least a first and a second transduced polynucleotide sequence encoding, respectively, a TCR V-alpha polypeptide and a TCR V-beta polypeptide of a TCR that specifically binds in a human HLA-restricted manner to an antigenic polypeptide, wherein the TCR V-alpha and V-beta polypeptides comprise paired sequences selected from any one paired TCR V-alpha and V-beta polypeptide sequences set forth in FIGS. 143.

In some embodiments the cell exhibits an induced level of Treg biological activity that is increased in response to MHC-restricted stimulation of the airT cell by an antigen recognized by the TCR polypeptide encoded by the at least one transduced polynucleotide, relative to a control level of Treg biological activity that is exhibited by the airT cell without MHC-restricted stimulation by the antigen, wherein the Treg biological activity comprises one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines, perforin/ granzyme, or anti-inflammatory products by the airT cell or induction in the airT cell of at least one of indoleamine 2,3-dioxygenase (IDO), competition for IL2 or adenosine, catabolism of tryptophan, expression of inhibitory receptors, or (iv) inhibition of either or both of activation and proliferation of effector T cells that do not recognize the antigen that is specifically recognized by the airT TCR comprising the TCR polypeptide that is encoded by the at least one transduced polynucleotide.

In some embodiments, (1) the antigen associated with pathogenesis of an autoimmune condition is IGRP(241-270) peptide, wherein the autoimmune condition is type 1 diabetes, and wherein the TCR is TCR T1D4 recognizing the IGRP(241-270) peptide in an HLA DRB1*0404-restricted manner, or (2) the antigen associated with pathogenesis of an autoimmune condition is IGRP(305-324) peptide, wherein the autoimmune condition is type 1 diabetes, and wherein the TCR is TCR T1D5 recognizing the IGRP(305-324) peptide in an HLA DRB1*0404-restricted manner.

Some embodiments of the methods and compositions provided herein include any one of the foregoing airT cells for use in the treatment, inhibition, or amelioration of an autoimmune condition, such as one selected from type 1 diabetes mellitus, multiple sclerosis, systemic lupus erythematosus, myasthenia gravis, rheumatoid arthritis, Crohn’s disease, bullous pemphigoid, pemphigus vulgaris, or autoimmune hepatitis, an allergic condition, such as one selected from allergic asthma, pollen allergy, food allergy, drug hypersensitivity, or contact dermatitis, or an inflammatory condition, such as one selected from pancreatic islet cell transplantation, asthma, hepatitis, inflammatory bowel disease (IBD), ulcerative colitis, graft-versus-host disease (GVHD), tolerance induction for transplantation, transplant rejection, or sepsis.

In some embodiments, the TCR polypeptide binds to an antigen associated with a disorder selected from type 1 diabetes mellitus, multiple sclerosis, myocarditis, rheumatoid arthritis (RA), and systemic lupus erythematosus (SLE).

In some embodiments, the antigen is selected from the group consisting of vimentin, aggrecan, cartilage intermediate layer protein (CILP), preproinsulin, islet-specific glucose-6-phosphatase catalytic subunit-related protein (IGRP), and enolase.

In some embodiments, the antigen comprises an epitope selected from the group consisting of Enol326, CILP297-1, Vim418, Agg520, and SLE3.

In some embodiments, the antigen comprises an epitope having the amino acid sequence of any one of SEQ ID NOs 1363-1376 and 1408-1415.

In some embodiments, the TCR polypeptide comprises: a CD3 alpha polypeptide having the amino acid sequence of any one of SEQ ID NOs 1377-1390; and/or a CD3 beta polypeptide having the amino acid sequence of any one of SEQ ID NOs 1377-1390.

Some embodiments of the methods and compositions provided herein include a pharmaceutical composition comprising any one of the foregoing airT cells and a pharmaceutically acceptable excipient.

Some embodiments of the methods and compositions provided herein include use of any one of the foregoing airT cells as a medicament.

In some embodiments there is provided a method of producing an artificial antigen-specific immunoregulatory T (airT) cell, comprising: (a) introducing into a CD4+ T cell (1) a FOXP3 guide RNA (gRNA) comprising a spacer sequence complementary to a sequence within a native forkhead box protein 3/winged helix transcription factor (FOXP3) gene in the cell, or a nucleic acid encoding the FOXP3 gRNA; (2) a DNA endonuclease capable of forming a complex with the FOXP3 gRNA of (1), or a nucleic acid encoding the DNA endonuclease; and (3) a FOXP3 locus donor template selected from (i) a nucleic acid molecule comprising a constitutively active promoter capable of promoting transcription of an endogenous FOXP3-encoding nucleotide sequence of the FOXP3 gene; and (ii) a nucleic acid molecule comprising a constitutively active promoter operably linked to a nucleotide sequence encoding a FOXP3 protein or a functional derivative thereof, under conditions and for a time sufficient for knock-out or inactivation of the native FOXP3 gene locus in the cell and insertion of all or a portion of the FOXP3 locus donor template nucleic acid; and (b) simultaneously or sequentially and in any order with (a), transducing the CD4+ T cell with at least one polynucleotide encoding an antigen-specific T cell receptor (TCR) polypeptide. In some embodiments step (b) is selected from: (i) transducing the CD4+ T cell with at least one retroviral vector comprising the polynucleotide encoding the antigen-specific T cell receptor (TCR) polypeptide, and (ii) introducing into the CD4+ T cell (1) a T cell receptor alpha (TRAC) guide RNA (gRNA) comprising a spacer sequence complementary to a sequence within a native TRAC gene locus in the cell, or a nucleic acid encoding the TRAC gRNA; (2) a DNA endonuclease capable of forming a complex with the TRAC gRNA of (1), or a nucleic acid encoding the DNA endonuclease; and (3) a TRAC locus donor template comprising the at least one polynucleotide encoding the antigen-specific T cell receptor (TCR) polypeptide, under conditions and for a time sufficient for knock-out or inactivation of the native TRAC gene locus in the cell and insertion of all or a portion of the TRAC locus donor template nucleic acid.

In some embodiments there is provided a method of producing an artificial antigen-specific immunoregulatory T (airT) cell, comprising: (a) introducing into a CD4+ T cell (1) a first T cell receptor alpha (TRAC) guide RNA (gRNA) comprising a first spacer sequence complementary to a first sequence within a native TRAC gene locus in the cell, or a nucleic acid encoding the first TRAC gRNA; (2) a first DNA endonuclease capable of forming a complex with the first TRAC gRNA of (1), or a nucleic acid encoding the first DNA endonuclease; and (3) a first TRAC locus donor template selected from (i) a nucleic acid molecule comprising a nucleotide sequence encoding a FOXP3 protein or a functional derivative thereof, and (ii) a nucleic acid molecule comprising a constitutively active promoter operably linked to a nucleotide sequence encoding a FOXP3 protein or a functional derivative thereof, under conditions and for a time sufficient for knock-out of the native TRAC gene locus in the cell and insertion of all or a portion of the first TRAC locus donor template nucleic acid; and (b) simultaneously or sequentially and in any order with (a), introducing into the CD4+ T cell (1) a second T cell receptor alpha (TRAC) guide RNA (gRNA) comprising a second spacer sequence complementary to a second sequence within a TRAC gene, or a nucleic acid encoding the second TRAC gRNA, wherein the second spacer sequence is not identical to the first spacer sequence; (2) a second DNA endonuclease capable of forming a complex with the second TRAC gRNA of (1), or a nucleic acid encoding the second DNA endonuclease, wherein the second DNA endonuclease is selected from a DNA endonuclease that is identical to the first DNA endonuclease and a DNA endonuclease that is not identical to the first DNA endonuclease; and (3) a second TRAC locus donor template comprising the at least one polynucleotide encoding the antigen-specific T cell receptor (TCR) polypeptide, under conditions and for a time sufficient for knock-out or inactivation of the native TRAC gene locus in the cell and insertion of all or a portion of the second TRAC locus donor template nucleic acid.

In some embodiments there is provided a method of producing an artificial antigen-specific immunoregulatory T (airT) cell, comprising: introducing into a CD4+ T cell (1) a T cell receptor alpha (TRAC) guide RNA (gRNA) comprising a spacer sequence that is complementary to a sequence within a native TRAC gene locus in the cell, or a nucleic acid encoding the TRAC gRNA; (2) a DNA endonuclease that is capable of forming a complex with the TRAC gRNA of (1), or a nucleic acid encoding the DNA endonuclease; and (3) a TRAC locus donor template which comprises the at least one polynucleotide that encodes the antigen-specific T cell receptor (TCR) polypeptide, under conditions and for a time sufficient for knock-out or inactivation of the native TRAC gene locus in the cell and insertion of all or a portion of the TRAC locus donor template by homology-directed repair. In some embodiments a first one of said insertion donor templates further comprises a nucleic acid sequence encoding a first chemically inducible signaling complex (CISC) component that is capable of specifically binding to a CISC inducer molecule, and a second one of said insertion donor templates further comprises a nucleic acid sequence encoding a second chemically inducible signaling complex (CISC) component that is different from the first CISC component and is capable of specifically binding to the CISC inducer molecule. In some embodiments at least one of the first and second insertion donor templates further comprises a nucleic acid sequence encoding a third CISC component that is different from the first and second CISC components and is capable of specifically binding to the CISC inducer molecule. In some embodiments wherein one or more of: (a) the DNA endonuclease is selected from a CRISPR/Cas, a TALEN, a meganuclease, megaTAL, or a zinc finger nuclease, (b) the constitutively active promoter is MND, insertion is by a mechanism selected from homology-directed repair or non-homologous end joining, (d) the first and second CISC components are selected in a mutually exclusive manner from IL2RB or IL2RG, (e) the third CISC component is FKBP, and (f) the CISC inducer molecule is rapamycin or an analog thereof.

Some embodiments of the methods and compositions provided herein include a method of producing any one of the foregoing artificial antigen-specific immunoregulatory T (airT) cells, comprising performing any one of the foregoing methods of producing an artificial antigen-specific immunoregulatory T (airT) cell.

In some embodiments there is provided a method for treating, inhibiting, or ameliorating a subject having a condition in need of antigen-specific immunosuppression, comprising administering to the subject a therapeutically effective amount of a plurality of the artificial immunoregulatory T (airT) cells, wherein said airT cells express at least one T cell receptor (TCR) that specifically recognizes the antigen for which antigen-specific immunosuppression is needed. In some embodiments the condition in need of antigen-specific immunosuppression is an autoimmune condition, an allergic condition, or an inflammatory condition. In some embodiments (i) the autoimmune condition is selected from type 1 diabetes mellitus, multiple sclerosis, systemic lupus erythematosus, myasthenia gravis, rheumatoid arthritis, Crohn’s disease, bullous pemphigoid, pemphigus vulgaris, or autoimmune hepatitis; (ii) the allergic condition is selected from allergic asthma, pollen allergy, food allergy, drug hypersensitivity, or contact dermatitis; and (iii) the inflammatory condition is selected from pancreatic islet cell transplantation, asthma, hepatitis, inflammatory bowel disease (IBD), ulcerative colitis, graft-versus-host disease (GVHD), tolerance induction for transplantation, transplant rejection, or sepsis. In some embodiments (i) the antigen associated with pathogenesis of the autoimmune condition is selected from an autoantigen set forth in any one or more of FIGS. 141-144, (ii) the antigen associated with pathogenesis of the allergic condition is selected from an allergenic antigen set forth in any one or more of FIGS. 141-144, and (iii) the antigen associated with pathogenesis of the inflammatory condition is selected from an inflammation-associated antigen set forth in any one or more of FIGS. 141-144.

Some embodiments of the methods and compositions provided herein include a method for treating or ameliorating a subject having a disorder comprising administering to the subject any one of the foregoing artificial immunoregulatory T (airT) cells.

In some embodiments, the disorder is selected from the group consisting of type 1 diabetes mellitus, multiple sclerosis, systemic lupus erythematosus (SLE), myasthenia gravis, rheumatoid arthritis (RA), Crohn’s disease, bullous pemphigoid, pemphigus vulgaris, or autoimmune hepatitis, an allergic condition, such as one selected from allergic asthma, pollen allergy, food allergy, drug hypersensitivity, or contact dermatitis, or an inflammatory condition, such as one selected from pancreatic islet cell transplantation, asthma, hepatitis, inflammatory bowel disease (IBD), ulcerative colitis, graft-versus-host disease (GVHD), tolerance induction for transplantation, transplant rejection, and sepsis. In some embodiments, the TCR polypeptide binds to an antigen associated with a disorder selected from type 1 diabetes mellitus, multiple sclerosis, myocarditis, rheumatoid arthritis (RA), and systemic lupus erythematosus (SLE).

In some embodiments, the TCR polypeptide binds to an antigen selected from the group consisiting of vimentin, aggrecan, cartilage intermediate layer protein (CILP), preproinsulin, islet-specific glucose-6-phosphatase catalytic subunit-related protein (IGRP), and enolase. In some embodiments, the TCR polypeptide binds to an antigen comprising an epitope having the amino acid sequence of any one of SEQ ID NOs 1363-1376 and 1408-1415.

In some embodiments, the TCR polypeptide comprises: a CD3 alpha polypeptide having the amino acid sequence of any one of SEQ ID NOs 1377-1390; and/or a CD3 beta polypeptide having the amino acid sequence of any one of SEQ ID NOs 1377-1390.

### BRIEF DESCRIPTION OF THE DRAWINGS

FIGS. 1A-11 relate to the engineering of human CD4+ T cells into airT cells using gene editing.

FIG. 1A, FIG. 1B and FIG. 1C depict exemplary schema for converting CD4+ T cells into airT cells of the present disclosure. FIG. 1A is a schematic diagram of FOXP3 locus before (top) and after (bottom) gene editing using FOXP3 TALEN or CRISPR/Cas9 with FOXP3 guide RNA. TALEN or CRISPR/Cas9 cleaves FoxP3 locus at exon 1, initiating site-specific double stranded DNA break. AAV provides donor template containing MND and GFP (to allow analysis of editing efficiency), which is inserted into exon 1 at the DNA break. After the homology-directed repair, the MND promoter drives expression of FoxP3 and GFP reporter. FIG. 1B depicts a timeline of steps of gene editing and cell analysis and efficacy of airT generation from input Tconv cells. FIG. 1C depicts representative flow plots showing correlation between Foxp3 and GFP on day 4 after editing. The three panels on the right-hand side of the figure show CD25, CD127, Helios, CD45RO, ICOS, and CTLA-4 expression in Foxp3+ GFP+ gated cells, respectively.

FIG. 2 depicts flow plots (bottom) showing GFP and Foxp3 expression on day 4 and day 11 after editing according to the timeline shown at top. These data show that Foxp3 editing in CD4+ T cells is efficient and results in high, stable expression of Foxp3.

FIG. 3A, FIG. 3B, FIG. 3C and FIG. 3D depict data comparing airT cells and activated natural T regulatory (nTreg) cells. FIG. 3A depicts a timeline of steps to generate edTreg and activated nTreg for comparison. CD4+ cells were isolated from PBMC using MACS CD4+ isolation kit and Tconv (CD25- CD127+) and Treg (CD25 high CD127-) cells were further sorted by flow. Sorted Tconv and Treg cells were activated with CD3/CD28 activator beads and beads were removed after 48 hr activation. Only Tconv cells were Foxp3-edited using Cas9/Foxp3 gRNA and AAV-MND-LNGFR-Foxp3 ki to generate edTreg/airT. nTreg cells were treated in the same manner without Foxp3 editing. LNGFR+ cells from Foxp3-edited Tconv cells were enriched using MACS LNGFR beads on day 10. LNGFR+ edTreg and nTreg cells were used for suppression assay. FIG. 3B depicts a comparison of efficacy in generation of edTreg and nTreg from 1×10⁷ PBMC. At day 0, 1×10⁷ PBMC. Tconv and nTreg cells activated on day 0 were expanded 10-30 times and 1-2 times, respectively, from day 0 to day 10. For edTreg, Treg yield on day 10 was calculated based on editing rate (10-30%). FIG. 3C depicts representative flow plots showing Treg phenotype in Foxp3-edited Tconv and nTreg cells on day 10. Top panels show (left-most panel) LNGFR expression in edited Tconv and (right) Foxp3, Helios, CD25, CD127, ICOS, and CTLA-4 expression in edited Treg (LNGFR+ gate, top panels) and nTreg (bottom panels). FIG. 3D (upper panels) depicts comparison of Foxp3, CTLA-4, and ICOS expression in edTreg/airT (blue) and nTreg (red). FIG. 3D (bottom table) shows the MFI.

FIG. 4A and FIG. 4B show that airT cells have superior in vitro suppressive activity to nTreg. FIG. 4A depicts data from an in vitro suppression assay comparing suppressive activities of edTreg/airT and nTreg on CD4+ T_{eff} cells at the indicated Treg:T_{eff} ratios. airT or nTreg cells were labeled with EF670, and CD4+ T_{eff} cells were labeled with Cell Trace Violet (CTV). T_{eff} cells were co-cultured with airT or nTreg at different ratios, 0:1 (T_{eff} only), 1:1, 1:2, 1:4, 1:8, 1:16, and 1:32 (Treg:T_{eff}). CD3/CD28 activator beads were added at 1:25 (bead to T_{eff} ratio) and cells were analyzed by flow after 4d incubation. Dilution of CTV in T_{eff} cells was measured as proliferation. FIG. 4B depicts percent suppression calculated as (% proliferation in T_{eff} only+beads - % proliferation in T_{eff} cells cultured with Treg) / (% proliferation in T_{eff} only+beads) × 100.

FIG. 5 depicts exemplary lentiviral islet-specific TCR constructs expressing rare islet-specific TCRs derived from Type 1 diabetes (T1D) subjects. Panel A depicts a table of lentiviral vectors encoding GAD65 or IGRP specific TCRs (4.13, T1D2, T1D4, T1D5-1, or T1D5-2), their epitope specificity, and TCR alpha or beta chain usage. Panel B depicts structure of lentiviral islet-specific TCR. TCR constructs include human TCR variable regions from the islet-specific TCRs and mouse TCR constant regions that allow to improve pairing between the transduced human TCR chains.

FIG. 6 depicts validation of islet Ag-specific TCR expression: murine TCRβ expression and proliferation of islet antigen-specific T cells. Panel A depicts flow plots for CD4+ T cells isolated, activated with CD3/CD28 beads, and transduced with LV islet-TCRs. Flow plots show mTCRβ expression gated on CD3/CD28-activated CD4+ cells day 9 post-transduction with lentivirus (LV) encoding islet-specific TCR. Panel B depicts flow plots for CD4+ T cells transduced with LV islet-TCRs labeled with CTV and co-cultured with APC (irradiated PBMC) and their cognate peptide or irrelevant peptide for 5 days. Flow plots showing cell proliferation of LV-transduced CD4+ T cells labeled with CTV following 5-day co-culture with antigen-presenting cells (APC; irradiated PBMC) and cognate or irrelevant peptide. Proliferation is shown as CTV dilution.

FIG. 7 shows generation of Foxp3-edited T cells with islet-specific TCR. Panel A depicts a timeline of generating edTreg cells with islet-specific TCRs. Panel B depicts representative flow plots showing mTCRβexpression and LNGFR/Foxp3 expression on CD4+ cells on day 7 after transduction with T1D4 or T1D5-1 TCR and Foxp3 editing. Right panels show expression of CD25, CD127, CTLA-4, and ICOS gated on LNGFR+ cells.

FIG. 8 relates to exemplary antigen-specific suppression assays of the present disclosure. Panel A depicts a timeline for generation of edTreg cells expressing islet-specific TCRs. edTreg cells with islet-specific TCRs (no LV TCR, T1D4, or T1D5-1 TCR) were enriched by LNGFR expression using MACS LNGFR beads. LNGFR+ cells were aliquoted and frozen down for further experiments. Panel B depicts a summary of method used to assess antigen-specific suppression assays. CD4+ T cells transduced with islet-specific TCRs (T1D4 or T1D5-1 TCR) were used as T_{eff} cells. T_{eff} cells and Treg cells were labeled with different reagents, for example CTV or EF670, and co-cultured with or without edTreg cells with 1:1 or 1:2 ratio in the presence of APC (autologous irradiated PBMC) and various peptides. Cells were stained and analyzed by flow after 1 d or 4 d incubation for measuring cytokine generation and proliferation of T_{eff} cells, respectively.

FIG. 9 and FIG. 10 depict suppressive activity of edTreg/airT on T_{eff} proliferation in the presence of APC and the indicated peptide(s). T_{eff} and Treg cells were labeled with CTV and EF670, respectively. CD4+ T cells transduced with T1D4-TCR (T1D4 T_{eff}) were co-cultured with or without edTreg expressing T1D4-TCR (T1D4 edTreg) or T1D5-1-TCR (T1D5-1 edTreg) in the presence of APC and various peptides (DMSO, IGRP 241, IGRP 305, or IGRP241+IGRP 305). 4 days after the co-culture, cells were stained and analyzed for T_{eff} proliferation as dilution of CTV. Flow plots show T_{eff} proliferation gated on CD3+ CD4+ CTV+ EF670- LNGFR-.

FIG. 11 depicts suppression of cytokine generation in T_{eff} by edTreg/airT. T_{eff} and Treg cells were labeled with CTV and EF670, respectively. T1D4 T_{eff} cells were cocultured with or without untransduced edTreg or T1D4 edTreg/airT cells in the presence of APC and peptides (DMSO or IGRP 241). 1 day after the co-culture, cells were contacted with BFA for 4 h, stained, and analyzed for cytokine generation from T_{eff} cells. Flow plots show TNF, IFNg, or IL-17 generation from T1D4 T_{eff} cells gated on CD4+ CTV+ EF670-.

FIGS. 12-17 relate to the development and characterization of antigen-specific human Foxp3-edited human CD4+ T cells.

FIG. 12 depicts (top) an exemplary scheme for generating human antigen-specific edTreg/airT from peripheral blood cells and (bottom) phenotype of FOXP3-edited human antigen-specific CD4+ T cells. In the bottom panels, representative flow plots (left) and percentage (right) of GFP expression in tetramer positive (Tr+; a mixture of MHC class II tetramers with flu or tetanus peptides) human CD4+ T cells at 4 days post-gene editing (n=5).

FIG. 13 depicts a characterization of FOXP3-edited human antigen-specific CD4+ T cells. Panel A depicts phenotype of FOXP3 edited human antigen-specific CD4+ T cells. Bar chart summarizes flow cytometry data (n=5); chart shows expression of Treg markers and intracellular IL- 2 production in Tmr+edTreg, Tmr+ Mock-edited cells, as well as in thymus-generated Treg (tTreg) obtained from an unrelated donor. Data shown are representative of 5 independent experiments. P values of statistically significant differences are indicated above bars. Panel B depicts human antigen-specific edTreg/airT suppresses proliferation of T_{eff} in vitro. Suppression assays conducted using Tmr+edTreg/airT or mock-edited Tmr+ cells co-cultured with T_{eff} from healthy controls, APCs, and soluble anti-CD3 and anti-CD28. Ratio of antigen presenting cells (irradiated CD4- PBMC): Tmr+edTreg or mock-edited Tmr+ cells: T_{eff} was 2:1:1. 1 µCi 3H was added 18 hours prior to the end of the 4 day assay and proliferation was measured by a scintillation counter. Bar graph indicated averaged results from three experiments with three donors.

FIG. 14 depicts successful generation of antigen-specific edTreg/airT by peptide stimulation followed by Foxp3 editing. Panel A depicts a timeline of steps of antigen-specific T cell expansion and gene editing. After 9 days of peptide stimulation to expand T cells specific for MP, HA, or Tetanus, cells were activated with CD3/CD28 activator beads for gene editing. Beads were added to the sorted cells to enhance expansion of antigen-specific Tregs. Panel B depicts flow plots show GFP and Foxp3 expression on day 15 after editing. GFP+ Foxp3+ cells were CD25+ CD 127 - and about 60% of cells were MP, HA, or TT specific by tetramers.

FIG. 15 depicts antigen-specific suppression by Foxp3-edited Tregs/airT. Panel A a timeline of steps of generating antigen-specific edTreg/airT cells for suppression assay. GFP+ cells were sorted and expanded with CD3/CD28 beads on day 15 after editing. Beads were removed after 7 d incubation and edTreg/airT cells were harvested and used for suppression assay after 11 days of expansion. Panel B depicts a summary of suppression assay design. CD4+CD25+ cells were isolated from autologous PBMC, labeled with EF670, and used as T_{eff} cells. CD4-CD25+ cells were irradiated and used as APC, and edTreg/airT cells were labeled with Cell Trace Violet (CTV). T_{eff} cells and APC were co-cultured with or without edTreg/airT cells in the presence of DMSO or peptide pool (MP+HA+TT). Panel C depicts after 7 days of co-culture, cells were stained and analyzed by flow. CD3+ CD4+ EF670+ CTV- cells were gated as T_{eff} cells. Panel D depict a dilution of EF670 in T_{eff} cells was measured as proliferation and 15% of EF670- cells from co-culture of T_{eff} cells with APC and the peptide pool was normalized as 100% proliferation. % suppression was calculated as (100-% Proliferation).

FIG. 16 depicts an expansion of islet-specific T cells of multiple specificities by peptide stimulation. Panel A depicts an exemplary timeline for generating islet-antigen specific edTreg/airT cells. Freshly isolated CD4+CD25- cells were stimulated by a pool of islet-specific peptides and APC (irradiated autologous CD4- CD25+ cells) for 14 days and expansion of islet-specific T cells was analyzed on day 13 by tetramer staining. Panel B depicts flow plots showing islet-specific T cells stained by individual tetramers or tetramer pool, gated on CD4+ cells.

FIG. 17 depicts generation of islet-specific Tregs of multiple specificities. Panel A depicts islet-specific T cells were stained by tetramers and sorted on day 14. Sorted tetramer+ cells were activated with CD3/CD28 beads for 72 h for Foxp3 editing. 3 days after editing, cells were stained and analyzed. Flow plots show Foxp3 and LNGFR expression in mock or edited cells (left) and CD25, CD127, and CD45RO expression in LNGFR+ gated cells (right). Panel B depicts cells were stained by individual tetramers or tetramer pool and flow plots show tetramer+ cells in LNGFR+ Foxp3+ edited cells.

FIGS. 18-33 relate to the generation of dual-edited human CD4+ T cells using bi-allelic targeting to engineer artificial Treg cells expressing Foxp3 and antigen-specific TCR, with endogenous TCR inactivation.

FIG. 18 depicts a schematic of an exemplary CD4+ T cell edited to possess Treg phenotype and to express exogenous Ag-specific TCR, but not endogenous TCR. In this scheme, the conversion of a conventional CD4+ T-cell into an antigen-specific Treg comprises three genetic alterations: 1) stable expression of the transcription factor FOXP3 to drive cells toward a Treg phenotype; 2) stable expression of a defined, antigen-specific rearranged T-Cell receptor (Ag-specific TCR) to direct Treg immunosuppressive activity; and 3) genetic deletion of the endogenous T-Cell Receptor (TCR) to ensure that immunosuppressive function is directed solely toward the desired antigen.

FIG. 19 depicts exemplary AAV constructs for CRISPR gene editing at the human and mouse TRAC loci. The list includes adeno-associated virus plasmid constructs generated for CRISPR-based homology directed repair, organized based on the relevant gRNA, and includes number designation.

FIG. 20 depicts an exemplary CRISPR-based approach for targeting of the human TRAC locus for knockout/knock-in. In particular, the image shows a schematic representation of the human TRAC locus showing the relative position of the four gRNA sequences tested (PC_TRAC_E1_gRNA1 to PC_TRAC_E1_gRNA4). The TRAC exon 1 is indicated by the lowermost bar from about position 1160 continuing past 1400. Common SNPs are indicated by about positions 1160 and 1400. The position of a previously published positive control gRNA sequence (TCRa G4old) is indicated at about position 1320.

FIG. 21 relates to guide RNA (gRNA) qualification of non-homologous end joining (NHEJ) for knockout of CD3 in human CD4+ primary T cells. Data are from FACS analysis. Panel A depicts flow plots show expression of CD3 2 days post-editing in mock-edited and TCR-edited CD4+ T cells using four different guide RNAs. TCRa_G4old, previously demonstrated to knockout CD3 expression, was used as a control. Panel B depicts histograms showing percent CD3 knockout.

FIG. 22 depicts results from Inference of CRISPR Edits (ICE) analysis of indel frequency. On-target site-specific activity was measured by ICE (Inference of CRISPR Edits) and confirmed specific indel induction for gRNA_1 and gRNA_4 in TRAC relative to predicted off-target sites.

FIG. 23 depicts results from ICE analysis of predicted off target sites for TRAC gRNAs. The top 3 predicted off target sites for TRAC gRNA 1 and TRAC gRNA 2 (based on frequency and position of mismatches) were tested for indel induction frequency by ICE sequence deconvolution analysis.

FIG. 24 depicts an exemplary experimental outline for performing dual AAV editing for assessment of bi-allelic knock-in. A. Diagram of AAV constructs used in this experiment; after editing, MND promoter drives expression of GFP/BFP. B. Timeline of experimental procedures. CD4+ T cells were bead-stimulated (CD3/CD28) for 3 days prior to editing. Three and six days post-editing, cells were evaluated for GFP and BFP expression by flow cytometry.

FIG. 25 depicts dual editing of the TRAC locus in human CD4+ cells leads to a double-positive population of cells. Panel A depicts flow plots show GFP and BFP expression in mock-edited, and mixed MND.GFP- and MND.BFP-edited cells (10% #3207 virus + 10% #3208 AAV) two days post-editing. Viral titers were 3.3×10^12 and 2.53×10^12 for #3207 and #3208, respectively. Panel B depicts histograms showing percent double-negative, GFP single-positive, mCherry single-positive and GFP/mCherry double-positive cells within the dual-edited cells.

FIG. 26 depicts schematic diagrams showing exemplary Split IL-2 CISC HDR knock-in constructs for selection of dual-edited cells. In the depicted constructs, CISC (chemically induced signaling complex) is split onto 2 different constructs and each CISC component is co-expressed with a different reporter, in this case either GFP or mCherry. Each construct contains half of a rapamycin-binding complex (either FKBP or FRB domain, with the chimeric endoplasmic reticulum targeting domain fused to one half of an IL-2R signaling complex (IL-2RB or IL-2RG) transmembrane and intracellular domains. Delivery of cDNA encoding each CISC component co-expressed with the GFP / mCherry tag to primary human CD4+ T cells allows selective expansion of cells that contain both CISC components and thus are also dual edited for GFP and BFP.

FIG. 27 depicts an exemplary timeline of steps for dual AAV editing of CD4+ T cells, expansion with rapalog, and analysis of enriched cells. Cells were bead stimulated (CD3/CD28) for 3 days prior to editing. Two days post-editing, cells were analyzed by flow for GFP and mCherry expression, and then expanded in media containing 50 ng/ml human IL-2 or 100 nM rapalog. Flow cytometry to assess enrichment of GFP, mCherry double-positive cells was carried out on days 6, 8, and 10 post-editing.

FIG. 28 depicts FACS analysis of initial dual editing rate. Panel A depicts flow plots show GFP and mCherry expression in mock-edited, MND.GFP.FRB.IL-2RB-edited (20% #3207 AAV), MND.mCherry.FKBP.IL-2RB (20% #3208 AAV)-edited and mix-edited (10% #3207 + 10% #3208) cells. Viral titers were 3.3×10¹² and 2.53×10¹² for #3207 and #3208, respectively. Panel B depicts histograms show percent of double-negative, GFP-positive, mCherry-positive and GFP/mCherry double-positive cells within the dual-edited cells.

FIG. 29 depicts exemplary data showing rapalog enrichment of dual-edited cells. Panel A depicts flow plots show GFP and BFP expression in mock-edited, and mixed MND.GFP- and MND.BFP- edited cells (10% #3207 virus + 10% #3208 AAV) two days post-editing. Viral titers were 3.3×10¹² and 2.53×10¹² for #3207 and #3208, respectively. Panel B histograms showing percent double-negative, GFP single-positive, mCherry-single positive and GFP/mCherry double-positive cells within the dual-edited cells.

FIG. 30 depicts histograms showing percent double-negative, GFP single-positive, and mCherry single-positive cells after contact with IL-2 and rapalog. These data show that single-positive and unedited populations do not significantly change with rapalog treatment.

FIG. 31 depicts data from FACS analysis of initial dual editing rates using two different donors. Panel A depicts a timeline of editing and analysis steps. Panel B depicts histograms showing percent double-negative, GFP-positive, mCherry-positive and GFP/mCherry double-positive cells within the dual-edited cells for each donor. Donor R003657 is male, Caucasian and 28 y.o. Donor R003471 is male, Caucasian and 29 years old.

FIG. 32 depicts data from FACS analysis of rapalog enrichment of Bi-Allelic R003471 cells. Panel A depicts flow plots showing expression of GFP and mCherry following 5 days enrichment in rapalog. Panel B depicts histograms showing percent GFP/mCherry double-positive cells after expansion in IL-2 or rapalog.

FIG. 33 depicts schematic diagrams showing exemplary split-CISC constructs for insertion of TCR and Foxp3 and enrichment of dualedited cells. CISC is split onto two different constructs and each CISC component is co-expressed with either an Ag-specific TCR (in the diagram, exemplary T1D4 TCR) or Foxp3. Each construct contains half of a rapamycin-binding complex (either FKBP or FRB domain, with the chimeric endoplasmic reticulum targeting domain fused to one half of an IL-2R signaling complex (IL-2RB or IL-2RG) transmembrane and intracellular domains. Delivery of cDNA encoding each CISC component co-expressed with the T1D4 TCR / Foxp3 to primary human CD4+ T cells allows selective expansion of cells that contain both CISC components and thus are also dual edited for T1D4 TCR and Foxp3.

FIGS. 34-37 relate to the generation of reagents for assessing antigen-specific airT cell function in in vivo models of autoimmunity.

FIG. 34 depicts a schematic representation of the murine TRAC locus showing the relative position of the three novel gRNA sequences tested (PC_mmTrac_El_gRNAl to PC_mmTrac_E1_gRNA3). The TRAC exon 1 is indicated in blue.

FIG. 35 depicts data from FACS analysis of CD3 knockout in murine CD4+ T cells. Panel A depicts flow plots show expression of murine CD3 two days post-editing in mock-edited and TCR-edited CD4+ T cells using three different guides. Panel B depicts histograms showing percent mCD3 knockout for each guide RNA.

FIG. 36 depicts an exemplary experimental outline for dual AAV editing for assessment of bi-allelic knock-in. Panel A depicts a diagram of AAV constructs used in this experiment; after editing, MND promoter drives expression of GFP/BFP. B. Timeline of experimental procedures. Murine CD4+ T cells were bead stimulated (CD3/CD28) for 3 days prior to editing. Three and five days post-editing, cells were evaluated for GFP and BFP expression by flow cytometry.

FIG. 37 depicts data from FACS analysis of single- and dual-editing rates in the murine TCRa locus. Flow plots show GFP and BFP expression 3 days post-editing in mock, MND.GFP (10% #3211), MND.BFP (10% #3212), and mix-edited cells (5% #3207 + 5% #3208). Mixed edited cells had a total of 1.97% GFP/BFP double-positive cells.

FIGS. 38-43 relate to airT cell function in an antigen-specific in vivo setting.

FIG. 38 depicts a schematic diagram of an experimental design to test the ability of MOG-specific edTreg/airT (shown in white) to suppress T effectors (T_{eff}) in a mouse model of multiple sclerosis, Experimental Autoimmune Encephalomyelitis.

FIG. 39 relates experiments showing that mouse FOXP3 TALENs catalyze efficient FOXP3 disruption and initiate non-disruptive recombination of donor template. Panel A depicts binding sites for the FOXP3 TALEN pair in the human FOXP3 gene. Panel B depicts target binding sites for the mouse FOXP3 TALEN pair in the murine FOXP3 gene. Panel C depicts indel frequency at FOXP3 TALEN cut site in human (left) and mouse (right) CD4+ T cells 5-7 days after transfection with mRNA encoding either control mRNA (encoding blue fluorescent protein), or TALENs specific for human FOXP3 or mouse FoxP3, respectively. Graph shows average frequency of indels after colony sequencing PCR amplicons surrounding gDNA target site; 20-40 colonies were sequenced per experiment.

FIG. 40 relates to generation of edTreg/airT from antigen-specific murine CD4+ T cells. Panel A depicts a schematic diagram of FOXP3 locus after successful gene editing using mouse FOXP3 TALENs and the mouse AAV FOXP3 MND-GFP knock-in (ki) donor template. After editing, the MND promoter drives expression of chimeric GFP-FoxP3 protein. Panel B depicts flow plots showing GFP expression in antigen-specific mouse CD4+ T cells at Day 2 post-editing. Panel C depicts average percent of GFP+ cells across multiple experiments (n = 10). D. Flow plot of murine edTreg/airT showing expression of relevant Treg markers.

FIG. 41 shows functional assessment of antigen specific vs. polyclonal edTreg/airT in a mouse model of Multiple sclerosis. Panel A depicts flow plots showing GFP expression in MOG-specific and polyclonal mouse CD4+ T cells at Day 2 post-editing after FACS sorting. Panel B depicts schematic diagram of murine EAE in vivo experimental design and timeline. 2D2 (MOG-specific) T_{eff} (30 K) were delivered with or without co-transferred edTreg/airT (30 K) generated from either 2D2 or C57B⅙ mice into RAG1-/- recipient mice; all strains were on C57B⅙ background. Analysis was performed at Day 7.

FIG. 42 depicts data showing that antigen-specific edTreg/airT delay expansion, activation and cytokine production of T_{eff}. Immunophenotype of T cells obtained from inguinal and axillary lymph nodes in recipient mice at day 7 post-cell transfer was assessed by flow cytometry. CD45+ = panCD45 (recognizing all CD45 isoforms and both CD45.1 and CD45.2 alloantigens). Shown are total number of total CD45+ CD4+ cells (A) and other indicated T cell subsets (B) and (C), expansion of GFP+ cells. Data is representative of results from 3 independent experiments; bar graphs show mean ± SD; p-values of statistically significant differences are indicated above bars.

FIG. 43 provides data showing that antigen-specific edTreg/airT cells suppress T_{eff} proliferation in vivo. Panel A depicts flow plots: to label actively dividing cells, the thymidine analog 5-Ethynyl-2′-deoxyuridine (EdU) was administered 2 hours prior to sacrifice in selected animals. EdU incorporation in T cells was determined by intracellular labeling with an anti-EdU antibody and flow cytometry. Flow plots are from T cells isolated from LNs 7 days post-cell transfer. Panel B depicts bar graphs summarize mean % of cells incorporating EdU in different cell subsets and (C) the % GFP + lymphocytes. Flow plots are representative of results from at least 3 independent experiments; bar graphs show mean ± SD; p-values of statistically significant differences are indicated above bars.

FIGS. 44-47 relate to experiments investigating antigen specific T cell function in a NSG adoptive transfer model of Type 1 diabetes. Engineered antigen-specific (BDC) or polyclonal (NOD) edTregs/airTs, or antigen-specific nTregs were infused into the mice followed by infusion of antigen-specific T_{eff} cells. Mice were monitored for diabetes up to 90 days following infusion. Graph shows the percent of diabetic mice that received effector cells plus the designated mock edited, Foxp3-edited, or nTreg cells from NOD and BDC2.5 mice.

FIG. 44 relates to Foxp3 editing in CD4+ T cells of antigen-specific NOD mice. Panel A depicts CAS9/CRISPR RNP cutting efficiency in BDC2.5 NOD mice using different guide RNAs. Panel B depicts AAV5-delivered repair template. After editing, the MND promoter will drive expression of chimeric GFP-FoxP3 protein. Panel C depicts flow plots showing GFP expression in mock-edited and GFP-Foxp3-edited antigen-specific mouse CD4+ T cells at day 2 post-editing.

FIG. 45 relates to phenotype of FOXP3-edited antigen-specific NOD CD4+ T cells. Left. Flow cytometry plots showing GFP and Foxp3 expression in edited cells. Middle. Flow cytometry plots showing IL-2, IFN-g and IL-4 expression in GFP-Foxp3-edited (upper plots) and mock-edited (lower plots) murine antigen-specific NOD CD4+ T cells. Right. Histograms showing % of cells positive for IL-2, IFN-γ and IL-4 four days post-editing.

FIG. 46 relates to an experiment investigating phenotype of input cells for NSG adoptive transfer model. Panel A depicts an experimental design showing amount and type of cells administered for each group of animals. Panel B depicts flow cytometry plots showing the phenotype of T_{eff}, edTreg/airT and nTreg cells injected into NSG mice.

FIG. 47 relates to antigen-specific T cell function in NSG adoptive transfer model. Panel A depicts an experimental design; engineered antigen-specific (BDC) or polyclonal (NOD) edTregs/airTs, or antigen-specific nTregs were infused into the mice, followed by infusion of antigen-specific T_{eff} cells. Mice were monitored for diabetes up to 90 days following infusion. Panel B depicts a graph shows the percent of diabetic mice that received effector cells plus the designated mock-edited, Foxp3-edited, or nTreg cells from NOD and BDC2.5 mice. Antigen-specific edTreg/airT exhibited significantly greater level of protection from T1D compared with mock-edited T cells, polyclonal edTregs/airTs or polyconal nTregs.

FIGS. 48-51 relate to engineering a mouse AAV donor template design to generate airT cell product with a selectable marker (LNGFR).

FIG. 48 depicts exemplary repair templates used in murine Foxp3 editing. AAV.Promoter-LNGF.P2A knock-in constructs were tested in murine T cells for stable expression of Foxp3.

FIG. 49 depicts phenotype of murine edTreg/airT using alternative homology donor cassettes. Flow cytometry plots show LNGFR, FOXP3, CD25, and CTLA-4 in mock-edited cells and cells edited with MND.LNGFR.P2A KI (3189) or PGK.LNGFR.P2A KI (3227).

FIG. 50 depicts data showing editing rate and expression of LNGFR in murine edited Treg/airT cells. Flow cytometry plots show LNGFR and GFP expression in mock, MND-GFPki (#1331) MND.LNGFR.P2A.KI (#3189) edited cells.

FIG. 51 depicts data showing enrichment of LNGFR+ edited T cells from B6 mice using an anti-LNGFR column. Flow cytometry plots show LNGFR expression of cells prior to purification on a Miltenyi anti-LNGFR column, cells in the flow through and cells eluted from the column.

FIG. 52 depicts a comparison of FOXP3-edited vs. FOXP3 lentiviral (LV) transduced human CD4 T cells. Panel A depicts a diagram of LV construct: MND promoter drives expression of a transcript encoding identical GFP-FOXP3 fusion protein as that of airT; transcript contains WPRE and poly(A) signals for efficient nuclear export and mRNA stability. Below are representative flow plots showing FOXP3 and GFP expression in mock-edited T cells or sorted tTreg (CD4+CD25++CD127-), airT and LV Treg (CD4+GFP+), all post ≥14-day expansion in vitro with CD3/CD28 beads. Panel B depicts mean viral copy number (±SD) of LV-transduced sorted cells (left; n = 6). Scatter plots (right) show the MFI of the GFP+ population for each sample (n = 5; P value from two-tailed Student’s T-test). Panel C depicts bar graphs showing mean % of cells (top), and MFI (bottom) by flow cytometry staining for the proteins indicated. Viable singlets were further gated on: CD4+ GFP+ (LV Treg and edTreg), CD4+FOXP3+ (tTreg), or CD4+ (mock). For markers with distinct bimodal distributions, MFI was calculated for the positive population only. Error bars show ± SD. An ordinary two-way ANOVA was performed, and P values adjusted with Tukey’s multiple comparisons test. P values in black indicate comparison with mock-edited cells; those in red were comparison of groups indicated by dashed lines. Panel D depicts percent suppression as a function of Treg or mock dilution (top). Histograms of proliferation dye at different ratios of Treg or mock to T_{eff} (bottom). % suppression = [(% divided with no Treg - % divided with Treg)/ % divided with no Treg] × 100. Panel E depicts a plot showing data points and simple linear regression of % GFP+ cells over time in culture after FACS purification; airT (n = 4) and LV Treg (n = 6); data from 6 experiments. Dashed lines indicate 95% confidence intervals; P value was obtained using an F Test.

FIGS. 53-73 provide additional schematics and data related to exemplary dual-editing strategies of the present disclosure for generation of antigen-specific, drug-selectable airT cells with knock-out of endogenous TCR.

FIG. 53 depicts schematics showing dual-editing strategies designed to: a) eliminate the endogenous TCR expression and b) generate selectable antigen-specific airTs. Delivery of expression cassettes for FOXP3 and a candidate islet antigen-specific TCR (T1D4) paired to the two halves of the IL-2 CISC/DISC (FKBP-IL2RG and FRB-IL2RB), can be directed to the same locus (Strategy 1) or two separate loci (Strategy 2). Targeting of the TRAC locus in CD4+ T cells allows for deletion of the endogenous TCR. Strategy 2 may result in higher initial dual editing rates but requires two nuclease target sites, leading to two double stranded breaks (DSBs) in the host cell genome that mediate HDR. Strategy 1 utilizes a single nuclease target site leading to a single DSB.

FIG. 54 depicts a schematic of AAV HDR donor constructs used in human T cell dual-editing. The first 7 constructs are IL-2 split-CISC repair templates with either GFP, mCherry, HA-tagged FOXP3 or T1D4 driven by the MND promoter. Each component of the split CISC includes a heterodimeric rapamycin binding complex (either FKBP and FRB domains), along with the chimeric endoplasmic reticulum targeting domain fused to one half of the IL2R signaling complex (either IL2RB or IL2RG) trans-membrane and intracellular domains. Each repair template is flanked by 300 bp homology arms matched to a gRNA targeting either the TRAC locus (gRNA_4) or FOXP3 locus (gRNA_T9) (#3207, 3208, 3240, 3243, 3251, 3252, 3273). The next four constructs (#3253, 3258, 3292, 0001) are used for in-frame knock-in of a promoter-less TCR cassette including components of the CISC, targeting the first exon of TRAC locus (gRNA_1). The final two constructs (#3280 and #3262) are split-DISC repair templates that include the CISC elements as well as cDNA encoding a free FRB domain that functions in cytoplasmic Rapamycin sequestration (which eliminates or reduces any negative impact of rapamycin on gene edited cells). These latter constructs also contain either mCherry or FOXP3 driven off the MND promoter.

FIG. 55 depicts dual editing rates within the human TRAC locus in the presence or absence of rapalog-based selection of CISC edited CD4+ T cells from donor R003657. Panel A depicts a timeline of editing (using RNP and AAV co-delivery), enrichment and analysis steps with donor R003657 CD4+ T cells using AAV #3207 and #3208. Panel B depicts flow plots show initial percent GFP/mCherry double positive cells in the mock vs. dual-edited samples, and percent GFP/mCherry double positive following 7 days enrichment in the presence of IL-2 or Rapalog (AP21967). Panel C depicts histograms show percent double-negative, GFP-positive, mCherry-positive and GFP/mCherry double-positive cells within the dual-edited cells following enrichment in IL-2 vs. Rapalog.

FIG. 56 depicts dual editing rates in the TRAC locus and rapalog-based selection of CISC-edited CD4+ T cells from donor R003471. Panel A depicts a timeline of editing, enrichment and analysis steps with donor R003471 CD4+ T cells using AAV #3207 and #3208. Panel B depicts flow plots show initial percent GFP/mCherry double-positive cells in the mock- vs. dual-edited samples, and percent GFP/mCherry double-positive following 7 days enrichment in the presence of IL-2 or Rapalog (AP21967). Panel C depicts histograms show percent double-negative, GFP-positive, mCherry-positive and GFP/mCherry double-positive cells within the dual-edited cells following enrichment in IL-2 vs. Rapalog.

FIG. 57 shows dual-editing of the TRAC locus in human CD4+ T cells generates rapalog-selectable, antigen-specific airTs. Panel A depicts a schematic showing AAV HDR donors construct used to introduce of the “split” IL-2 CISC elements for selection of dual-edited cells. CISC components (IL2RG vs. IL2RB) are split between 2 constructs and co-expressed with either HA-FoxP3 cDNA or the islet-specific TCR, T1D4 (AAVs #3240 and #3243 respectively). Each repair template is flanked by identical homology arms that cannot be cleaved by the gRNA targeting the TRAC locus. Only edited CD4+ T cells incorporating one copy of each construct are predicted to selectively expand under Rapalog treatment. Panel B depicts a timeline of key steps for dual AAV/RNP-based editing of CD4+ T cells, expansion with Rapalog and analysis of enriched cells. Cells were bead stimulated (CD3/CD28) for 3 days prior to editing. Two days post-editing, cells were analyzed by flow for HA-FoxP3 and TCR expression, and then expanded in media containing 50 ng/ml human IL-2 or 100 nM Rapalog. Flow cytometry to assess enrichment of HA-FoxP3, TCR double-positive cells was carried out on days 5 and 8 post-editing. Panel C depicts rapalog enrichment of dual-edited cells. Left panel: Flow plots for HA-FoxP3 and TCR in dual-edited cells following 8 days expansion in IL-2 or Rapalog; Right panel: quantitation of percent HA-FoxP3 / TCR double-positive cells following expansion in IL-2 or Rapalog for 5 and 8 days.

FIG. 58 provides data showing that decreasing serum concentration increases total- and dual-editing rates within the TRAC locus. Panel A depict a timeline showing steps for dual AAV editing of CD4+ T cells and expansion with Rapalog. Human CD4+ T cells were edited using TRAC gRNA_4 and #3243 and #3240 AAV constructs (Single-locus dual editing). Immediately following electroporation to deliver the RNP, the cells were placed in either 20%, 2.5%, 1% or 0% FBS containing media (recovery media) and infected with AAV. After ~ 16 hours, the media was replaced with 20% FBS containing media and FACS analysis done on day 3 to determine editing rate. Cells recovered in 2.5% FBS containing media were expanded in the presence of either IL-2 or Rapalog for an additional 7 days. Panel B depicts flow plots show T1D4 and FOXP3 expression in mock-edited, single-edited and mixed edited cells (10% #3243 and 10% #3240 AAV) three days post editing. Viral titers were 4.2E¹¹ and 1.3E¹² for #3243 pAAV.MND.T1D4.FRB.IL2RB and #3240 pAAV.MND.FOXP3-HA.FKBP.IL2RG respectively. Panel C depicts histograms show percent double-negative, FOXP3-HA-positive, T1D4-positive and FOXP3/T1D4 double-positive cells within the dual-edited cells.

FIG. 59 shows IL-2 vs. Rapalog enrichment of dual-edited cell populations. TRAC locus dual-editing was performed as shown in FIG. 5. Panel A depicts flow plots show T1D4 and FOXP3 expression in mock-edited vs. FOXP3/T1D4 (#3240/3243) dual-edited cells treated with either 50 ng/mL IL-2 or 100 nM Rapalog (AP21967) for 7 days. Data are shown only for the 2.5% FBS recovery media condition. Panel B depicts histograms show percent double-negative, FOXP3-HA-positive, T1D4-positive and FOXP3/T1D4 double-positive cells within the dual-edited cells following enrichment.

FIG. 60 relates to a strategy for testing two-loci dual-editing of human CD4+ T cells. Panel A depicts a diagram of AAV HDR-donor constructs designed to introduce split IL-2 constructs for selection of dual-edited cells using a two loci dual-editing approach. CISC components are split between 2 constructs and co-expressed with either mCherry or GFP (#3207 and #3251 respectively). Repair templates are flanked by homology arms matched to gRNAs targeting either the TRAC or FOXP3 locus, respectively. Only edited CD4+ T cells that incorporate both expression cassettes (into the appropriate locus) are predicted to selectively expand under Rapalog treatment. Panel B depicts a timeline showing steps for dual AAV editing of CD4+ T cells and expansion with Rapalog. Human CD4+ T cells were edited using human TRAC gRNA_4, human FOXP3 gRNA_T9 and #3251 (MND.mCherry.FKBP.IL2RG) and #3207 (MND.GFP.FRB.IL2RB) AAV constructs (two-loci dual editing). Immediately following electroporation, the cells were placed in either 20% or 2.5% FBS containing media (recovery media). After ~16 h, the media was replaced with 20% FBS containing media and FACS analysis done on day 3 to determine editing rate. Cells recovered in 2.5% FBS containing medium were further grown in the presence of either IL-2 or Rapalog for an additional 7 days to monitor enrichment.

FIG. 61 shows that recovery in 2.5% FBS containing medium improves dual-editing rates measured at Day 3 post-editing. Two-loci dual editing was performed as shown in FIG. 59. Panel A depicts flow plots show GFP and mCherry expression in mock-edited and dual-edited cells in 20% FBS vs. 2.5% FBS recovery media at 3 days post-editing. Viral titers were 6.55E^10 and 2.50E^12 for #3251 pAAV.MND.mCherry.FKBP.IL2RG and #3207 pAAV.MND.GFP.FRB.IL2RB respectively, and 10% culture volume of each virus was used for the editing reactions. Panel B depicts histograms show percent double-negative, GFP-positive, mCherry-positive and GFP/mCherry double-positive populations within the dual-edited cells.

FIG. 62 shows robust enrichment of two-loci dual-edited cells treated with rapalog selection. Two-loci dual-editing was performed as shown in FIG. 59. Panel A depicts flow plots show GFP and mCherry expression in mock-edited and GFP/mCherry (#3207/3251) edited cells (edited in 2.5% serum) treated with either 50 ng/mL IL-2 vs. 100 nM Rapalog (AP21967) for 10 days. Panel B depicts histograms show percent double-negative, GFP-positive, mCherry-positive, and GFP/mCherry double-positive cells within the edited population following treatment in IL-2 vs. Rapalog for 10 days.

FIG. 63 relates to engineering of two-loci dual-editing of human CD4+ T cells. Editing conditions and timeline for dual AAV editing of CD4+ T cells and expansion with Rapalog. Human CD4+ T cells were edited using human TRAC gRNA_4, human FOXP3 gRNA_T9 and #3251 (MND.mCherry.FKBP.IL2RG) and #3207 (MND.GFP.FRB.IL2RB) AAV (two-loci dual editing). Editing conditions were varied according to the table with different % of viral stock and either in the presence of the HDR enhancer or DMSO. Immediately following electroporation, the cells were placed in 2.5% FBS containing media (recovery media). After ~16 h, the media was replaced with 20% FBS containing media and FACS analysis done on day 3 to determine editing rate. Cells recovered in 2.5% FBS containing medium were further grown in the presence of either IL-2 or Rapalog for an additional 10 days to monitor enrichment.

FIG. 64 depicts a graph showing that matched 10% volume of AAV HDR donors leads to improved dual editing. Editing was performed as outlined in FIG. 62. Graphs show percent double-negative, GFP-positive, mCherry-positive and GFP/mCherry double-positive populations within the dual-edited cells 3 days post-editing with varying amounts of #3207 and #3251 AAV in the presence of 30 uM HDR enhancer or DMSO.

FIG. 65 provides data showing robust enrichment of two-loci dual-edited CD4+ T cells with rapalog selection; with optimal results using 2.5% FBS media and matched 10% volume of AAV donor. Editing was performed as outlined in FIG. 62. Graphs show the cells from FIG. 10, (edited in 2.5% serum, matched 10% virus +/- HDR enhancer) as percent double negative, GFP positive, mCherry positive, and GFP/mCherry double positive cells within the editing population following contact with IL-2 or Rapalog for 10 days.

FIG. 66 provides a diagram of exemplary split-CISC constructs for insertion of islet-specific TCR and FOXP3 and enrichment of dual-edited cells using a two-loci dual-editing strategy. The IL-2 CISC (chemically induced signaling complex) is split onto 2 different constructs and co-expressed with either T1D4 TCR or FOXP3 (#3243 and #3252 respectively). Each construct contains half of a heterodimeric rapamycin binding complex (FKBP and FRB domains), along with the chimeric endoplasmic reticulum targeting domain fused to one half of the IL-2R signaling complex (IL-2RB or IL-2RG) trans-membrane and intracellular domains. Delivery of cDNA encoding each CISC component co-expressed with the T1D4 TCR / FOXP3 to primary human CD4+ T cells allows us to only expand cells that contain both CISC components and thus are also dual edited for T1D4 TCR and FOXP3 expression.

FIG. 67 shows an exemplary strategy for single locus dual-editing with capture of TRAC promoter. Schematic of AAV HDR-editing constructs designed for dual-editing within the TRAC locus to introduce: (top) (#3240 FOXP3 expression and split CISC and (bottom) (#3258) in-frame knock-in to TRAC exon 1 to drive expression of T1D4 TCR and split CISC using the TRAC endogenous promoter.

FIG. 68 shows that TRAC locus HDR editing disrupts TCR expression and mediates robust transgene expression via the endogenous TRAC enhancer-promoter. Panel A depicts an editing strategy for in-frame integration of a mCherry-Split-CISC cassette at the endogenous TRAC locus. By using a gRNA targeting the TRAC exon 1, in-frame integration of a marker fluorophore (mCherry) followed by the FRB-IL2RB CISC separated by a P2A element (construct #3253) allows for expression driven by the endogenous TRAC promoter, while disrupting expression of the endogenous TCR. Panel B depicts a timeline showing steps for AAV #3253 editing of CD4+ T cells. Cells were bead-stimulated (CD3/CD28) for 3 days prior to editing. Panel C depicts an analysis: seven days post-editing, cells were analyzed by flow for CD3 and mCherry expression. Flow cytometry plots show significant expression of mCherry with concomitant loss of CD3 in edited cells compared to mock-edited and AAV-only controls.

FIG. 69 shows comparison of mCherry expression mediated via the TRAC endogenous promoter vs. MND promoter. Gene editing was performed as shown in FIG. 67 using alternative HDR donors (#3253 vs. #3208) to assess the relative expression activity from the TRAC endogenous promoter vs. MND promoters, respectively. Flow cytometry plots shows that the level of mCherry expression when driven off the endogenous promoter (P2A.mCherry.FRB.IL2RB (#3253)) is lower than compared to when driven by the MND promoter (MND.mCherry.FKBP.IL2RG (#3208). The bottom row of panels shows data from a repeat experiment performed using the #3253 donor.

FIG. 70 show exemplary alternative dual-editing strategies for targeting the TRAC and/or FOXP3 loci and that utilize in-frame knock-in constructs to capture the TRAC endogenous promoter. Schematic of exemplary AAV donor constructs for testing single-locus and two-loci dual-editing strategies and to generate antigen-specific airT with IL-2 CISC selection capacity. T1D4 TCR is shown as a representative TCR that can be replaced by alternative TCRs based upon disease target and other relevant features for therapeutic application. IL-2 DISC constructs are similarly applied.

FIG. 71 relates to dual-editing of human CD4+ T Cells using decoy-CISC (split-DISC) constructs. Panel A depicts a diagram of Split IL-2 DISC HDR knock-in construct (#3280) for selection of dual-edited cells in Rapamycin. To generate the split decoy-CISC (split-DISC), the free FRB domain for cytoplasmic Rapamycin sequestration was added to the MND.mCherry.FKBP.IL2RG construct to generate (MND.mCherry.FKBP.IL2RG.FRB (#3280)). Each repair template (#3280 and #3207, not shown) is flanked by identical homology arms matched to a gRNA targeting the TRAC locus. Edited CD4+ T cells incorporating one copy of each construct are predicted to selectively expand under Rapalog or Rapamycin treatment. Panel B depicts a timeline showing steps for dual AAV editing of CD4+ T cell using AAV #3280 and #3207), expansion with Rapalog/Rapamycin and analysis of enriched cells. Cells were bead-stimulated (CD3/CD28) for 3 days prior to editing. Two days post-editing, cells were analyzed by flow for GFP and mCherry expression, and then expanded in media containing 50 ng/ml human IL-2, 100 nM Rapalog or 10 nM Rapamycin. Panel C depicts flow plots show the percentage of GFP/mCherry double-positive cells on day 3 post-editing.

FIG. 72 shows that dual editing of human CD4+ T cells with split-DISC constructs generates Rapamycin-selectable cells. Dual-editing was performed as described in FIG. 70. Panel A depicts flow plots show percent double-positive GFP/mCherry cells following 8 days in the presence of 50 ng/mL human IL-2, 100 nM Rapalog (AP21967), 10 nM Rapamycin, or no treatment. Panel B depicts histograms quantitate percent double-negative, GFP-positive, mCherry-positive and GFP/mCherry double-positive cells within the dual-edited cells following enrichment in IL-2, Rapalog (AP21967), Rapamycin or no treatment.

FIG. 73 show exemplary constructs for in vivo testing of dual-edited Tregs (split-DISC). Diagram of FOXP3 split IL-2 DISC HDR knock-in construct (#3262) to be paired with a T1D4 CISC construct (#3243) for Rapamycin selection of dual-edited cells. CISC components are split between 2 constructs and co-expressed with either HA-FoxP3 or T1D4 TCR. The FOXP3 CISC construct also contains the FRB domain and is predicted to protect mTOR signaling in the presence of Rapamycin (FOXP3 DISC construct). Each repair template is flanked by identical homology arms matched to a gRNA targeting the TRAC locus. Edited CD4+ T cells incorporating one copy of each construct may selectively expand under both Rapalog and Rapamycin treatment.

FIGS. 74-94 provide additional schematics and data related to generation and characterization of murine airT cells.

FIG. 74 depicts repair templates used in murine Foxp3 editing. Diagram of alternative AAV.GFP.KI and AAV.LNGFR.P2A constructs that were developed and tested in murine T cells for editing efficiency, FOXP3 expression and suppressive function.

FIG. 75 depicts a schematic showing methods used for generation of murine airT using the MND.GFP.KI (or alternative) HDR donor construct.

FIG. 76 relates to generation and enrichment of murine airT cells utilizing alternative promoters to express endogenous Foxp3. Flow cytometry plots showing LNGFR and GFP expression prior to and post LNGFR enrichment via FACS sorting in mock, MND.GFP.KI (#1331), MND.LNGFR.P2A (#3189 and 3261), PGK.LNGFR.P2A (#3227) and EF-1a-LNGFR.P2A (#3229) edited cells. Upper plots show initial editing rates and lower plots show enrichment post FACS sorting. Data indicate that airT can be generated with each of the candidate donor constructs.

FIG. 77 depicts expression levels of Foxp3 in murine airT using alternative homology donor cassettes. Panel A depicts flow cytometry plots showing LNGFR and GFP expression in HDR-edited splenic T cells. Panels show un-manipulated C57BL/6 control cells; mock-edited, MND.GFP.KI (#1331), MND.GFP.KI with UCOE (#3213), and PGK.GFP.KI (#3209)- edited C57 BL/6 murine T cells, respectively. Panel B depicts flow histograms showing FOXP3 expression from the data in panel A. Panel C depicts a bar chart showing FOXP3 MFI in nTreg and edTreg/airT generated with the indicated alternative HDR-donor constructs. MND promoter containing donors mediate the highest levels of FOXP3 expression.

FIG. 78 depicts design of, and results from, an in vitro suppression assay using murine tTreg or airT. A. airT cells used for in vitro suppression assay were enriched by FACS sorting at day 2 post editing and resuspended into RPMI media containing 10% FBS. nTregs (CD4+CD25+), T_{eff} (CD4+CD25-) and antigen presenting cells (CD4-CD25-) were isolated from the combined spleen and lymph node cells of 8 to 10 weeks-old C56BL/6 mice by column enrichment. Enriched 5×10⁶ T_{eff} were resuspended in 2 ml of PBS and labeled with cell trace violet (CTV) for 15 minutes at 37° C., then washed and resuspended in media before their addition in the suppression assay. To set up the assay, 1.25 × 10⁵ irradiated APCs (2500 rad) were co-cultured with 0.25 × 10⁵ T_{eff} and a titrated number of nTregs and airT in the presence of 1 mg/ml anti-CD3 in a U bottom 96 well tissue culture plate with total volume of 300 ul media and incubated at 37° C. CO₂ incubator for four days. At day 4, cells were washed twice with PBS and stained with live/dead indicator, anti-CD4, anti-CD45 and anti-CD25, and analyzed by FACS (LSRII) for the suppression of T_{eff} proliferation by airT. B) Representative flow data showing a reduction of T_{eff} proliferation in the presence of airT cells.

FIG. 79 depicts results from testing murine airT suppressive function in vitro. Flow cytometry plots showing cell trace violet labeled CD4+ T cells in the presence and absence of mock-, MND.GFP.KI- (#1331), or MND.LNGFR.P2A- (#3261-edited T cells, or nTregs from C57 BL/6 mice. These data demonstrate that murine airT (generated with the MND.GFP.KI or MND.LNGFR.P2A HDR donors) and nTregs exhibit comparable, robust in vitro suppressive function.

FIG. 80 depicts in vitro suppressive function of murine airT with alternative promoters. Flow cytometry plots showing cell trace violet-labeled CD4+ T cells in the presence and absence of mock-edited, MND.GFP.KI- (#1331), MND.LNGFR.P2A- (#3261), PGK.LNGFR.P2A- (#3227), and EF-la.LNGFR.P2A (#3229)-edited T cells, or nTregs from C57 BL/6 mice. Murine airT with MND promoter exhibit suppressive function that is comparable to nTreg. In contrast, airT using the PGK or EF-1a promoters exhibit only limited or no suppression.

FIG. 81 depicts the design of an experiment to compare sorted vs. column-purified enriched LNGFR+ edited cells in an NSG adoptive transfer model. The table lists the number of recipient NSG host animals, and source and number of adoptively transferred control, airT or nTreg cells in each of the 5 experimental cohorts

FIG. 82 depicts flow analysis of LNGFR.P2A-edited NOD BDC2.5+ murine cells prior to and post-column purification. Panel A depicts flow cytometry plots showing LNGFR expression in mock-, and MND.LNGFR.P2A- (#3189)-edited cells. Panel B depicts flow cytometry plots showing LNGFR expression in MND.LNGFR.P2A (#3189)-edited cells post enrichment via sorting. FACS sorting consistently enriched to edTreg products of >90% purity for use in in vitro and in vivo studies.

FIG. 83 depicts a flow analysis of edited murine cells before and after column enrichment. Flow cytometry plots showing MND-LNGFR.P2A (#3189) edited cells prior to- and post-column enrichment. In this example of enrichment, 72 × 10⁶ cells with initial editing rate of ~7% were added to an anti-LNGFR column yielding 2 × 10⁶ edTreg with >84% purity.

FIG. 84 depicts an experimental design and results assessing islet antigen-specific airT function in the NSG adoptive transfer model: comparison of FACS-sorted and column-enriched airT. Islet antigen-specific (BDC) airT (generated using the HDR donors, 3261 or 3389), or antigen-specific nTregs were adoptively transferred by retro-orbital (R.O.) delivery into adult, 8-10 wk old recipient NSG mice, followed by infusion of antigen-specific T_{eff} cells. Mice were monitored for development of diabetes for up to 60 days. Graph shows the percent of diabetic mice after receiving effector cells plus the designated mock-edited, MND.LNGFR.P2A-edited (FACS sorted or column enriched), or nTreg cells from NOD BDC2.5 mice. Column-enriched Ag-specific MND.LNGFR.P2A airT reduced diabetes incidence in NSG mice and shows comparable function to FACS-sorted airT. Higher doses of column-enriched MND.LNGFR.P2A airT or nTreg fully protected recipient animals from development of diabetes.

FIG. 85 depicts a comparison of in vivo function of airT generated using alternative promoters in the NSG adoptive transfer model. Engineered antigen-specific (BDC) airT (generated using either the MND or PGK promoter; donor constructs 1331 or 3209, respectively), or antigen-specific nTregs were adoptively transferred into NSG recipient mice followed by infusion of antigen-specific T_{eff} cells. Mice were monitored for development of diabetes for up to 60 days. Graph shows the percent of diabetic mice after receiving effector cells (5 × 10⁴) plus the designated mock edited, MND.GFP.KI (#1331), PGK.GFP.KI (#3209) airT or nTreg cells (5 × 10⁴) from NOD BDC2.5 mice. Antigen-specific airT with the MND promoter prevented diabetes development in all recipient mice. nTreg prevented disease in ⅘ recipient mice. In contrast, antigen-specific airT that incorporated the PGK promoter had little or no protective effect. These data directly demonstrate that protection from T1D is specific to airT generated using the MND promoter to drive Foxp3 expression supporting the use of this architecture in human trials for T1D or other immune diseases.

FIG. 86 shows that islet Ag-specific MND.GFP.KI airT persist in vivo in the target organ (pancreas) for at least 60 days and exhibit a stable phenotype. Flow cytometry plots showing FOXP3 and GFP expression in MND.GFP.KI (#1331) NOD BDC2.5 airT recovered in the pancreas at day 60 following adoptive transfer in NSG mice. Data shows results from two mice. Recipient mice also exhibit expansion of endogenous Treg or iTreg (FOXP3+. GFP- CD4 T cells) derived from the input T_{eff} population (likely secondary to additional beneficial bystander impacts of airT delivery).

FIG. 87 depicts design and results of CRISPR-based targeting of the murine Rosa26 Locus for Knock-in/knock-out. Panel A depicts the Rosa26 locus was selected as a model of a safe-harbor HDR integration site for murine T cells. Position of the two novel gRNAs (gRNA_1 and gRNA_2) within the murine Rosa26 locus. gRNAs from Pesch et. al. and Wu et. al. comprise previously published gRNAs within this locus region. Panel B depicts on-target site-specific activity as measured by ICE (Inference of CRISPR Edits) demonstrates specific indel induction using R26 gRNA_1 in Rosa26 after Cas9-RNP delivery to primary mouse CD4+ T-Cells.

FIG. 88 depicts an experimental outline for HDR editing at the mouse Rosa26 locus. Panel A depicts a diagram of AAV construct #3245 used in this experiment. After HDR-based editing in mouse T cells, the MND promoter drives expression of GFP. Panel B depicts a timeline of experimental procedures. Murine C57BL/6J CD4+ T cells were isolated and bead stimulated (CD3/CD28) for 3 days prior to editing. Cells were evaluated for GFP by flow cytometry at days 3 and 8 post editing.

FIG. 89 depicts data demonstrating HDR-based editing within the Rosa26 locus in murine CD4+ T cells. CD4+ T cells were edited as outlined in FIG. 88 and assessed at Day 3 by flow cytometry. Panel A depicts flow plots showing GFP expression in mock-edited, AAV #3245 alone and AAV #3245/RNP-edited cells at 3 days post editing. Panel B depicts histograms show percent viability, % GFP positive cells and high GFP+ cells within the edited population.

FIG. 90 shows that murine T cells maintain stable expression of GFP following HDR editing of the Rosa26 locus. CD4+ T cells were edited as outlined in FIG. 88 and assessed at Day 8 by flow cytometry. Panel A depicts flow plots show GFP expression in mock edited, AAV #3245 alone and AAV #3245/RNP edited cells 8 days post editing. Panel B depicts histogram shows % GFP positive cells within the edited population.

FIG. 91 depicts schematics of AAV HDR donor constructs for expression of murine Foxp3- and P2A- linked LNGFR within the Rosa26 locus in murine T cells. Repair templates are flanked by 300bp homology arms matched to R26_gRNA_1 cleavage site and contain alternative promoters (MND or PGK) driving expression of mFOXP3 and LNGFR. In addition, a cassette containing a Foxp3 4X CDK phosphorylation site mutant is included as this construct is predicted increase the stability of Foxp3.

FIG. 92 relates to lentiviral CISC constructs used to transduce murine CD4+ T cells and test selective expansion with Rapalog. Panel A depicts a diagram of lentiviral construct #1272. This construct was developed to assess proof-of-concept for enrichment of murine T cells using human CISC components in the presence of Rapalog. After transduction of mouse T cells, the MND promoter drives expression of mCherry linked to IL-2 CISC components (FKBP-IL2RG and FRB-IL2RB). Panel B depicts a timeline of experimental procedures. Murine C57BL/6J CD4+ T cells were bead stimulated (CD3/CD28) for 3 days prior to transduction. Cells were evaluated for mCherry by flow cytometry at days 2 and 5 post-transduction.

FIG. 93 shows that murine CD4+ T cells transduced with lentiviral CISC show robust enrichment in Raplog. Panel A depicts flow plots show mCherry expression 2 days following mock or lentiviral transduction (#1272) of murine CD4+ T cells. Panel B depicts flow plots show mCherry expression in mock murine cells treated with IL-2, IL-7 and IL-15, or lentiviral (#1272) transduced murine cells that are treated with either IL-2, IL-7 and IL-15, Rapalog alone, or Rapalog + bead stim.

FIG. 94 shows that airT cells suppress proliferation of CD8+ T cells, as well as, CD4+ T cells.

FIG. 95 depicts a schematic of a process for generating antigen-specific airT cells by stimulation with a model antigen peptide (MP) and editing for FoxP3 expression.

FIG. 96 depicts antigen-specific suppression by MP peptide-specific airT cells. Briefly: T_{eff}: day 23 T cells stimulated by MP peptide (right) or HA peptide (left); Treg: day 23 edited cells specific for MP peptide (right) or HA peptide (left), edited by CRISPR/Cas9 and AAV Foxp3-MND-LNGFRki; APC: irradiated autologous CD4-CD25+ cells DMSO or HA peptide 5 ug/ml; 6 day incubation.

FIG. 97 shows that airT cells show suppressive activity on T_{eff} proliferation. Briefly: 3 day incubation; for bead suppression: T_{eff} + Treg (untd edTreg, T1D5-1 airT, or T1D5-1 mock); For Ag-specific suppression: T1D5-1 T_{eff} + Treg; T_{eff} gate: CD4+CD11c-CTV+EF670-mTCRb+ gate.

FIG. 98 depicts suppression of cytokine production in T_{eff} by airT. Briefly: T1D4 T_{eff}; Treg (d10): T1D4 mock or T1D4 airT; and Peptide 1 µg/ml; for a 3 day incubation.

FIG. 99 depicts antigen-specific and bystander suppression on T_{eff} by airT. Briefly: T_{eff} 1.25×10⁴; Treg 2.5×10⁴; APC 1×10⁵; and Peptide 5ug/ml.

FIG. 100 depicts antigen-specific and bystander suppression on T_{eff} by airT. Briefly: T_{eff} 1.25×10⁴; Treg 2.5×10⁴; APC 1×10⁵; and Peptide 5ug/ml.

FIG. 101 shows bystander suppression of T_{eff} cytokine production. Briefly: T1D5-2 T_{eff}; Treg (d10): T1D4 mock or T1D4 edTreg; and Peptide 1ug/ml; and 3 day incubation.

FIG. 102 shows dose response of TCR: proliferation assay. Briefly: mTCR expression data: day 8 post-transduction; Proliferation assay: day 11 cells, and 4 day incubation.

FIG. 103 shows validation of islet Ag-specific TCR expression: mTCRb expression & proliferation assay. Briefly: T cells: day 9 post transduction, labeled with Cell Trace Violet; APC: irradiated CD4-CD25+ cells; and 5 day incubation.

FIG. 104 shows that antigen-specific GFP+ airT can be detected in the pancreas. See also FIG. 107 and FIG. 116.

FIG. 105 relates to generation and enrichment of murine LNGFR+ airT cells for in-vivo suppression studies. See also FIG. 114.

FIG. 106 shows that Ag-specific MND.LNGFR.P2A-airT completely prevented diabetes in NSG mice. See also FIG. 115, 134 and 135.

FIG. 107 shows that antigen-specific GFP+ airT can be detected in the pancreas.

FIG. 108 shows schematics and data related to an exemplary IL-2 CISC of the present disclosure.

FIG. 109 shows that in vivo rapamycin contact promotes CISC cell persistence.

FIG. 110 shows a schematic of an exemplary edited cell of the present disclosure.

FIG. 111 relates to gRNA selection for TRAC locus targeting.

FIG. 112 relates to a dual editing strategy with IL-2 Split-CISC components targeted to the TRAC locus.

FIG. 113 shows that CISC-engagement selects for dual-edited cells in-vitro.

FIG. 114 depicts a flow analysis of LNGFR.P2A-edited NOD BDC2.5+ murine cells prior to and post column purification. Panel A depicts a flow cytometry plots showing LNGFR expression specifically in MND.LNGFR.P2A (#3261)-edited cells but not in mock cells. Panel B depicts a flow cytometry plots showing LNGFR expression in MND.LNGFR.P2A (#3261)-edited cells in the flow through (F.T.) and eluted sample post enrichment via column purification. Column enrichment led to an airT product of 74.5% purity for use in in vivo studies.

FIG. 115 depicts an assessment of islet antigen-specific airT function in the NSG adoptive transfer model: Islet antigen-specific (BDC) airT (generated using the HDR donor 3261), or antigen-specific nTregs (50 K), were adoptively transferred by retro-orbital (R.O.) delivery into adult, 8-10 wk old recipient NSG mice followed by infusion of 50 K antigen-specific T_{eff} cells. Panel A depicts a flow cytometry plots showing the CD4 and CD25 profile of nTreg and LNGFR+ expression in MND.LNGFR.P2A (#3261)-edited cells. Panel B depicts a graph: mice were monitored for development of diabetes for up to 49 days. Graph shows the percent of diabetic mice after receiving effector cells plus the designated mock-edited, MND.LNGFR.P2A-edited (column enriched), or nTreg cells from NOD BDC2.5 mice. Column-enriched Ag-specific MND.LNGFR.P2A-airT completely prevented diabetes in NSG mice.

FIG. 116 shows that islet Ag-specific MND.GFP.KI airT persist in vivo in the target organ (pancreas) for at least 49 days and exhibit a stable phenotype. Flow cytometry plots showing LNGFR and FOXP3 expression in NOD BDC2.5 airT recovered in the pancreas at day 49 following adoptive transfer in NSG mice.

FIG. 117A depicts flow plots of mTCRb expression gated on CD4+ cells day 9 post-transduction.

FIG. 117B depicts flow plots of CD4+ T cells transduced with RA Ag-specific TCRs labeled with CTV and co-cultured with APC (irradiated PBMC) and their cognate peptide or DMSO for 3 days.

FIG. 118B depicts a polyclonal suppression assay and an antigen-specific suppression assay using enolase-specific edTreg.

FIG. 118C depicts a graph of percentage suppression of Teff proliferation by no Treg, untd edTreg, Enol edTreg, or mock in the presence of a-CD3/CD28 (black) or APC and enolase peptide (grey) calculated from percentage proliferation in FIG. 118B.

FIG. 119A depicts flow plots of mTCRb expression in untransduced edTreg and CILP297-1 edTreg gated on LNGFR+ Foxp3+ in edited cells transduced with no LV and LV CILP297-1-TCR, respectively.

FIG. 119B depicts a polyclonal suppression assay and an antigen-specific suppression assay using CILP-specific edTreg.

FIG. 119C depicts a graph of percentage suppression of CILP Teff proliferation by no Treg, untd edTreg, CILP edTreg, or mock in the presence of a-CD3/CD28 (black) or APC and CILP peptide (grey) calculated from percentage proliferation in FIG. 119B.

FIG. 120A depicts flow plots of mTCRb expression in untransduced edTreg and Vim418 edTreg gated on LNGFR+ Foxp3+ in edited cells transduced with no LV and LV Vim418-TCR, respectively.

FIG. 120B depicts a polyclonal suppression assay and an antigen-specific suppression assay using vimentin-specific edTreg.

FIG. 120C depicts a graph of percentage suppression of Vim Teff proliferation by no Treg, untd edTreg, Vim edTreg, or mock in the presence of a-CD3/CD28 (black) or APC and Vimentin peptide (grey) calculated from percentage proliferation in FIG. 120B.

FIG. 121A depicts flow plots show mTCRb expression in untransduced, Agg520, and Vim418 edTreg gated on LNGFR+ Foxp3+ in edited cells transduced with no LV, LV Agg520-TCR, and LV Vim418-TCR, respectively.

FIG. 121B depicts a polyclonal suppression assay using Agg520 Teff and edTreg or mock specific to Agg520 or Vim418.

FIG. 121C depicts a graph of percentage suppression of Agg520 Teff proliferation by no Treg, untd edTreg, Agg edTreg/mock, or Vim edTreg/mock calculated from percentage proliferation in FIG. 121B.

FIG. 121D depicts an antigen-specific and a bystander suppression assay using Agg520 Teff and edTreg or mock specific to Agg520 or Vim418.

FIG. 121E depicts a graph of percentage suppression of Agg520 Teff proliferation by no Treg, edTreg or mock calculated from percentage proliferation in FIG. 121D.

FIG. 122A depicts flow plots of mTCRb expression in untransduced, CILP297-1, and Vim418 edTreg gated on LNGFR+ Foxp3+ in edited cells transduced with no LV, LV CILP297-1-TCR, and LV Vim418-TCR, respectively.

FIG. 122B depicts a polyclonal suppression assay using CILP297-1 Teff and edTreg or mock specific to CILP297 or Vim418.

FIG. 122C depicts a graph of percentage suppression of CILP Teff proliferation by no Treg, untd edTreg, CILP edTreg or mock, or Vim edTreg or mock calculated from percentage proliferation in FIG. 122B.

FIG. 122D depicts an antigen-specific and bystander suppression assay using CILP297-1 Teff and edTreg specific to CILP297 and Vim418.

FIG. 122E depicts a graph of percentage suppression of CILP Teff proliferation by no Treg, edTreg or mock calculated from percentage proliferation in FIG. 122D.

FIG. 123A depicts flow plots of mTCRb expression and LNGFR/Foxp3 expression in edited cells expressing SLE3-TCR on day 7.

FIG. 123B depicts a polyclonal suppression assay and an antigen-specific suppressing assay using SLE-specific edTreg.

FIG. 124A depicts a schematic diagram of AAV HDR-donor constructs designed to introduce split-CISC elements into the TRAC locus using a single locus dual editing approach.

FIG. 124B depicts a timeline for key steps for dual AAV editing of CD4+ T cells and expansion with Rapalog.

FIG. 125A depicts flow plots of T1D4 and FOXP3 expression in mock edited, single edited and dual-edited cells (using 10% volume of both #3243 and #3240 AAV) at Day 3 post editing.

FIG. 125B depicts flow plots of T1D4 and CD4 expression in mock edited, and mixed edited cells.

FIG. 125C depicts histograms of percent double negative, FOXP3-HA positive, T1D4 positive and FOXP3/T1D4 double positive cells within the dual edited cells.

FIG. 125D depicts histograms of percent CD3 knockout in FOXP3/T1D4 dual edited cells vs. mock edited cells.

FIG. 126A depicts flow plots of viability and T1D4 and FOXP3 expression in dual-edited cells treated with either 50 ng/mL IL-2 (upper panels) or 100 nM Rapalog (AP21967; lower panels) for 7 days.

FIG. 126B depicts flow plots of CTLA4 expression of T1D4/FOXP3 double positive vs. double negative cell populations treated with either 50 ng/mL IL-2 (upper panels) or 100 nM Rapalog (AP21967; lower panels) for 7 days.

FIG. 127A depicts flow plots of viability (right plots) and T1D4 and FOXP3 expression (left plots) in dual-edited cells following treatment with 50 ng/mL IL-2 (upper plots) vs. 100 nM AP21967 (lower plots) after recovery in IL-2 medium.

FIG. 127B depicts a graph of fold enrichment of double positive T1D4/FOXP3 cells treated with either 50 ng/mL IL-2 or 100 nM Rapalog (AP21967) over a 10 day period with the last 3 days being in recovery media containing IL-2.

FIG. 128A depicts a diagram of Split IL-2 DISC HDR knock-in construct (#3280), for selection of dual-edited cells in either Rapamycin or Rapalog.

FIG. 128B depicts a timeline of key steps for dual AAV editing of CD4+ T cell using AAV #3280 and #3207, expansion with Rapalog/Rapamycin and analysis of enriched cells.

FIG. 129A depicts flow plot of mCherry and GFP expression in dual edited cells (10% culture volume of #3280 and #3207 AAV donors, respectively) four days post editing. Viral titers were 3.30E+12 and 3.1E+10 for #3280 and #3207 respectively. Dual-edit 4 million total cells initial dual positive rate: 4.47%. gRex vessel was seeded with 7.6 million total cells, 340,000 double-positive.

FIG. 129B depicts flow plots of viability (upper panel) and GFP and mCherry expression (lower panel) following the seeding of 7.6 million edited cells in gREX and 7 day expansion in the presence of AP21967 leading to 32-fold expansion of double-positive cells. Total double positive cells in gRex: 11.1 million.

FIG. 130A depicts a timeline of steps for dual AAV editing of CD4+ T cell using AAV #3280 and #3207, expansion with Rapalog and analysis of enriched cells.

FIG. 130B depicts flow plots of mCherry and GFP expression in dual edited cells (10% #3280 and 10% #3207 AAV). Viral titers were 3.30E+12 and 3.1E+10 for #3280 MND.mCherry.FKBP.IL2RG.FRB and #3207 pAAV.MND.GFP.FRB.IL2RB respectively. Edit 10 million total cells, initial dual positive rate: 2.37%. Seeded gRex with 9.1 million total cells 216,000 double-positive.

FIG. 131 depicts flow plots of viability and GFP and mCherry expression following the seeding of edited cells in gREX and 7 day expansion in the presence of AP21967. The results after 7 day expansion included Total double positive cells in gRex: 9.7 million; about 45-fold expansion from original 216,000 double positive cells.

FIG. 132A depicts a design for in vitro suppression assay using mouse edTreg or nTreg.

FIG. 132B depicts representative flow date showing a reduction of BDC2.5+ Teff proliferation in the presence of BDC2.5+ edTreg cells.

FIG. 133 depicts flow cytometry plots showing cell trace violet labeled CD4+ T cells in the presence and absence of mock, MND.LNGFR.p2A (#3261) edited Treg or nTregs from NOD BDC2.5+ mice. Murine Islet TCR+ edT_{reg} (generated with the MND.LNGFR p2A (#3261) HDR donors) and tTregs exhibit antigen-specific in vitro suppressive function. 50 K Teff + anti-CD3 (1 ug/ml) + 200 K irradiated APCs (2500 rad). Analysis @ Day 4. CTV = cell trace violet. Data shown: 1:1 (Teff to Treg ratio).

FIG. 134 depicts a graph of the percent of diabetic mice after receiving effector cells plus the designated mock edited, MND.LNGFR.P2A edited or nTreg cells from NOD BDC2.5 mice. Similar to nTregs, MND.LNGFR p2A edTregs completely prevented the onset of diabetes while mock edited control cells did not show any impact on disease onset.

FIG. 135 depicts a graph of the percent of diabetic mice after receiving effector cells plus the designated mock edited, MND.LNGFR.P2A edited or nTreg cells from NOD BDC2.5 mice in a repeat experiment. Column enriched Ag-specific LNGFR p2A edTregs completely prevented (Day 33) diabetes in NSG mice

FIG. 136A, FIGS. 136B and 136 Ceach show a TABLE listing amino acid sequences of TCR alpha and beta CDR3 and J regions of TCR that specifically recognize antigens associated with pathogenesis of autoimmune, allergic, or inflammatory conditions.

FIG. 137 shows a TABLE listing amino acid sequences of TCR alpha and beta CDR3 and J regions of TCR that specifically recognize antigens associated with pathogenesis of autoimmune, allergic, or inflammatory conditions.

FIG. 138 shows a TABLE listing amino acid sequences of J regions of TCR that specifically recognize antigens associated with pathogenesis of autoimmune, allergic, or inflammatory conditions.

FIG. 139A shows a TABLE listing nucleotide sequences encoding TCR alpha and beta chain V regions of TCR that specifically recognize antigens associated with pathogenesis of autoimmune, allergic, or inflammatory conditions;

FIG. 139B shows a TABLE listing amino acid sequences of J regions of TCR that specifically recognize antigens associated with pathogenesis of autoimmune, allergic, or inflammatory conditions.

FIG. 140A shows a TABLE listing nucleotide sequences encoding TCR alpha and beta chain V regions of TCR that specifically recognize antigens associated with pathogenesis of autoimmune, allergic, or inflammatory conditions;

FIG. 140B shows a TABLE listing amino acid sequences of TCR alpha and beta J regions of TCR that specifically recognize antigens associated with pathogenesis of autoimmune, allergic, or inflammatory conditions.

FIG. 141 shows a TABLE listing amino acid sequences of antigenic epitopes recognized by specific TCR for a CYP2D6 antigen associated with autoimmune hepatitis type 2.

FIG. 142 shows a TABLE listing amino acid sequences of antigenic epitopes recognized by specific TCR for a BP230 or a BP180 antigen associated with bullous pemphigoid.

FIG. 143A shows a TABLE listing amino acid sequences of polypeptide antigens associated with pathogenesis of autoimmune, allergic, and/or inflammatory conditions, containing antigenic epitopes recognized by specific TCR and amino acid sequences of TCR alpha and beta CDR3 regions of TCR that specifically recognize the antigens.

FIG. 143B shows a TABLE listing amino acid sequences of polypeptide antigens associated with pathogenesis of autoimmune, allergic, and/or inflammatory conditions, containing antigenic epitopes recognized by specific TCR and amino acid sequences of TCR alpha and beta CDR3 regions of TCR that specifically recognize the antigens.

FIG. 144 shows a TABLE listing amino acid sequences of polypeptide antigens associated with pathogenesis of autoimmune, allergic, or inflammatory conditions, containing antigenic epitopes recognized by specific TCR and amino acid sequences of TCR alpha and beta CDR3 regions of TCR that specifically recognize the antigens.

FIG. 145 shows a TABLE listing certain nucleic acid sequences useful with embodiments provided herein including guide RNA (gRNA), and an AAV vector containing TOXP3 editing sequences.

### DETAILED DESCRIPTION

Some embodiments of the methods and compositions provided herein relate to artificial antigen-specific immunoregulatory T (airT) cells. AirT cells may also be referred to as “edTreg” or “Edited Treg” cells. Some embodiments include an artificially engineered T cell (e.g., a T lymphocyte) comprising a CD4+CD25+ T cell having an artificial modification of a forkhead box protein 3/winged helix transcription factor (FOXP3) gene, and that constitutively expresses a FOXP3 gene product at a FOXP3 expression level that is equal to or greater than the FOXP3 expression level of a naturally occurring regulatory T (Treg) cell; and at least one transduced polynucleotide encoding an antigen-specific T cell receptor (TCR) polypeptide.

In some embodiments, the airT cells are capable of mediating antigen-specific immunosuppression when induced by a specific antigen that is recognized by the TCR, such as an autoantigen, an allergen, or another antigen associated with the pathogenesis of an inflammatory condition characterized by an excessive immune response. Significantly, production of the present airT cells does not require the time, costs, and inefficiencies associated with isolation of relatively rare (1-4% of human PBMC) natural Treg cells as a starting material for gene editing, thus affording certain advantages for the generation of therapeutically effective amounts of desired cells for adoptive immunotherapy.

In some embodiments, the airT cell expresses a functional TCR that specifically recognizes an antigen associated with pathogenesis of an autoimmune condition, an allergic condition, or an inflammatory condition, such as a TCR comprising any of the TCR polypeptide sequences disclosed herein or any of the TCR polypeptides encoded by the TCR-encoding polynucleotide sequences disclosed herein, including those set forth in the Drawings.

In some embodiments, the airT cell expresses a functional TCR that specifically recognizes an antigen associated with pathogenesis of an autoimmune condition, an allergic condition, or an inflammatory condition, such as any of the polypeptide autoantigens, allergens, and/or inflammation-associated antigens comprising the polypeptide antigen amino acid sequences disclosed herein, or any polypeptide antigens that are immunologically cross-reactive with the polypeptide autoantigens, allergens, and/or inflammation-associated antigens comprising the polypeptide antigen amino acid sequences disclosed herein, including those set forth in the Drawings.

Certain of the herein disclosed embodiments relate to gene editing strategies for the generation of the airT cells that include surprisingly advantageous functional linkage of (i) stable FoxP3 expression that results from targeted FoxP3 gene editing, including the introduction of a constitutive promoter to drive FoxP3 expression in cells that did not previously express FoxP3, wherein the FoxP3 expression is at a level equal to or greater than the FoxP3 expression level of a naturally occurring regulatory T (Treg) cell, to maintain a stable FoxP3-controlled immunoregulatory (immunosuppressive) program of the airT cell, and (ii) stable expression of an exogenously sourced TCR in the same cells by gene editing to introduce into the airT cell the particular presently disclosed nucleotide sequences encoding TCR that recognize antigens associated with pathogenesis of an autoimmune condition, an allergic condition, or an inflammatory condition, to permit selection and expansion of engineered T cells characterized by stable immunosuppressive potential that co-segregates with desired TCR expression. Without wishing to be bound by theory, it is believed that by artificially engineered stable FoxP3 expression, some embodiments of the presently described airT cells include safe and effective adoptive transfer immunotherapy cells for indications where antigen-specific immunosuppression is desirable, such as autoimmune, allergic, or other inflammatory conditions, without the risks associated with natural Treg plasticity (e.g., reversion to T effector behavior).

Production of the presently disclosed airT cell advantageously and in some embodiments does not include first isolating natural Treg cells, which as noted above, are naturally present in peripheral blood at a low frequency, representing only about 1-4% of human peripheral blood mononuclear cells. Instead, as described herein, generation of airT cells can be achieved by isolating CD4+ T cells, which although heterogeneous with respect to other cell surface markers may comprise approximately 25-60% of human PBMC and thus represent a relatively abundant starting material for gene editing according to the various strategies provided herein.

The present antigen-specific immunoregulatory T (airT) cell compositions and methods will, in certain embodiments, find uses in the treatment and/or amelioration of certain autoimmune conditions, allergic conditions, and/or inflammatory conditions, including in adoptively transferable immunotherapy, where stable airT cell viability and maintenance of antigen-specific immunoregulatory function provide unprecedented advantages.

In certain embodiments the airT cell described herein is unexpectedly capable of inducing an antigen-specific immunosuppressive response when stimulated by an antigen associated with pathogenesis of an autoimmune condition, an allergic condition, or an inflammatory condition such as one of the antigens disclosed herein. Such antigen-specifically induced immunosuppression may comprise one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines or anti-inflammatory products, for example, elaboration of one or more inhibitory mechanisms including release of immunosuppressive cytokines or perforin/granzyme, induction of indoleamine 2,3-dioxygenase (IDO), competition for IL2 or adenosine, catabolism of tryptophan, and expression of inhibitory receptors by the airT cell, by the airT cell, and (iv) inhibition of either or both of activation and proliferation of effector T cells that do not recognize the antigen that is specifically recognized by the airT TCR comprising the TCR polypeptide that is encoded by the at least one transduced polynucleotide. In some embodiments such antigenic stimulation of the airT cell is HLA-restricted.

In some embodiments, the generation of the present airT cells, which stably express FoxP3 as described herein, overcomes certain disadvantages associated with prior methodologies in which FOXP3 transgene expression was achieved by retroviral or lentiviral gene transfer. The resulting virally FoxP3-transduced cell populations were genetically heterogeneous by virtue of having randomly integrated FOXP3 transgenes of varying stability and varying expression levels at various genomic sites. Despite at least transiently exhibiting Treg characteristics such as phenotypic markers and cytokine expression profile, such transduced populations were also potentially compromised by carrying a concomitant risk of genotoxicity, as well as vulnerability to silencing by local regulatory elements at sites of viral integration.

To avoid these risks, some embodiments provided herein include the use of specifically targeted gene editing for artificial modification of the FOXP3 gene instead of relying on viral FOXP3 gene transfer and, optionally specifically targeted TCR gene editing. Certain embodiments described herein utilize lentiviral gene delivery to introduce candidate autoimmune-related TCRs into CD4 T cells, followed by FOXP3 gene editing of the cells to force stable FoxP3 expression. In some related embodiments, this approach is combined with gene editing methods to simultaneously delete the endogenous TCR gene (e.g., via inactivation, also referred to herein as “knockout”).

As an alternative strategy distinct from lentiviral TCR delivery, some embodiments described herein relate to simultaneous gene editing at different alleles of the same gene locus e.g., single-locus bi-allelic dual editing in which dual-editing is achieved at a single locus (e.g., with a single guide RNA and AAV donor homology constructs).

As another alternative, some embodiments described herein relate to simultaneous gene editing at two different gene loci, e.g., two-loci dual editing in which a distinct gene editing event takes place at each of two loci (e.g., with two different guide RNAs and locus-specific AAV donor homology cassettes).

By these approaches, engineered FOXP3 and TCR genes may be delivered to a single specific gene locus or to two different specific loci. As also described herein, in some embodiments this strategy may further include incorporating split chemical-induced signaling complex (split CISC) components that permit selective expansion of only those T cells that express both the inserted TCR and the Foxp3 genes in the same cell, thereby enriching for airT cells.

### Chemical-Induced Signaling Complex (CISC)

As described herein, some embodiments exploit a split chemical-induced signaling complex (split CISC) strategy by which gene-edited airT cells may be generated and selectively expanded on the basis of successful expression in the same cells of both (i) a constitutively expressed FoxP3 gene-edited gene product, the expression of which is associated with cell surface expression of a first CISC component that specifically binds to a CISC inducer molecule, the first CISC component being present as a transmembrane fusion protein having a first extracellular CISC inducer molecule binding domain, a transmembrane domain, and a first intracellular activation signal transduction domain; and (ii) a transduced heterologous TCR gene-edited gene product, the expression of which is associated with cell surface expression of a second CISC component that is different than the first CISC component and specifically binds to the CISC inducer molecule, the second CISC component being present as a transmembrane fusion protein having a second extracellular CISC inducer molecule binding domain, a transmembrane domain, and a second intracellular activation signal transduction domain that is different than the first intracellular activation signal transduction domain.

In certain embodiments, CD4+ T cells are enriched from a biological sample such as peripheral blood mononuclear cells (PBMC) prior to gene editing (e.g., dual editing) as described herein. In certain embodiments enriched CD4+ T cells are non-specifically activated (e.g., with solid-phase immobilized anti-CD3 and anti-CD28 antibodies) prior to gene editing (e.g., dual editing) as described herein.

In some embodiments, exposure of dual-edited T cells as described herein to the CISC inducer molecule results in binding of the inducer molecule to the extracellular domains of both the first and second CISC components and heterodimer formation by the first and second CISC components to activate a functional signal transduction complex that is formed by the first and second intracellular activation signal transduction domains. As a consequence, the population of airT cells in which are expressed both the first and second CISC components, and hence both FoxP3 and the heterologous TCR, is selectively expanded.

In some embodiments, the two gene editing events that give rise to expression in the present airT cells, of the first CISC component concomitant with the FoxP3 gene product and of the second CISC component concomitant with the TCR gene product, may be designed to take place in different alleles of the same gene locus (e.g., bi-allelic dual editing), or at two different gene loci (e.g., two-loci dual editing). In some embodiments, a third CISC component that specifically binds to the CISC inducer molecule may also be co-expressed with either the FoxP3 gene product or the TCR gene product. The third CISC component remains at an intracellular locale when expressed and acts as a decoy to bind and thereby avoid toxicities associated with certain CISC inducer molecules that may reach the cell interior.

Details of CISC systems, including structures of first, second and third CISC components and of CISC inducer molecules are described elsewhere herein and in WO/2018/111834 and WO/2019/210078, which are both expressly incorporated by reference in their entireties. Briefly, WO/2018/111834 describes compositions and methods for genetically editing host cells by knock-in (insertion) of genetic constructs encoding a ligand-dimerizable fusion protein chemical-induced signaling complex (CISC). Cellular expression of both fusion protein subunits followed by exposure of the host cells to the chemical ligand permits ligand-induced dimerization of the CISC to transduce a cellular activation signal. The CISC system thus provides selection and expansion (e.g., activation-induced proliferation) of cells that have undergone gene modification to incorporate both of the CISC components, to select cells in which gene editing has occurred. WO/2019/210078 describes gene editing compositions and methods in which nucleic acid sequences encoding first and second CISC subunit components are introduced to host cells as part of gene editing at a single targeted FOXP3, TRAC, or AAVS1 gene locus. Chemical ligand-induced dimerization of the CISC can induce a biological signal transduction event for selection and expansion of edited cells. Optionally and in some related embodiments a nucleic acid encoding a third CISC subunit component is also expressed in the host cells; the third CISC component remains intracellularly expressed as a decoy to decrease potential harmful effects on the cell of internalized CISC ligand.

Exemplary first and second CISC subunit components may comprise functional intracellular signal transduction domains of IL2-receptor beta and gamma subunits (IL2RB, IL2RG). An exemplary third CISC component may comprise a functional rapamycin-binding domain of FK506-binding protein (FKBP).

### FOXP3/ airT Phenotypic Markers and Suppressor Function

FOXP3 gene editing may include artificial modification of a native FOXP3 gene locus and/or may also include artificial modification of a chromosomal site other than a native FOXP3 gene locus. For example, gene editing may include knock-in (e.g., insertion) of a nucleic acid molecule comprising an exogenous FOXP3-encoding polynucleotide operably linked to a constitutive promoter at a chromosomal site other than a native FOXP3 gene locus, such as a T cell receptor alpha chain (TRAC) gene locus, a T cell receptor beta chain (TCRB) locus or an adeno-associated virus integration site 1 (AAVS1) or another gene locus. Certain embodiments thus surprisingly provide the herein described airT cells, which are capable of mediating antigen-specific immunosuppression, when artificial FoxP3 gene sequences are introduced to a genomic site other than the native FOXP3 gene locus (e.g., in the TRAC locus) and are able constitutively to express a FOXP3 gene product at a level that is equal to or greater than the FOXP3 expression level of a naturally occurring Treg cell.

The two gene editing events that give rise in certain embodiments to expression in the present airT cells, of the FoxP3 gene product concomitant with the first CISC component and of the TCR gene product concomitant with the second CISC component concomitant, may be designed to take place in different alleles of the same gene locus (e.g., bi-allelic dual editing), or at two different gene loci (e.g., two-loci dual editing). In some embodiments the presently disclosed airT cell is surprisingly capable of expressing the FOXP3 gene product at an expression level sufficient for the airT cell to maintain a CD4+CD25+ phenotype for at least 21 days in vitro, or for at least 60 days in vivo following adoptive transfer to an immunocompatible mammalian host in need of antigen-specific immunosuppression, while functionally expressing a herein-disclosed TCR that specifically recognizes an antigen associated with pathogenesis of an autoimmune condition, an allergic condition, or an inflammatory condition, or a TCR that specifically recognizes a herein-disclosed antigen associated with pathogenesis of an autoimmune condition, an allergic condition, or an inflammatory condition.

The CD4+CD25+ airT cell disclosed herein thus in certain embodiments relates to a genetically engineered cell obtained by artificial modification of a FOXP3 gene in a CD4+CD25⁻ T cell. in some embodiments, the artificial modification causes the airT cell to constitutively express a FOXP3 gene product at a FOXP3 expression level that is equal to or greater than the FOXP3 expression level of a naturally occurring regulatory T (Treg) cell. In some embodiments, the airT cell may also express the CD25, CD152, and/or ICOS cell surface markers at levels which are characteristic of immunoregulatory cells such as natural Treg. Unlike natural Treg, however, in some embodiments the present airT cells may exhibit a HeliosLo cell surface phenotype, e.g., an expression level of the Helios cell surface marker that is decreased, in a statistically significant manner, relative to the Helios expression level in naturally occurring Treg cells.

Exemplary details of gene editing strategies to induce FoxP3 expression in T cells are described herein and in WO/2018/080541 and in WO/2019/210078, which are expressly incorporated by reference in their entireties. Exemplary details of forced FOXP3 expression by gene editing including knock-in (insertion) of a full length, codon-optimized FoxP3 cDNA into the FOXP3 or AAVS1 locus may be found in WO/2019/210042, which is expressly incorporated by reference in its entirety.

Briefly, WO/2018/080541 describes CD4+ T cells in which stable expression of endogenous FoxP3 is engineered by gene editing using Cas9, ZFN, or TALEN to knock-in (e.g., by insertion) a constitutive promoter that is an EF1a, PGK, or MND promoter. FoxP3 expression may be achieved by targeted knock-in (insertion), at the FOXP3 gene locus, of a polynucleotide comprising a regulatory sequence operably linked to a coding sequence for the first expressed FOXP3 exon. The regulatory sequence may comprise a promoter which in some embodiments may be the MND, PGK, or EF1a promoter, or another inducible, weak, or constitutive promoter. Exemplary edited FOXP3+ cells may comprise a fully methylated FOXP3 gene intronic regulatory T cell-specific demethylation region (TSDR) upstream of the knocked-in promoter integration site.

WO/2019/210078 describes forced expression of FoxP3 in CD4+ T cells to achieve cells having a Treg-like phenotype, methods of selecting for such cells to obtain a Treg-enriched preparation, and methods for expanding populations of such cells in vitro. WO/2019/210078 also describes compositions and methods for targeted gene editing at the FOXP3, AAVS1, and/or TCRalpha (TRAC) loci, including guide RNA (gRNA) sequences specific for each of these loci and donor templates for gene editing by HDR. WO/2019/210078 also describes a CISC system in which a chemical-ligand dimerization of first and second CISC components results in an activation signal that effects T cell proliferation and hence selective expansion of edited T cells. WO/2019/210078 describes first and second CISC components in which the CISC inducer molecule is rapamycin or any of a large number of disclosed rapamycin analogues, derivatives, and mimetics, and in which the activation signal transduction domains of the CISC components comprise functional portions of the cytoplasmic domains of the IL-2 receptor beta (IL2Rb, also referred to as IL2RP) and IL-2 receptor gamma (IL2Rg, also referred to as IL2R□) subunits of the IL-2 receptor (IL2R).

Certain methods for phenotypic and functional characterization of Treg cells including cells in which FoxP3 overexpression has been induced are known in the art (e.g., WO/2018/080541, WO/2019/210078, McMurchy et al., 2013 Meths. Mol. Biol. 946: 115-132; Thornton et al., 2019 Eur. J. Immunol. 49:398-412; Aarts-Riemens et al., 2008 Eur. J. Immunol. 38: 1381-1390; McGovern et al., 2017 Front. Immunol. 8: Art. 1517; which are each expressly incorporated by reference in its entirety) and are described herein. These and related methodologies are applicable to characterization of the present airT cells as described herein.

Unlike natural Treg cells, in the presently disclosed airT cells the intronic Treg-specific demethylated region (TSDR) in the FoxP3 gene locus comprises cytosine-guanine (CG) dinucleotides having cytosine (C) nucleotides at certain positions that are predominantly methylated. For example, in the present airT cells at least 80%, 85%, 90%, 95%, 96%, 97%, 98%, or 99% of the TSDR C nucleotides at nucleotide positions that comprise a demethylated C nucleotide in a naturally occurring Treg cell are methylated. Methylation analysis of the FoxP3 TSDR is known to be routine in the art by any of several different methodologies (e.g., Salazar et al., 2017 Front. Immunol. 8:219; Ngalamika et al., 2014 Immunol. Invest. 44(2): 126-136 which is expressly incorporated by reference in its entirety).

Despite this difference in TSDR epigenetic modification between airT cells and natural Treg cells, the present airT cells are capable of mounting an immunosuppressive response to TCR stimulation by a specifically recognized antigen. The antigen-specific immunosuppressive properties of the presently disclosed airT cells were therefore unexpected in view of the report by Wright et al. (2009 Proc. Nat. Acad. Sci. USA 106: 19078) that co-transfection of CD4+ cells with FoxP3 and TCR constructs in viral vectors did not produce Treg-like cells that were functionally capable of exhibiting antigen-specific suppression. Without wishing to be bound by theory, it is believed that the presently disclosed airT cells thus provide unforeseen advantages that may derive at least in part from the manner in which they are prepared, including by artificial gene editing as described herein.

### T Cell Receptor (TCR)

A transduced polynucleotide encoding an exogenously sourced TCR to be expressed in an airT cell may involve artificial modification of a native TCR gene locus (e.g., TRAC) and/or may also involve artificial modification of a chromosomal site other than a native TCR gene locus, for example, gene editing by knock-in (insertion) of a nucleic acid molecule comprising an exogenous TCR-encoding polynucleotide at a chromosomal site other than a native TCR gene locus, such as the FOXP3 gene locus or AAVS 1 or another gene locus.

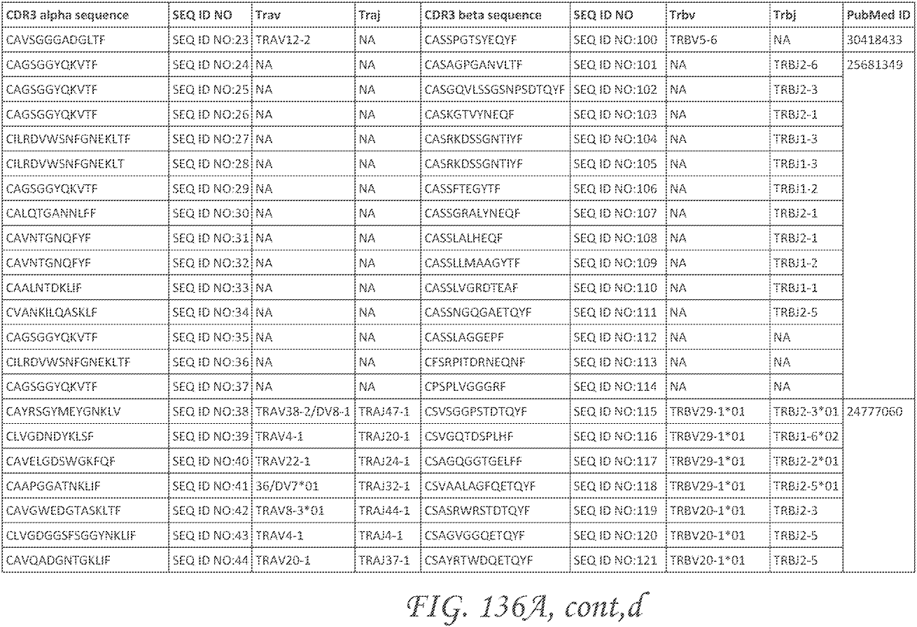

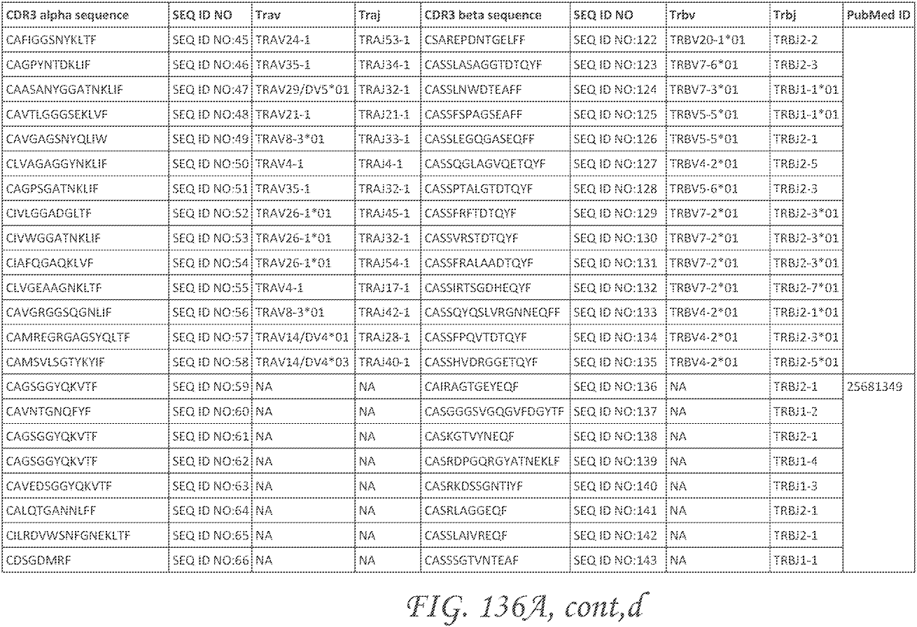

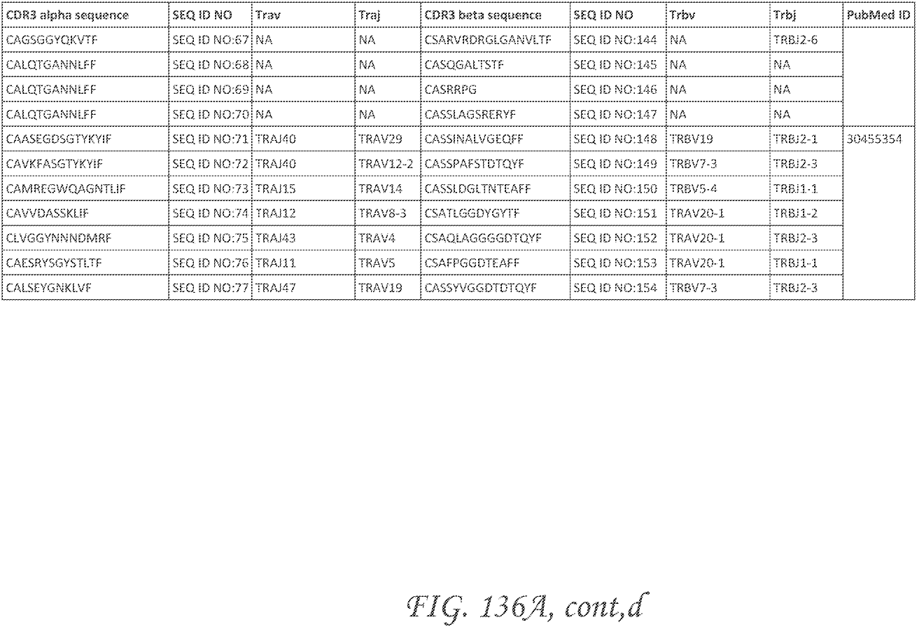

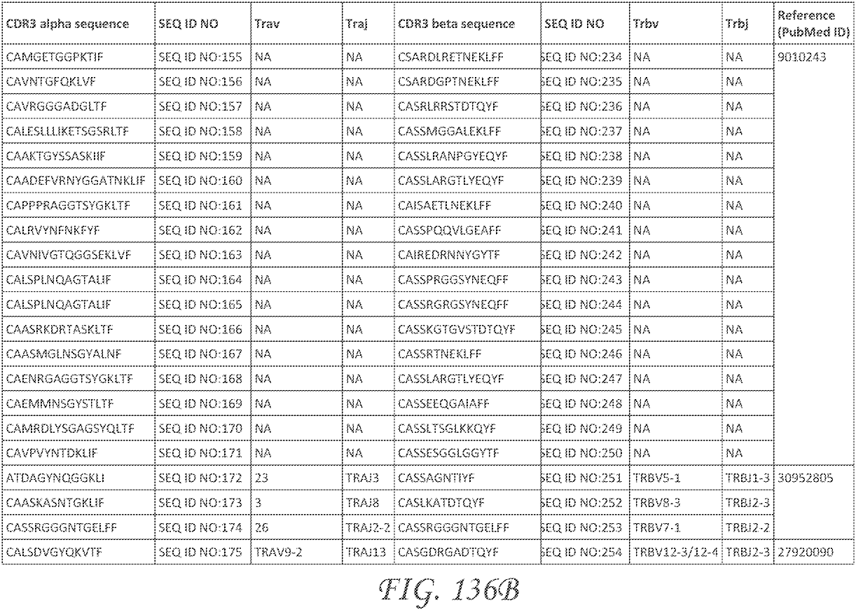

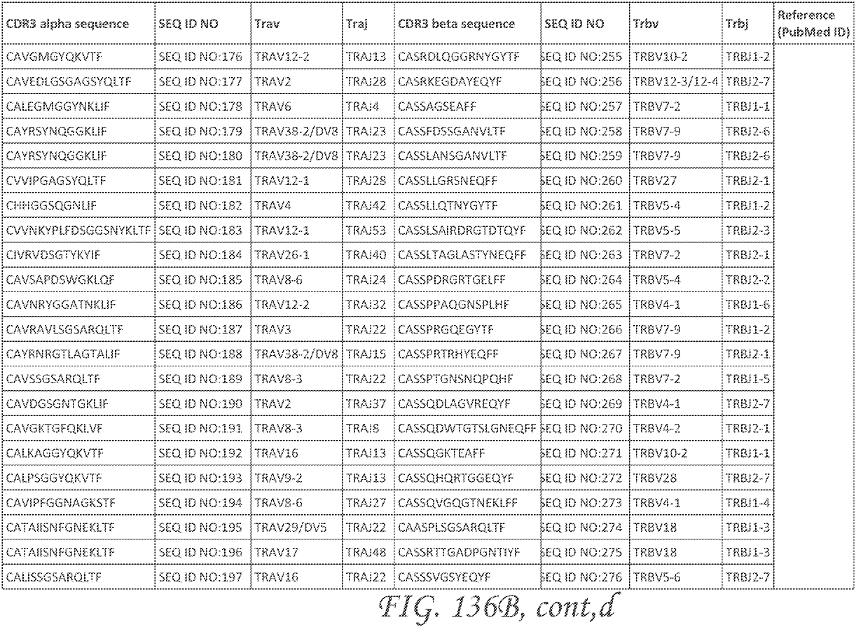

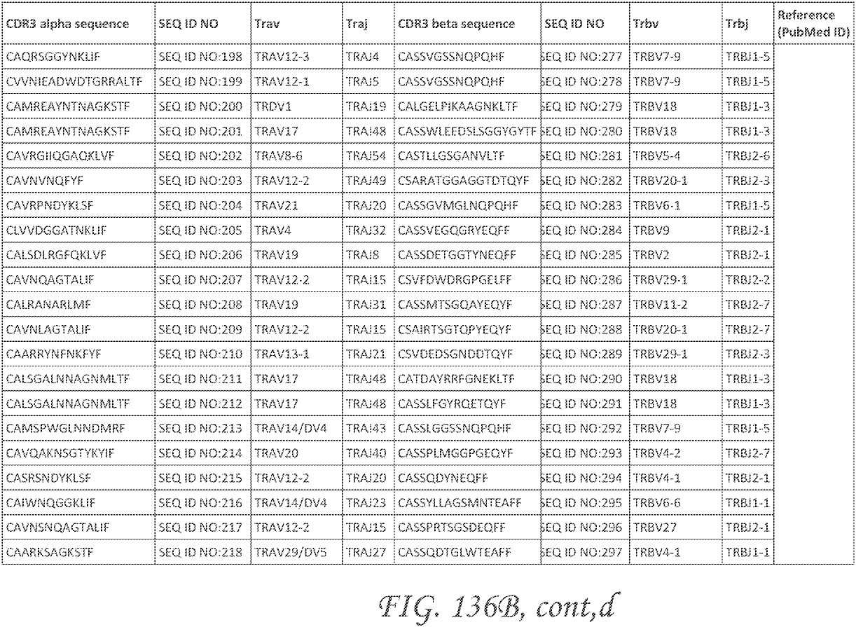

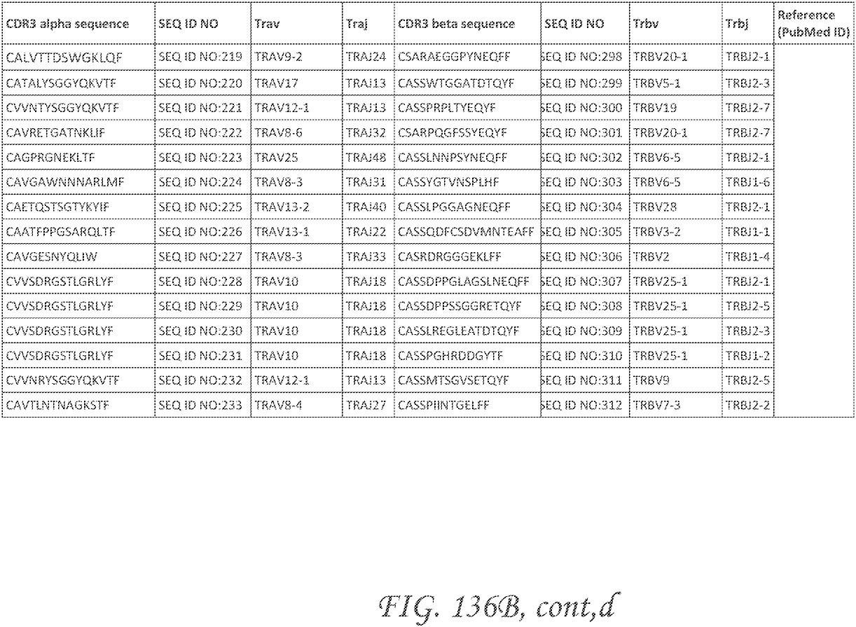

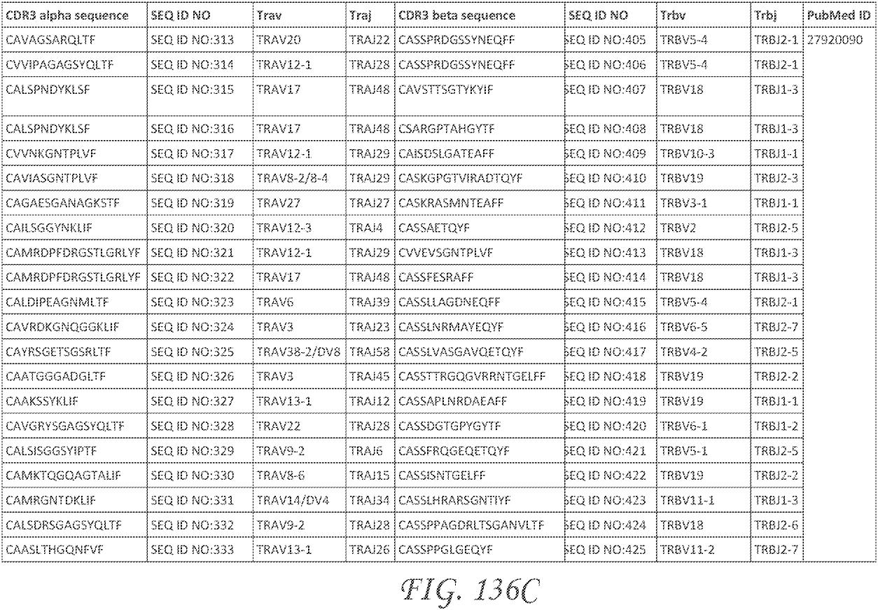

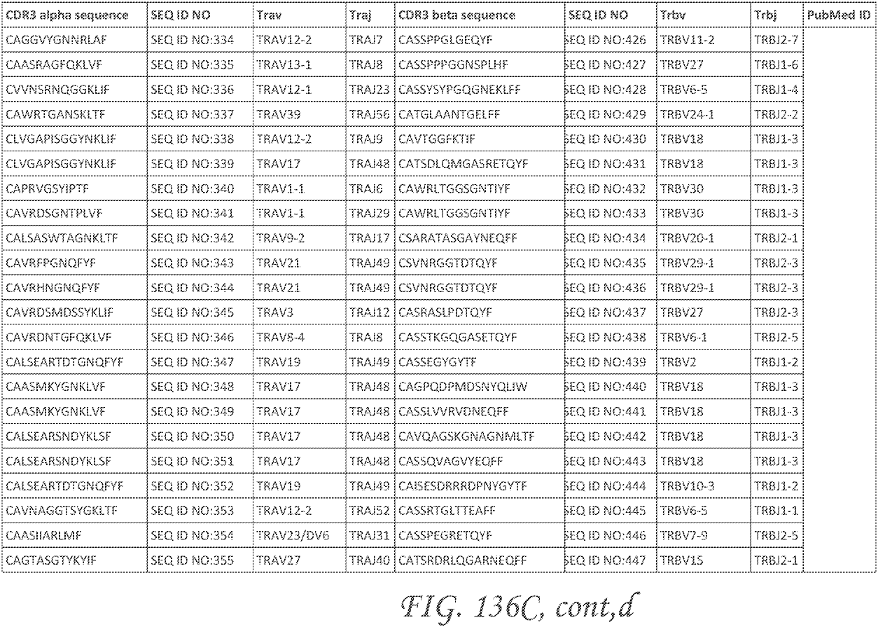

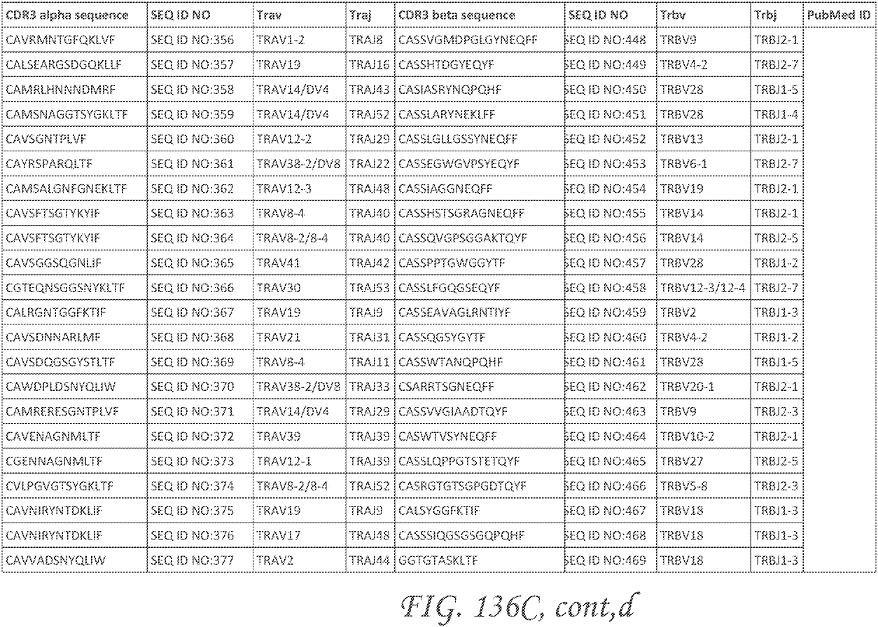

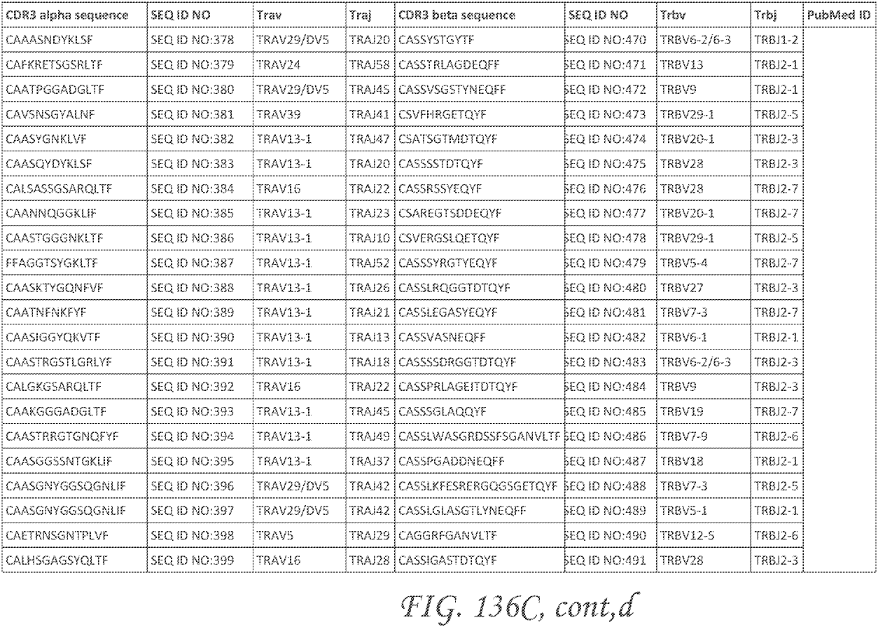

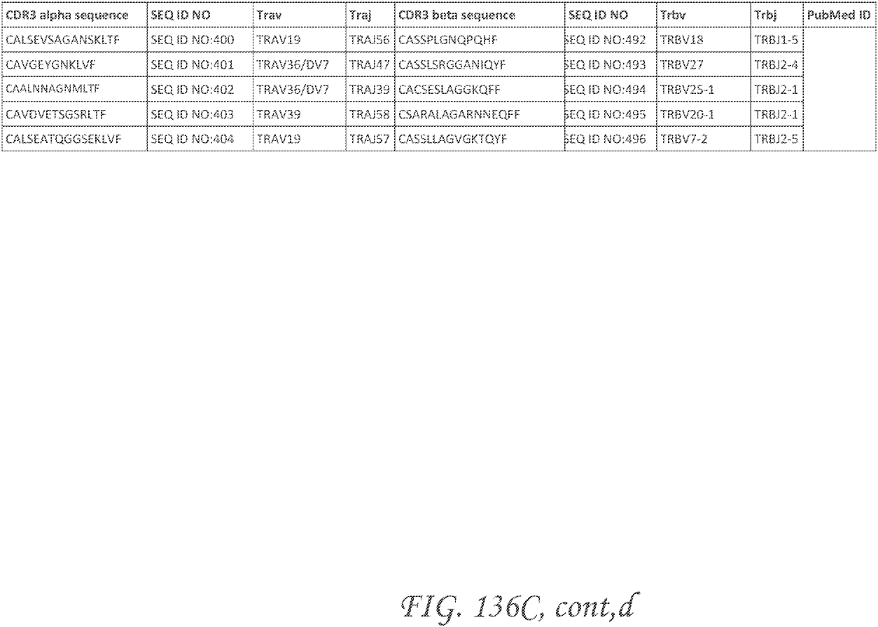

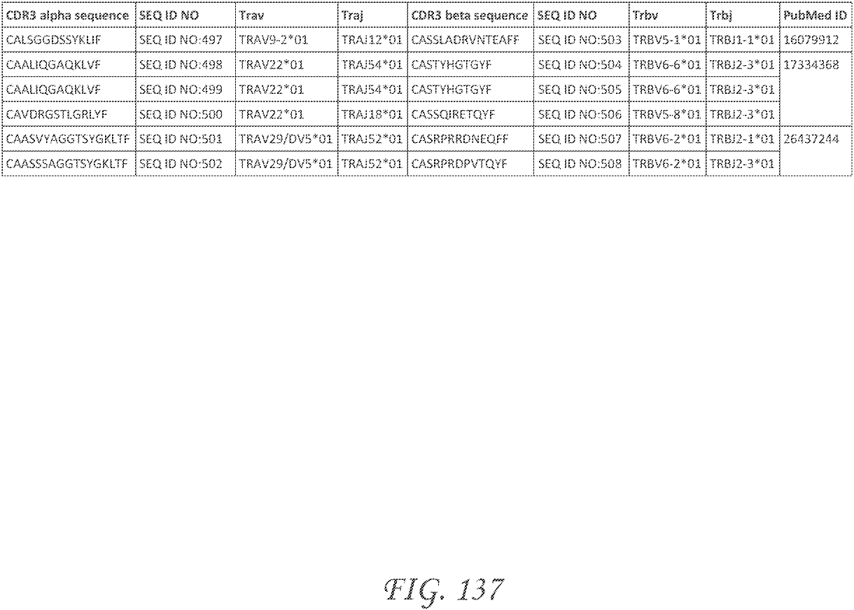

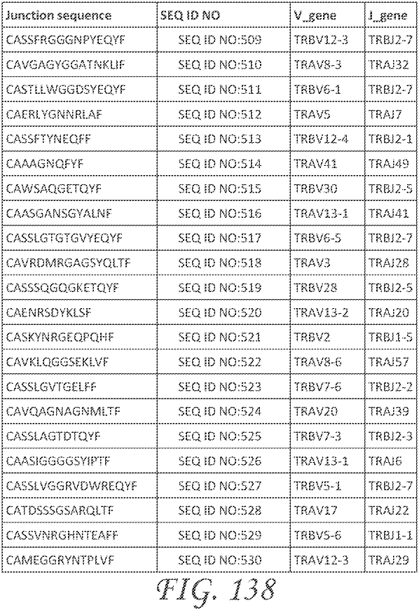

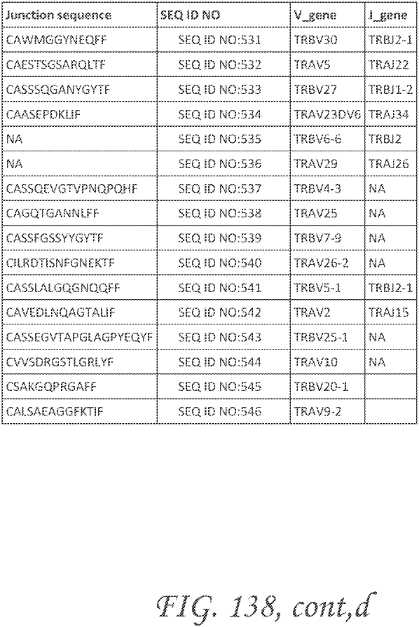

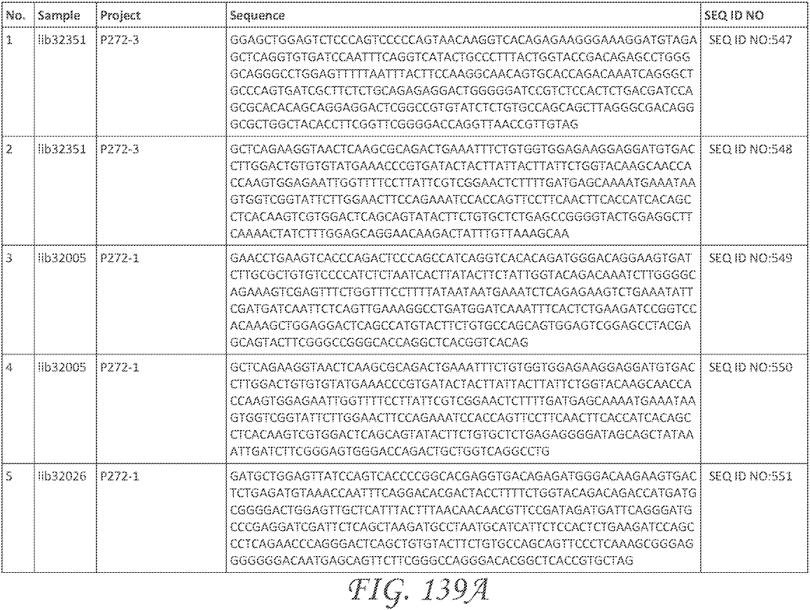

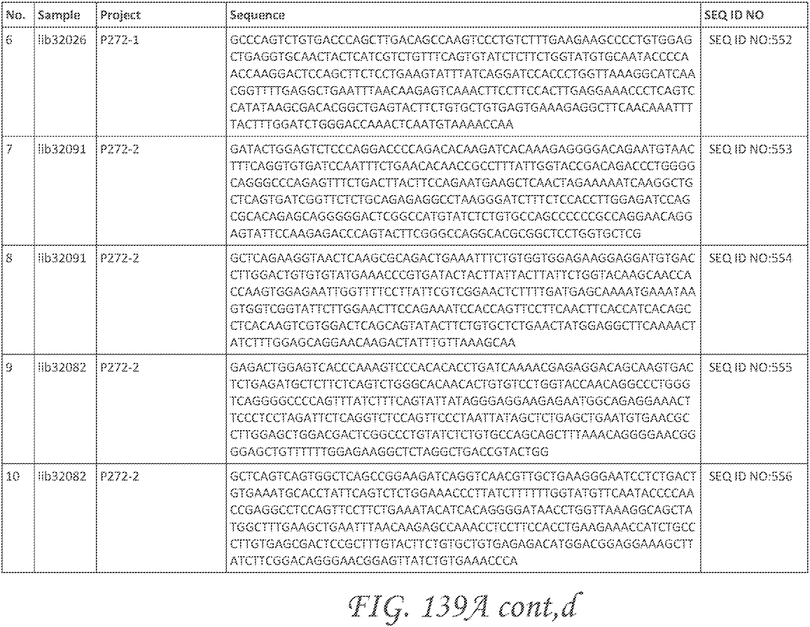

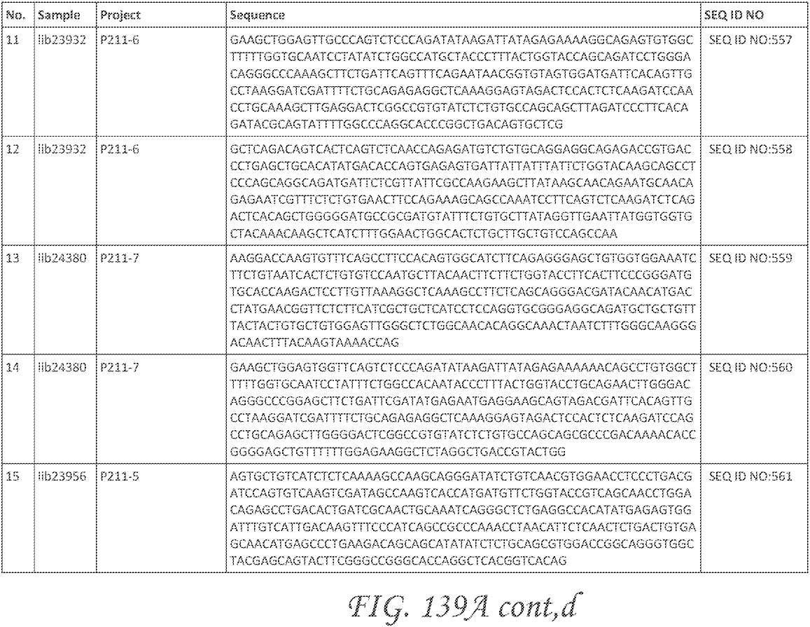

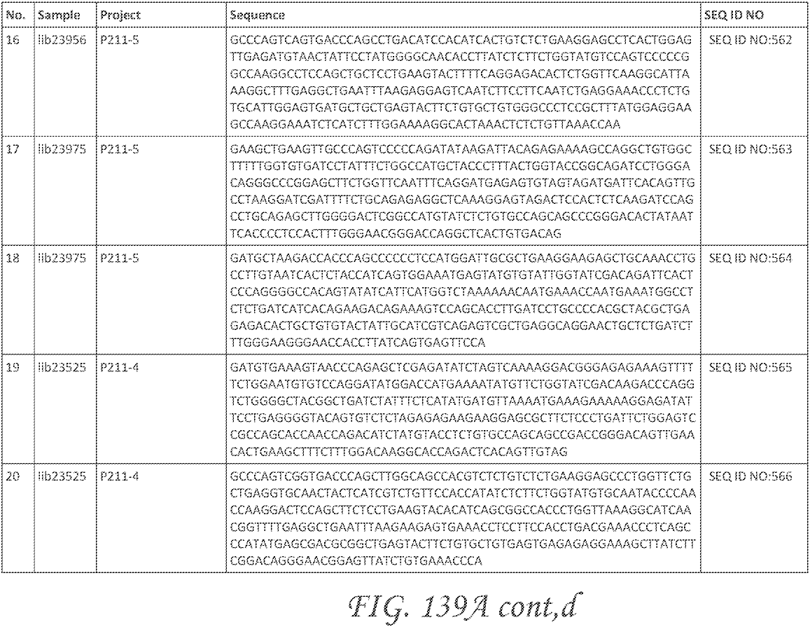

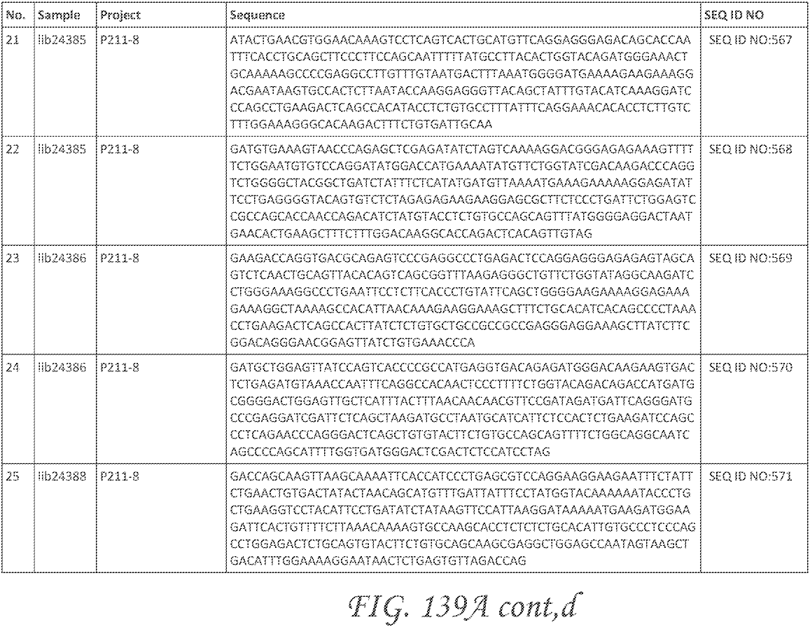

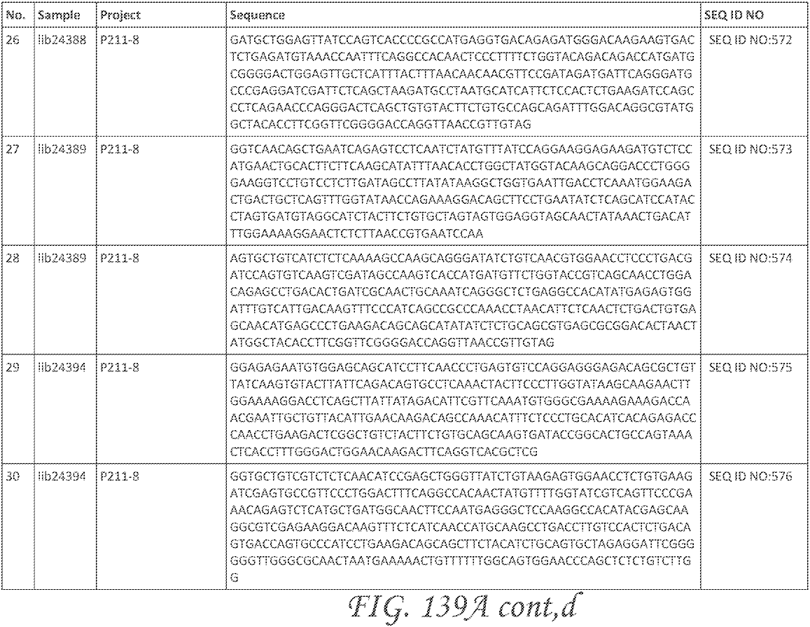

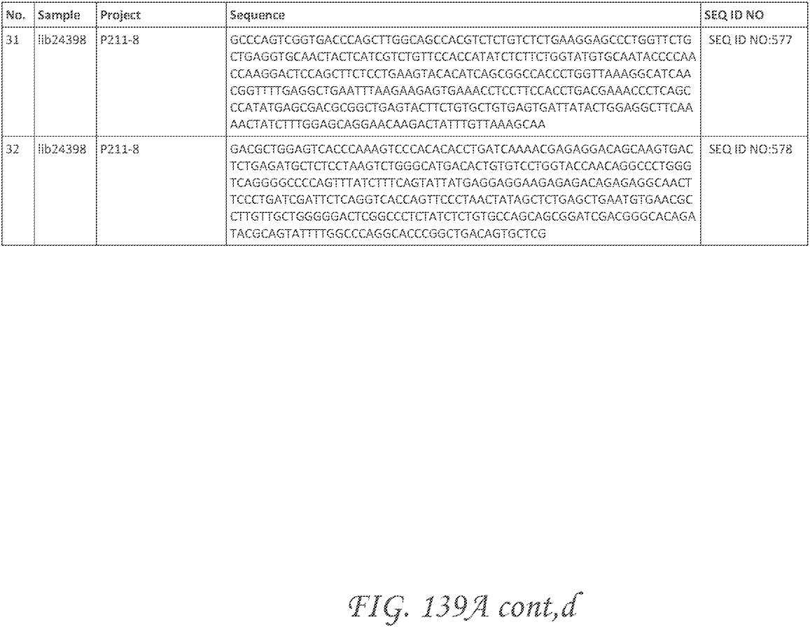

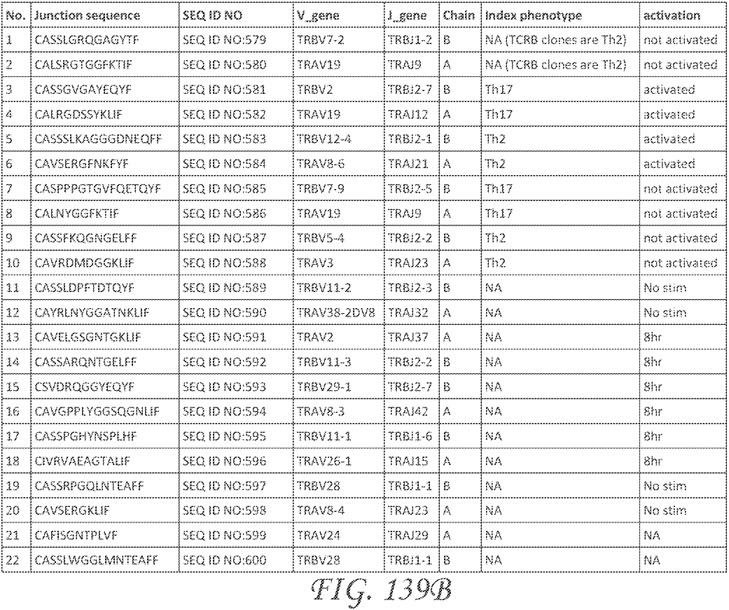

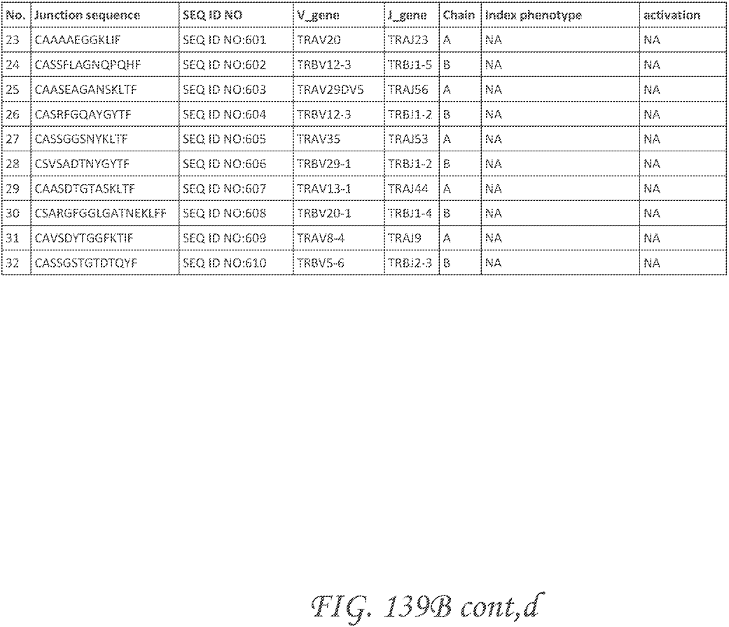

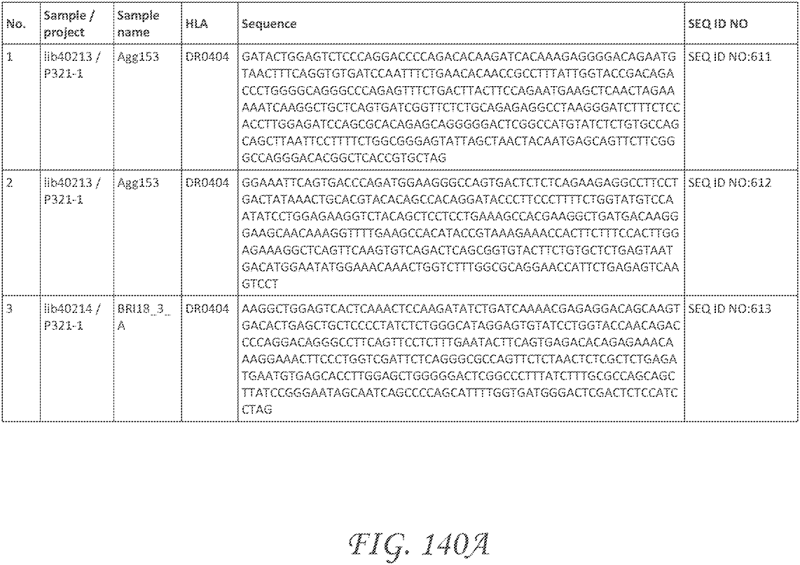

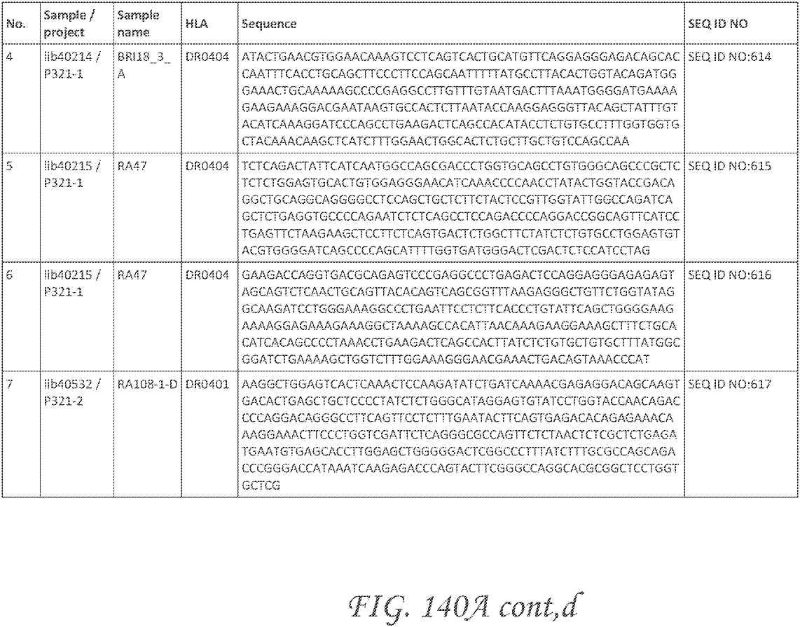

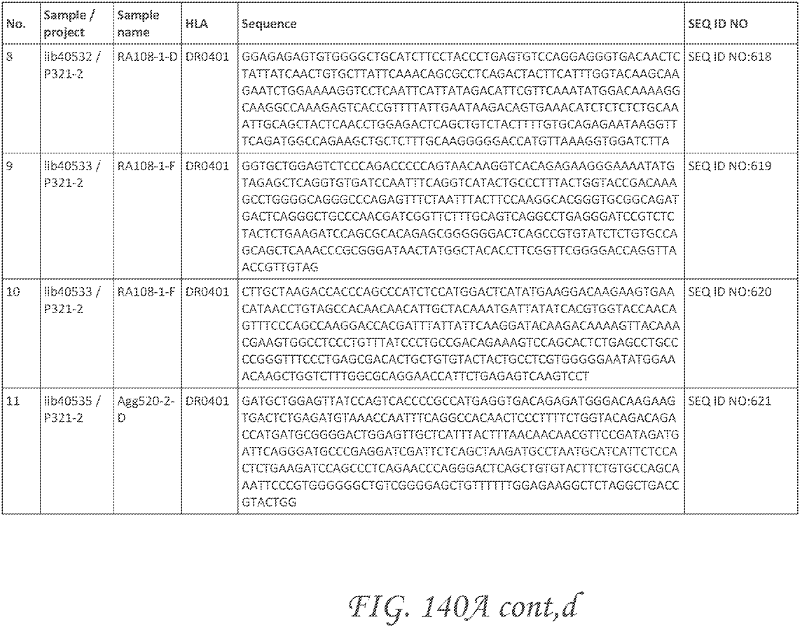

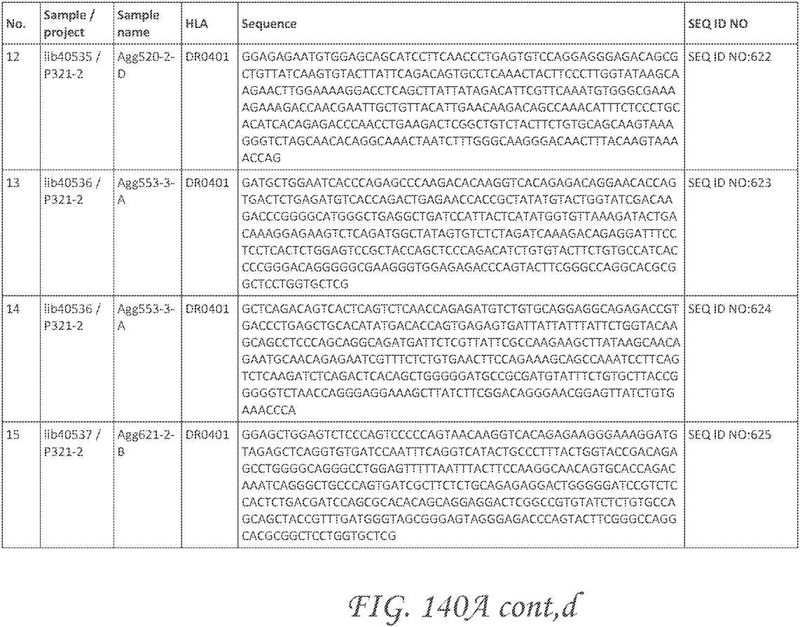

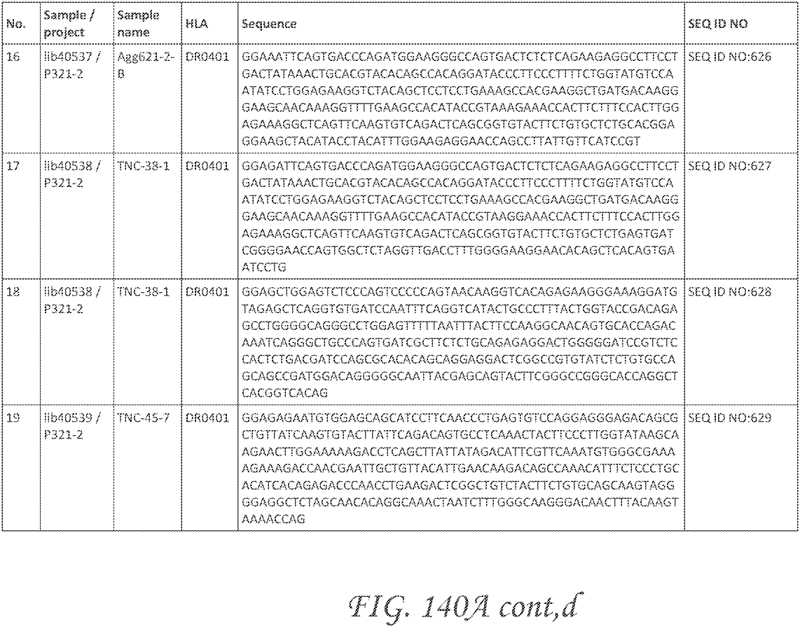

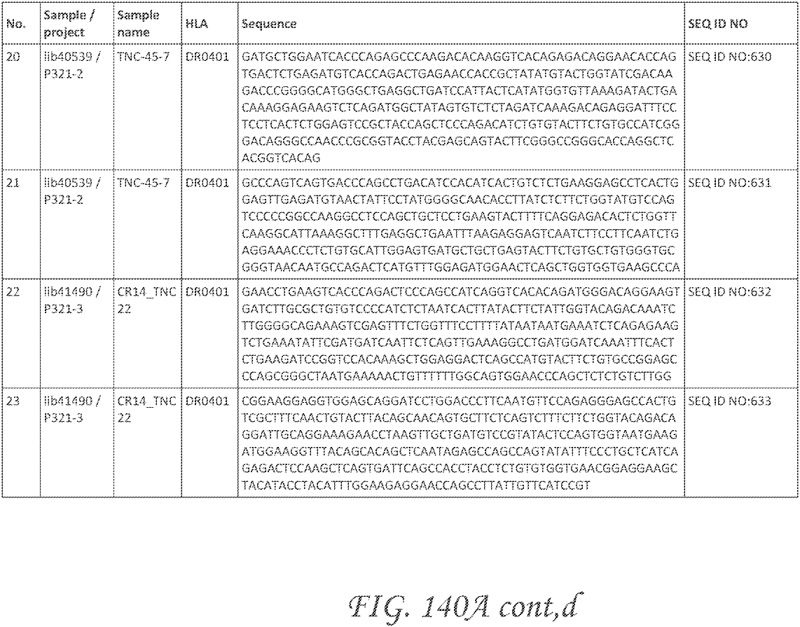

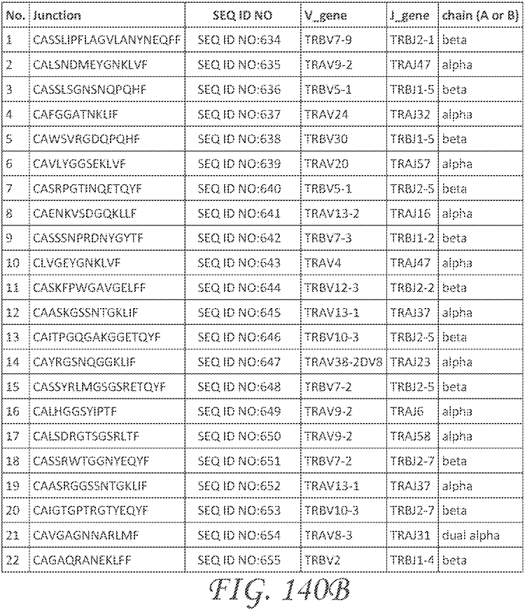

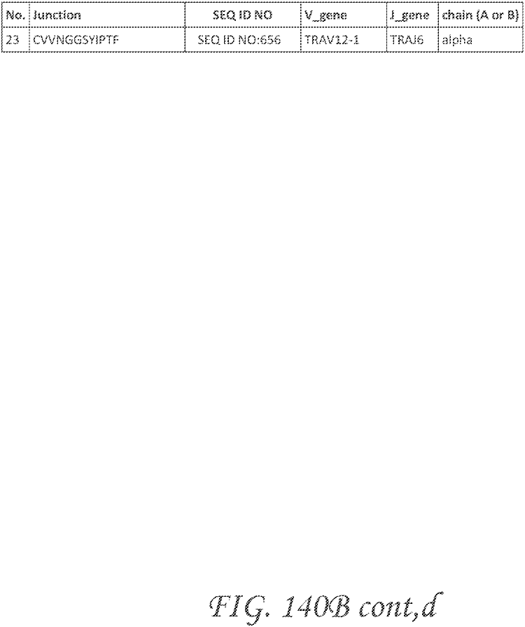

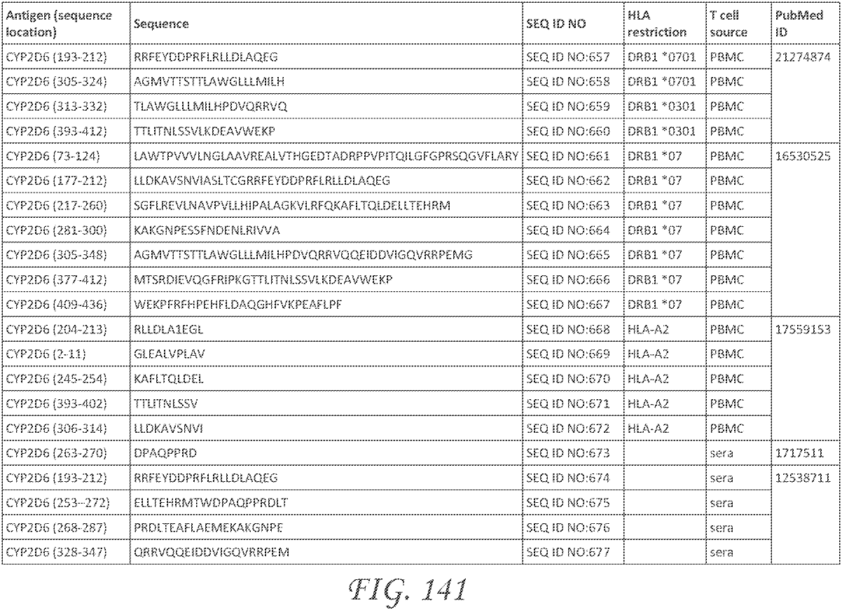

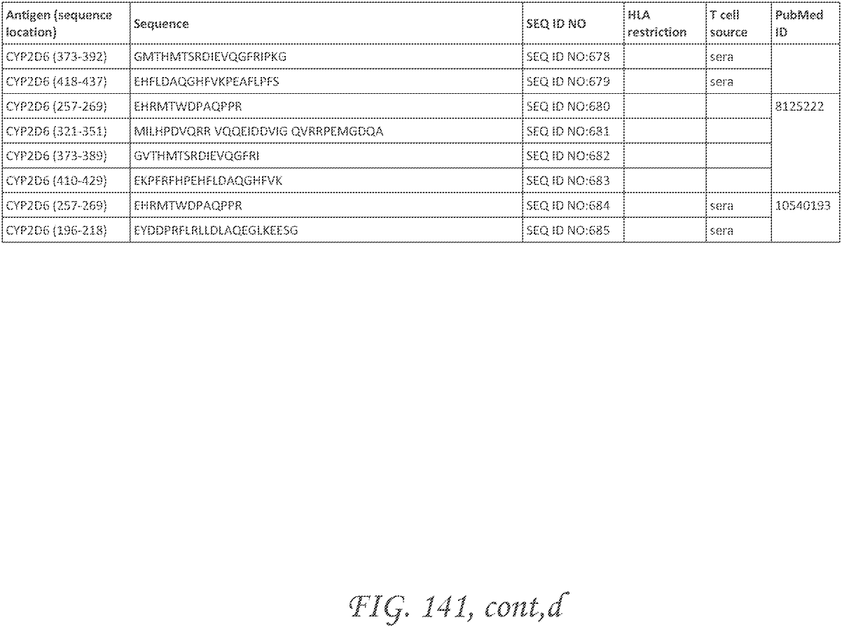

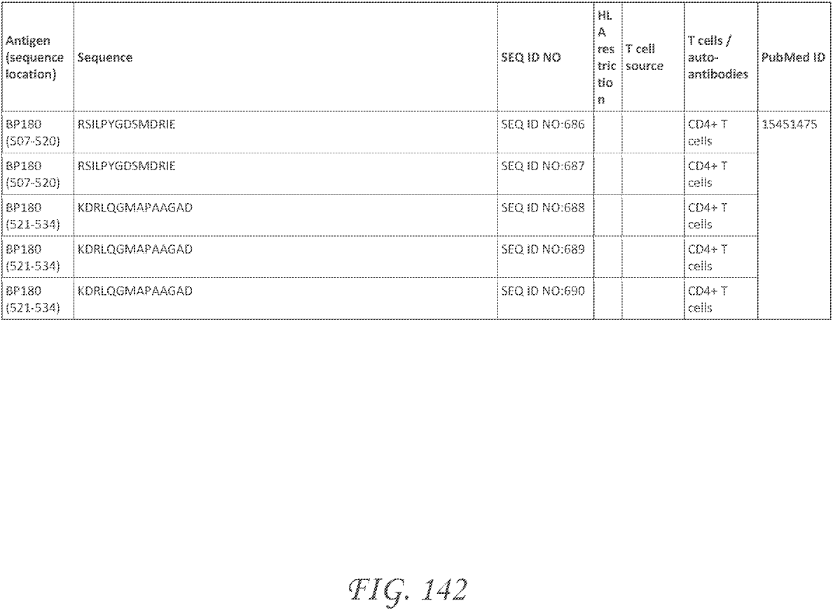

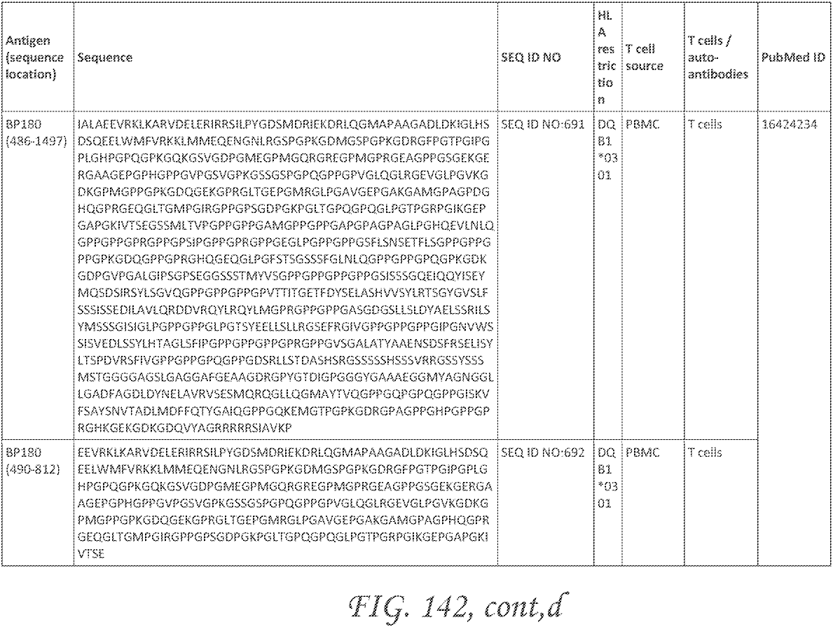

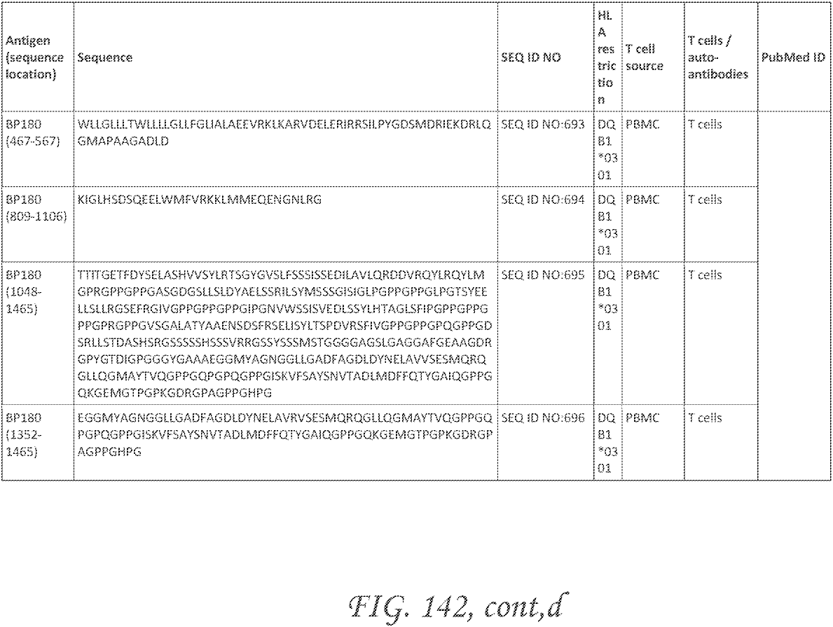

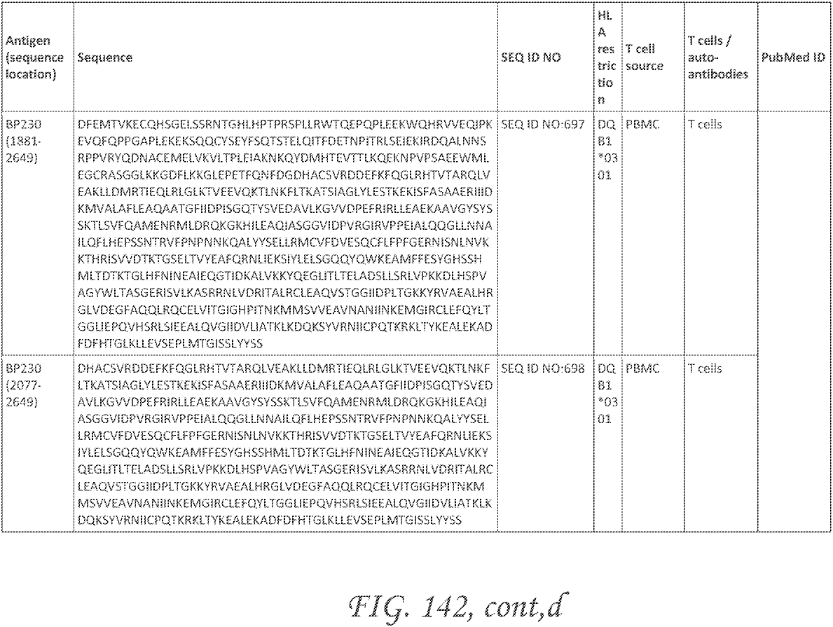

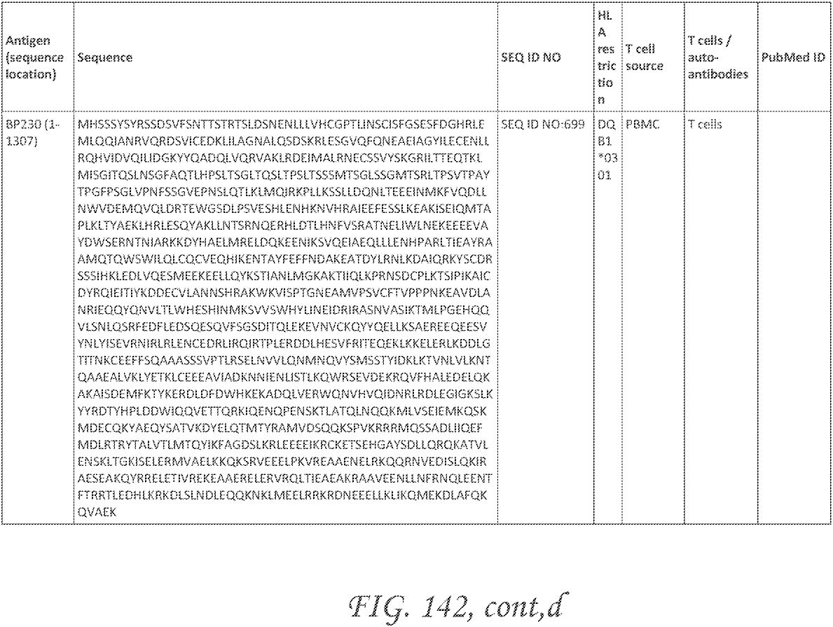

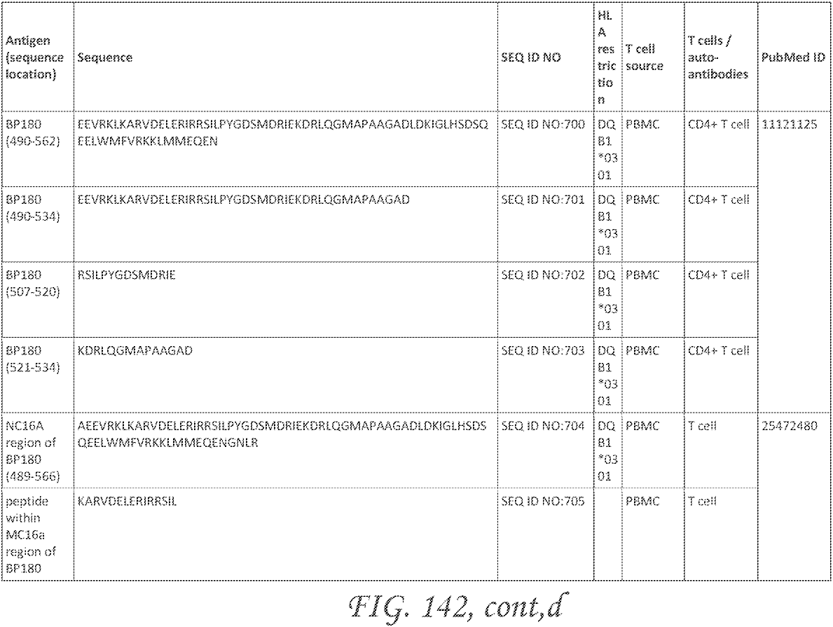

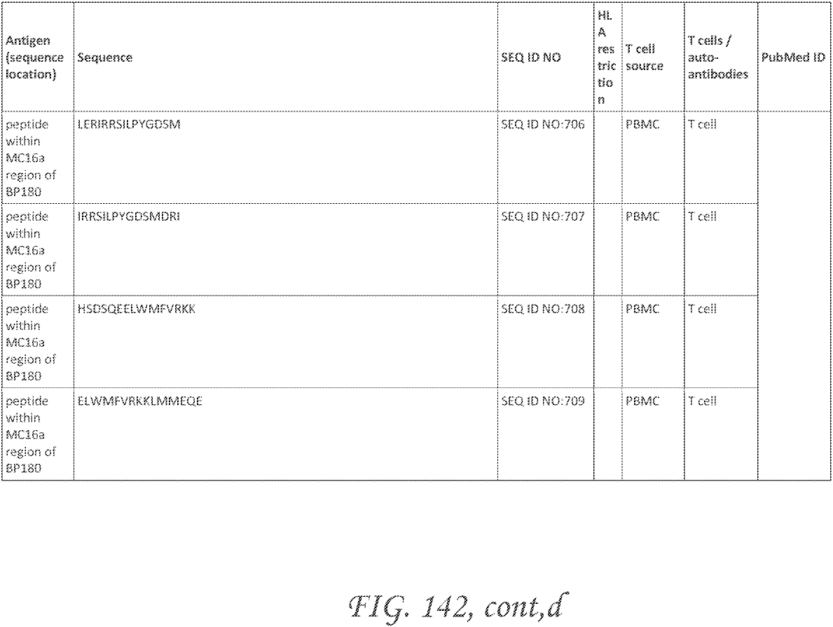

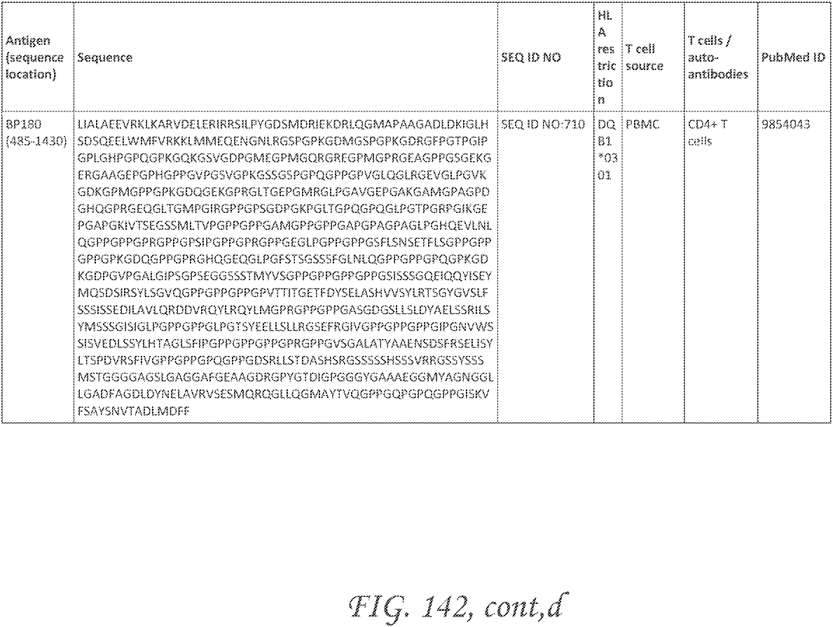

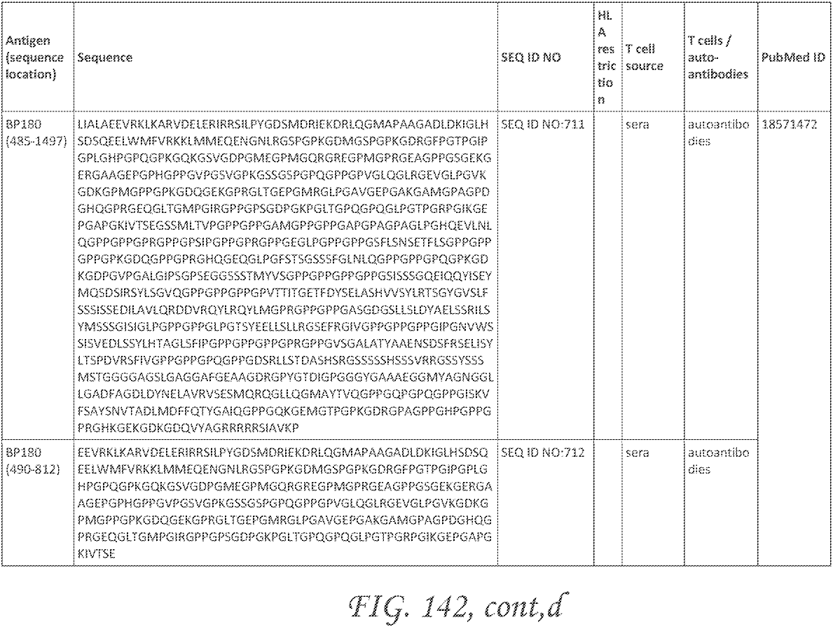

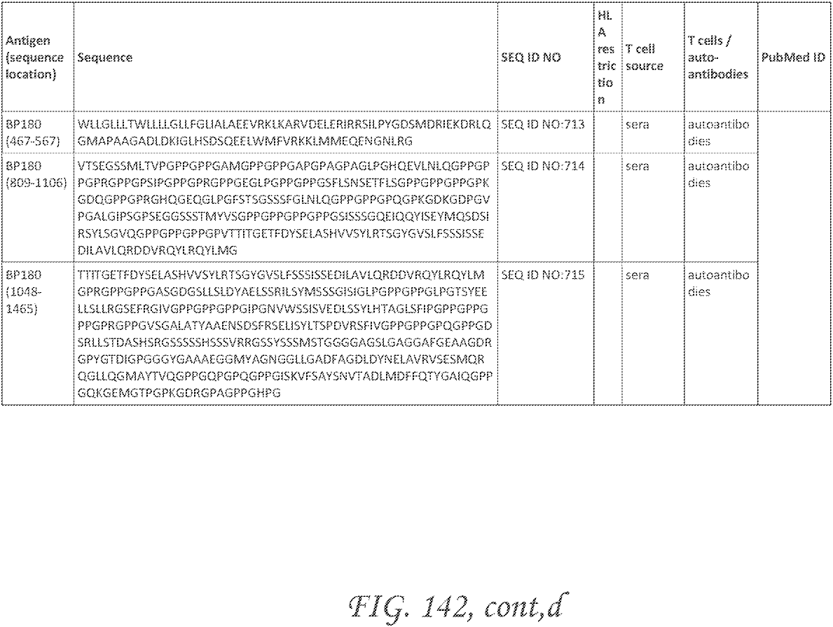

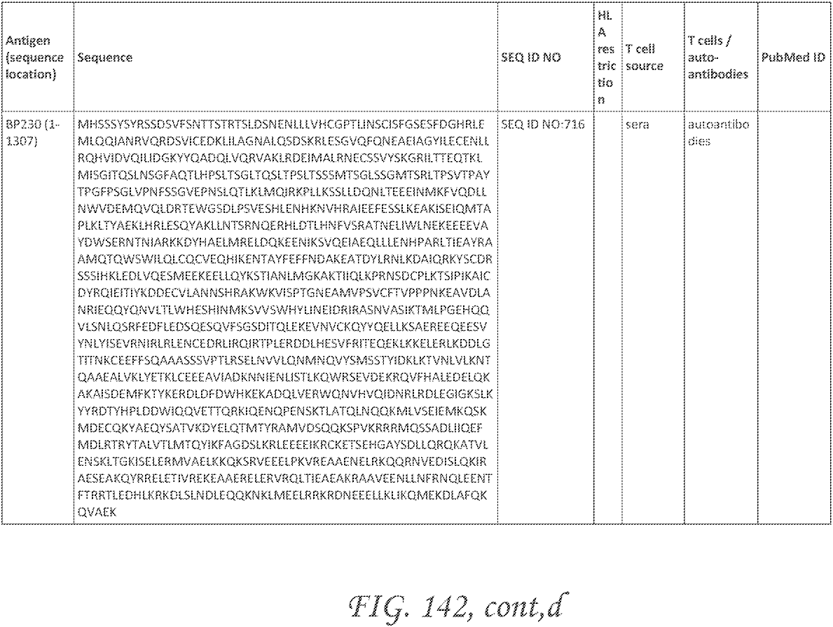

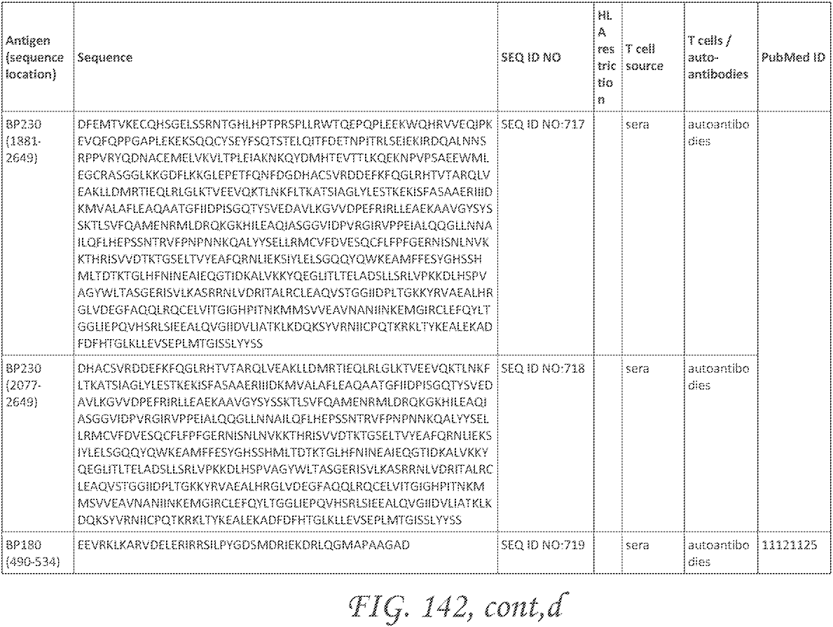

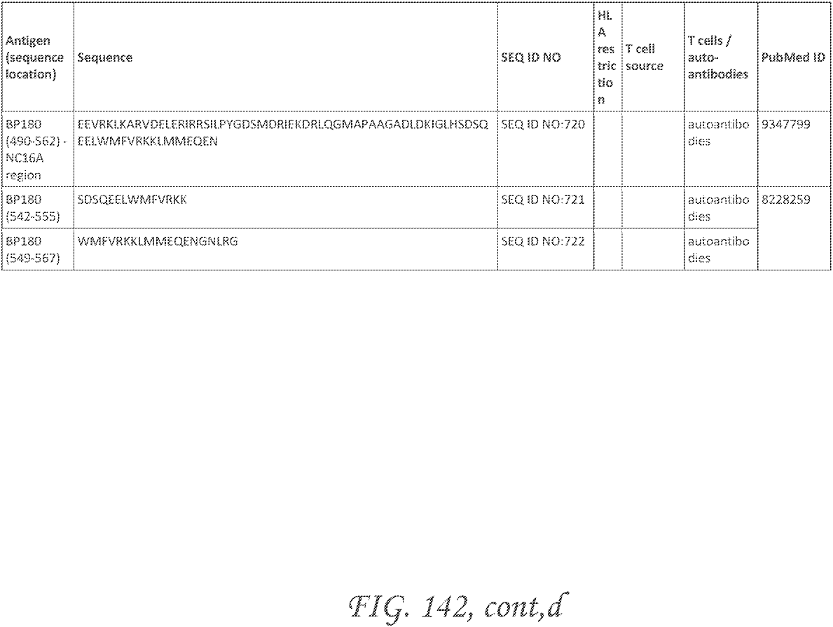

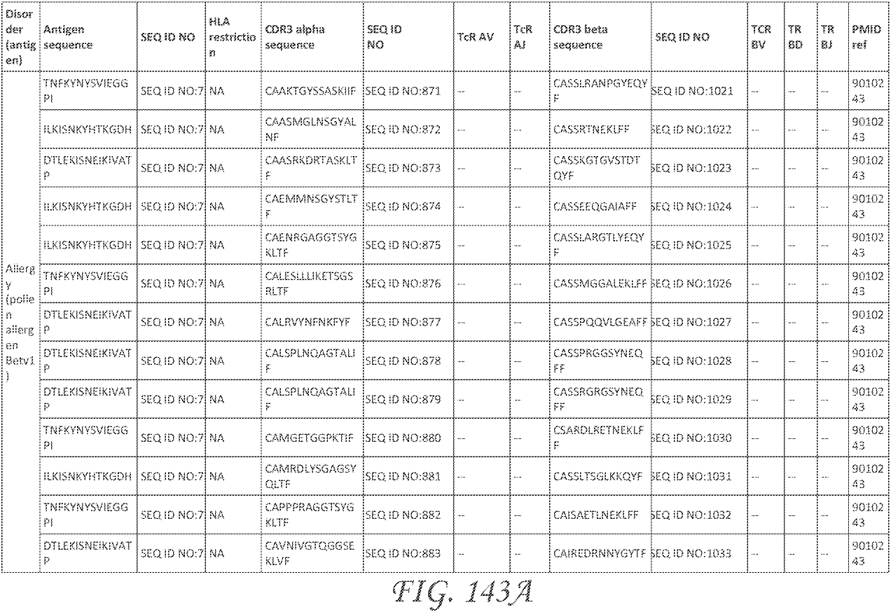

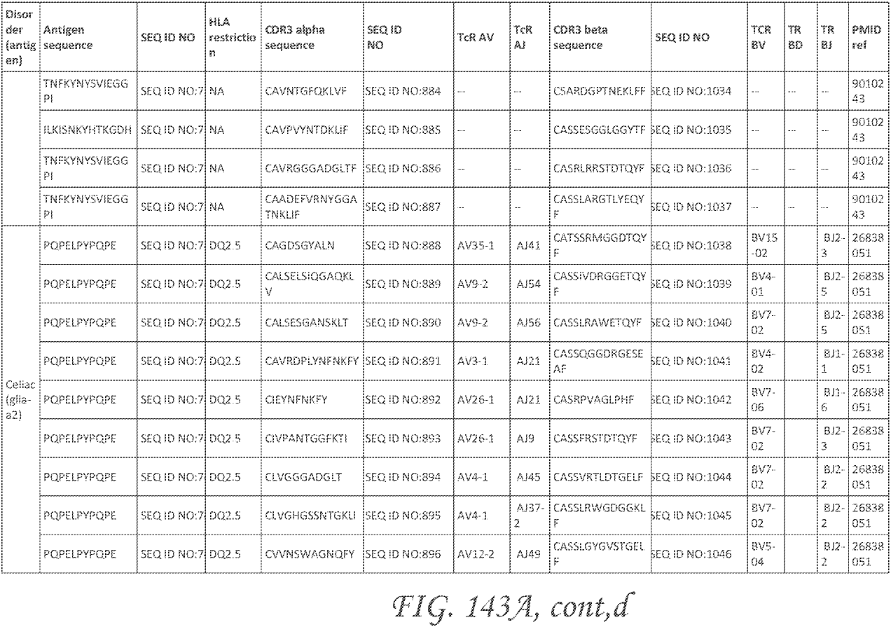

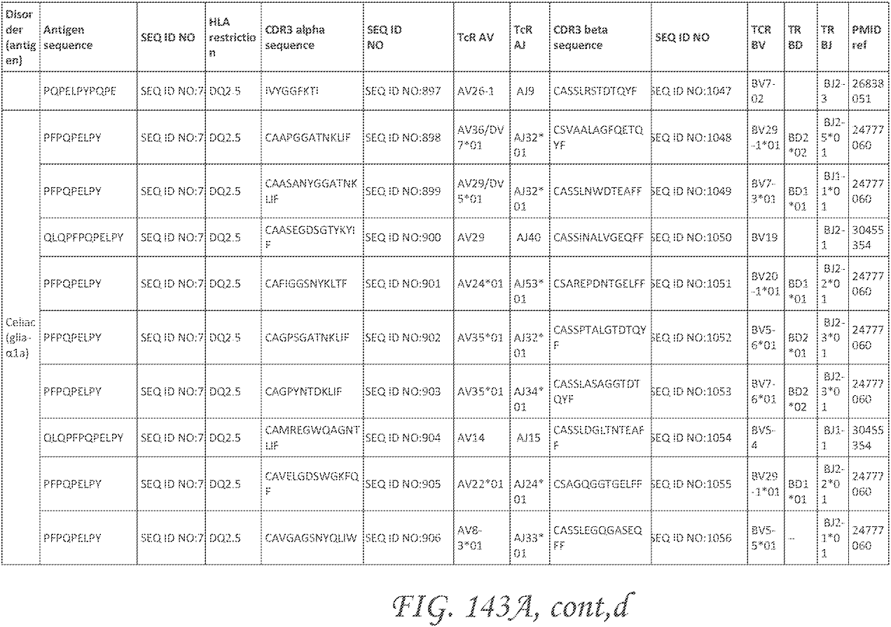

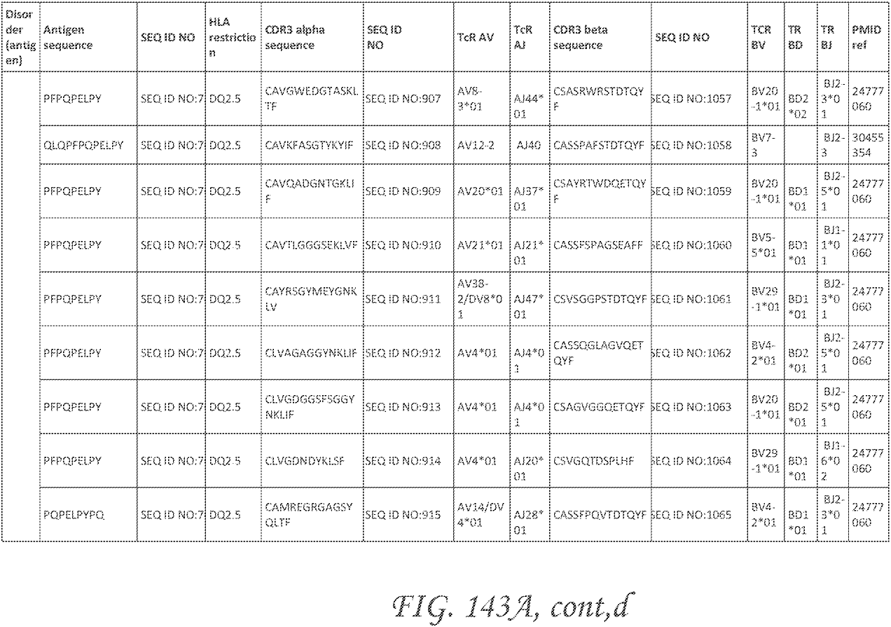

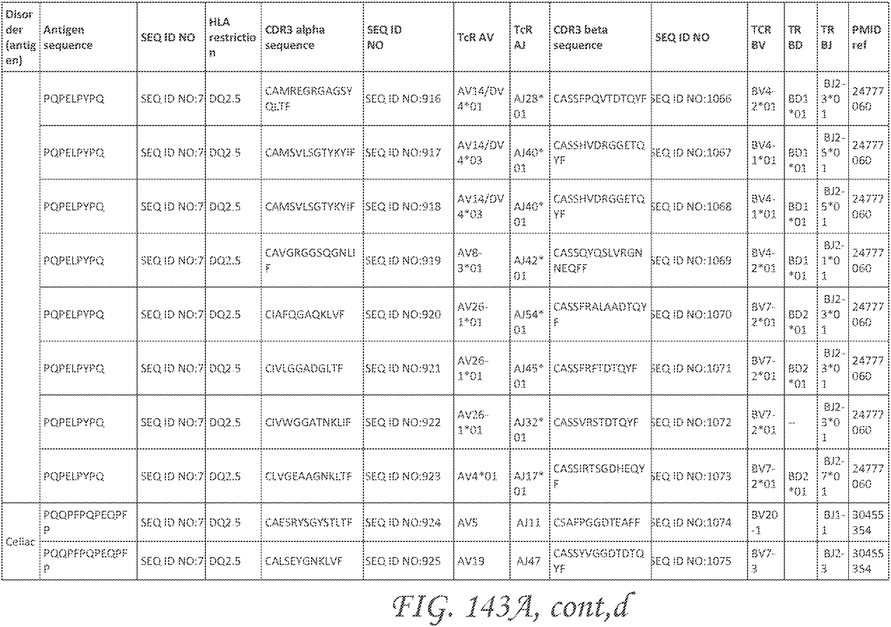

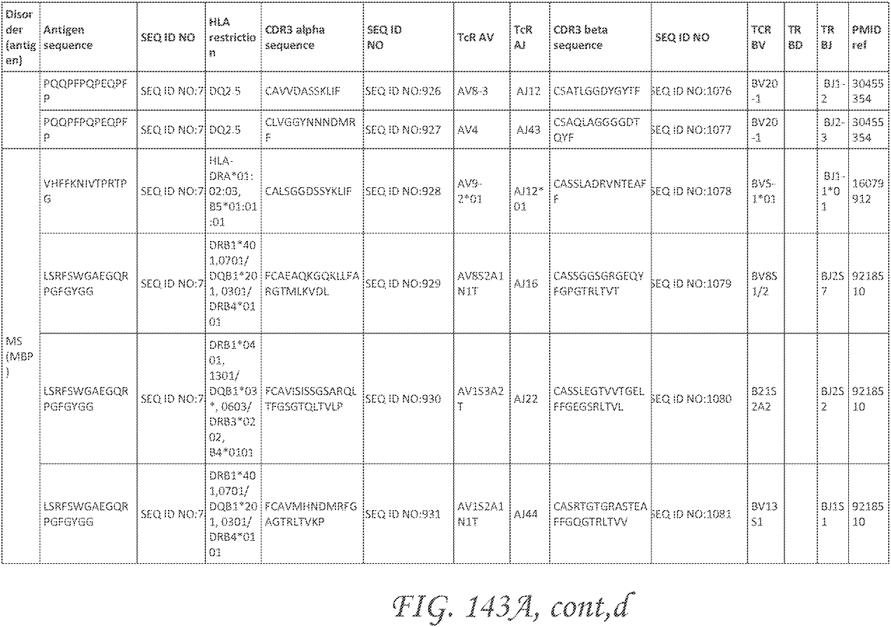

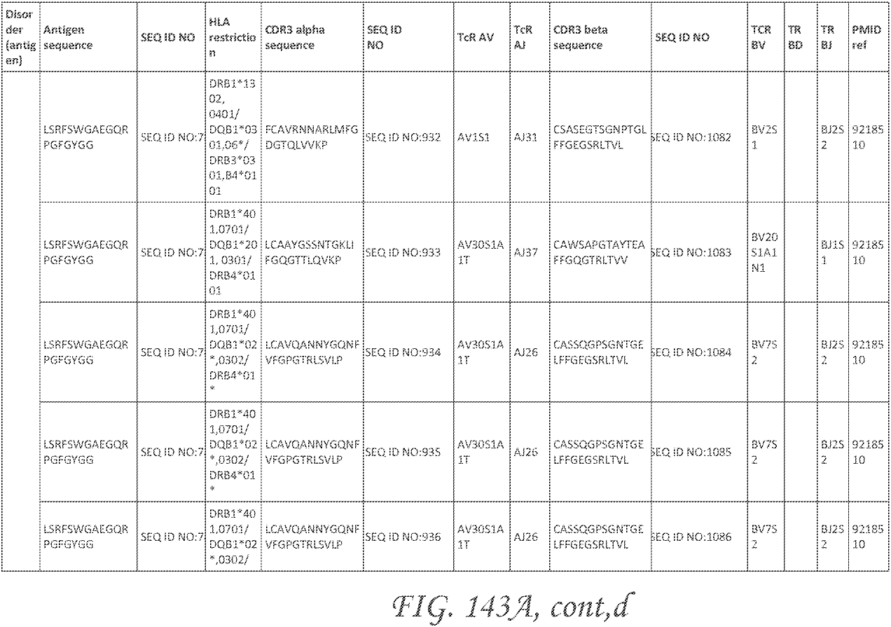

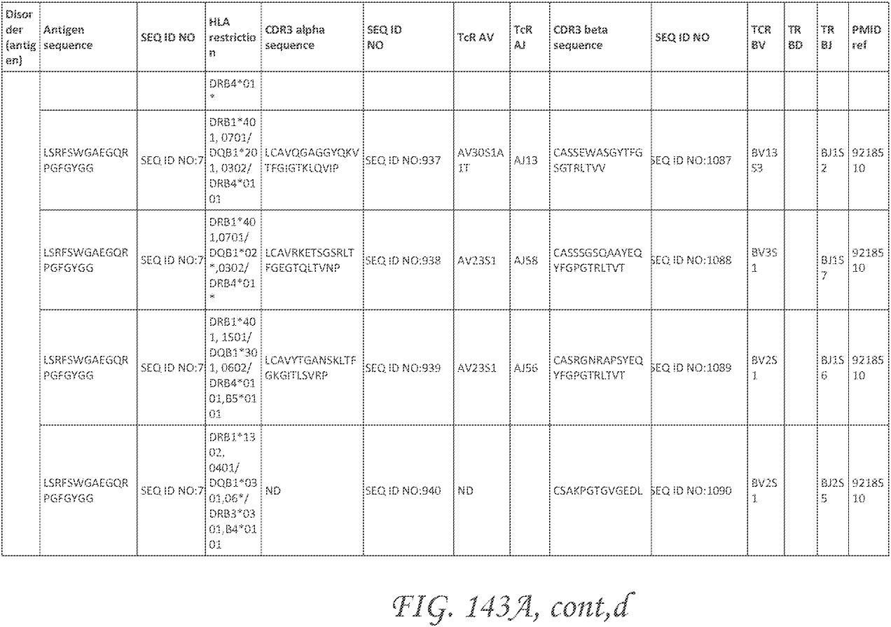

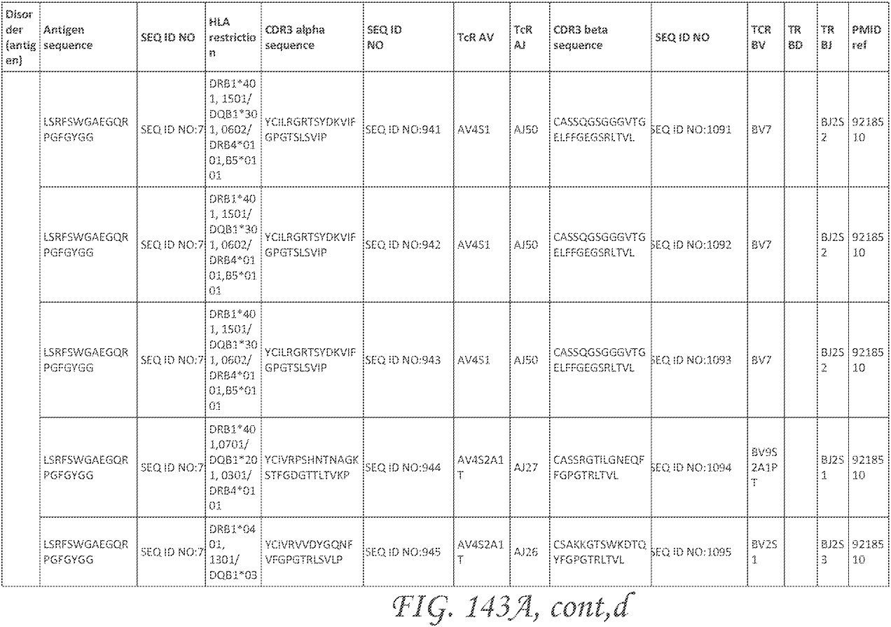

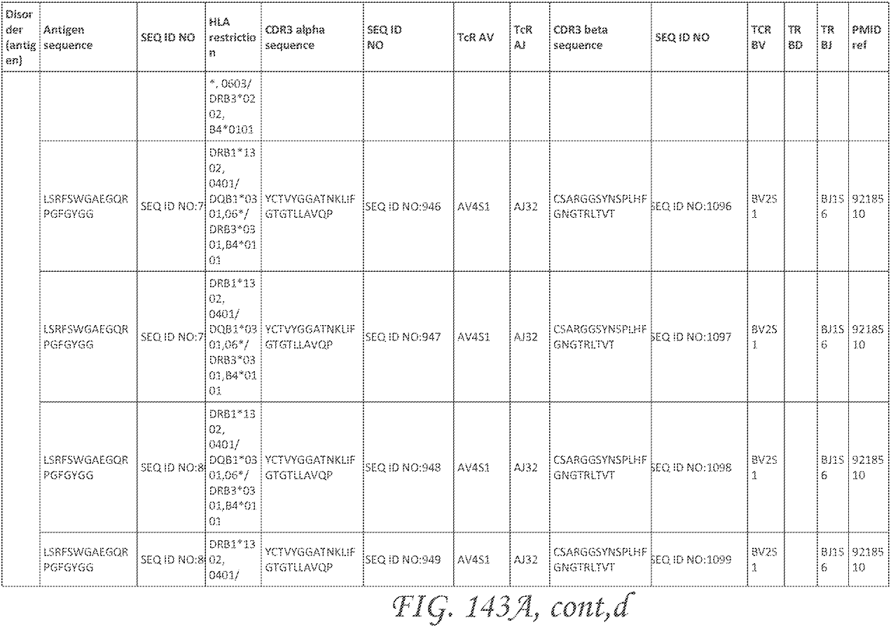

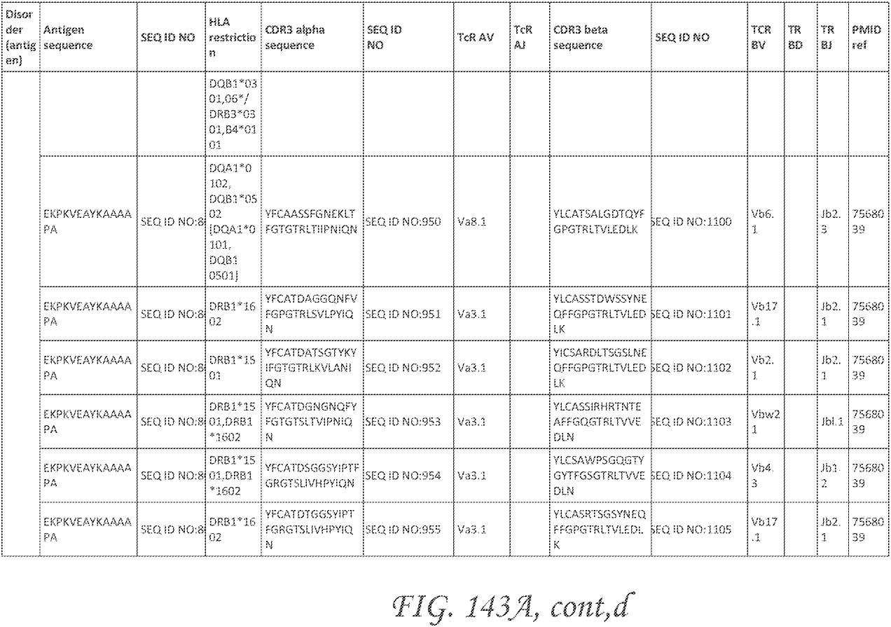

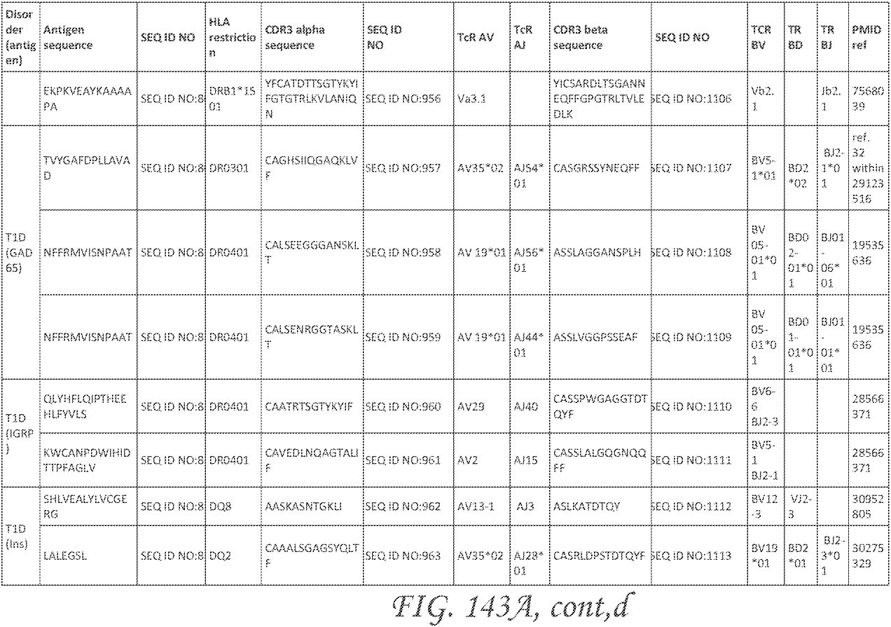

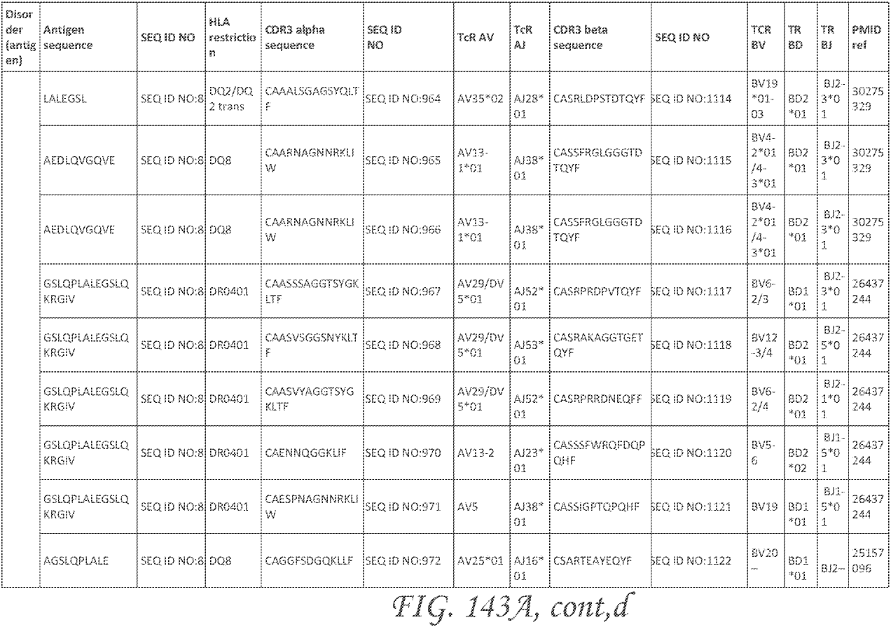

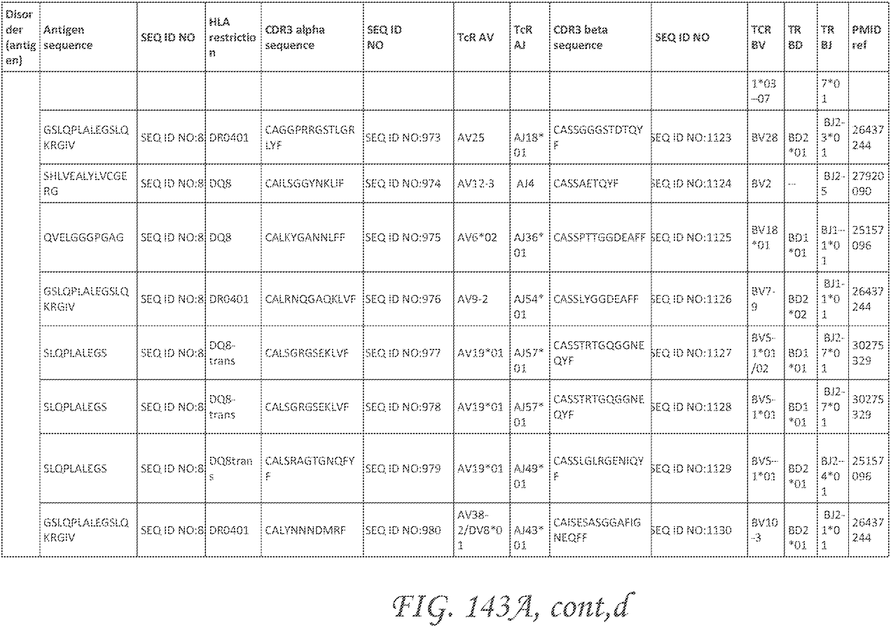

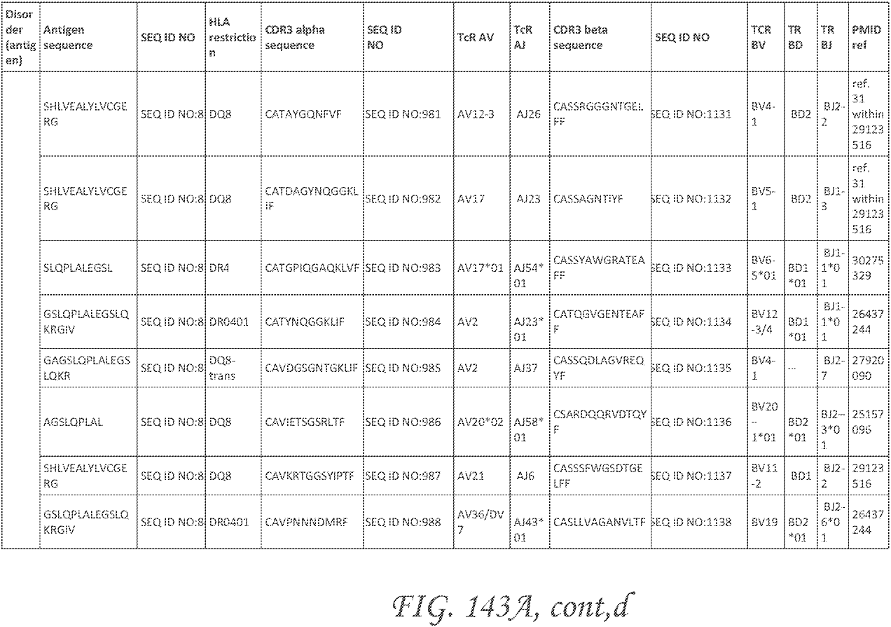

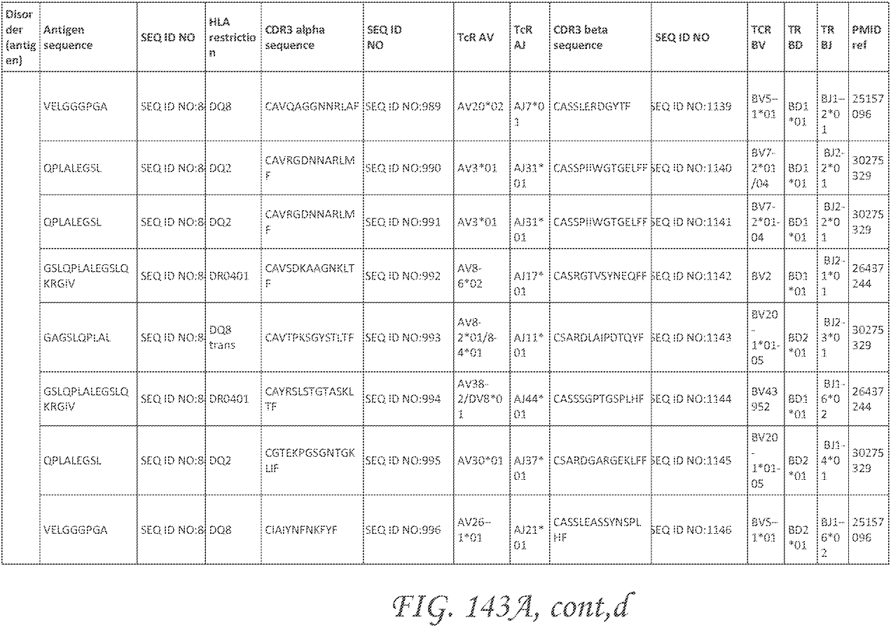

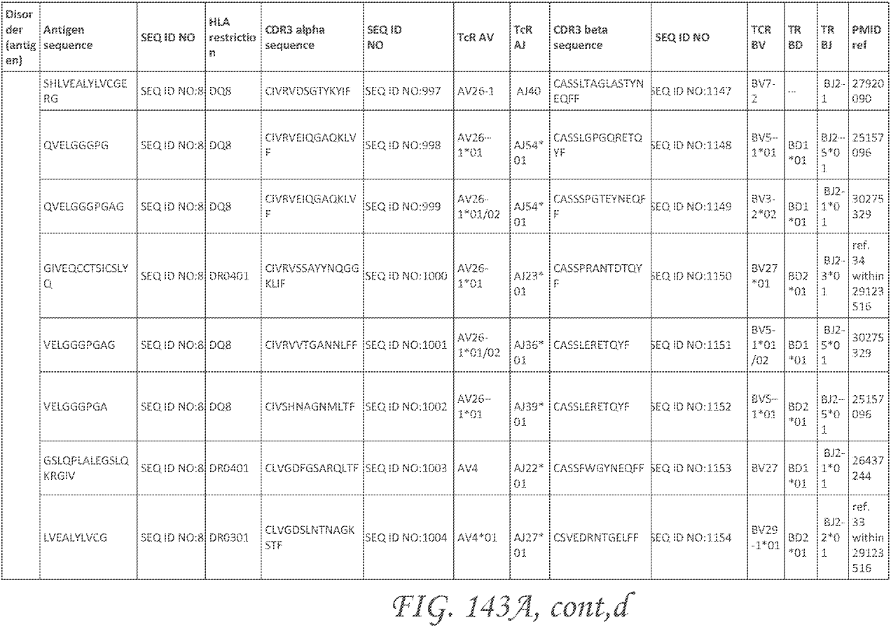

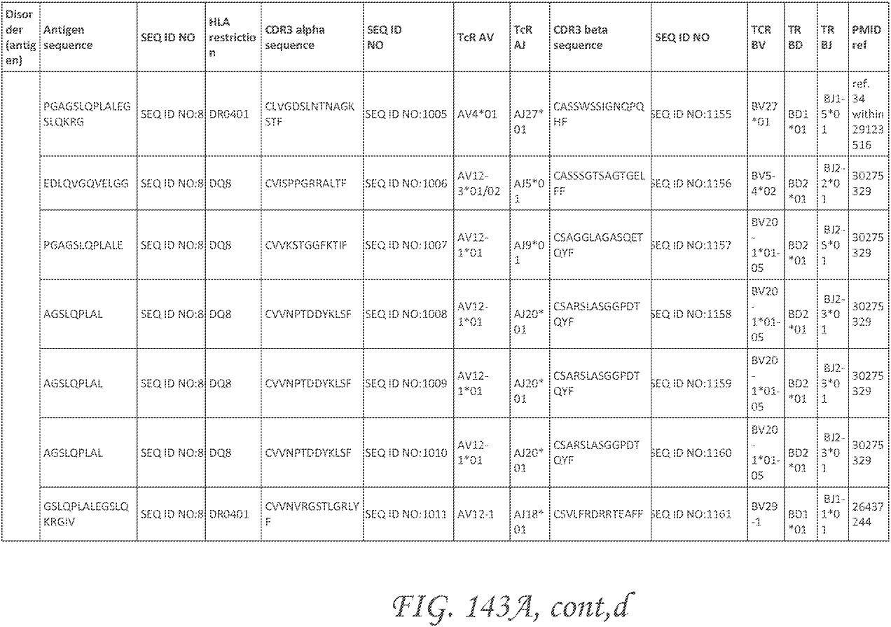

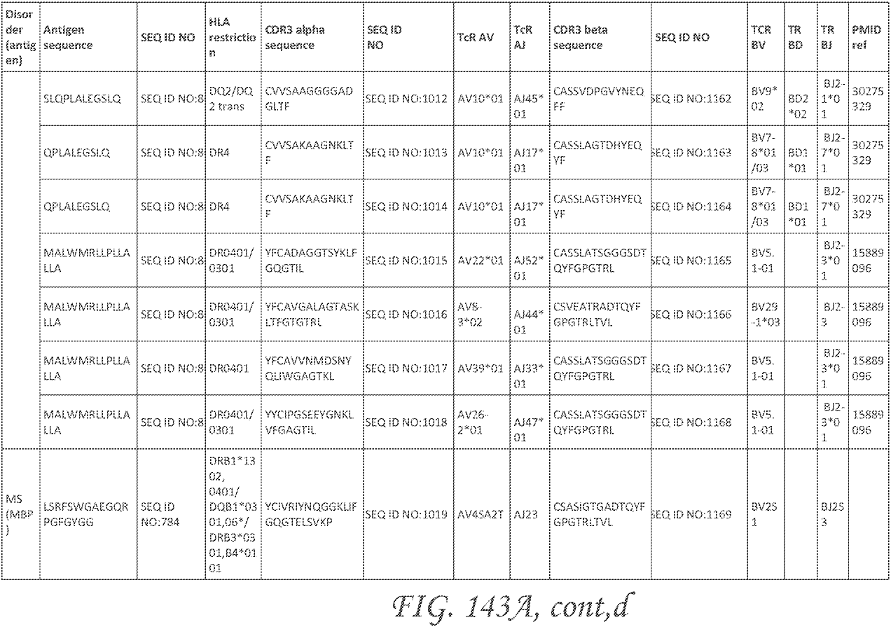

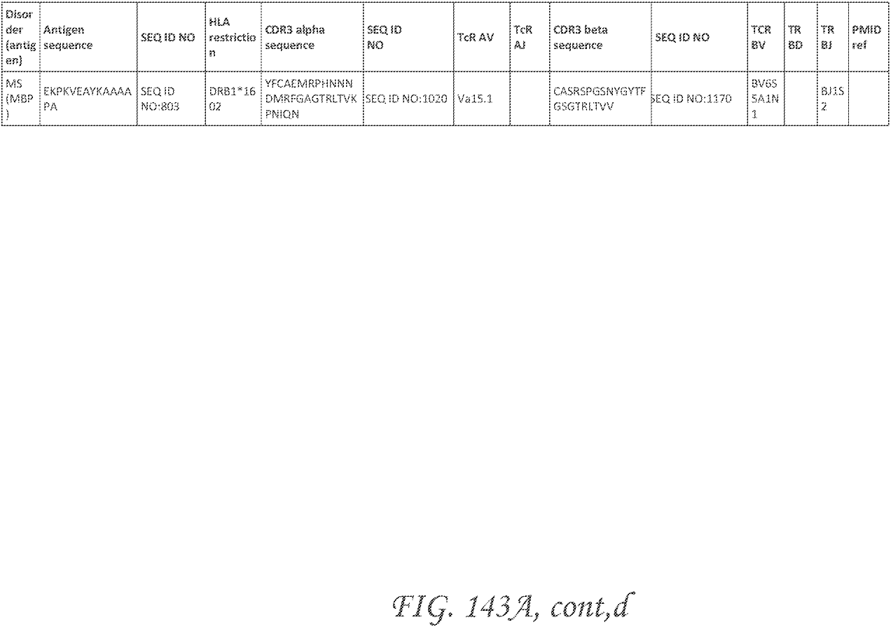

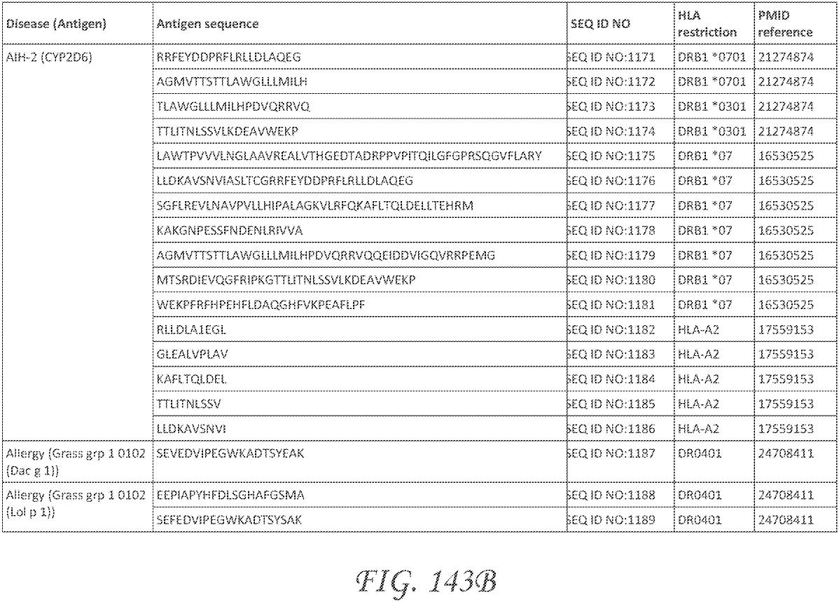

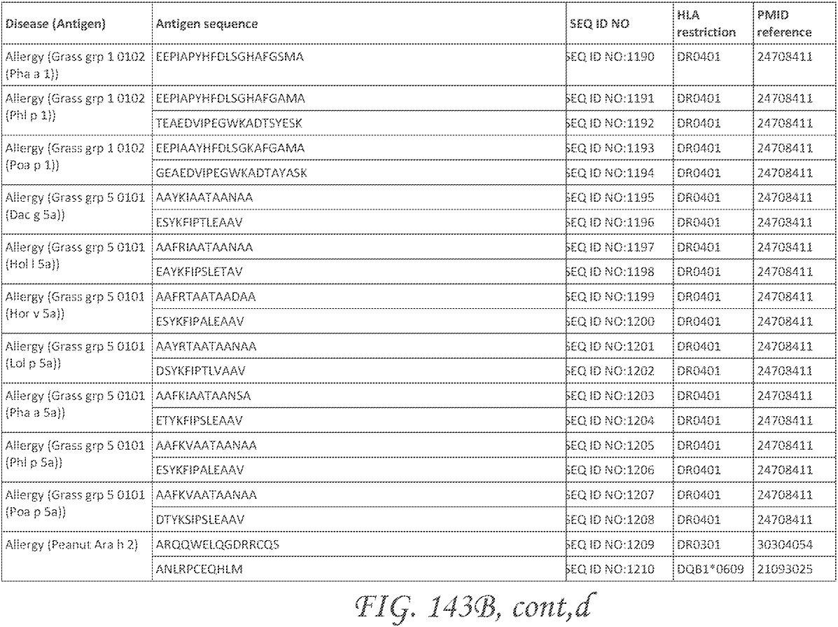

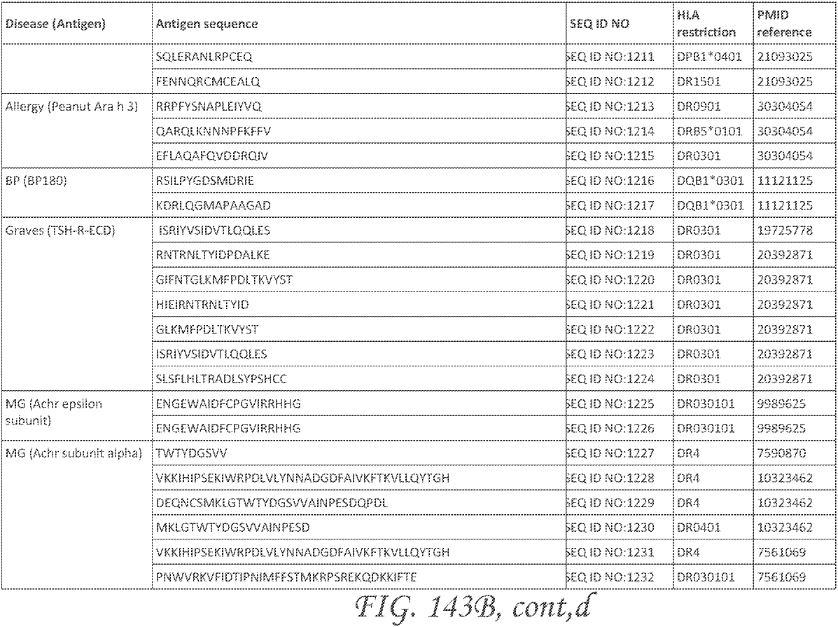

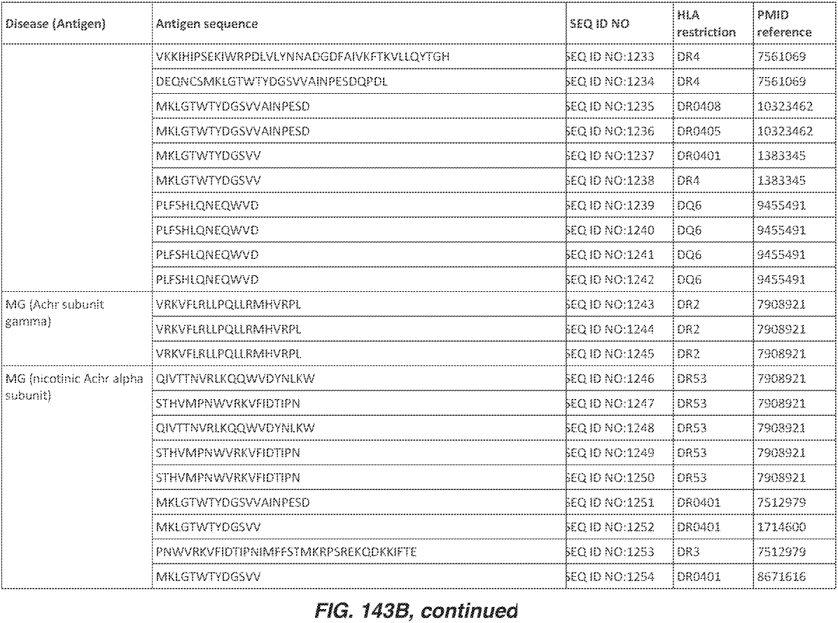

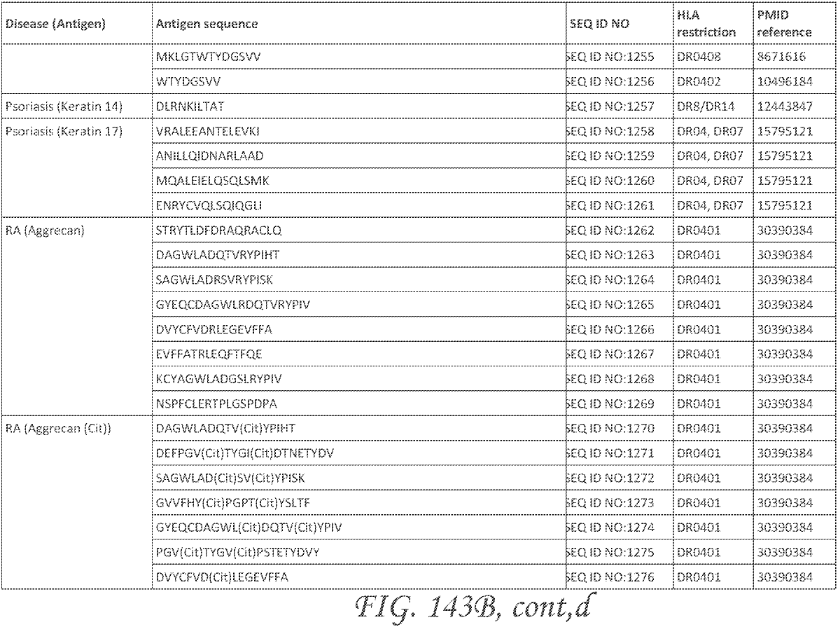

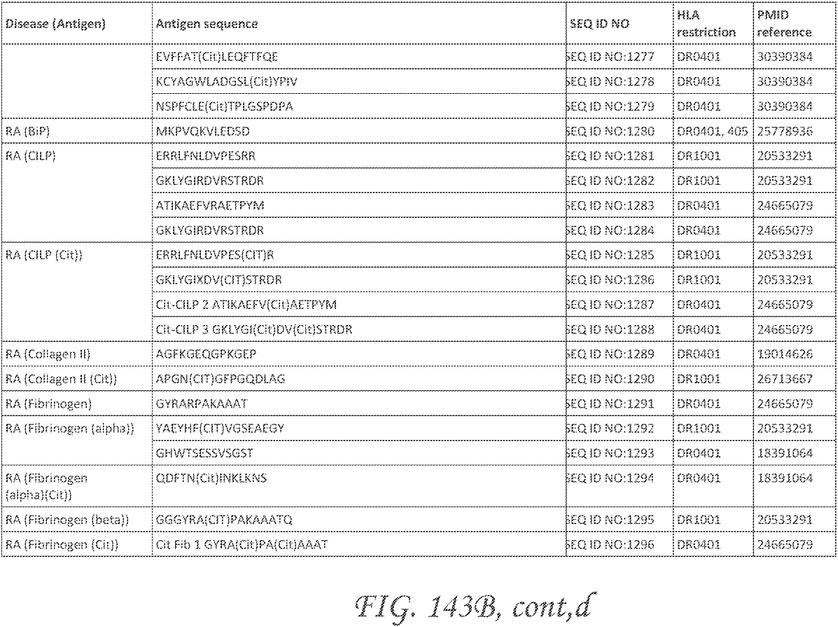

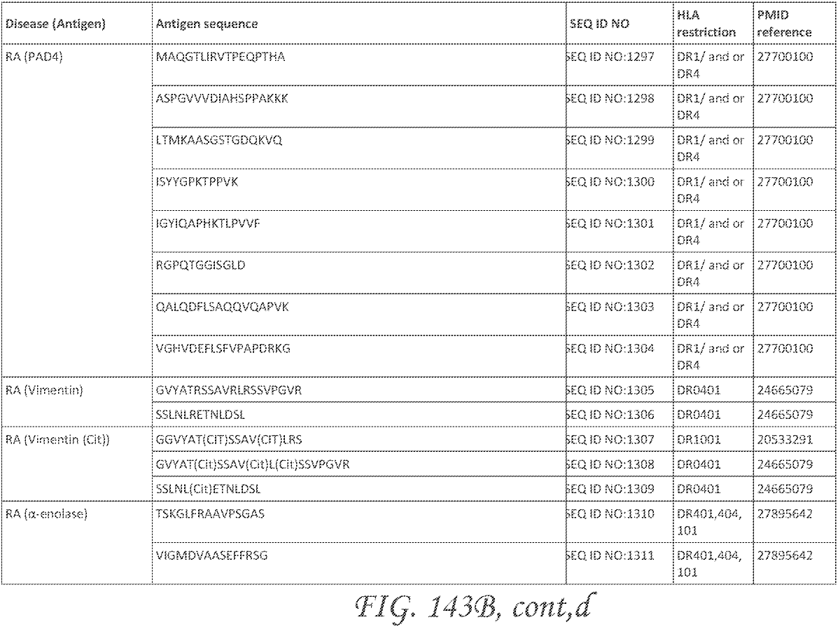

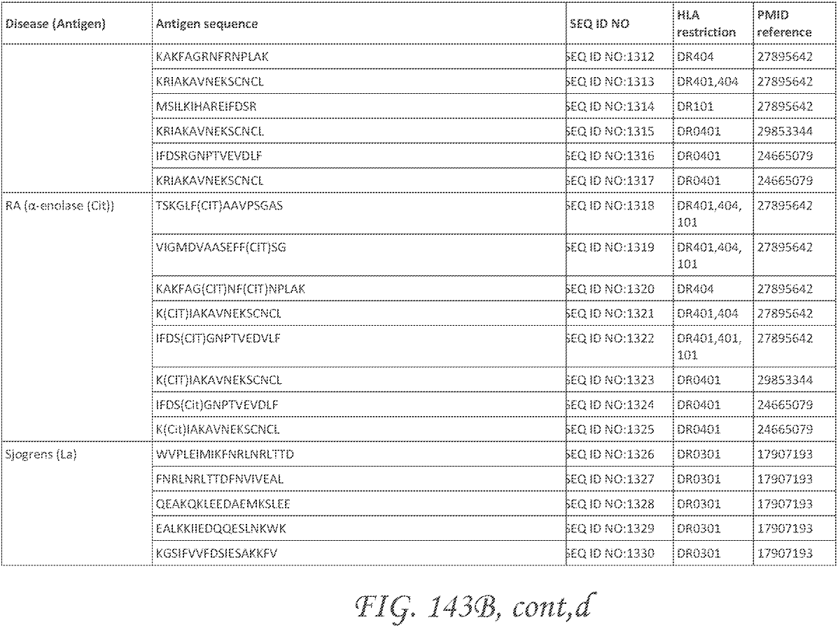

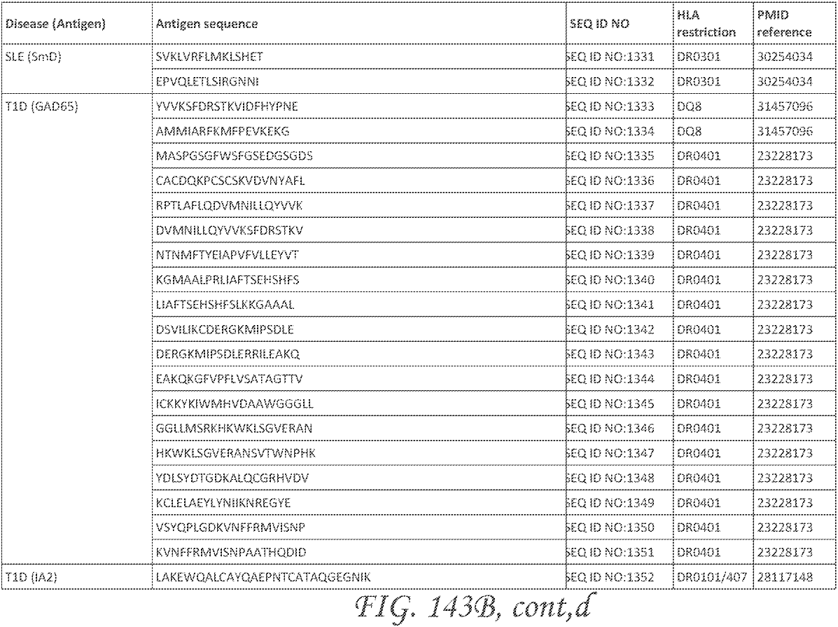

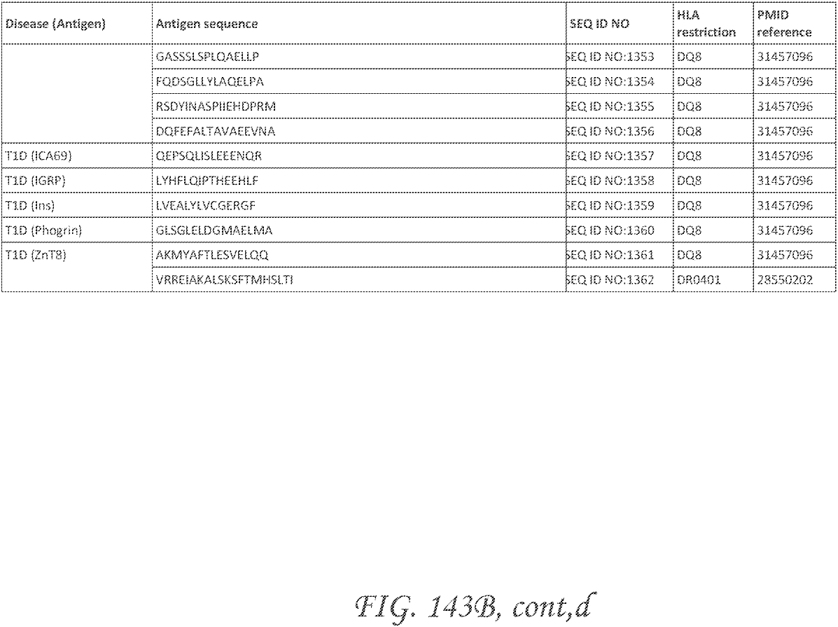

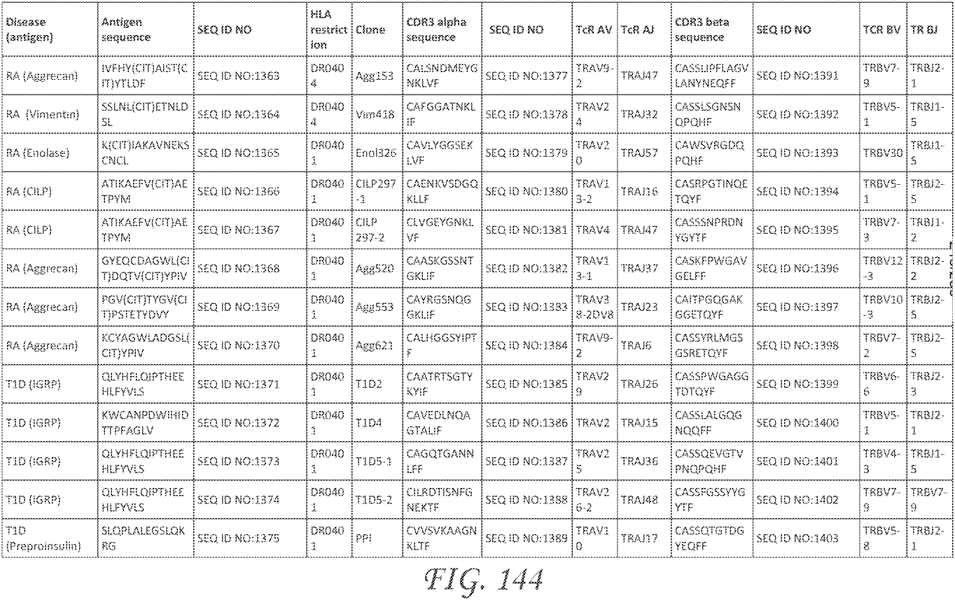

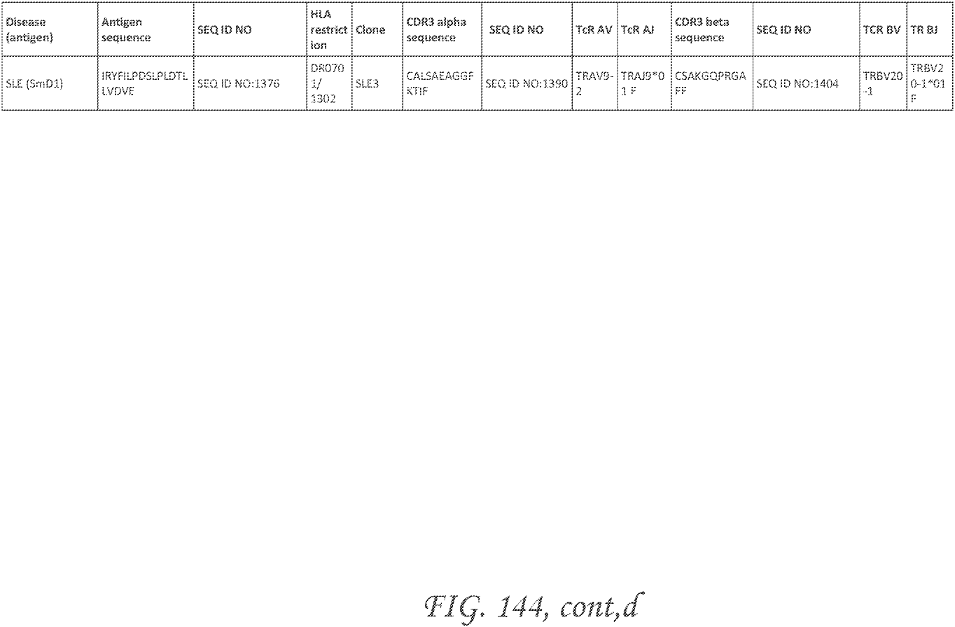

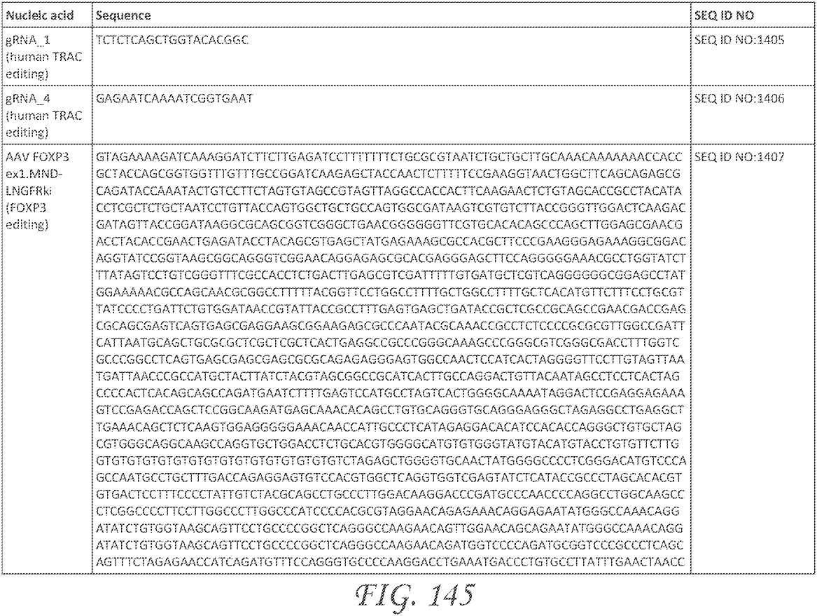

The two gene editing events that give rise in certain embodiments to expression in the present airT cells, of the TCR gene product concomitant with the first CISC component and of the FoxP3 gene product concomitant with the second CISC component concomitant, may be designed to take place in different alleles of the same gene locus (e.g., bi-allelic dual editing), or at two different gene loci (e.g., two-loci dual editing). Exemplary TCR amino acid and encoding nucleotide sequences are disclosed herein (e.g., FIGS. 136-144) for TCR that specifically recognize antigens associated with the pathogenesis of autoimmune, allergic, and/or inflammatory conditions.

### Gene Editing

As used herein, the term “chromosomal gene knockout” refers to a genetic alteration, inactivation, or introduced inhibitory agent in a host cell that prevents (e.g., reduces, delays, suppresses, or abrogates) production, by the host cell, of a functionally active endogenous polypeptide product. Alterations resulting in a chromosomal gene knockout or inactivation can include, for example, introduced nonsense mutations (including the formation of premature stop codons), missense mutations, gene deletion, or strand breaks, as well as the heterologous expression of inhibitory nucleic acid molecules that inhibit endogenous gene expression in the host cell.

In certain embodiments, a chromosomal gene knock-out or gene knock-in (e.g., insertion) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. Examples of endonucleases for use in gene editing include zinc finger nucleases (ZFN), TALE-nucleases (TALEN), CRISPR-Cas nucleases, meganucleases, or megaTALs.

The nucleic acid strand breaks caused by the endonuclease are typically double-strand breaks (DSB) that may be commonly repaired through the distinct mechanisms of homology directed repair (HDR) by homologous recombination, or by non-homologous end joining (NHEJ). (NHEJ: Ghezraoui et al., 2014 Mol Cell 55(6):829-842; HDR: Jasin and Rothstein, 2013 Cold Spring Harb Perspect Biol 5(11):a012740, PMID 24097900) During HDR/ homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. HDR is favored by the presence of a donor template at the time of DSB formation and is a preferred gene editing mechanism according to certain herein described embodiments.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair (HDR). Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout or inactivation comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Pat. Publication No. US 2011/0301073, which atypical RVDs are incorporated by reference herein in their entirety). TALENs can be used to direct site-specific double-strand breaks (DSB) in the genome of T cells. Non- homologous end joining (NHEJ) ligates DNA from both sides of a double-strand break in which there is little or no sequence overlap for annealing, thereby introducing errors that knock out gene expression. Alternatively, homology directed repair (HDR) can introduce a transgene at the site of DSB providing homologous flanking sequences are present in the donor template containing the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a donor template transgene with homologous flanking sequences can be introduced at the site of DSB via homology directed repair (HDR). The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference).

In certain embodiments, a gene knockout or inactivation comprises an insertion, a deletion, a mutation or a combination thereof, and made using a CRISPR/Cas nuclease system. US/2016/033377 which is expressly incorporated by reference in its entirety, teaches methods for enhancing endonuclease based gene editing, including AAV-expressed guide RNAs for use in CRISPR/Cas (e.g., Cas9) gene editing systems. Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby expressly incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, FasL, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Pat. Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out or inactivate one or more target genes, but to also introduce (knock in) heterologous or exogenous polynucleotides when used in combination with an exogenous donor template encoding a polypeptide of interest.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knockout or inactivation comprises a knockout or inactivation of a TCR component gene selected from a TCR α variable region gene, a TCR β variable region gene, a TCR constant region gene, or a combination thereof.

### T Cells and TCR

The present CD4+CD25+ airT cell comprises an artificial modification of a FOXP3 gene as described herein and further comprises at least one transduced polynucleotide encoding an antigen-specific T cell receptor (TCR) polypeptide. Preferably and in certain embodiments, the native TCR gene has been knocked out, for example by a targeted gene editing knock out in the TCR alpha (TRAC) gene locus. As used herein “knocked out” can refer to the inactivation of a gene and/or the gene product, for example, such as by deletion of the gene or a portion of the gene, by insertion of nucleic acids into the gene to interrupt transcription and/or translation of the gene and/or its product. Also preferably and in certain embodiments, the transduced polynucleotide encoding the TCR has been knocked in by gene editing to a specific gene locus, such as the TRAC gene locus or another targeted locus.

The present airT cells thus comprise artificial immunoregulatory T cells that in preferred embodiments are produced by selective editing of one or more specific gene loci in T lymphocytes as described herein. Preferred T cells are of mammalian origin, for example, T cells obtained from humans, non-human primates (e.g., chimpanzees, macaques, gorillas, etc.), rodents (e.g., mice, rats, etc.), lagomorphs (e.g., rabbits, hares, pikas, etc.), ungulates (e.g., cattle, horses, pigs, sheep, etc.), or other mammals. In certain preferred embodiments the T cells are human T cells.

A T cell or T lymphocyte is an immune system cell that matures in the thymus and produces a T cell receptor (TCR), e.g., an antigen-specific heterodimeric cell surface receptor typically comprised of an alpha-beta heterodimer or a gamma-delta heterodimer. T cells of a given clonality typically express only a single TCR clonotype that recognizes a specific antigenic epitope presented by a syngeneic antigen-presenting cell in the context of a major histocompatibility complex-encoded determinant. T cells can be naive (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+ CD25+ (Foxp3+) regulatory T cells and Treg17 cells, as well as Tr1, Th3, CD8+CD28-, and Qa-1 restricted T cells.

As used herein, “T cell receptor” (TCR) refers to an immunoglobulin superfamily member having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997. The TCR is capable of specifically binding to an antigen peptide bound to a major histocompatibility complex encoded (MHC) receptor. A TCR can be found on the surface of a T cell or may be released into the extracellular milieu in soluble form, and generally is comprised of a heterodimer having α and β chains (also known as TCR α and TCRβ, respectively), or γ and δ chains (also known as TCRγ and TCRδ, respectively), each having chain-characteristic constant (C) regions and highly polymorphic variable (V) regions in which reside complementarity determining regions (CDR) that are largely responsible for specific antigen recognition and binding by the TCR. In certain embodiments, a polynucleotide encoding a TCR can be codon optimized to enhance expression in a particular host cell, such as, for example, a cell of the immune system, a hematopoietic stem cell, a T cell, a primary T cell, a T cell line, a NK cell, or a natural killer T cell (Scholten et al., Clin. Immunol. 119:135, 2006).

Exemplary T cells that can express TCRs encoded by heterologous genetic material introduced in the cells according to certain embodiments of this disclosure include CD4+ T cells, CD8+ T cells, and related subpopulations thereof (e.g., naive, central memory, stem cell memory, effector memory). In preferred embodiments the exemplary T cells are CD4+ T cells, the TCR-encoding genetic material is introduced by gene editing (e.g., homology directed repair following a specifically targeted double-strand break in genomic DNA), and the TCR specifically recognizes an antigen associated with pathogenesis of an autoimmune condition, an allergic condition, or an inflammatory condition, such as the specific TCRs that are structurally defined herein or TCRs that specifically recognize the particular autoantigen, allergen, or inflammatory disease antigen T cell epitopes that are disclosed herein.

Like other antigen-binding members of the immunoglobulin superfamily (e.g., the immunoglobulins, also referred to as antibodies), the extracellular portion of TCR chains (e.g., α-chain, β-chain) contain two immunoglobulin domains, a variable domain (e.g., α-chain variable domain or Vα, β-chain variable domain or Vβ; typically amino acids 1 to 116 based on Kabat numbering (Kabat et al., “ Sequences of Proteins of Immunological Interest, US Dept. Health and Human Services, Public Health Service National Institutes of Health, 1991, 5th ed.)) at the N-terminus, and one constant domain (e.g., α-chain constant domain or Cα, typically 5 amino acids 117 to 259 based on Kabat, β-chain constant domain or Cβ, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat′l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, non-human primate, mouse, rat, rabbit, or other mammal.

The term “variable region” or “variable domain” refers to the structural domain of an immunoglobulin superfamily binding protein (e.g., a TCR α-chain or β-chain (or γ chain and δ chain for γδ TCRs)) that is involved in specific binding of the immunoglobulin superfamily binding protein (e.g., TCR) to antigen. The variable domains of the α chain and β chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are synonymous with “hypervariable region” or “HVR,” and are known in the art to refer to sequences of amino acids within immunoglobulin (e.g., TCR) variable regions, which confer antigen specificity and/or binding affinity and are separated from one another in primary amino acid sequence by framework regions. In general, there are three CDRs in each TCR α-chain variable region (αCDR1, aCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3 is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php).

As used herein, “CD4” is an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHC class II molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and to promote immune responses.

In certain embodiments, a TCR is found on the surface of T cells (or T lymphocytes) and associates with a CD3 complex. “CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD3δ chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine-based activation motif or ITAM, whereas each CD3ζ chain has three such motifs. Without wishing to be bound by theory, it is believed that the ITAMs are important for the signaling capacity of a TCR complex. CD3 as used in the present disclosure may be from various animal species, including human, non-human primate, mouse, rat, or other mammals.

As used herein, “TCR complex” refers to a complex formed by the association of CD3 with TCR. For example, a TCR complex can be composed of a CD3γ chain, a CD3β chain, two CD3ε chains, a homodimer of CD3ζ chains, a TCRα chain, and a TCRβ chain. Alternatively, a TCR complex can be composed of a CD3γ chain, a CD3β chain, two CD3ε chains, a homodimer of CD3ζ chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, “Chimeric antigen receptor” (CAR) refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell such as a T cell. CARs can include an extracellular portion comprising an antigen-binding domain (e.g., obtained or derived from an immunoglobulin or immunoglobulin-like molecule, such as a TCR antigen binding domain derived or obtained from a TCR specific for an autoantigen, an allergen, or an inflammatory disease-associated antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein).

Many polypeptides may, as encoded by a polynucleotide sequence, comprise a “signal peptide” (also known as a leader sequence, leader peptide, or transit peptide). Signal peptides target newly synthesized polypeptides to their appropriate location inside or outside the cell. A signal peptide may be removed from the polypeptide during biosynthesis or after subcellular localization or extracellular secretion of the polypeptide is completed. Polypeptides that have a signal peptide are referred to herein as a “pre-protein” and polypeptides having their signal peptide removed are referred to herein as “mature” proteins or polypeptides.

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids or 2-35 amino acids, for instance, about four to about 20 amino acids or 4-20 amino acids, about eight to about 15 amino acids or 8-15 amino acids, about 15 to about 25 amino acids or 15-25 amino acids. Exemplary linkers include glycine-serine linkers as are known in the art.

Exemplary TCR V region sequences and polynucleotide sequences coding therefor are disclosed herein, including in the Examples and Drawings, for TCR that specifically recognize antigens associated with autoimmune, allergic, and inflammatory conditions as provided herein. Also disclosed herein, including in the Examples and Drawings, are polypeptide sequences containing TCR-recognized antigenic epitopes of antigens associated with autoimmune, allergic, and inflammatory conditions as provided herein.

An “antigen” typically refers to an immunogenic molecule that provokes an immune response. This immune response may involve production of antibodies that specifically bind to the antigen, activation of specific immunologically competent cells (e.g., T cells such as T-helper, T-effector, Treg, etc.), or both. Although an antigen may frequently be thought of as a “non-self” structure to which a host immune system responds by recognizing the antigen as foreign, in the present disclosure “antigen” is not intended to be so limited and may in certain embodiments also include any autoantigen, which refers to a “self” molecular, cellular, organ, or tissue structure to which a host immune system may react inappropriately in the context of autoimmune disease. An antigen (immunogenic molecule) may be, for example, a peptide, glycopeptide, polypeptide, glycopolypeptide, polynucleotide, polysaccharide, lipid or the like. It is readily apparent that an antigen can be synthesized artificially, produced recombinantly, or derived from a biological sample. Exemplary biological samples that can contain one or more antigens include tissue samples, cells, biological fluids, biopsies, primary cultures, or combinations thereof. Antigens can be produced by cells that have been modified or genetically engineered to express an antigen, or that endogenously (e.g., without modification or genetic engineering by human intervention) express a mutation or polymorphism that is immunogenic.

In certain embodiments a T cell as provided herein may be used as a host cell that may be modified to include one or more heterologous polynucleotides comprising regulatory sequences (e.g., promoters, enhancers, etc.) and/or nucleic acid sequences encoding a desired TCR and/or nucleic acid sequences encoding all or a portion of a FoxP3 transcription factor as described herein. Methods for transfecting/transducing T cells with desired nucleic acids have been described (e.g., U.S. Pat. Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein. Particularly preferred embodiments relate to artificial modification of a T cell genome by targeted gene editing as described herein.

Any appropriate method can be used to transfect or transduce the cells, for example, the T cells, or to administer the polynucleotides or compositions of the present methods. Known methods for delivering polynucleotides to host cells include, for example, use of cationic polymers, lipid-like molecules, and certain commercial products such as, for example, IN-VIVO-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, as also described herein and known to the art.

A nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise. The term “isolated polynucleotide” as used herein shall mean a polynucleotide of genomic, cDNA, or synthetic origin or some combination thereof, wherein by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

In certain embodiments the present airT cell may be gene edited so as to express a FoxP3 gene product that is encoded by a FoxP3-encoding nucleotide sequence that is operably linked to a constitutive promoter, wherein constitutive expression of the FoxP3 gene product refers to a FOXP3 expression level that is equal to or greater than the FOXP3 expression level of a naturally occurring regulatory T (Treg) cell. In certain preferred embodiments the constitutive promoter is the MND promoter, and in certain preferred embodiments the MND promoter has been knocked-in to the native FOXP3 gene locus by HDR gene editing. In certain embodiments the constitutively active promoter is knocked-in downstream of an intronic regulatory T cell (Treg)-specific demethylation region (TSDR) in the native FOXP3 gene locus. In certain embodiments, a nucleic acid molecule comprising an exogenous FOXP3-encoding polynucleotide operably linked to the constitutive promoter is knocked-in by HDR gene editing to the native FOXP3 gene locus. In certain embodiments, a nucleic acid molecule comprising an exogenous FOXP3-encoding polynucleotide operably linked to the constitutive promoter is knocked-in by HDR gene editing at a chromosomal site other than the native FOXP3 gene locus, such as a TRAC gene locus or an AAVS1 locus or another gene locus. Accordingly, in these and related embodiments, the present disclosure for the first time teaches certain unexpected advantages that are associated with artificial gene editing by which FOXP3 gene expression is regulated by the constitutively active promoter, and in particularly preferred embodiment by the constitutively active MND promoter, for the production of the presently described engineered artificial immunoregulatory T (airT) cells.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate or phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, or phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, AntiCancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby expressly incorporated by reference in their entireties. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

In other related embodiments, polynucleotide variants may have substantial identity to a polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity or a sequence identity that is within a range defined by any two of the aforementioned percentages as compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a polypeptide variant of a given polypeptide which is capable of a specific binding interaction with another molecule and is encoded by the variant polynucleotide is not substantially diminished relative to a polypeptide encoded by a polynucleotide sequence specifically set forth herein.

In certain other related embodiments, polynucleotide fragments may comprise or consist essentially of various lengths of contiguous stretches of sequence identical to or complementary to a sequence encoding a polypeptide as described herein. For example, polynucleotides are provided that comprise or consist essentially of at least or at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes a polypeptide, or variant thereof, disclosed herein, as well as, all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding a polypeptide described herein or at both ends of a polynucleotide encoding a polypeptide described herein.

In another embodiment, polynucleotides are provided that are capable of hybridizing under moderate to high stringency conditions to a polynucleotide sequence encoding a polypeptide, or variant thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5 X SSC, 0.5% SDS, 1.0 mM EDTA (pH 8.0); hybridizing at 50° C.-60° C., 5 X SSC, overnight; followed by washing twice at 65° C. for 20 minutes with each of X, 0.5X and 0.2X SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C. 70° C.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of or about of 10,000, 5000, 3000, 2,000, 1,000, 500, 200,100, or 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M.O. (1978) A model of evolutionary change in proteins - Matrices for detecting distant relationships. In Dayhoff, M.O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D.G. and Sharp, P.M., CABIOS 5:151-153 (1989); Myers, E.W. and Muller W., CABIOS 4:11-17 (1988); Robinson, E.D., Comb. Theor 11:105 (1971); Santou, N. Nes, M., Mol. Biol. Evol. 4:406-425 (1987); Sneath, P.H.A. and Sokal, R.R., Numerical Taxonomy -the Principles and Practice of Numerical Taxonomy, Freeman Press, San Francisco, CA (1973); Wilbur, W.J. and Lipman, D.J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=-4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a FoxP3, TCR, or antigenic peptide as described herein, or an antibody that specifically binds to such a peptide, as described herein. Some of these polynucleotides bear minimal sequence identity to the nucleotide sequence of the native or original polynucleotide sequence that encode FoxP3, TCR, or antigenic polypeptides described herein. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the FoxP3, TCR, or antigenic polypeptides described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences that encode a FoxP3, TCR, or antigenic polypeptide disclosed herein, or a variant thereof, to alter one or more properties of the encoded polypeptide, such as (e.g., for TCR or antigenic peptides) the binding affinity of the peptide or the variant thereof for a cognate ligand, or (e.g., for FoxP3) the immunosuppressive effects. The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically 14 to 25 nucleotides or about 14 to about 25 nucleotides or so in length is employed, with about 5 to about 10 residues or 5 to 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence encoding the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated or contacted with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U. S. Pat. No. 4,237,224, expressly incorporated herein by reference in its entirety.

In another approach for the production of polypeptide variants, recursive sequence recombination, as described in U.S. Pat. No. 5,837,458 which is expressly incorporated by reference in its entirety, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y.(2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) which are each incorporated by reference in its entirety.

Standard techniques may be used for recombinant DNA, oligonucleotide synthesis, and tissue culture and transformation (e.g., electroporation, lipofection). Enzymatic reactions and purification techniques may be performed according to manufacturer’s specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Autoimmune, Allergic, and Inflammatory Conditions and Associated Antigens

As noted above, the present airT cells may find uses in the treatment and/or amelioration of certain autoimmune, allergic, and inflammatory conditions. Clinical signs and symptoms of, and diagnostic criteria for, such conditions are known in the art. Non-limiting examples of such conditions for which the present airT cells may be beneficially administered to a human patient or other mammalian host in need of antigen-specific immunosuppression, which may refer to an individual in whom there may be present a clinically inappropriate array of pro-inflammatory mediators (e.g., cytokines, lymphokines, hormones, and the like) and/or locally or systemically elevated levels of inflammatory cells, include: type 1 diabetes mellitus, multiple sclerosis, systemic lupus erythematosus, myasthenia gravis, rheumatoid arthritis, Crohn’s disease, inflammatory bowel disease, bullous pemphigoid, pemphigus vulgaris, autoimmune hepatitis, asthma, allergy (e.g., specific hypersensitivity to food, plant, animal, environmental, drug, chemical, or other allergens), tolerance induction for transplantation (e.g., pancreatic islet cell transplantation), graft-versus-host disease (GVHD) following stem cell (e.g., hematopoietic SC) transplantation, and the like.

Antigens associated with these conditions, and in particular, portions of such antigens in which epitopes recognized by TCR reside, are known and are set forth in the Drawings. Also set forth in the Drawings are TCR V-region sequences of TCR that have been described on the basis of their ability to recognize the herein disclosed antigens associated with autoimmune, allergic, and inflammatory conditions.

Methods for the identification and characterization of antigens associated with pathogenesis of an autoimmune condition, an allergic condition, or an inflammatory condition, including determination of autoreactive T cell epitopes, are known in the art. For instance, exemplary methods for identifying pancreatic islet autoantigenic polypeptides, including peptide fragments thereof that are recognized by T cells from type 1 diabetes (T1D) subjects, are described in Cerosaletti et al. (2017 J. Immunol. 199:323, which is expressly incorporated by reference in its entirety). Other polypeptide antigens associated with pathogenesis of an autoimmune condition, an allergic condition, or an inflammatory condition, including determination of autoreactive T cell epitopes, are disclosed herein including in the Drawings.

Cerosaletti et al. (2017) also describe exemplary and non-limiting methodologies for determining the structures of T cell receptors (TCR) that recognize antigens associated with pathogenesis of an autoimmune condition, an allergic condition, or an inflammatory condition. TCR structural features, including partial or complete TCR alpha chain variable (V-alpha) and/or beta chain variable (V-beta) region amino acid sequences and encoding polynucleotide sequences therefor are disclosed herein including in the Drawings for a variety of TCRs specific for different antigens associated with pathogenesis of an autoimmune condition, an allergic condition, or an inflammatory condition.

### Compositions and Methods of Use

Accordingly, certain presently disclosed embodiments contemplate administration of the herein described airT cells as adoptively transferred immunotherapeutic cells to provide antigen-specific immunosuppression for such a condition in which excessive and/or clinically deleterious antigen-specific immune activity is present. For example, by way of illustration and not limitation, according to certain embodiments there are contemplated immunotherapeutic protocols involving the adoptive transfer to a subject (e.g., a patient having an autoimmune, allergic, or other inflammatory condition) of the presently disclosed airT cells. Adoptive transfer protocols using unselected or selected T cells are known in the art (e.g., Schmitt et al., 2009 Hum. Gen. 20:1240; Dossett et al., 2009 Mol. Ther. 17:742; Till et al., 2008 Blood 112:2261; Wang et al., 2007 Hum. Gene Ther. 18:712; Kuball et al., 2007 Blood 109:2331; US2011/0243972; US2011/0189141; Leen et al., 2007 Ann. Rev. Immunol. 25:243; US2011/0052530, US2010/0310534; Ho et al., 2006 J. Imm. Meth. 310:40; Ho et al., 2003 Canc. Cell 3:431) and may be modified according to the teachings herein for use with transfer cell populations containing desired airT cells generated as described herein.

Administration of the airT cells can be carried out via any of the accepted modes of administration of agents for serving similar utilities. The airT cells can be prepared in a pharmaceutical composition by combining with an appropriate physiologically acceptable carrier, diluent or excipient, such as an aqueous liquid optionally containing suitable salts, buffers and/or stabilizers. Administration of airT cells may be achieved by a variety of different routes such as intravenous, intrahepatic, intraperitoneal, intragastric, intraarticular, intrathecal, or other routes, and in preferred embodiments by intravenous infusion.

Preferred modes of administration depend upon the nature of the condition to be treated or prevented, which in certain embodiments will refer to a deleterious or clinically undesirable condition the extent, severity, likelihood of occurrence and/or duration of which may be decreased (e.g., reduced in a statistically significant manner relative to an appropriate control situation such as an untreated control) according to certain methods provided herein. An amount that, following administration, detectably reduces, inhibits, or delays such a condition, for instance, the local or global level autoimmune, allergic, or other harmful inflammatory activity, is considered effective. Persons skilled in the relevant arts will be familiar with any number of diagnostic, surgical and other clinical criteria to which can be adapted to evaluation of the effects of administration by adoptive transfer of the immunoregulatory airT cell compositions described herein. See, e.g., Humar et al., Atlas of Organ Transplantation, 2006, Springer; Kuo et al., Comprehensive Atlas of Transplantation, 2004 Lippincott, Williams & Wilkins; Gruessner et al., Living Donor Organ Transplantation, 2007 McGraw-Hill Professional; Antin et al., Manual of Stem Cell and Bone Marrow Transplantation, 2009 Cambridge University Press; Wingard et al. (Ed.), Hematopoietic Stem Cell Transplantation: A Handbook for Clinicians, 2009 American Association of Blood Banks.

Accordingly, in some embodiments the airT cell may express an antigen-specific T cell receptor (TCR) that comprises the antigen-specific TCR polypeptide encoded by the at least one transduced polynucleotide that encodes said TCR polypeptide and is capable of antigen-specifically induced immunosuppression in response to HLA-restricted stimulation by an antigen that is specifically recognized by the TCR polypeptide. Determination of the presence of immunosuppression may be accomplished by any of a wide variety of criteria with which those skilled in the art will be familiar. See, e.g., Sakaguchi et al., 2020 Ann. Rev. Immunol. 38:541 which is expressly incorporated by reference in its entirety.

For example, by way of illustration and not limitation, multiple mechanisms contributing to suppressive phenotype of Treg cells have been described, such as CTLA-4 immune checkpoint, expression of immunosuppressive cytokines such as IL-10 and TGF-β, cytotoxicity of target cells through the perforin/granzyme pathway, induction of indoleamine 2,3-dioxygenase (IDO) and the catabolism of tryptophan in target cells, as well as consumption of adenosine by expression of CD73, and competition with effector T (T_{eff}) cells for IL-2 since Treg cells constitutively express CD25 (the a subunit of the high affinity receptor for IL-2). See, e.g., Verbsky, J.W., and Chatila, T.A. (2014). Chapter 23 - Immune Dysregulation Leading to Chronic Autoimmunity. In Stiehm’s Immune Deficiencies, K.E. Sullivan, and E.R. Stiehm, eds., (Amsterdam: Academic Press), pp. 497-516; Campbell et al. 2020 Cell Metab. 31(1): 18-25; Dominguez-Villar et al., 2018 Nat. Immunol. 19:665-673; Sakaguchi et al., 2008 Cell 133(5):775-787.

In some embodiments, antigen-specifically induced immunosuppression thus may comprise one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines or anti-inflammatory products, for example, elaboration of one or more inhibitory mechanisms including release of immunosuppressive cytokines or perforin/granzyme, induction of indoleamine 2,3-dioxygenase (IDO), competition for IL2 or adenosine, catabolism of tryptophan, and expression of inhibitory receptors by the airT cell, by the airT cell, and (iv) inhibition of either or both of activation and proliferation of effector T cells that do not recognize the antigen that is specifically recognized by the airT TCR comprising the TCR polypeptide that is encoded by the at least one transduced polynucleotide.

In certain instances, adoptive transfer airT cell immunotherapy doses (and optionally, at least one other therapeutic agent dose) may be provided between 1 day and 14 days over a 30 day period. In certain instances, doses (and optionally, at least one other therapeutic agent dose) may be provided 1, 2, 3, 4, 5, 6, 7, 8, 9, 10, 11, 12, 13, or 14 days over a 60 day period. Alternate protocols may be appropriate for individual subjects. A suitable dose is an amount of a compound that, when administered as described above, is capable of detectably altering or ameliorating symptoms, or decreases at least one indicator of autoimmune, allergic or other inflammatory immune activity in a statistically significant manner by at least 10-50% relative to the basal (e.g., untreated) level, which can be monitored by measuring specific levels of blood components, for example, detectable levels of circulating immunocytes and/or other inflammatory cells and/or soluble inflammatory mediators including proinflammatory cytokines.

In general, an appropriate dosage and treatment regimen provides the airT cells in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Decreases (e.g., reductions having statistical significance when compared to a relevant control) in preexisting immune responses to an antigen associated with an autoimmune, allergic, or other inflammatory condition as provided herein generally correlate with an improved clinical outcome. Such immune responses may generally be evaluated using standard leukocyte and/or lymphocyte cell surface marker or cytokine expression, proliferation, cytotoxicity or released cytokine assays, which are routine in the art and may be performed using samples obtained from a subject before and after therapy.

For example, an amount of airT cells that is administered is sufficient to result in clinically relevant reduction (e.g., a decrease that is clinically remarkable, preferably as may be detectable in a statistically significant manner relative to an appropriate control condition) in symptoms of autoimmune diseases, including but not limited to type 1 diabetes, rheumatoid arthritis (RA), systemic lupus erythematosus (SLE), multiple sclerosis, inflammatory bowel disease (IBD), psoriatic arthritis, Crohn’s disease, ulcerative colitis, seronegative spondyloarthropathies, Behcet’s disease, vasculitis, or other autoimmune diseases.

Accordingly, in some embodiments a reduction in one or more relevant clinical criteria as known in the art for assessing type 1 diabetes (T1D) may be identified following adoptive transfer, to a T1D patient, of airT cells expressing a TCR that specifically recognizes an epitope of an antigen having relevance to a T1D-associated autoantigen. Exemplary T1D-associated antigens and TCR structures that specifically recognize such antigens, which are typically autoantigens, are described herein.

Common defining criteria for stage two T1D may include detection of two or more pancreatic islet-specific autoantibodies in the patient and evidence of dysglycemia during an oral glucose-tolerance test. Dysglycemia may in some embodiments be defined as a fasting glucose level of 110 to 125 mg per deciliter (6.1 to 6.9 mmol per liter), a two-hour postprandial plasma glucose level of at least 140 mg per deciliter (7.8 mmol per liter) and less than 200 mg per deciliter (11.1 mmol per liter), or an intervening postprandial glucose level at 30, 60, or 90 minutes of greater than 200 mg per deciliter. In some embodiments clinical T1D may be defined as the presence of symptoms of diabetes (e.g., increased thirst, increased urination, and/or unexplained weight loss, compared to normal subjects known to be free of any risk for having or presence of T1D) and a blood sugar level equal to or greater than 200 milligrams per deciliter (mg/dL), a fasting blood sugar level that is equal to or greater than 126 mg/dL, or a two-hour oral glucose tolerance test (OGTT) result that is equal to or greater than 200 mg/dL or a hemoglobin Alc level that is 6.5% or higher (e.g., Khokhar et al., 2017 Clin. Diabetes 35(3):133.)

Reduction in RA symptoms may be evidenced, for example by way of illustration and not limitation, as reduction of any one or more of fatigue, loss of appetite, low fever, swollen glands, weakness, swollen joints, joint pain, morning stiffness, warm, tender, or stiff joints when not used for as little as an hour, bilateral joint pain (fingers (but not the fingertips), wrists, elbows, shoulders, hips, knees, ankles, toes, jaw, and neck may be affected); loss of range of motion of affected joints, pleurisy, eye burning, eye itching, eye discharge, nodules under the skin, numbness, tingling, or burning in the hands and feet. Criteria for diagnosis and clinical monitoring of RA patients are well known to those skilled in the relevant art. See, e.g,. Hochberg et al., Rheumatology, 2010 Mosby; Firestein et al., Textbook of Rheumatology, 2008 Saunders. Criteria for diagnosis and clinical monitoring of patients having RA and/or other autoimmune diseases are also well known to those skilled in the relevant art. See, e.g., Petrov, Autoimmune Disorders: Symptoms, Diagnosis and Treatment, 2011 Nova Biomedical Books; Mackay et al. (Eds.), The Autoimmune Diseases-Fourth Edition, 2006 Academic Press; Brenner (Ed.), Autoimmune Diseases: Symptoms, Diagnosis and Treatment, 2011 Nova Science Pub. Inc.

Standard techniques may be used for recombinant DNA, peptide and oligonucleotide synthesis, immunoassays and tissue culture and transformation (e.g., electroporation, or lipofection). Enzymatic reactions and purification techniques may be performed according to manufacturer’s specifications or as commonly accomplished in the art or as described herein. These and related techniques and procedures may be generally performed according to conventional methods well known in the art and as described in various general and more specific references in microbiology, molecular biology, biochemistry, molecular genetics, cell biology, virology and immunology techniques that are cited and discussed throughout the present specification. See, e.g., Sambrook, et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir andCC Blackwell, eds., 1986); Roitt, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

Each embodiment described in this specification is to be applied mutatis mutandis to every other embodiment unless expressly stated otherwise.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### EXAMPLES

### Example 1—Generation of airT Cells

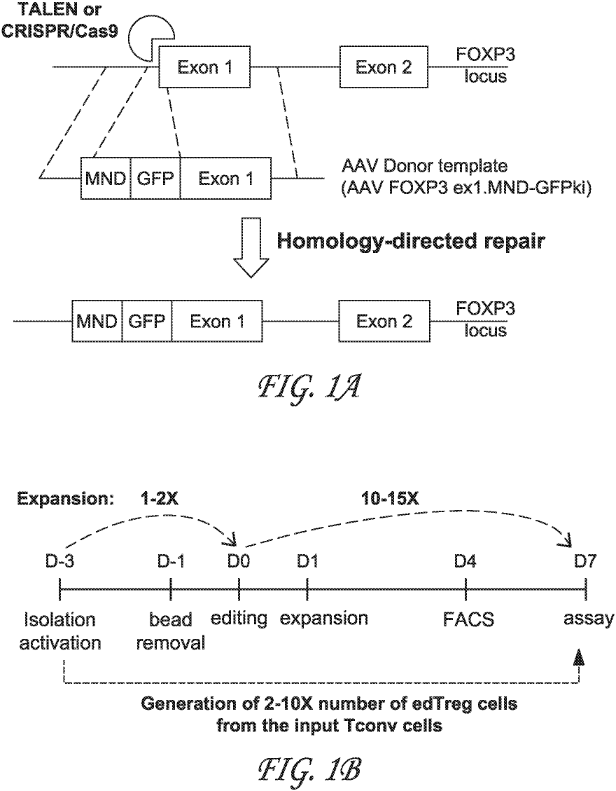

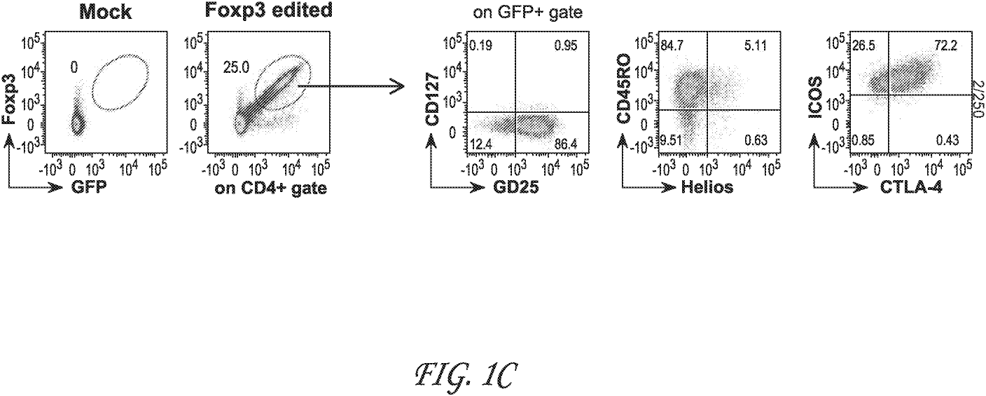

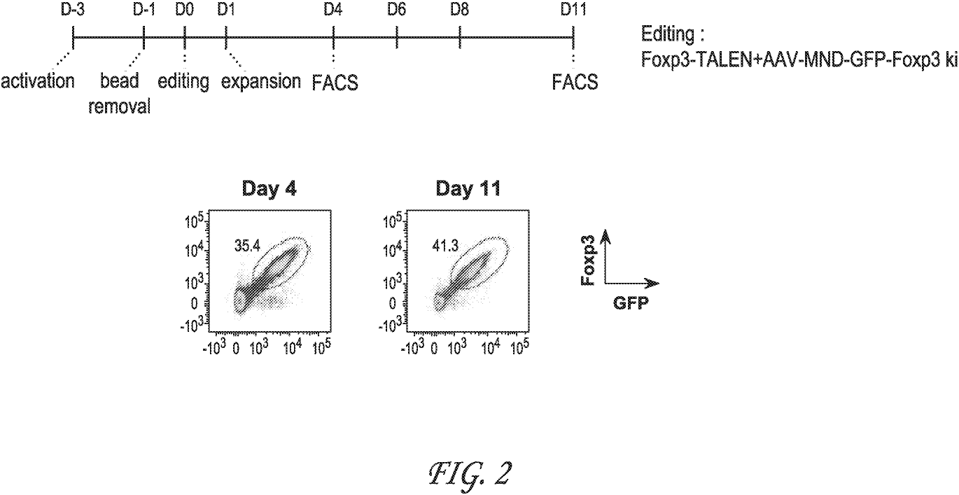

A platform was developed to generate stable engineered Treg (edTregs; airT) by converting conventional human CD4 T cells into Treg-like cells through Foxp3 gene editing (FIG. 1A, FIG. 1B, FIG. 1C, FIG. 2). This platform included the use of lentiviral TCR gene transfer to generate antigen-specific edTregs.

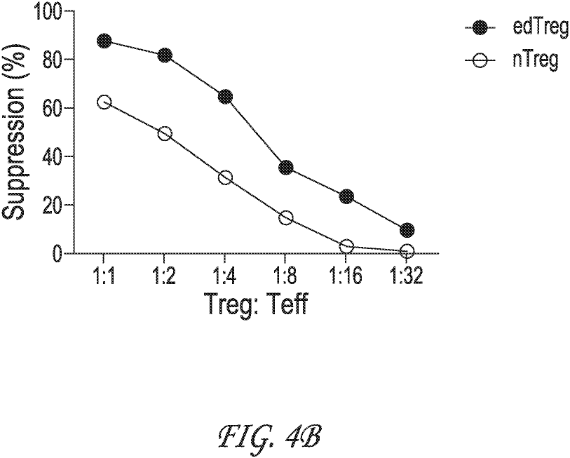

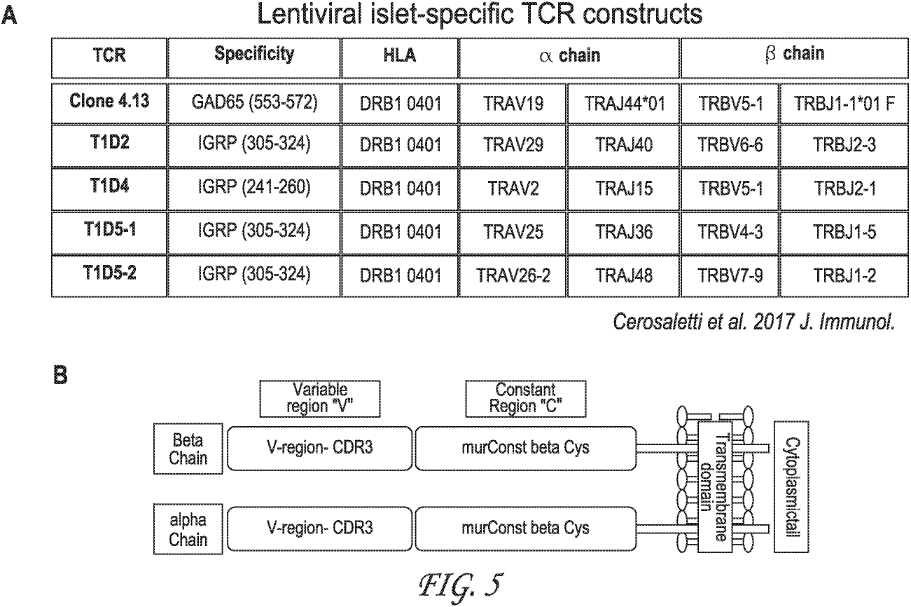

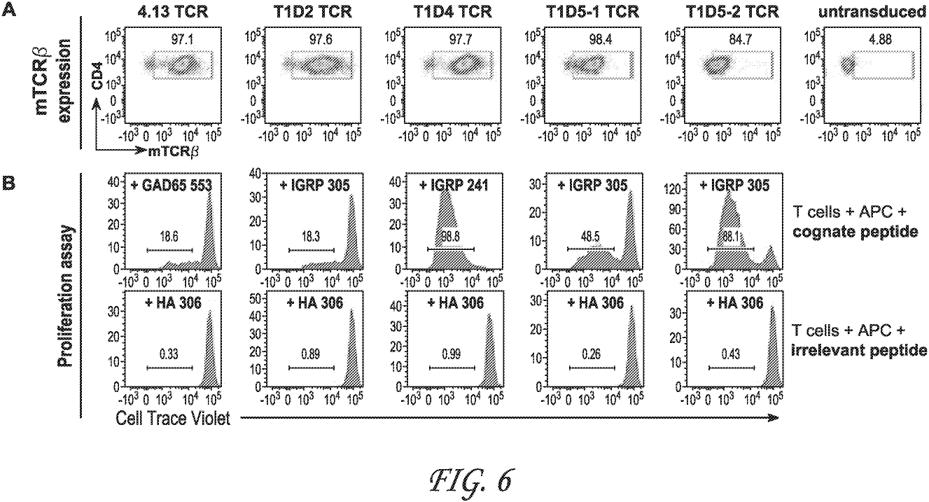

Antigen-specific T cells were identified by activating PBMC with a peptide pool, followed by assessment of CD154 expression. This method utilized single cell RNA-seq for identifying TCR clonotypes expanded in T1D subjects and was used to generate full TCR sequences (Cerosaletti et al. 2017 J. Immunol. PMID: 28566371). Based on islet-specific TCR sequences identified from this study, lentiviral TCR constructs for TCR gene transfer were generated. These TCR constructs express human TCR variable regions from islet-specific TCRs and mouse TCR constant regions allowing improved pairing between the transduced human TCR chains (FIG. 5). Islet-specific TCR expression was validated by T cell proliferation assays using the TCR cognate peptides (or irrelevant peptides) with antigen presenting cells (APCs). T cells transduced with islet-specific TCRs proliferated only in response to their cognate peptides and APC (FIG. 6).

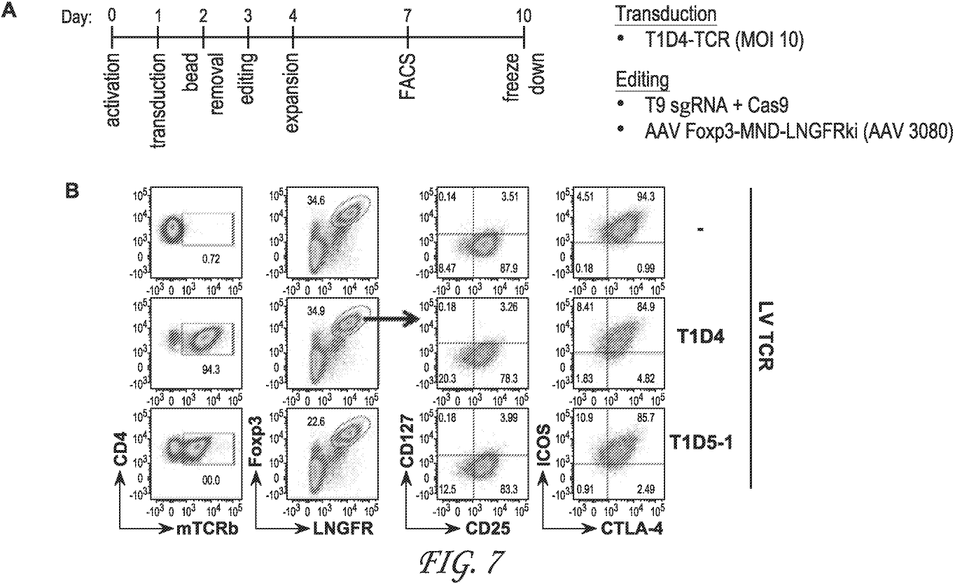

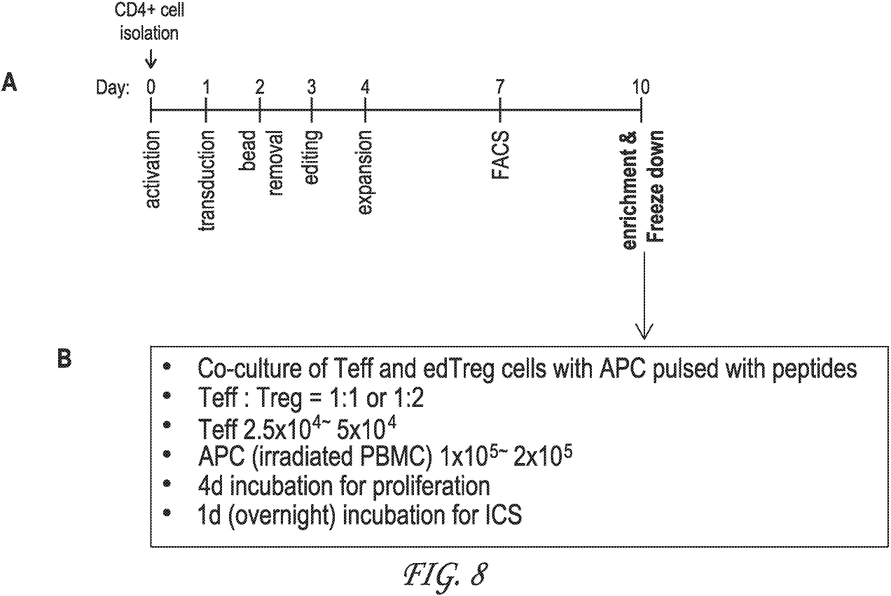

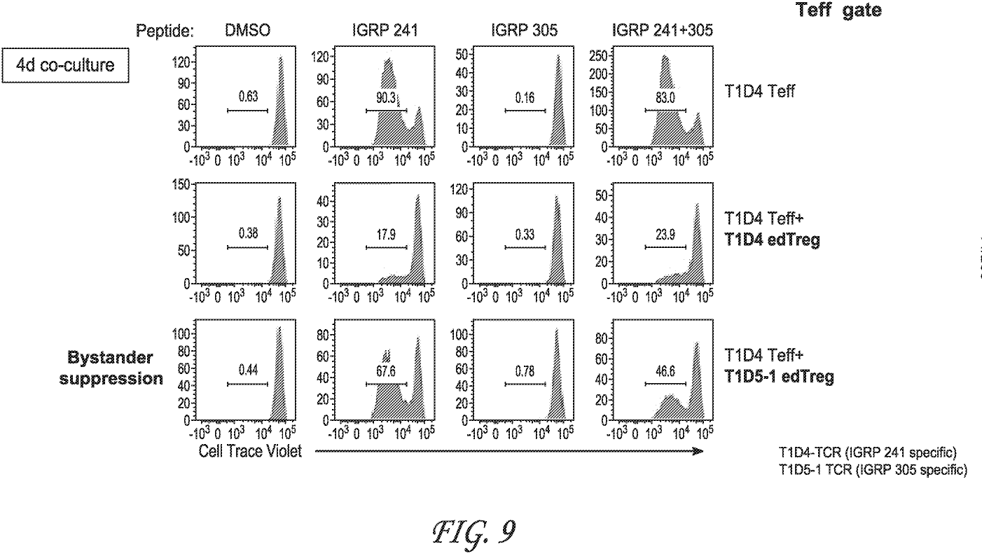

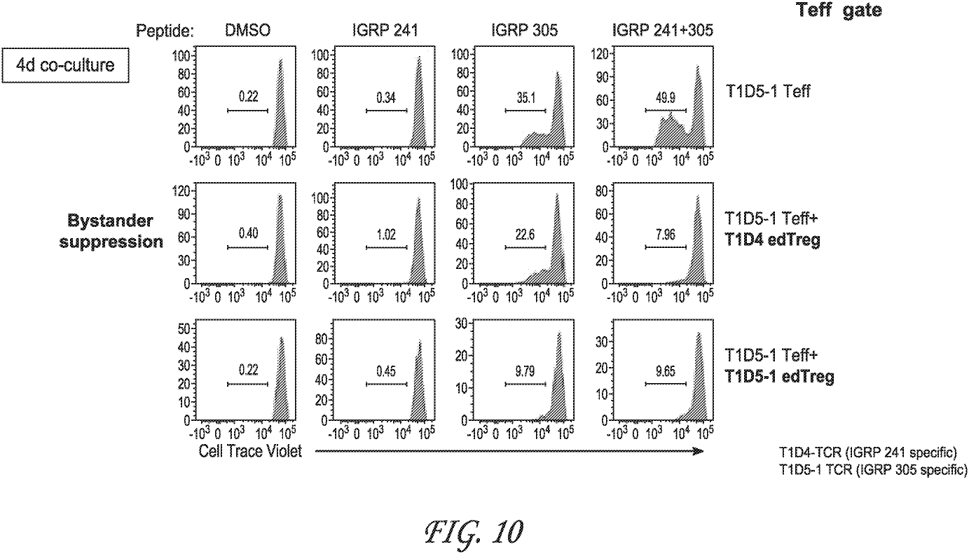

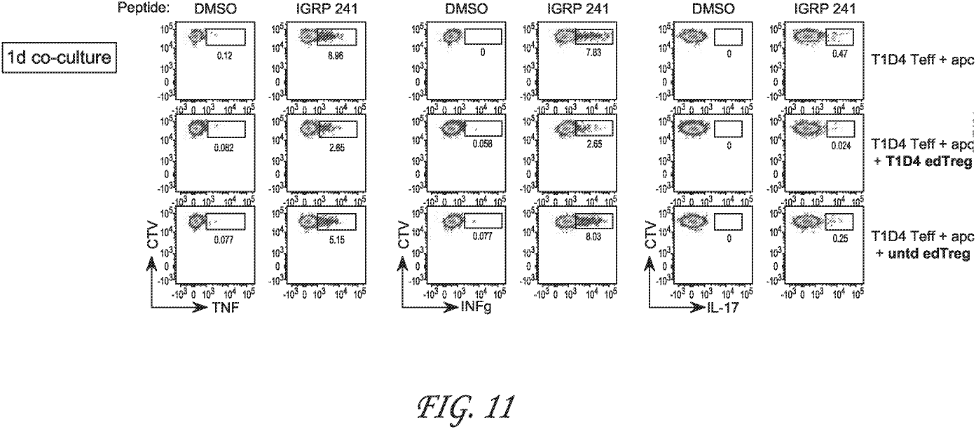

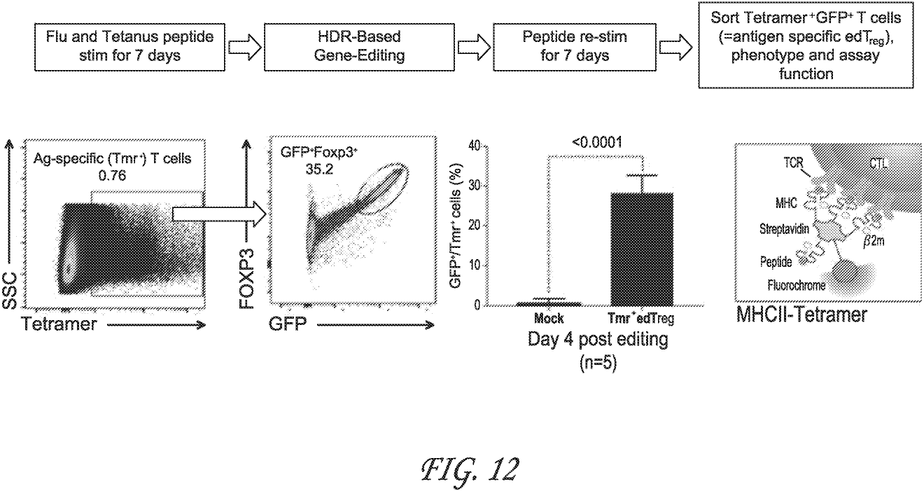

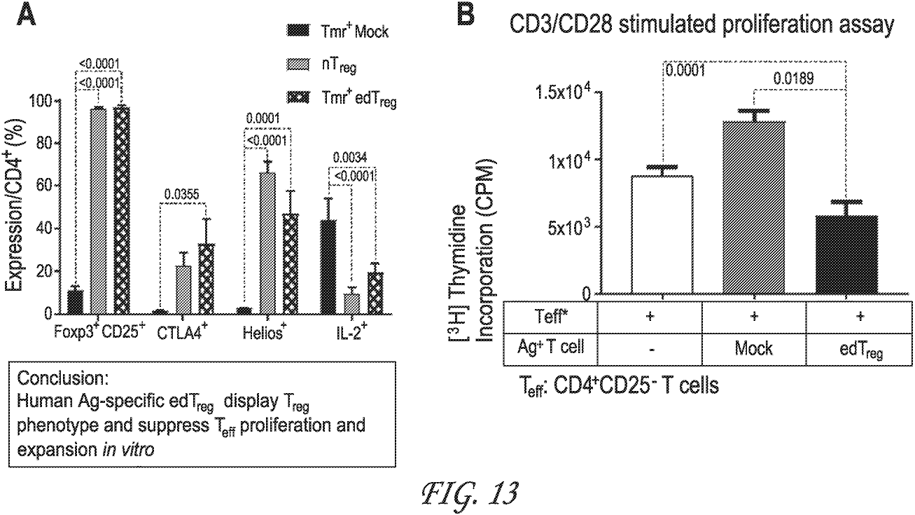

To generate antigen-specific airT cells, Foxp3 locus was edited in CD4 T cells that had been transduced with islet-TCRs, which resulted in the successful generation of airT cells expressing islet-specific TCRs. airT expressing islet-specific TCR exhibited a Treg phenotype: CD25+, CD127-, CTLA4+, ICOS+ (FIG. 7). Notably, airT expressing islet-specific TCRs show antigen-specific and bystander suppressive function by in vitro suppression assays (FIGS. 7-11). AirT cells also inhibited production of inflammatory cytokines such as TNF, IFN-g, IL-17 or IL-2 by T_{eff} cells in an airT-antigen-specific manner (FIG. 11).

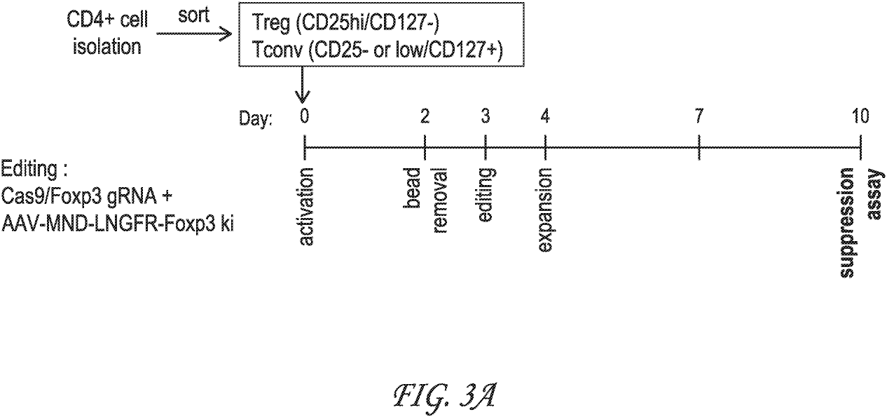

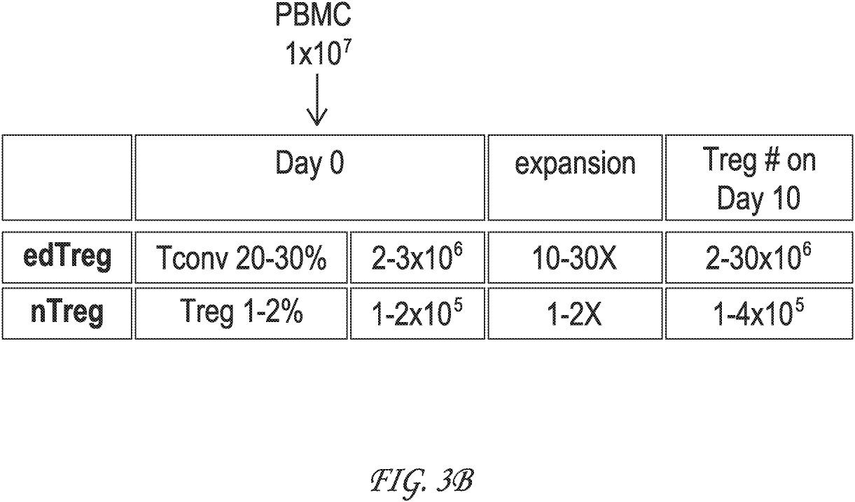

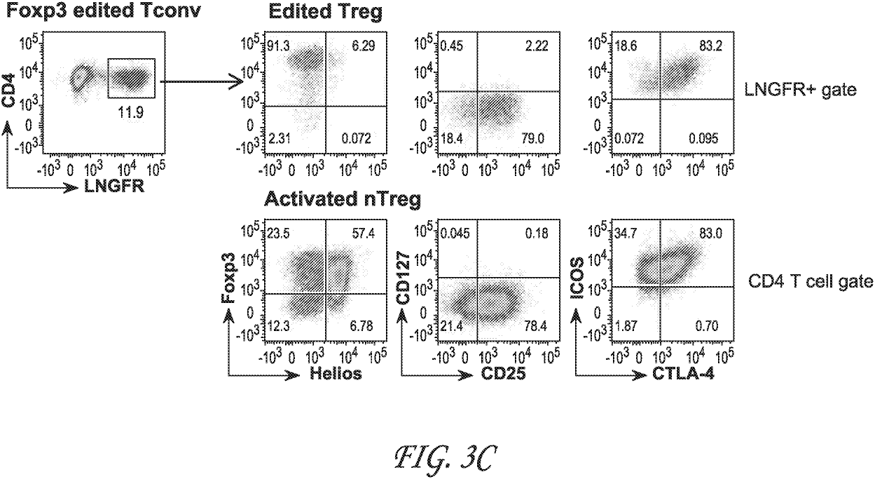

Treg phenotype, generation efficacy, and suppressive capacity of airT was investigated in comparison to expanded nTreg. airT cells could be generated up to 3 times of input number of PBMC, while the number of nTreg cells after 10 days of expansion was only 1-4% of the input cells. airT cells also exhibited a phenotype similar to nTreg, but showed higher expression of Foxp3, CTLA-4 and ICOS as compared to nTreg (FIG. 3).

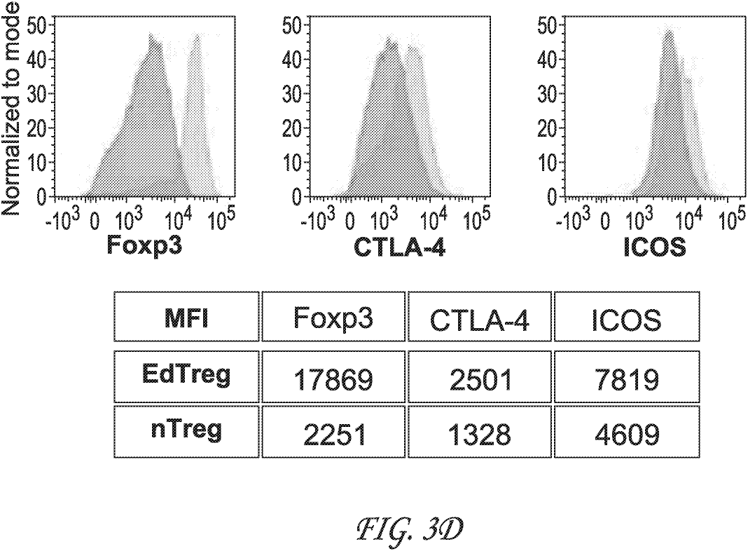

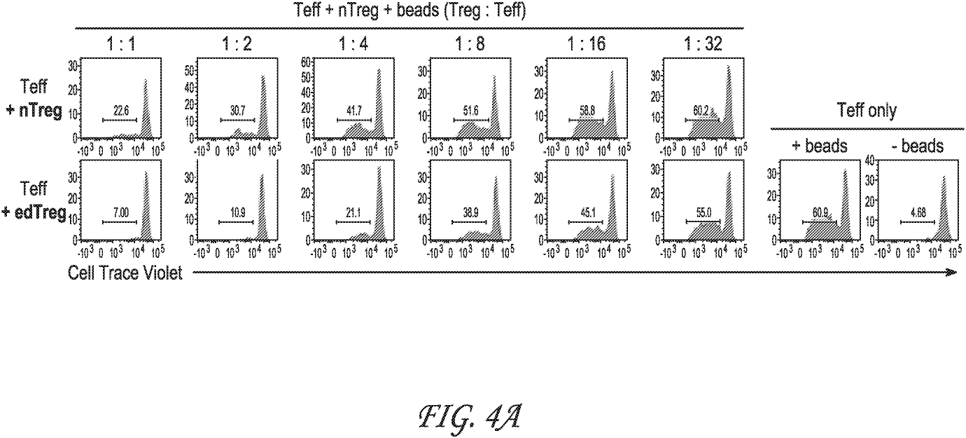

Notably, airT had similar or superior in vitro suppressive activity on effector T cell proliferation to expanded nTreg (FIG. 4).

### Example 2—Antigen-Specific Human T Cells Adopt a Treg Phenotype After FOXP3 Editing and are Immunosuppressive in vitro

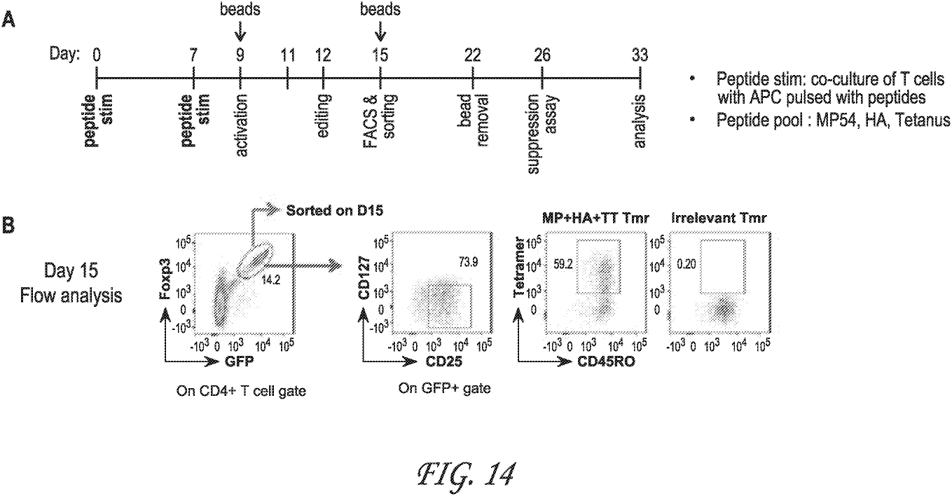

As an alternative approach to generate antigen-specific FOXP3-edited CD4+ T cells, methods were developed to isolate, edit, and expand antigen-specific effector T cells from healthy subjects or individuals with autoimmune disease. To investigate feasibility of FOXP3 editing in association with expansion of antigen-specific human T cells, CD4+ T cells from HLA DRB1*0401 human donors were expanded in the presence of influenza (flu) and tetanus antigens prior to gene editing. Following the editing procedure, the cells were further expanded in the antigen cocktail for 4 to 7 days. At this time, the average editing rate (GFP+) was 28 ± 2.1 % (FIG. 12). Antigen-specific cells were purified by FACS after labelling with a mixture of PE-conjugated flu and tetanus MHC-II tetramers. These tetramer-positive airT (Tmr+airT) recapitulated the immunophenotype of activated tTreg for canonical markers of regulatory T cells: upregulating expression of FOXP3, CD25, CTLA4, and Helios; and suppressing IL-2 production, unlike Tmr+ Mock cells analyzed in parallel (FIG. 13A). Tmr+airT were able to suppress polyclonal activated autologous CD4+ T_{eff} in vitro, unlike the Tmr+Mock cells, indicating immunosuppressive function (FIG. 13B). These results demonstrate that CD4+ T cells from human peripheral blood can be enriched for target antigen specificity by tetramer-based flow sorting and modified by gene-editing to impart tTreg-like phenotypic and suppressive properties that retain antigen specificity.

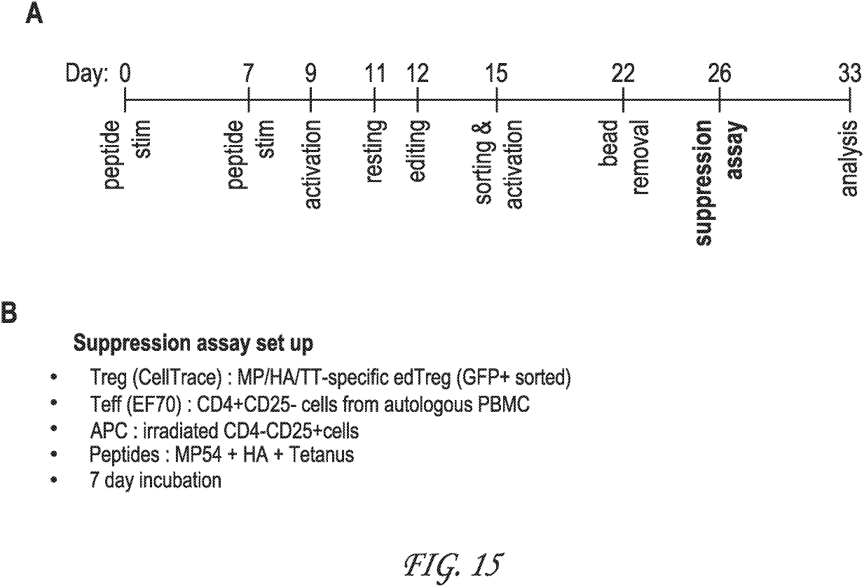

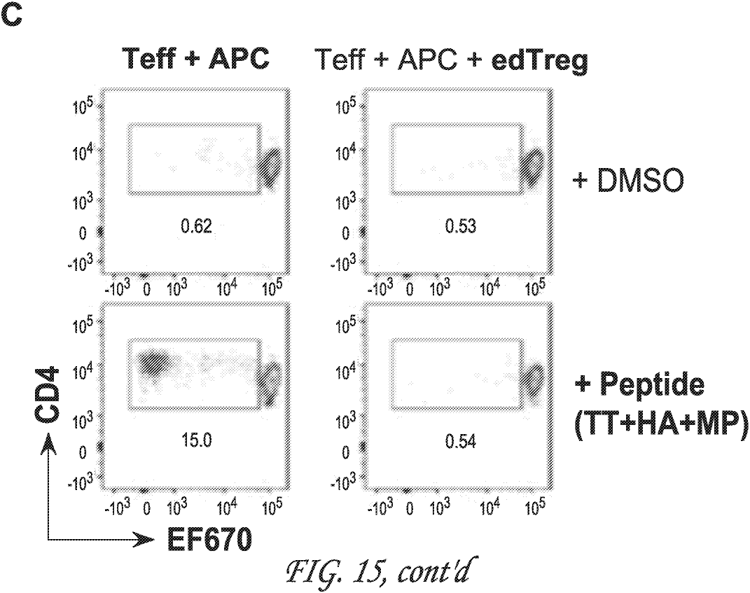

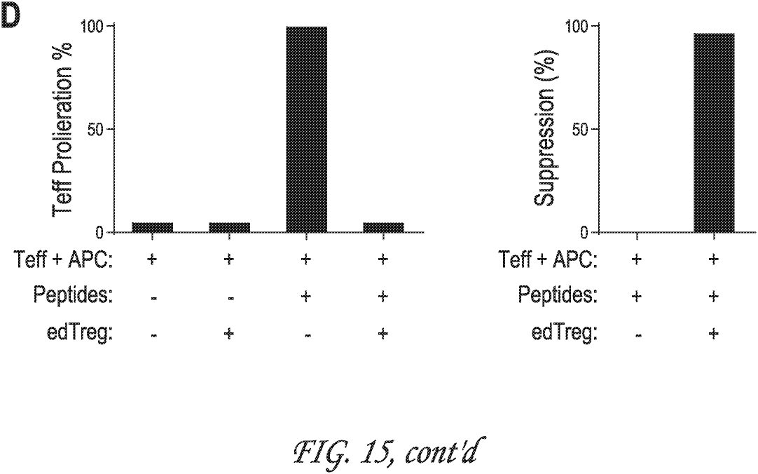

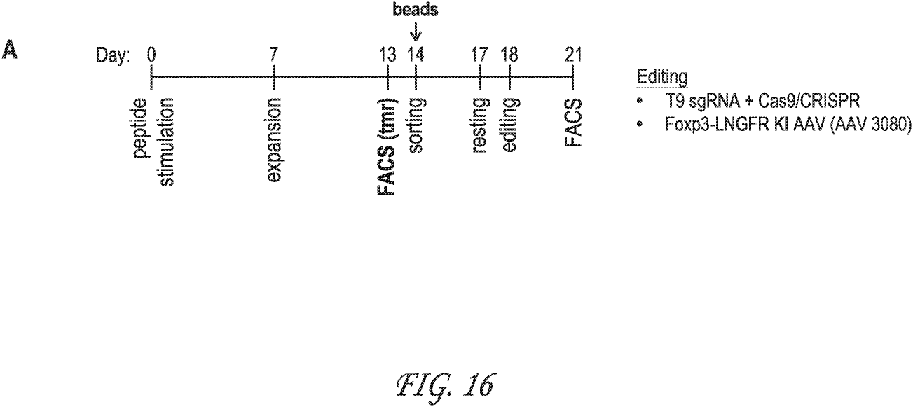

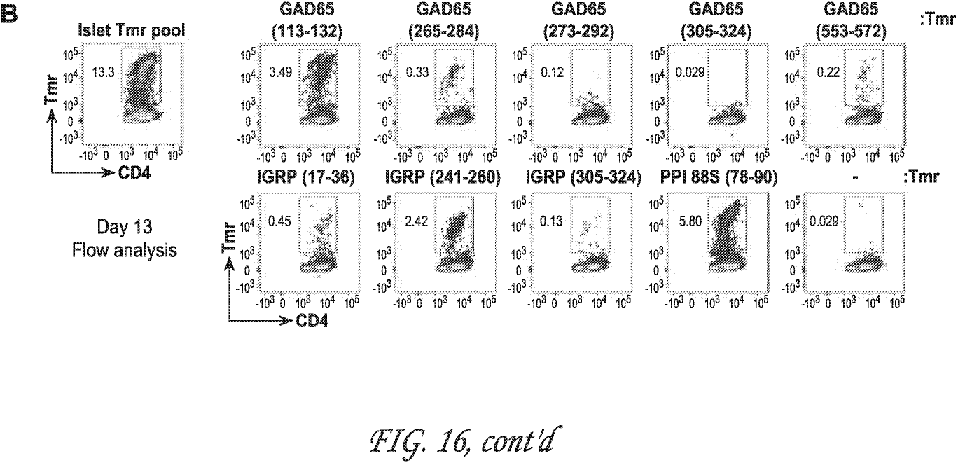

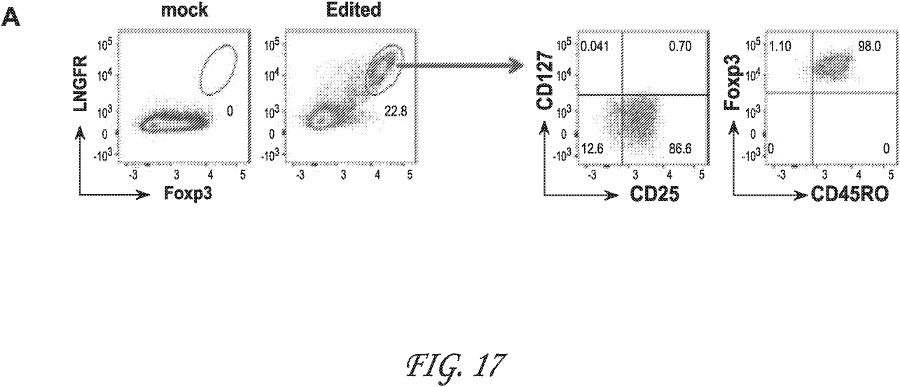

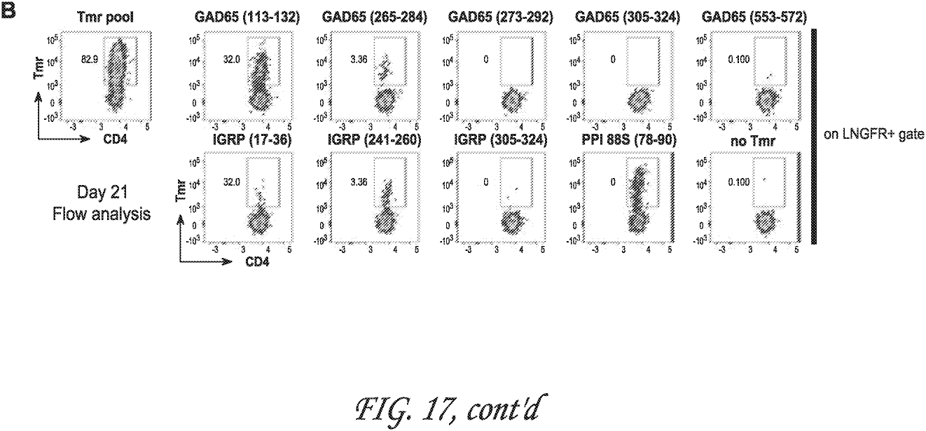

These methods were further developed to enrich for antigen-specific T cells by stimulating T cells with model antigens (MP, HA, and TT). After around 2 weeks of expansion, cells were stained with tetramers to identify antigen-specific T cells, and then the FoxP3 locus was edited (FIG. 14). Using this method, antigen-specific Tregs were generated, and these airT cells exhibited in vitro suppressive activity in antigen-specific manner (FIG. 15). In addition, islet-specific T cells were enriched by peptide stimulation method using a pool of islet-specific peptides and islet-specific T cells of multiple specificities were isolated by tetramer staining. Again, islet-specific airT cells were generated by Foxp3 gene editing in these cells (FIG. 16, FIG. 17).

### Example 3—Bi-Allelic HDR Editing for the Generation of Dual-Edited Human CD4+ T Cells

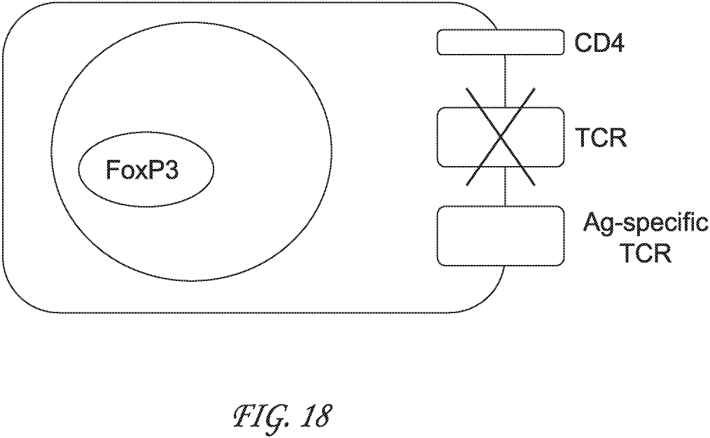

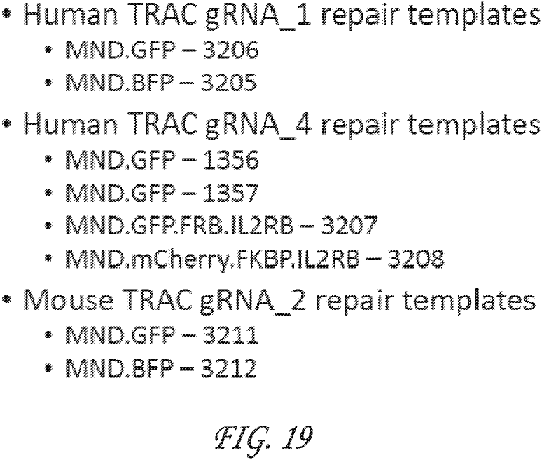

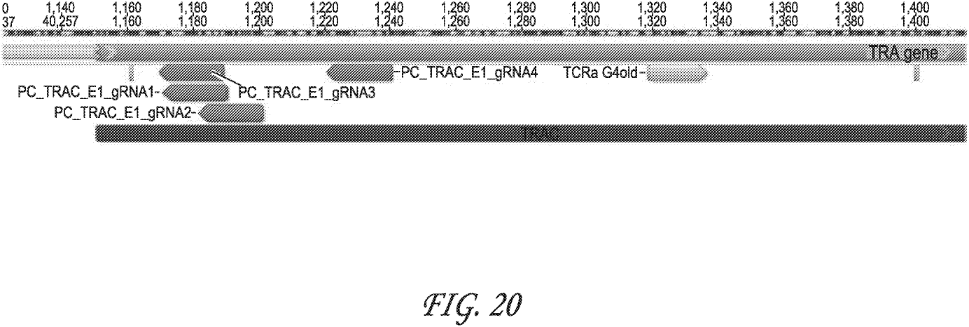

FIG. 18, FIG. 19 and FIG. 110 summarize experimental approaches used to demonstrate the ability to introduce two separate expression cassettes into the human TRAC locus (FIG. 18), as well as the constructs used in these studies (FIG. 19).

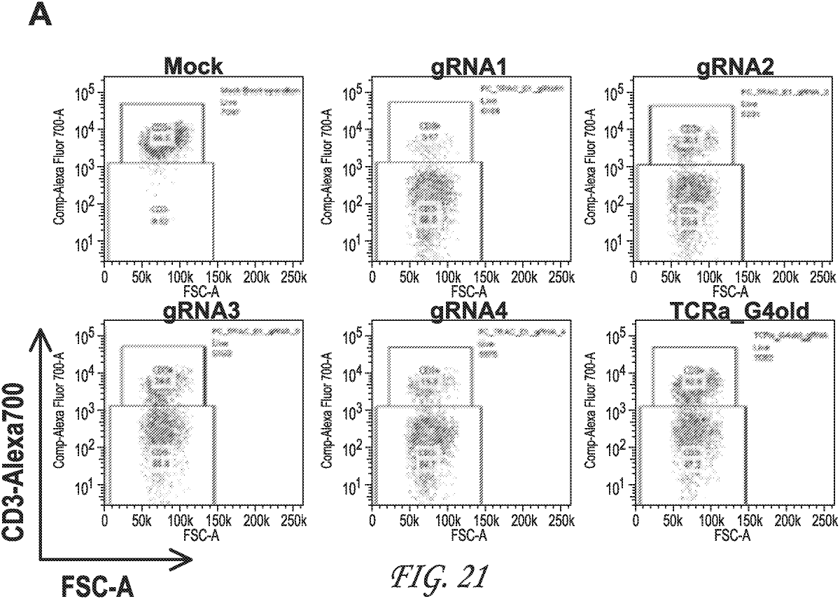

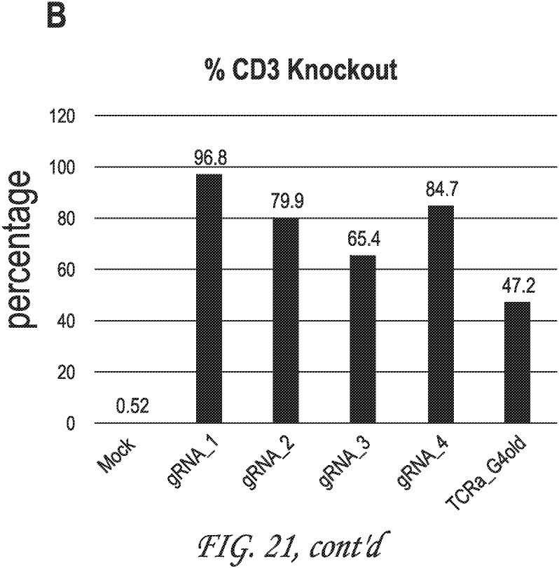

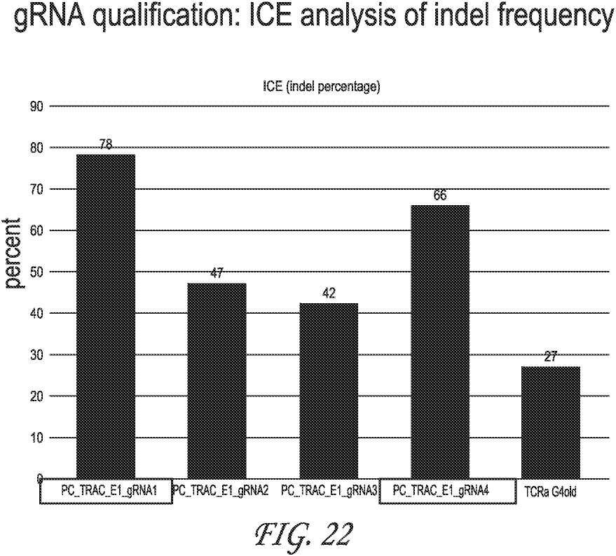

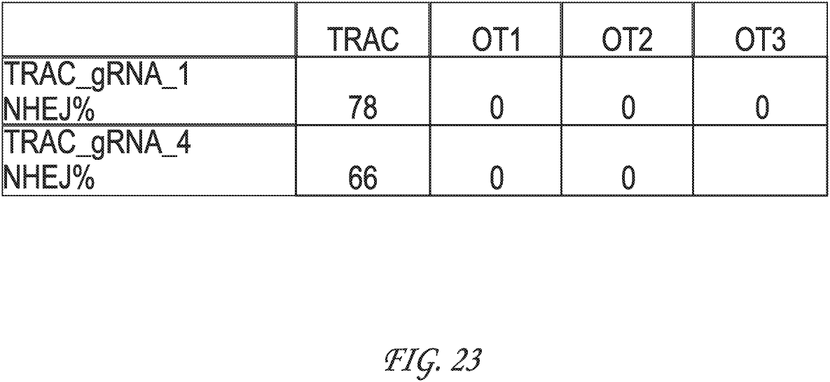

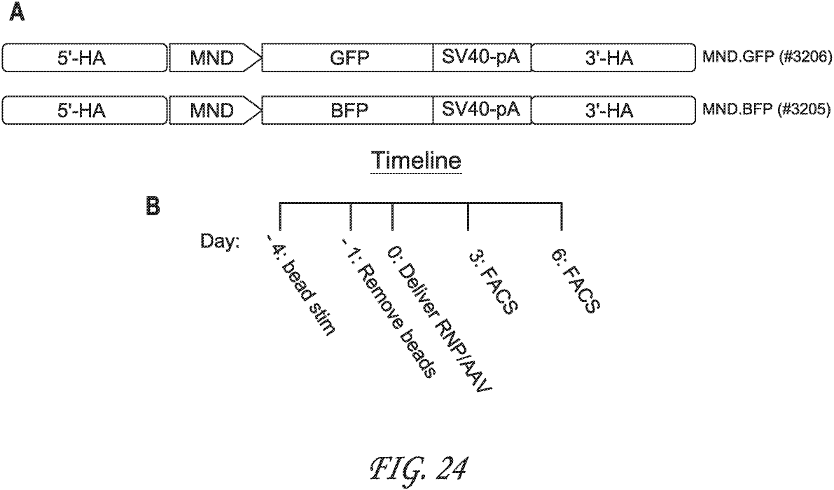

Using a CRISPR-based approach, the efficacy of four novel gRNAs targeting the first exon of human TRAC locus was tested for induction of full TCR knockout (FIG. 20). CD3 expression was evaluated using flow cytometry 48 hr after RNP delivery and demonstrated 96.8% and 84.7% CD3 knockout using gRNA_1 and gRNA_4, respectively (FIG. 21). On-target site-specific activity was measured by ICE (Inference of CRISPR Edits) and confirmed specific indel induction for gRNA_1 and gRNA_4 in TRAC relative to predicted off-target sites (FIG. 22). Next, to test the specificity of the novel guides, the top three off-target sites for each gRNA (as predicted based on bioinformatics looking at the most similar sequences in the human genome) were assessed. These were then directly analyzed in human T cells via amplification of the off-target site from T cells transfected with the nuclease. The amplicons were sequenced and analyzed by ICE program. The level of cleavage activity observed for the candidate off-taret sites was 0% cleavage. In contrast, on-target site activity in the same assay was 78% for gRNA_1 and 66% for gRNA_4 at the target TRAC site (FIG. 23). This illustrates that these novel donor templates are highly-specific for the TRAC locus.

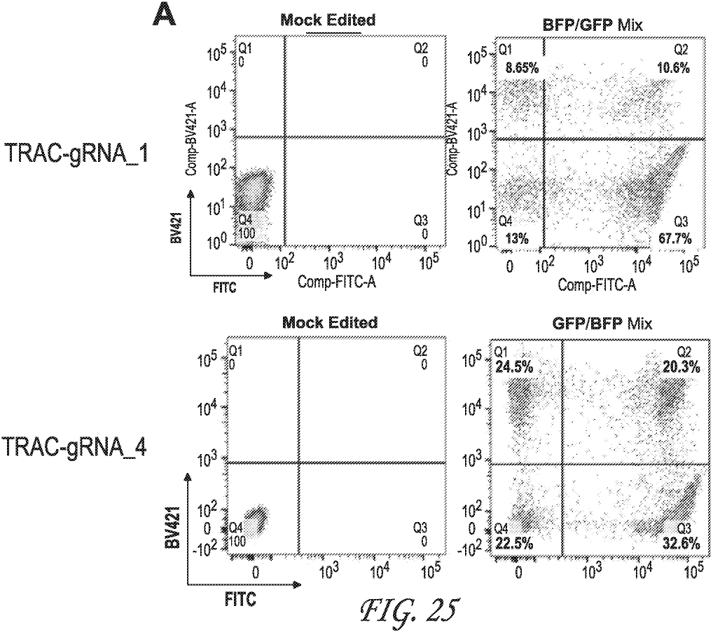

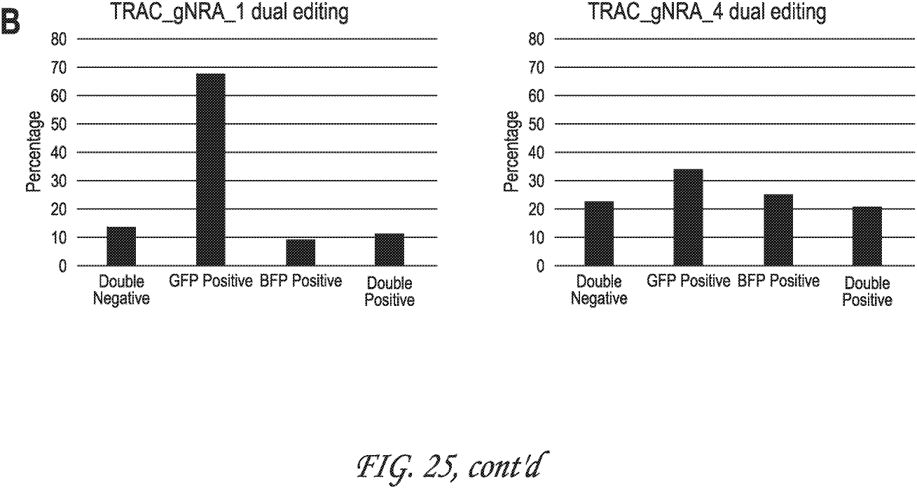

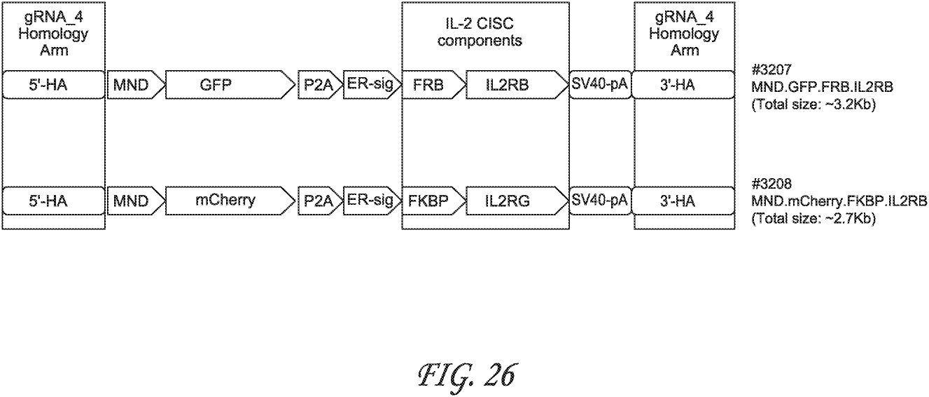

Next, the ability to dual edit human CD4+ T cells was tested using constructs that allow easy tracking of successfully edited cells. MND-GFP and MND-BFP cassettes were generated, flanked by identical 300 bp homology arms matched to TRAC gRNA_1 or gRNA_4 (FIG. 24A), and were used to test the ability to generate biallelic TRAC edited T cells with stable expression of both GFP and BFP. The timeline for cell expansion, editing and analysis is shown in FIG. 24B and the resulting FACS analysis demonstrated 20.3% and 10.6% BFP/GFP double-positive cells using gRNA_1 and gRNA_4, respectively, confirming successful integration of both repair cassettes after induction of a single double strand break (FIG. 25).

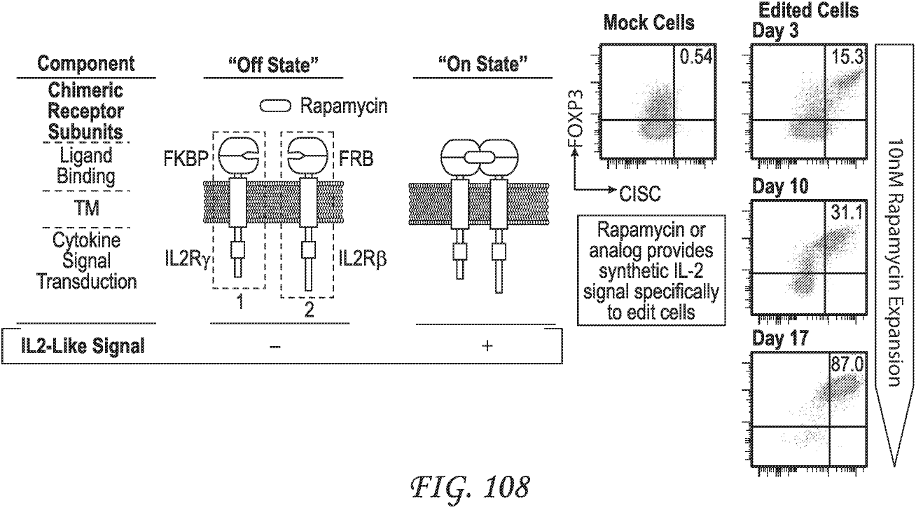

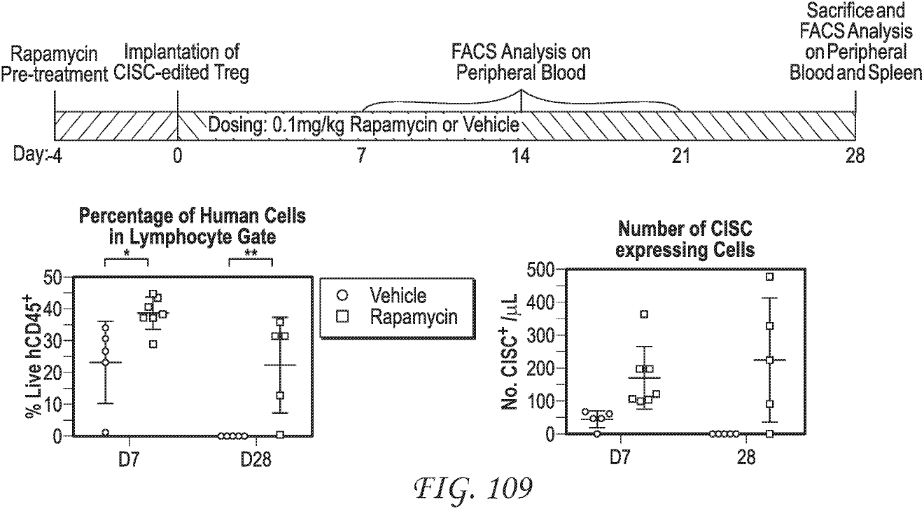

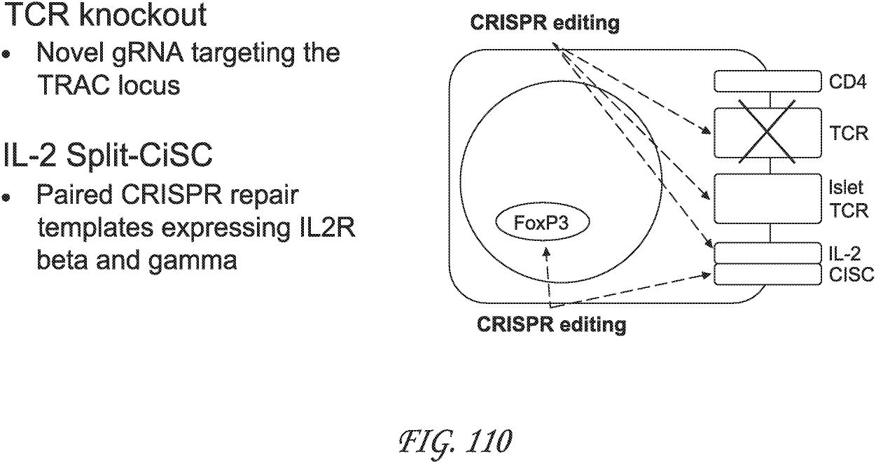

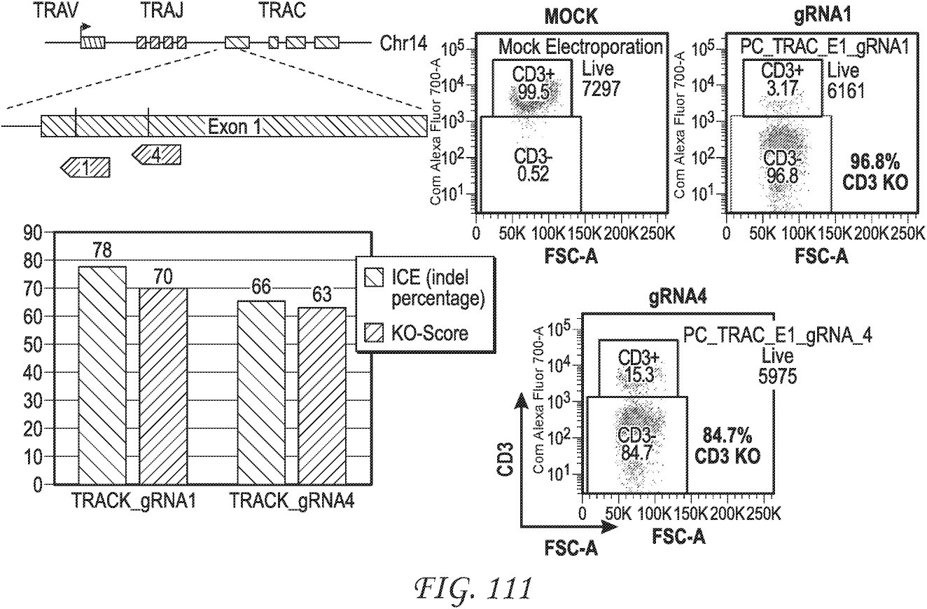

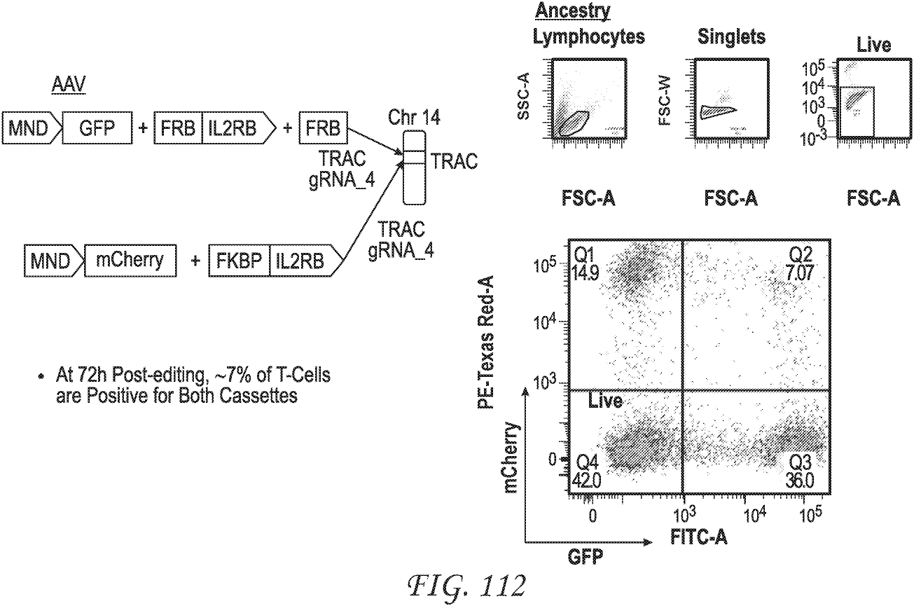

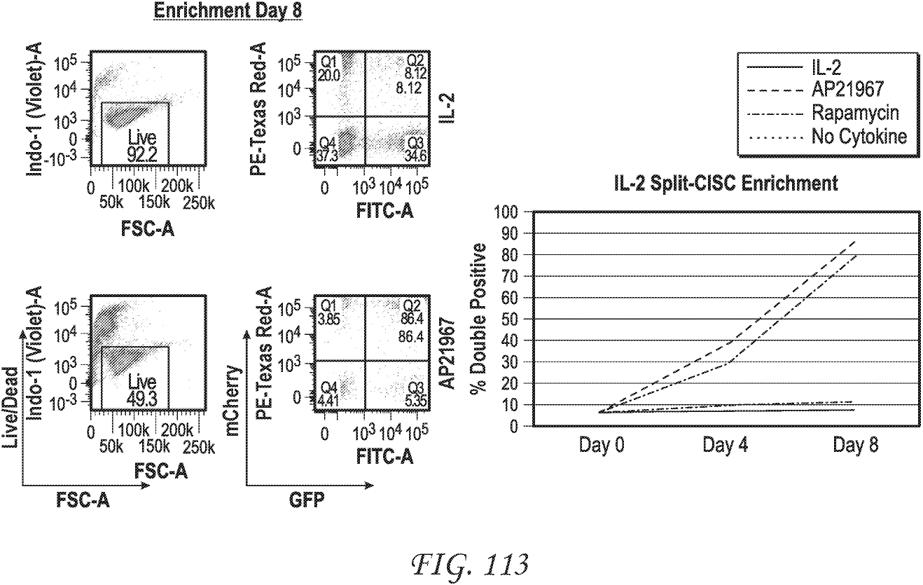

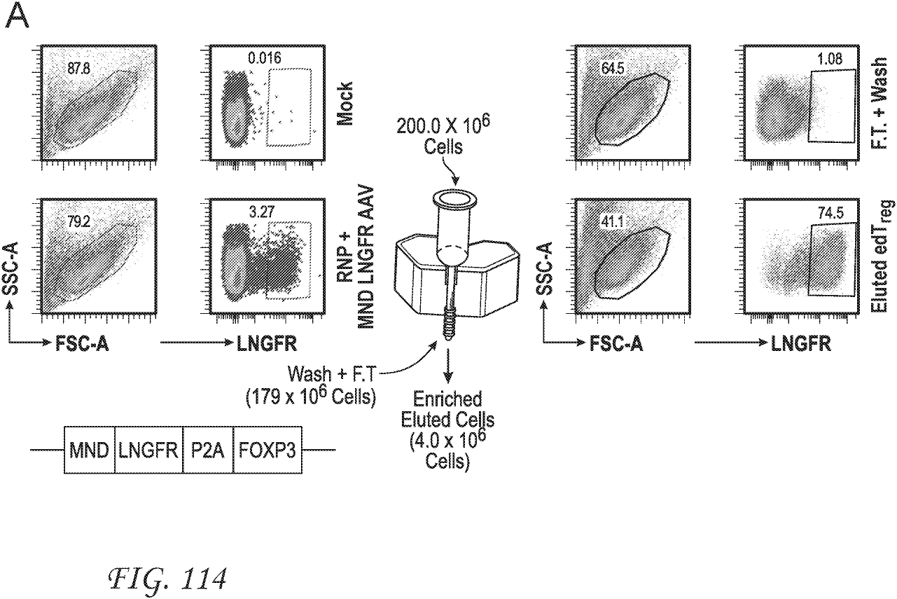

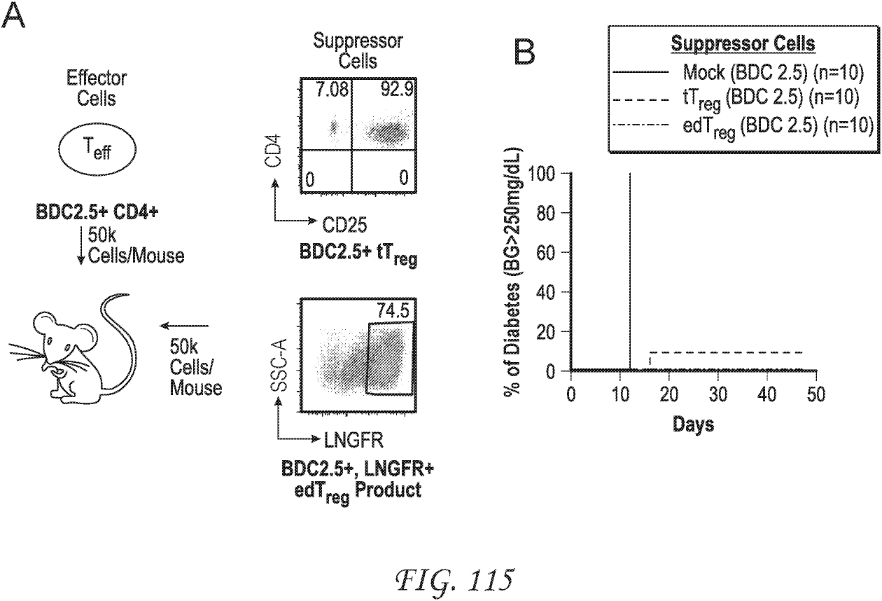

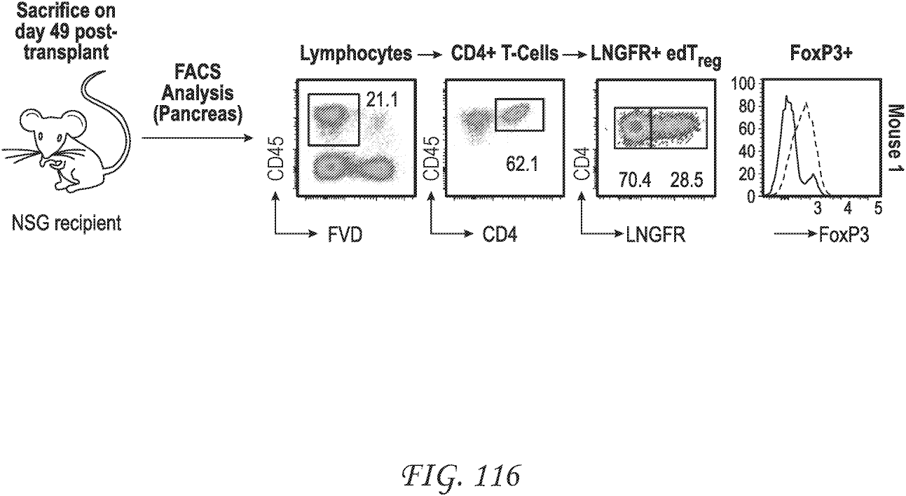

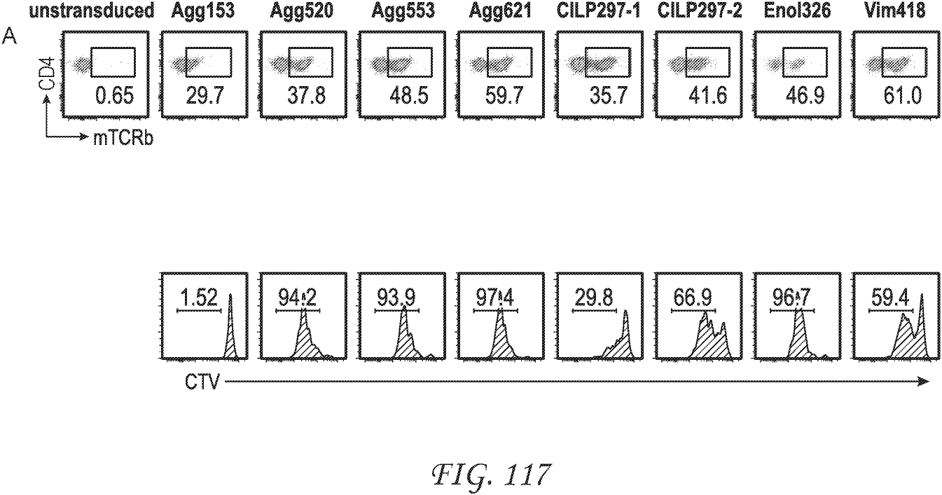

In order to obtain sufficient numbers of cells for therapeutic use, it may be useful, in some contexts, to selectively expand engineered cells in vitro. To do this in the context of dual-edited cells, split IL-2 CISC HDR knock-in constructs were generated for enrichment and selection. The method of using IL-2 CISC components has been described for the enrichment of edited CD4+ T-cells in the presence of rapamycin or a heterodimerizing rapamycin homolog, AP21967 (rapalog). See e.g., FIG. 108. In such methods, FRB-IL2RB and FKBP-IL2RG components were contained in the same cassette to select for single integration events.

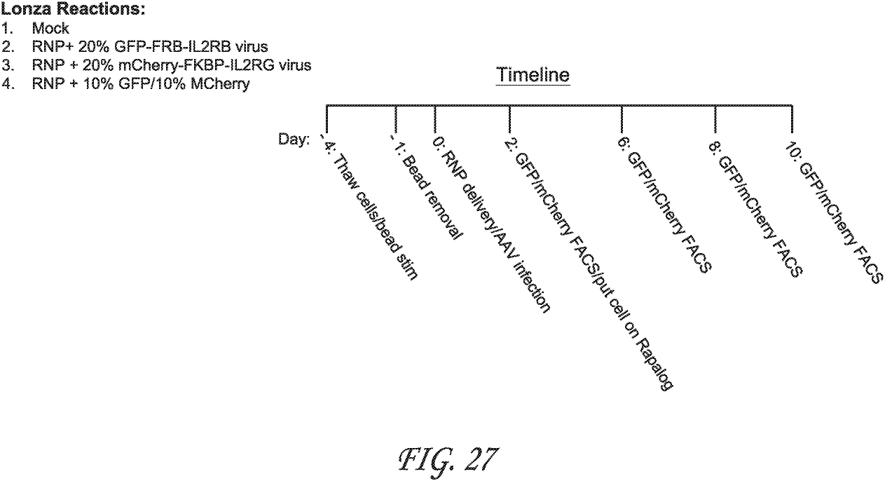

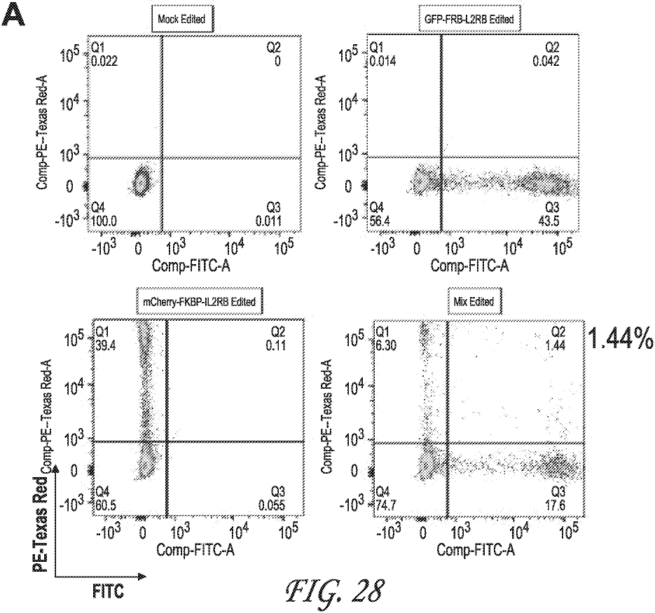

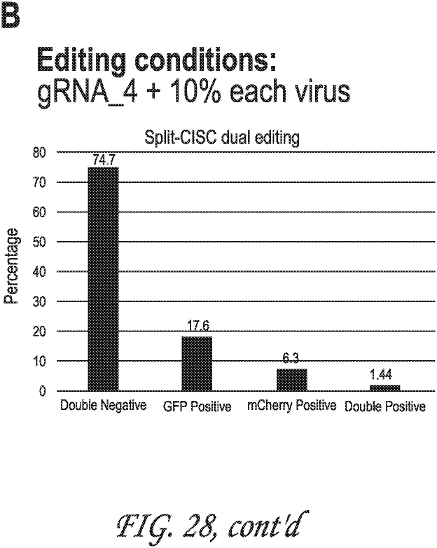

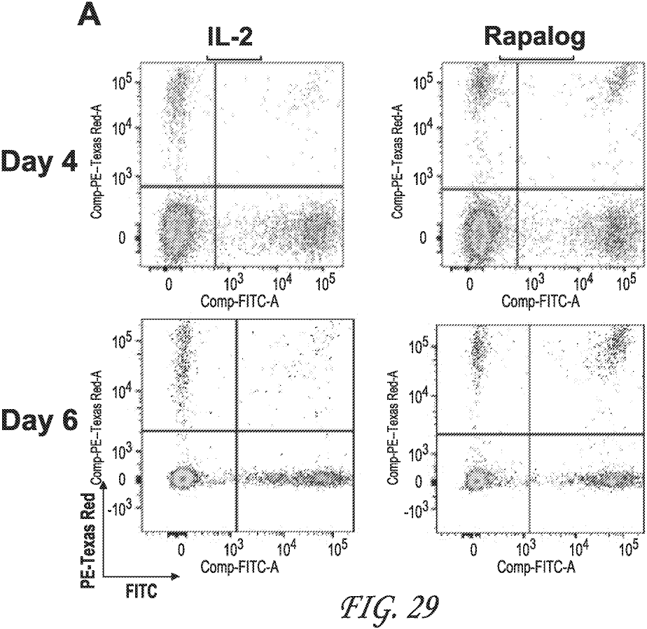

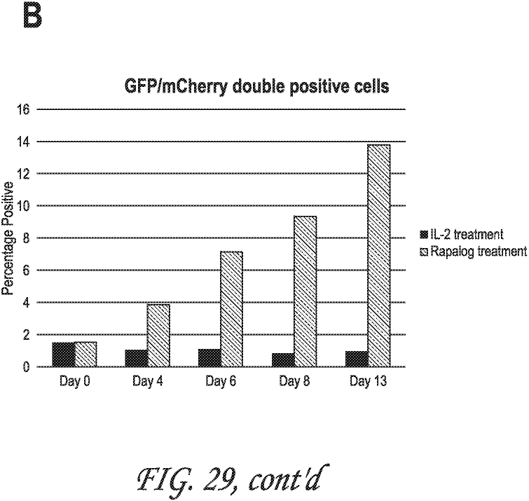

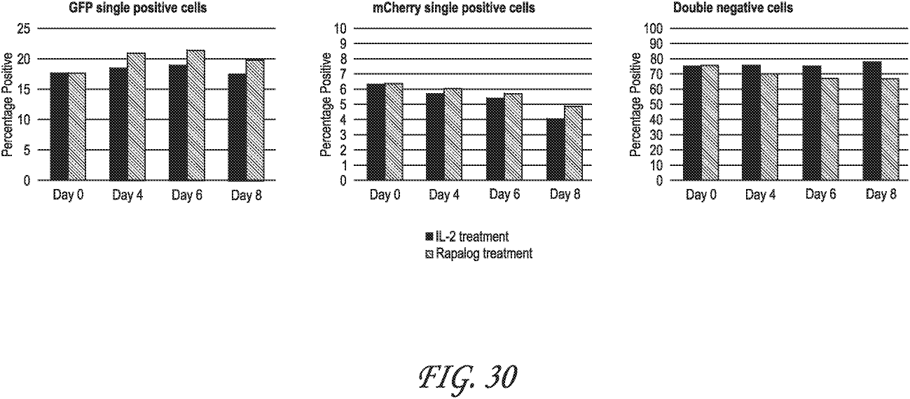

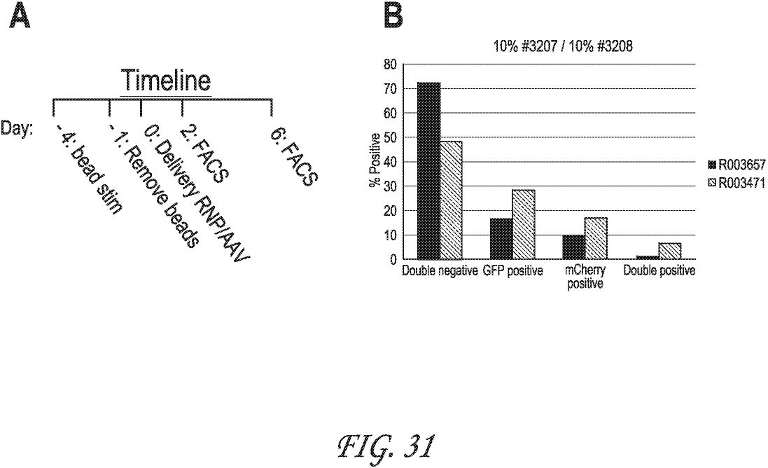

For these studies, FRB-IL2RB and FKBP-IL2RG components were split into two separate cassettes, one containing GFP and the other containing mCherry, to allow for selection of two independent integrations. Constructs are shown in FIG. 26 and the timeline and editing conditions for this experiment are shown in FIG. 27. Although the initial dual editing rate with these constructs was 1.44% double positive GFP/mCherry cells, potentially due to the increased HDR template size, FIG. 28, dual edited cells could be significantly enriched-for using rapalog. In the presence of 100 nM rapalog treatment, GFP/mCherry double positive cells increased from 1.4% to 9% over 8 days (FIG. 29). Importantly, GFP single-positive, mCherry single-positive and double-negative cells percentages remained the same in the presence of rapalog, suggesting that expansion only takes place when a functional IL-2 CISC protein is present through dual expression of FRB-IL2RB and FKBP-IL2RG. As expected, no expansion was observed in the presence of IL-2 (FIG. 30).

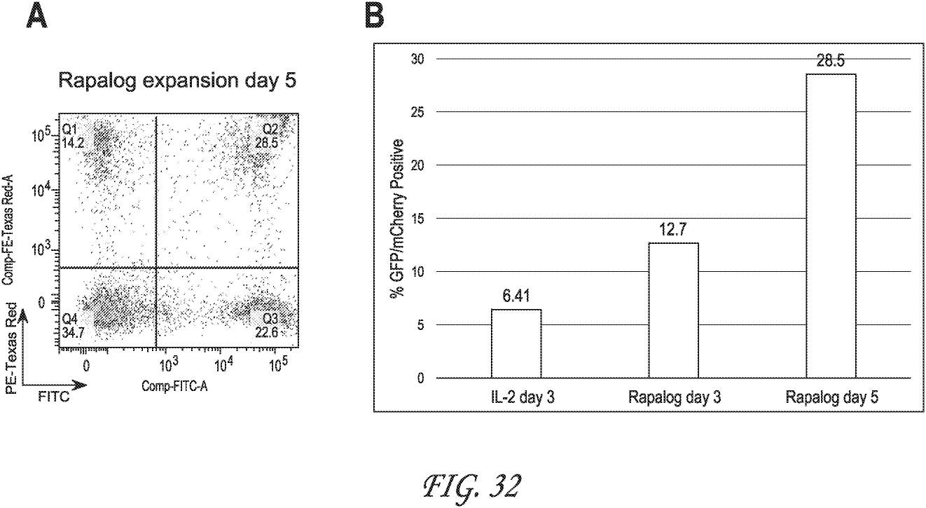

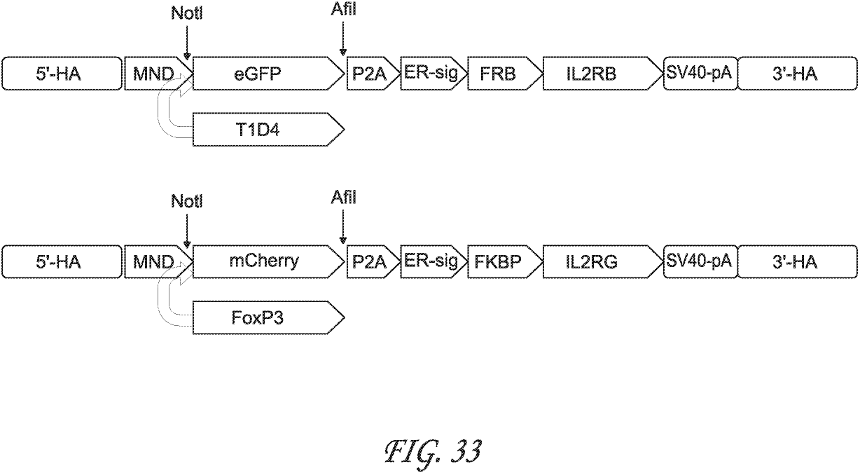

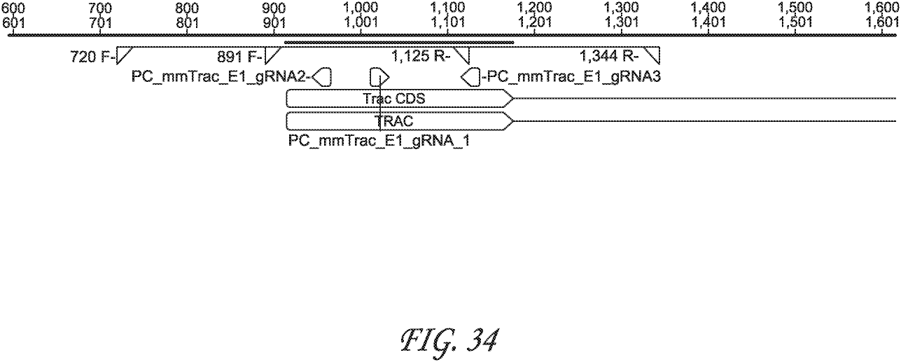

The reproducibility between experiments and variance between donors was tested (FIG. 31). Cells from the same donor from the previous experiment (shown in FIG. 29) were edited and compared with cells from an additional male, Caucasian donor of similar age. The percent dual editing of R003657 donor was 1.1%, which is similar to what was observed previously (FIG. 29). Bi-allelic editing of the second donor, R003471, was 6.4%. Overall, the editing rate changed between donors, but the ratio between GFP-positive, mCherry-positive and double-positive cells remained similar, suggesting variability may be based on how well the donor can be edited. Importantly, dual-edited cells from both donors were successfully enriched-for in the presence of Rapalog, yielding 13.8% and 28.5% GFP/mCherry double-positive cells for donors R003657 and R003471, respectively (FIG. 32).

The results of these studies suggested that incorporation of the split IL-2 CISC in dual HDR editing provides a means of efficient selection and enrichment of dual-edited cells and could provide a method to obtain edited cell numbers necessary for therapeutic use.

In view of the successful bi-allelic editing using MND-eGFP-FRB-IL2RB and MND-mCherry-FKBP-IL2RG cassettes, and enrichment of dual-edited cells using Rapalog, constructs were generated to introduce FoxP3 and a pancreatic islet antigen-specific TCR (T1D4) in combination with the IL-2 CISC components to generate antigen-specific Foxp3+ airT cells (FIG. 33).

### Example 4: Bi-Allelic Targeting for the Generation of Dual Edited Murine CD4+ T-cells

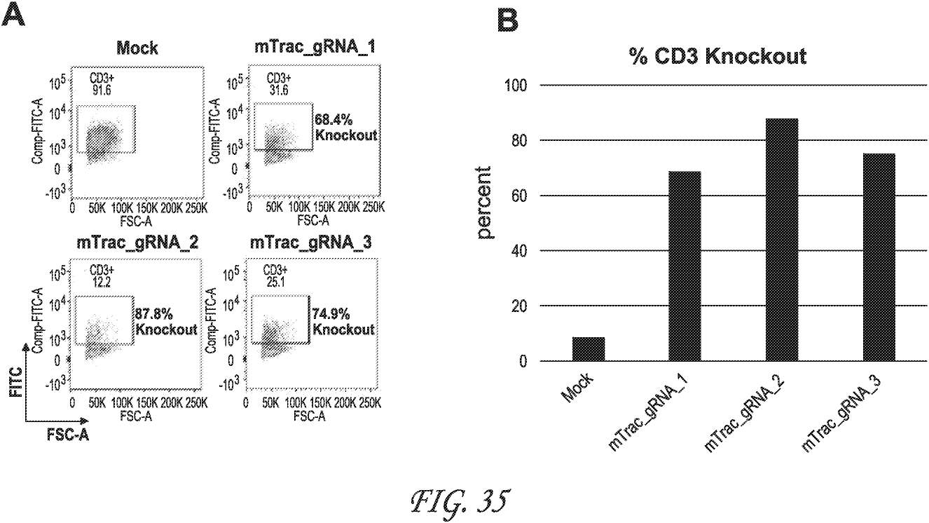

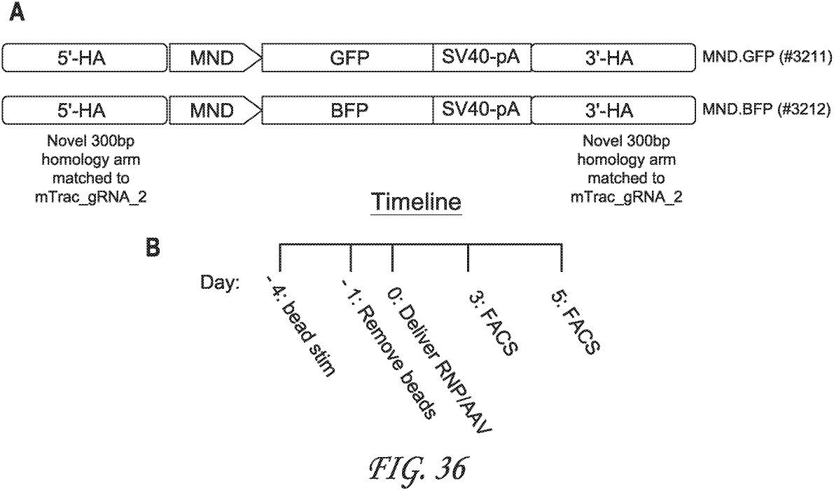

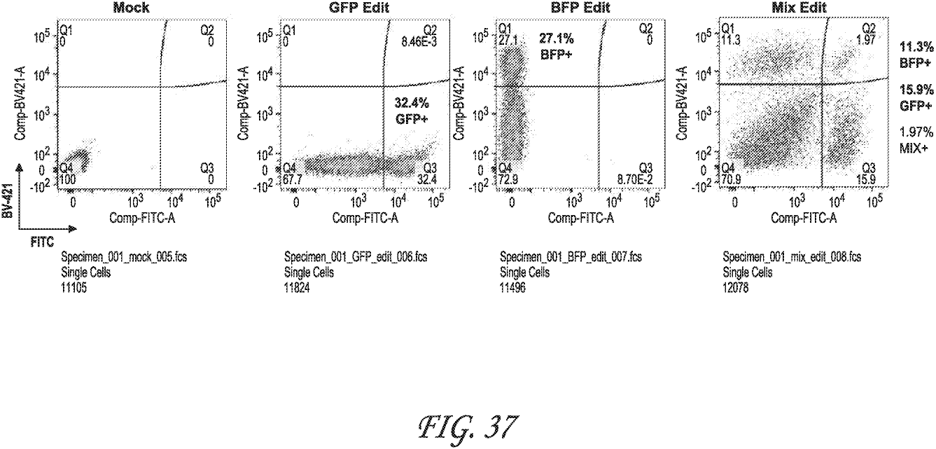

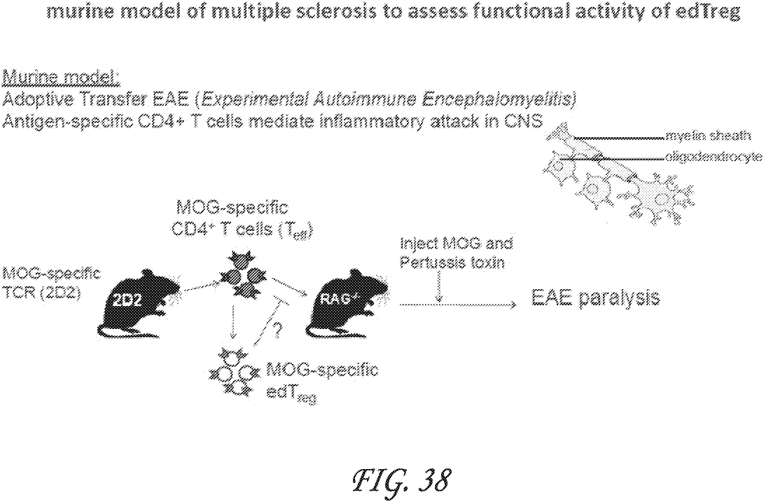

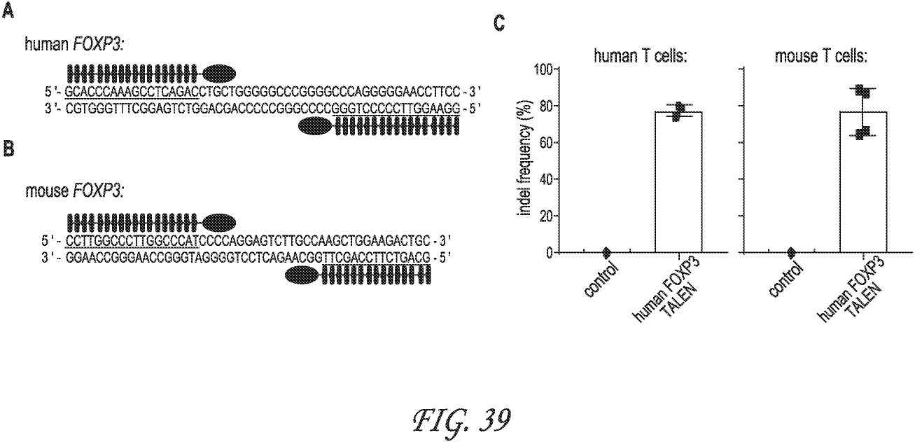

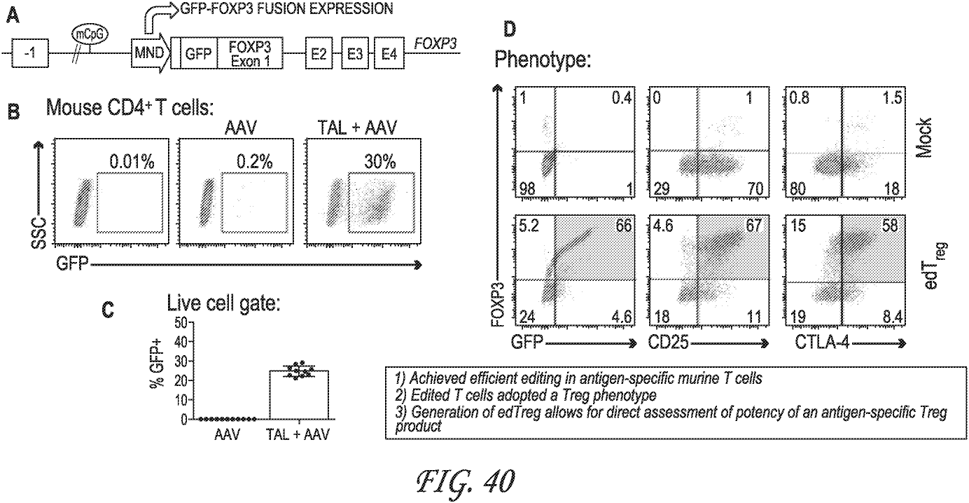

In order to perform studies to assess the efficacy of Ag-specific FoxP3 airT in animal models of diabetes or other autoimmune conditions, analogous tools for editing into the murine Trac locus were generated. Three novel gRNA target sequences within the first exon of murine Trac locus were selected and tested for CD3 knockout in mouse (C57/B6) CD4+ primary T cells (FIG. 34). FIG. 35 shows that mTrac_gRNA_2 resulted in the best knock-out of 87.8%, as measured by flow analysis of mCD3 expression following 2-days post transfection. As with the human construct, MND-GFP and MND-BFP constructs were generated to enable convenient tracking of edited cells. The construct and timeline for this experiment is shown in FIG. 36. As was the case with dual-editing human cells at the TRAC locus, the dual editing efficiency in murine cells was relatively low (1.97%) (FIG. 37).

### Example 5: airT Function in an Antigen-Specific Murine Model of Multiple Sclerosis

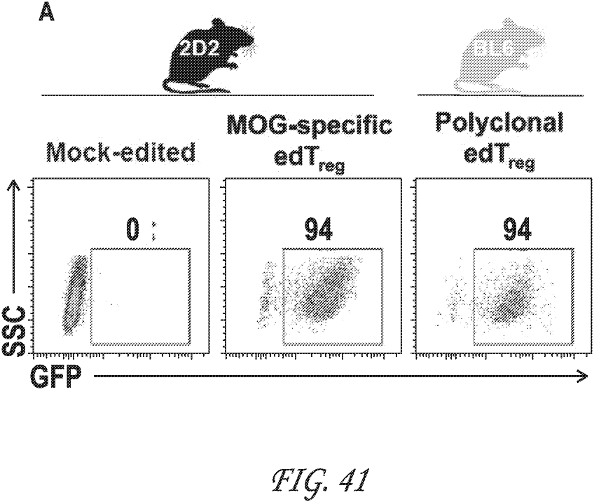

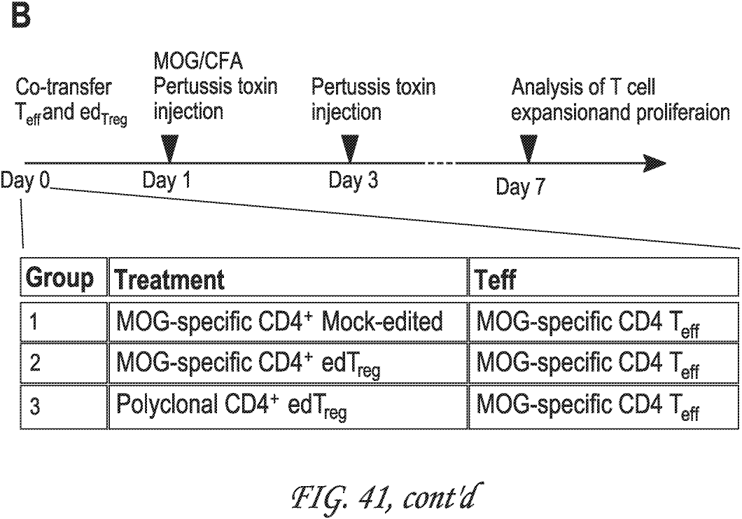

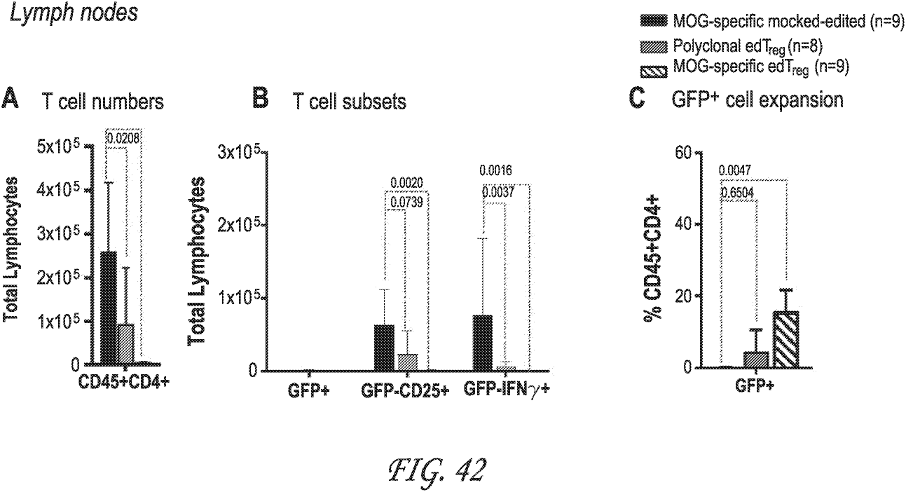

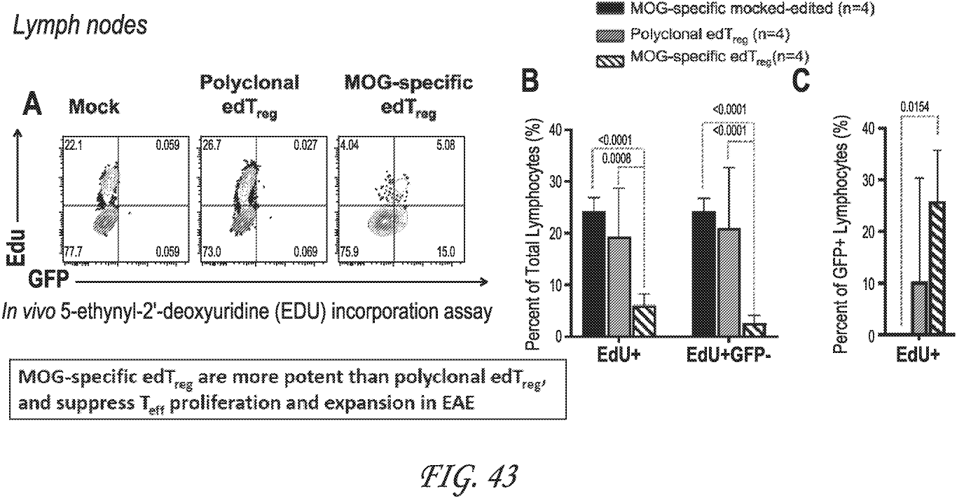

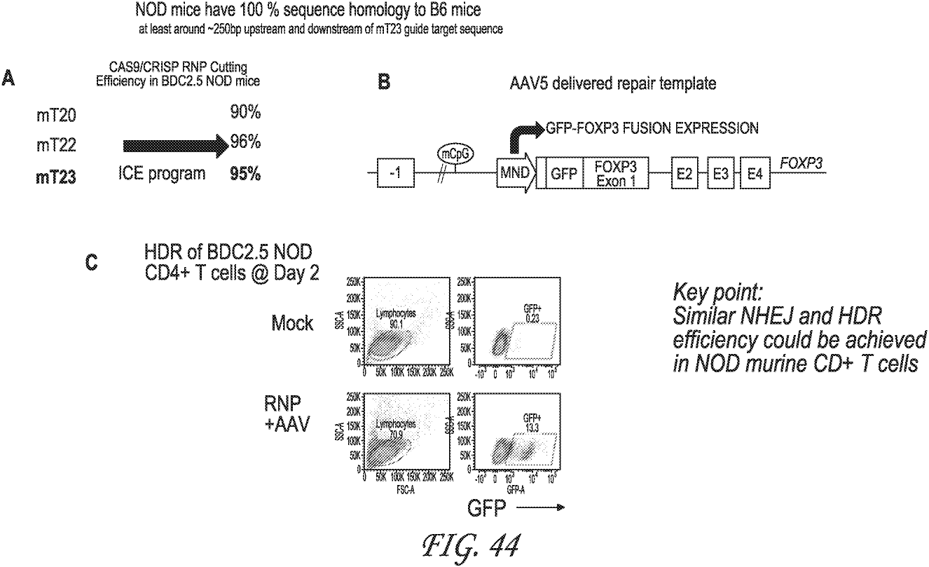

In order to investigate airT function in an antigen-specific in vivo setting, T cells for editing were selected from myelin oligodendrocyte glycoprotein peptide fragment 35-55 (MOG)-specific TCR-transgenic mice (C57B⅙-Tg(Tcra2D2,Tcrb2D2)1Kuch, abbreviated 2D2. MOG challenge of 2D2 transgenic mice leads to experimental autoimmune encephalomyelitis (EAE), a mouse model of multiple sclerosis. EAE in 2D2 mice is not controlled by endogenous 2D2 tTreg present within the central nervous system (CNS), possibly due to high levels of inflammatory cytokines produced by pathogenic T_{eff}. Adoptive transfer of antigen-specific 2D2 airT may suppress T_{eff} expansion in the periphery before these activated effectors migrate to the CNS (FIG. 38). To test this hypothesis, TALEN and AAV donor template reagents were designed that would mimic closely the GFP knock-in editing strategy used to generate the GFP+ human airT. After improved procedures for murine T cell stimulation and mRNA electroporation were designed, murine T cells were transfected with mRNA encoding TALEN pairs specific for the first coding exon of mouse Foxp3. Seven to 9 days post-transduction, approximately 80% of alleles contained indels based on colony sequencing of PCR-amplified gDNA (FIG. 39) indicating efficient target site cleavage. An AAV donor template was cloned that substituted mouse Foxp3 homology arms for the human sequences used in the previous HDR experiments; homology was proximal to, but not overlapping with, the mouse TALEN binding sites. With slight modifications of the conditions used for human CD4+ T cell editing, including using AAV5 capsid for donor template transduction, editing rates of approximately 25-30% (GFP+ cells) were consistently achieved using 2D2 mCD4+ T cells isolated from spleen and lymph nodes (LNs). GFP+ cells were phenotypically FOXP3+ CD25+ CTLA-4 (FIG. 40). CD4+ T cells were also isolated from 2D2neg littermate (C57BL/6) and edited, resulting in airT with a polyclonal pool of TCR specificities for comparison with 2D2 airT. Next, 3.0 ×10⁴ CD4+ T_{eff} cells from 2D2 mice, along with 3.0 ×10⁴ mock or airT, were adoptively transferred into lymphopenic Rag 1-/- mice. Recipient mice were challenged with MOG35-55 peptide in adjuvant followed by pertussis toxin to disrupt the blood-brain barrier. FIG. 41 shows the experimental timeline of cell transfer, immunization, and cell analysis. In this model, recipient animals develop symptoms of EAE (drooping tail progressing to tail, and then hind limb, paralysis) beginning at approximately 7-10 days post cell transfer. To assess effector T cell priming, recipient mice were euthanized at day 7 post-transfer and inguinal and axillary LNs were collected. The percentage of LN CD45+ CD4+ T cells were 2-fold lower in recipient of antigen-specific 2D2 airT compared to recipients of mock-edited cells, and 1.7-fold lower compared with recipients of polyclonal C57B⅙ airT. The absolute number of CD4+ CD45+ T cells was markedly reduced in both airT cohorts relative to the mock control, with 2D2 airT and C57B⅙ airT having 49-fold fewer and 18-fold fewer CD4+ CD45+ T cells, respectively. In all cohorts, the majority of CD45+ CD4+ cells were GFP-, and fewer GFP- T cells from mice receiving 2D2 edTreg expressed inflammatory markers CD25 and IFN-γ (FIG. 42). To determine if the observed effect was due to reduced T_{eff} proliferation, a subset of animals were injected with the thymidine analog 5-ethynyl-2′-deoxyuridine (EdU) 2 hours prior to sacrifice and its incorporation into gDNA was detected after a “click” reaction by flow cytometry (FIG. 43). 2D2 airT reduced the overall percentage of GFP- cells that had incorporated EdU by 22% and 18% relative to groups treated with mock or polyclonal airT, respectively. Importantly, GFP+ cells from 2D2 airT (-25%) and, to a lesser extent, from C57B⅙ airT (-10%) recipient cohorts incorporated EdU, consistent with the ability of tTreg to proliferate in vivo in response to self-antigen-stimulation. These combined findings show that murine airT function in vivo to restrain pathogenic T_{eff} priming and that antigen-specific airT exhibit greater activity and expansion in comparison with polyclonal airT.

### Example 6: airT Function in an Antigen-Specific Murine Model of Type 1 Diabetes (TIP)

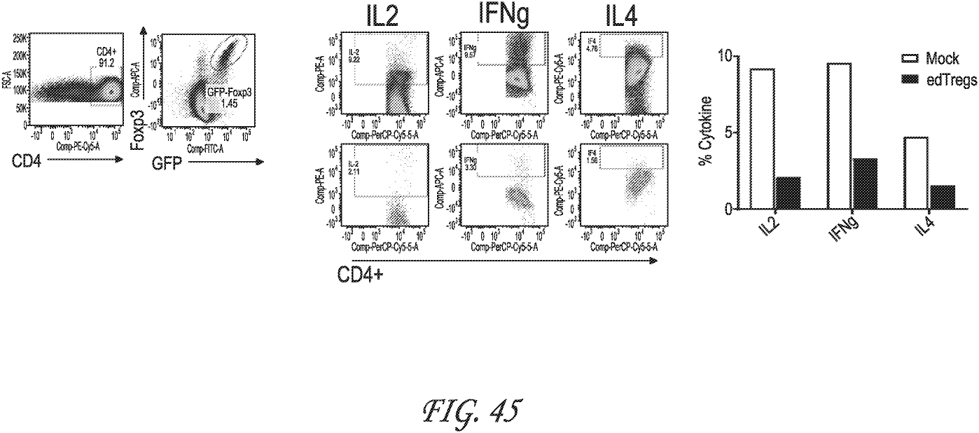

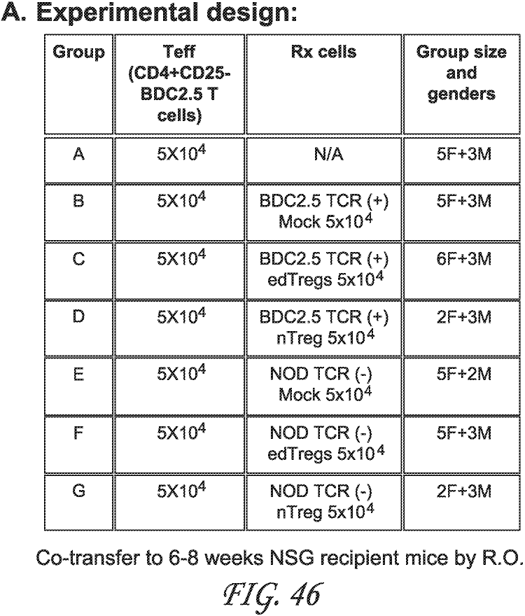

To investigate the efficacy of antigen-specific airT cells in an in vivo model of autoimmune T1D, a BDC2.5NOD-NSG adoptive transfer model was used as a tool to determine if Foxp3-edited antigen specific T cells could delay or reverse the onset of disease. NOD mice: (NOD/ShiLtJ strain) were used as a polygenic model for autoimmune Type 1 Diabetes (T1D). In this model, onset of diabetes is marked by moderate glycosuria and by a non-fasting plasma glucose higher than 250 mg/dl. Diabetic mice are hypoinsulinemic and hyperglucagonemic, indicating a selective destruction of pancreatic islet beta cells. It is currently the most widely used polyclonal autoimmune animal model to study spontaneous T1D. BDC2.5NOD mice: [NOD.Cg-Tg(TcraBDC2.5,TcrbBDC2.5)1Doi/DoiJ] carry both rearranged TCR alpha and beta genes from the cytotoxic CD4+ T cell clone BDC-2.5. Mature T cells in these mice express only the BDC2.5 TCR. On the NOD background, mice carrying the transgenes have a reduced incidence of diabetes relative to NOD/ShiLtJ controls. However, following transfer of CD4+CD25- BDC-2.5 T cells into immunodeficient recipient mice, recipient mice will quickly develop overt diabetes. In previous published studies, nTregs from antigen-specific BDC2.5NOD mice effectively prevent and reverse autoimmune diabetes in NOD mice relative to the nTregs from polyclonal WT NOD mice. Thus, in these experiments, these animal models were utilized as a tool to determine if Foxp3-edited antigen-specific T cells could delay or reverse the onset of autoimmune T1D compared to nTregs from WT NOD. The ability to generate airT in NOD mice was tested. Previous studies demonstrate the ability to generate airT cells in murine CD4+ T cells from B6 mice using HDR editing of the Foxp3 locus (WO 2018/080541 and US 2019/0247443 which are each incorporated by reference in its entirety). Here, these studies were expanded and show that the same AAV donor templates have similar NHEJ and HDR efficiency in NOD murine CD4+ T cells (FIG. 44). Importantly, the Foxp3-edited BDC2.5NOD CD4+ T cells have a Treg phenotype expressing increased Foxp3, and less inflammatory cytokines as compared to mock cells (FIG. 45).

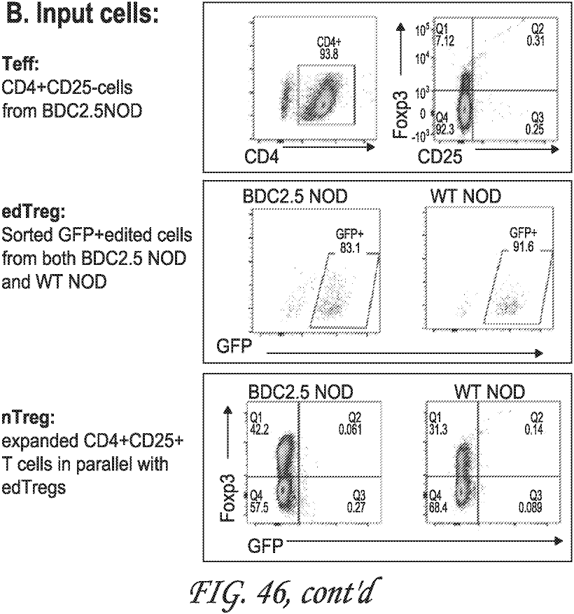

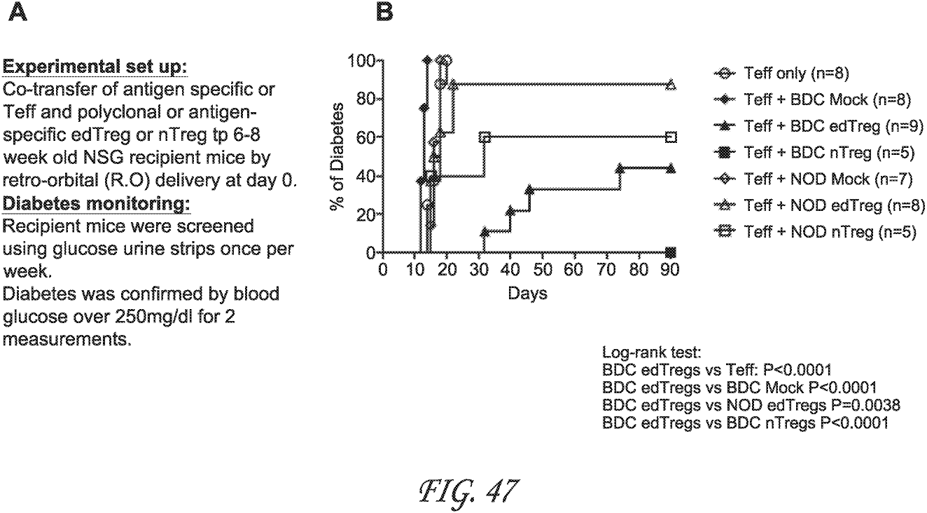

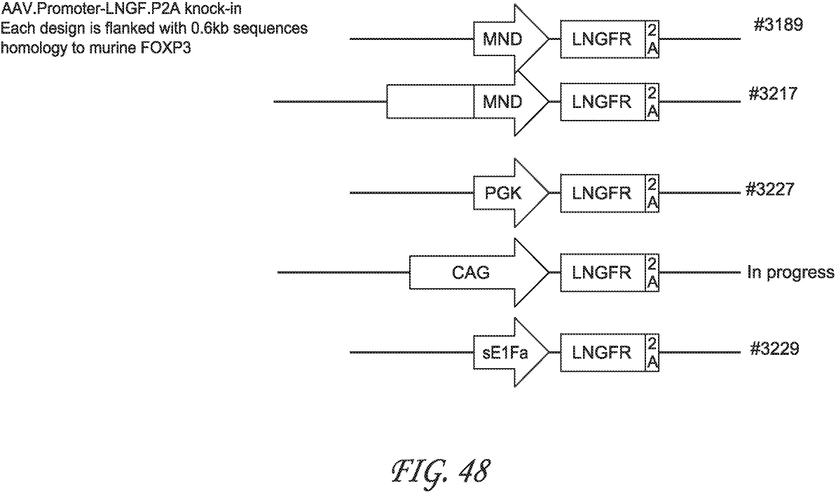

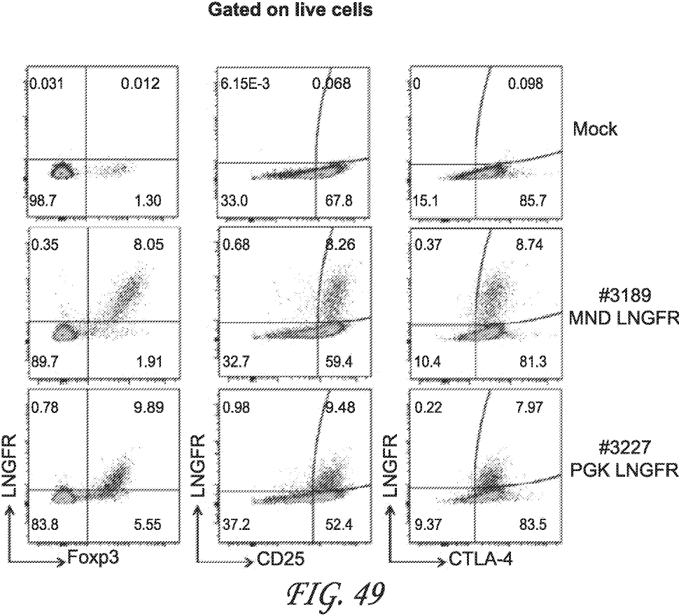

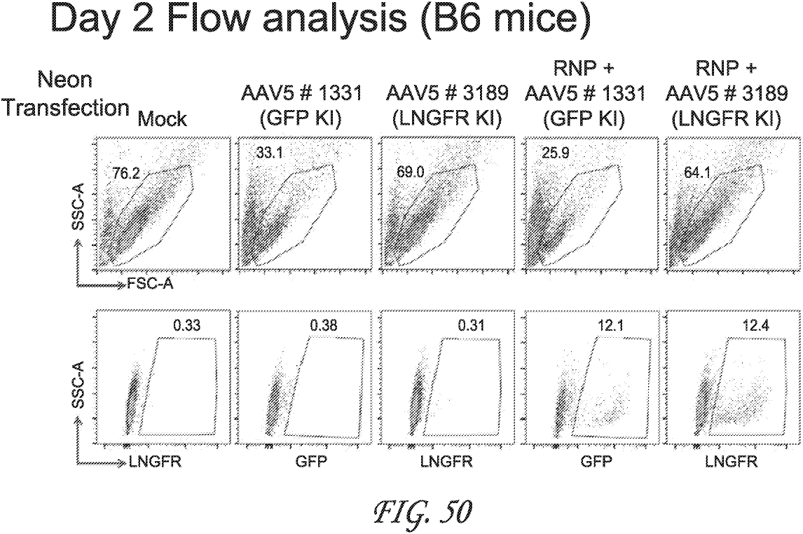

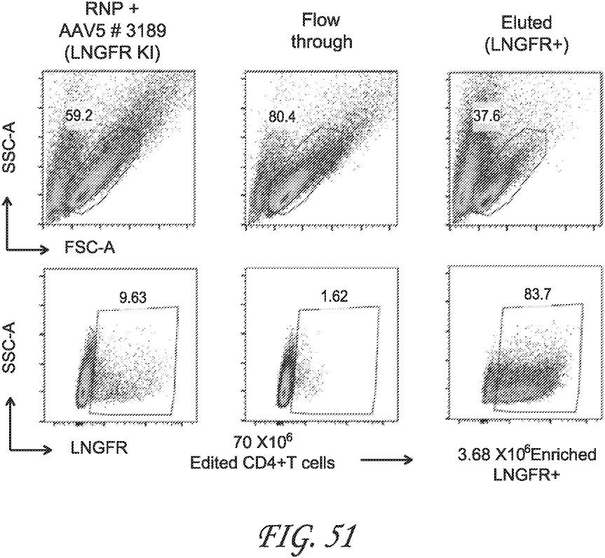

Antigen-specific airT cell function in an NSG adoptive transfer model may be more efficacious than non-antigen specific airT cells at delaying or reversing the onset of autoimmune T1D. nTregs were included in this study as this population has previously been shown to reduce the onset of T1D. The experimental design and the phenotype of the input T_{eff}, airT and nTreg cells are shown in FIG. 46. Like nTreg, airT lead to a reduction in percentage of diabetes compared to mock airT or animals receiving T_{eff} only (FIG. 47). Importantly, administration of BDC airT leads to a statistically significant decrease in percentage diabetes compared to polyclonal NOD airT. This finding demonstrates that Ag-specific airT more effectively prevent diabetes development compared with polyclonal airT. Of note, previous studies have shown that the N-terminal GFP-FOXP3 fusion protein functions as a hypomorph and can actually accelerate autoimmune diabetes within the immunocompetent NOD background. Consistent with this idea, while antigen-specific nTregs performed better than antigen-specific airT in these studies, a significant suppressive effect was still observed with airT expressing GFP-FOXP3 fusion protein. Testing the airT expressing FOXP3 without the N-terminal GFP fusion (including airT cells expressing a clinically relevant cis-linked LNGFR selectable marker; see below), an improved protective effect would be expected.

Taken together, these findings clearly showed that in the BDC2.5 NOD T cell into NSG adoptive transfer model, Ag-specific airT function to prevent diabetes development as compared with polyclonal airT.

### Example 7: Engineering AAV Donor Template Design to Generate airT Product With LNGFR Selectable Marker

The ability to enrich murine cells following editing is important for generating sufficient numbers of edited cells to perform in vivo experiments without sorting. To address this, a cis-linked LNGFR selectable marker has been developed for use in purification of murine edTeg products. FIG. 48 shows the design of repair templates used in murine Foxp3 editing. Each template contains a LNGF.P2A knock-in (ki) but varies in terms of the promoter. In addition, the presence and absence of 07UCOE was tested with MND promoter. Following transfection of RNP+AAV5 #1331 (MND-GFP), #3189 (MND-LNGFR), and #3227 (PGK-LNGFR) in B6 CD4+ T cells, the editing efficiency of GFP and LNGFR KI was very similar (FIG. 49, FIG. 50). In addition, an 8.7-fold enrichment of LNGFR+ cells was demonstrated using an anti-LNGFR microbeads and magnetic field separation (FIG. 51). These data suggest LNGFR can successfully be used as a selection and enrichment method of murine CD4+ T cells.

### Example 8—Methods

### Foxp3 Editing

CD4+ T cells were isolated from PBMCs using MACS CD4+ T cell isolation kit and activated with CD3/CD28 activator beads (1:1, bead to cell ratio) and IL-2, IL-7, and IL-15. Beads were removed after 48 hr activation and cells were rested for another 16-24 hr. For Foxp3 editing using Cas9/CRISPR, cells were transfected by electroporation with RNP complex combined with Cas9 and guide RNA and then transduced with AAV template (AAV FOXP3 ex1.MND-LNGFRki). For Foxp3 editing using TALEN nuclease, cells were transfected by electroporation with TALEN RNA targeting FOXP3, followed by transduction with AAV template (AAV FOXP3 ex1.MND-GFPki). Cells were expanded in media with IL-2 after editing.

### Generation of airT Cells With Islet-TCR

CD4+ T cells were isolated from PBMCs and activated with CD3/CD28 activator beads and IL-2, IL-7, and IL-15. Transduction with lentiviral vectors encoding GAD65 or IGRP specific TCRs (4.13, T1D2, T1D4, T1D5-1, or T1D5-2) was performed at MOI 10 with protamine sulfate after 24 hr activation. Beads were removed after total 48 hr incubation from the initial activation. Cells were rested for another 16-24 hr and then edited. For Foxp3 editing, cells were electroporated with RNP complex combined with Cas9 and guide RNA and then transduced with AAV FOXP3 ex1.MND-LNGFRki template. Cells were expanded in media with IL-2 after editing and editing rate and TCR-transduction were measured 3-4 days after editing. airT cells were enriched by LNGFR expression using MACSelect LNGFR beads, aliquoted, and frozen down for future experiments.

### Generation of Antigen-Specific airT Cells

For generating T cells specific for Flu or Tetanus, CD4+ CD25- cells were isolated from PBMCs and co-cultured with APC (irradiated autologous CD4-CD25+ cells) and MP, HA, and TT peptides in the presence of IL-2. CD4+ T cells were stimulated twice with peptides and APC for 9 days and then activated with CD3/CD28 beads for Foxp3 editing using TALEN and AAV FOXP3 ex1.MND-GFPki template. 3 days after editing, GFP+ cells were sorted by flow and expanded with CD3/CD28 beads. Beads were removed after 7 days expansion and airT cells were harvested for suppression assay after another 4 days incubation.

For generating islet-specific T cells by peptide stimulation, CD4+CD25-cells were isolated from PBMCs and co-cultured with APC and a pool of islet-specific antigens (total 9 peptides from IGRP, GAD65, and PPI). After 2 weeks of expansion, cells were harvested and stained with tetramers specific for 9 islet peptides for sorting. Sorted islet-specific CD4+ T cells were activated with CD3/CD28 activator beads for Foxp3 editing using Cas9/CRISPR and AAV FOXP3 ex1.MND-LNGFRki template. Cells were stained by tetramers and analyzed by flow 3 days after editing.

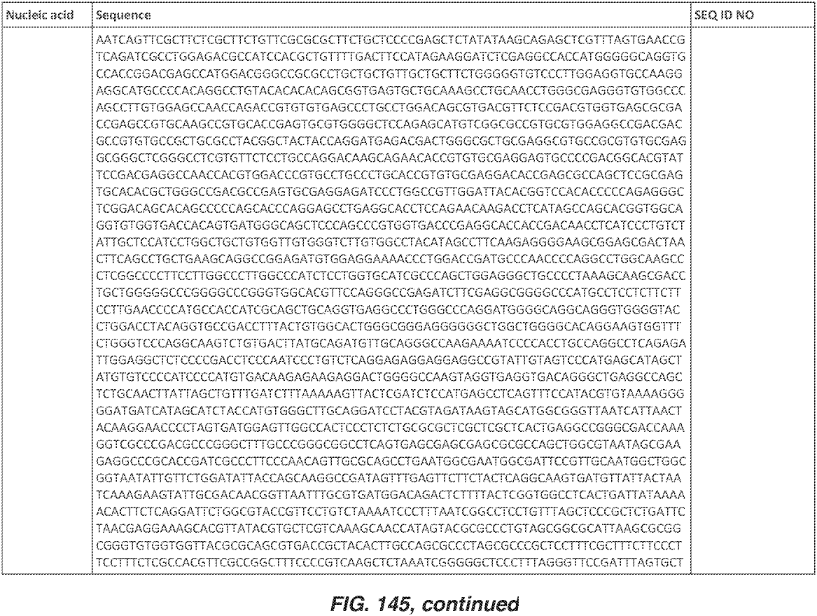

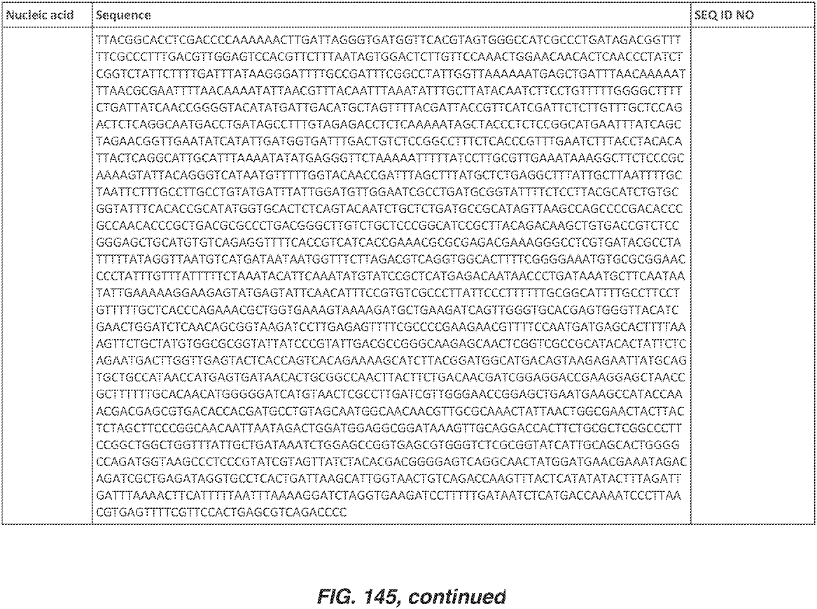

Certain nucleic acid sequences useful with embodiments provided herein are listed in the TABLE depicted in FIG. 145.

### Example 9—Comparison of airT With T Cells Expressing Lentiviral (LV)-Delivered FOXP3

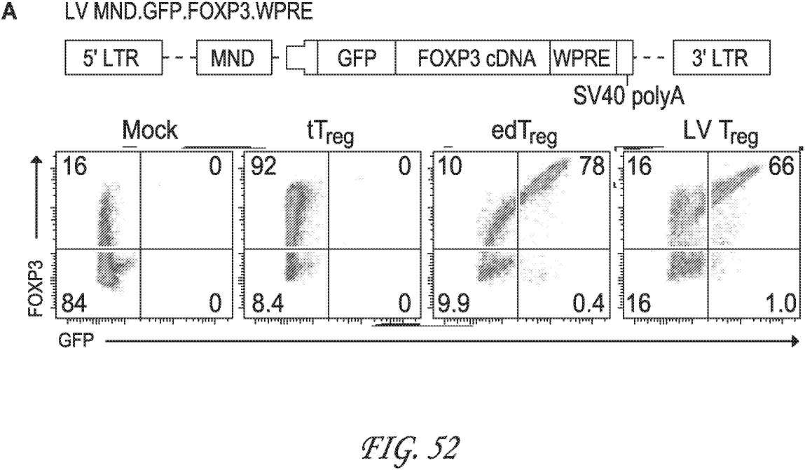

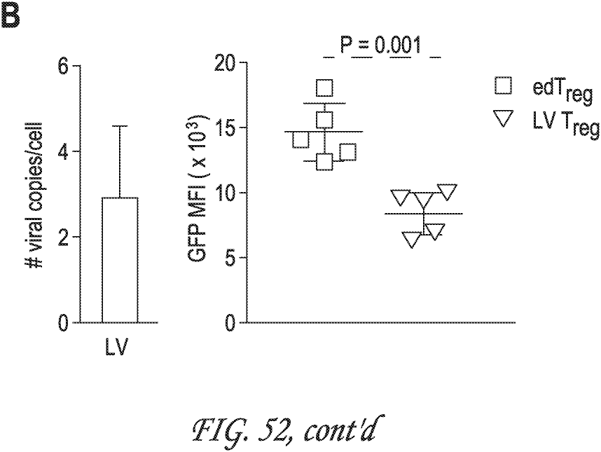

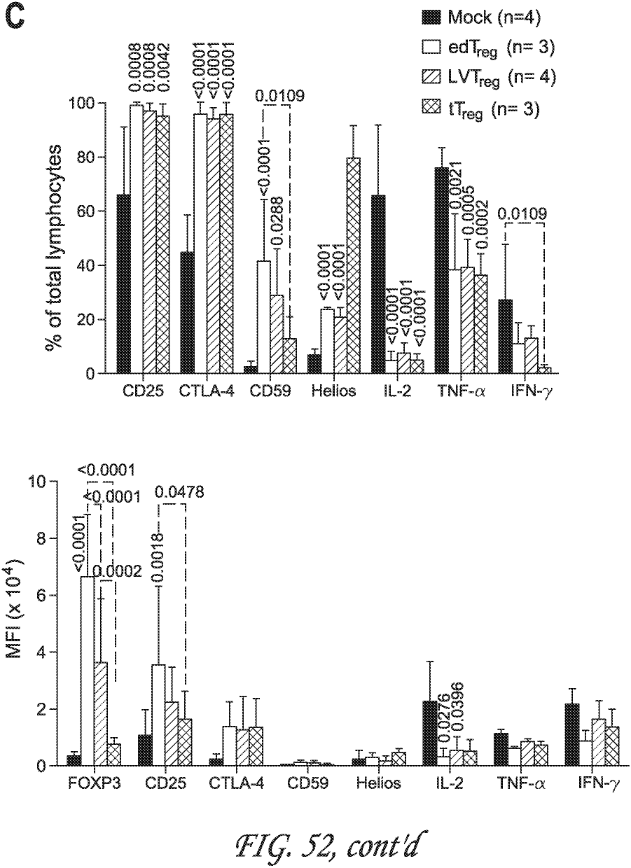

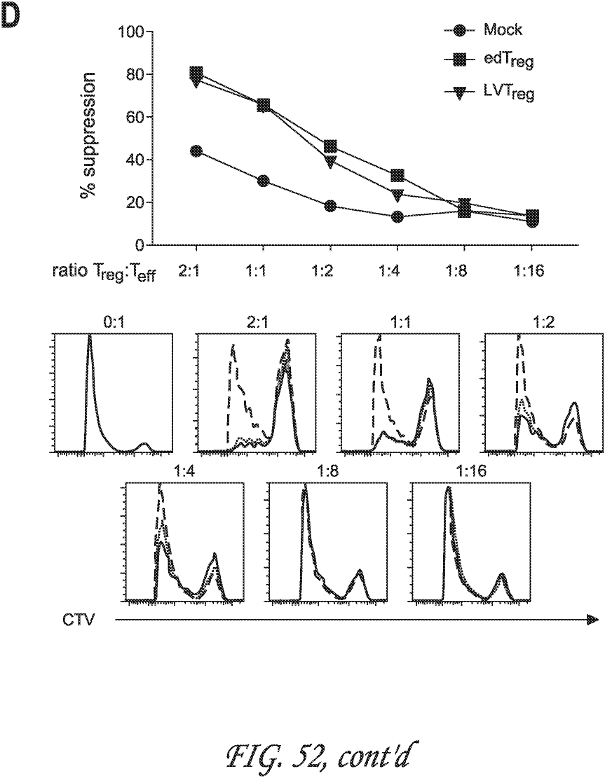

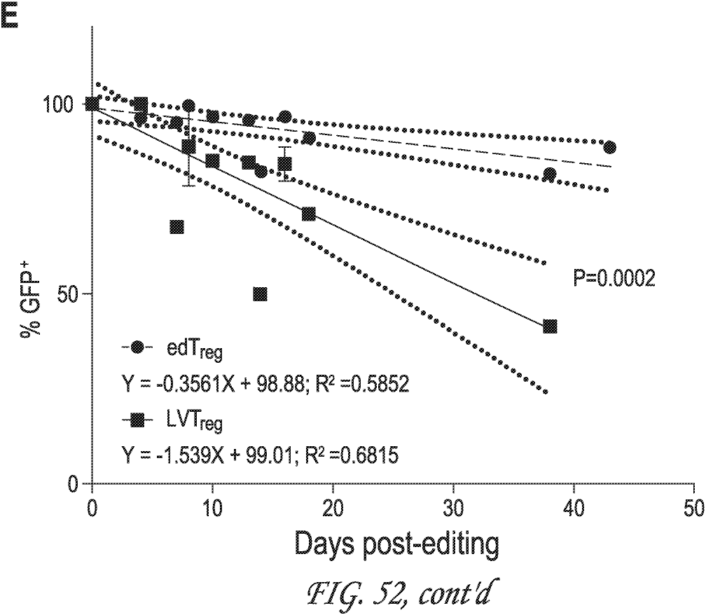

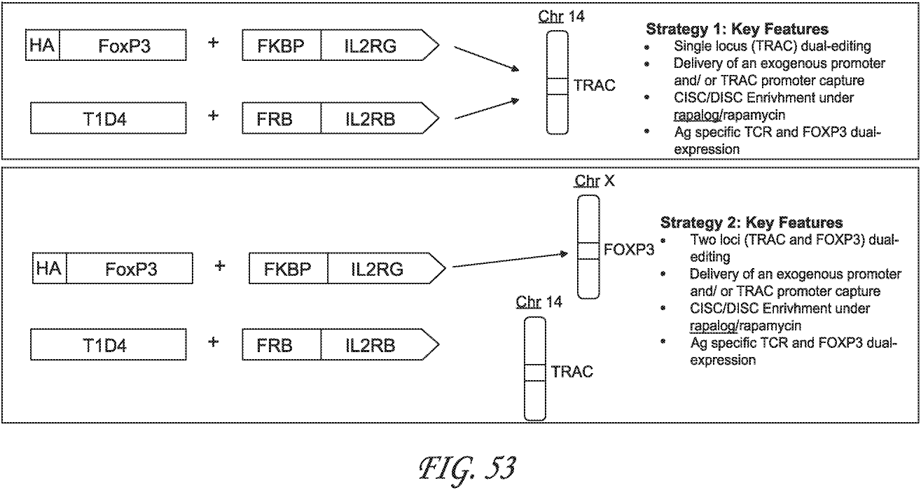

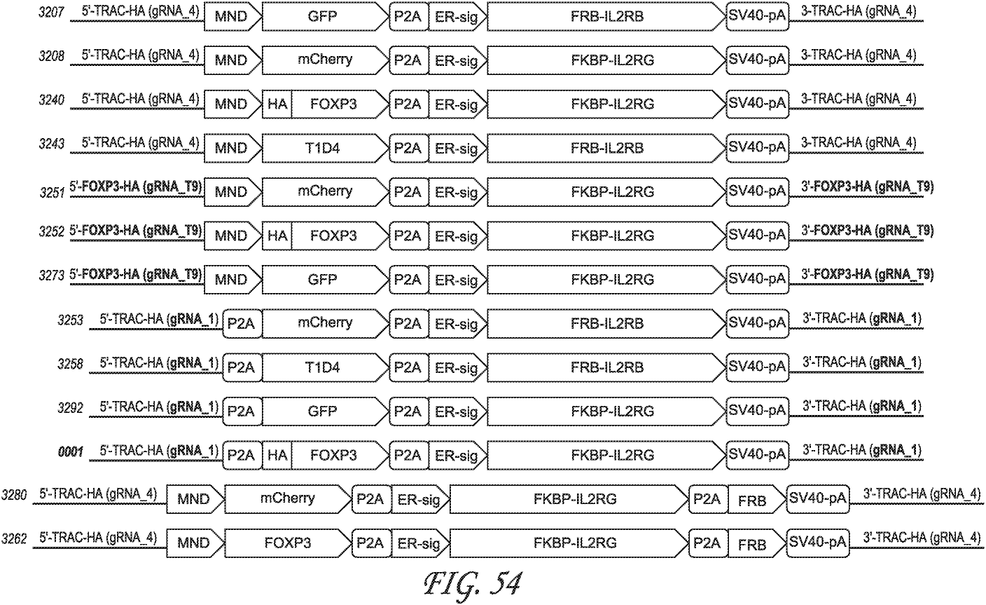

LV transfer of a FOXP3 cDNA expression cassette into conventional T cells has been shown to confer a Treg-like phenotype and suppressive characteristics in vitro and in vivo (Allan et al. Mol Ther 16:194-202 (2008); Passerini et al. Sci Transl Med 5:215ra174 (2013)). As a comparison for the presently disclosed editing strategy, a LV construct was generated to deliver a cDNA encoding the same GFP-FOXP3 fusion protein made by the airT cells (FIG. 52A). The gene editing and viral transduction procedures produced similar proportions of GFP+FOXP3+cells (FIG. 52A). LV-treated cells (LV Treg) had an average of 3.0 lentiviral copies per GFP+ cell genome; airT have only one targeted insertion per cell in this experiment, edited T cells from male donors. Despite their copy number differences, the MFI of GFP+ cells were consistently lower in LV Treg than in airT (FIG. 52B), consistent with more efficient expression from a genomic vs. cDNA context, or LV integration in transcriptionally less permissive loci. Both methods of enforcing FOXP3 expression skewed the T_{eff} towards tTreg phenotypes, including upregulation of CD25, CTLA-4, and Helios, and down-regulation of IL-2, TNF-α, and IFN-y (FIG. 52C). Except for FOXP3, the percentage of cells expressing regulatory T cell markers, as well as the mean expression (as assessed by MFI) per cell, were similar between airT and LV Treg. airT and LV Treg exhibited a similar ability to suppress polyclonal T_{eff} proliferation in vitro (FIG. 52D). Importantly, however, FACs purified LV Treg cells lost GFP expression over 5 weeks in culture compared to airT (FIG. 52E; starting % GFP+ >99% for both). This latter finding demonstrates that HDR editing more effectively maintains high-level FOXP3 expression compared with LV delivery using the identical promoter construct. These findings are unexpected based upon previous reports using LV delivery of FOXP3 and support the concept that HDR editing of the FOXP3 locus provide a more stable platform for sustained expression of FOXP3 in CD4 T cells.

### Example 10—Dual-Editing Strategies

FIG. 53 provides an overview of the HDR gene-editing strategies developed to generate antigen-specific airT via HDR-editing-only approaches. These novel approaches eliminated the requirement for use of LV for TCR delivery and are designed to generate airT that concurrently: a) lack endogenous TCR expression; b) express an islet-specific T1D (or another disease-relevant, antigen-specific TCR); and c) can be enriched in vitro and in vivo using the novel CISC/DISC IL-2 platform. As shown in FIG. 53, strategies were developed to achieve the above goals by either targeting a single locus (TRAC) or two-loci (TRAC and FOXP3). Successful application of both of these strategies is illustrated below.

FIG. 54 provides a schematic overview and construct number designation for each of the AAV HDR donor constructs used in the studies described below for either single and two locus dual-editing approaches for generation of Ag-specific edTreg; with or without IL2-CISC/DISC selection capacity.

### Dual Editing of Human CD4+ T Cells - Examples of Single Locus Approach

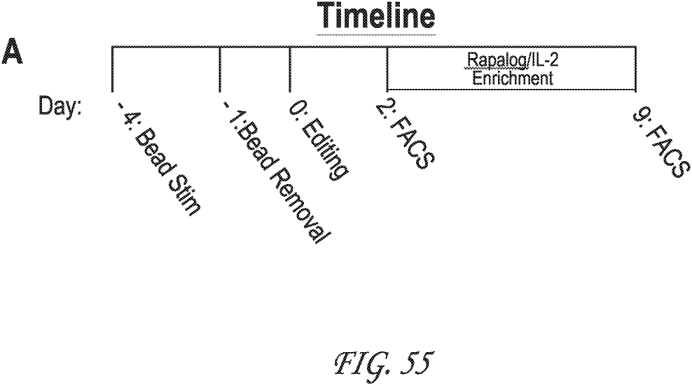

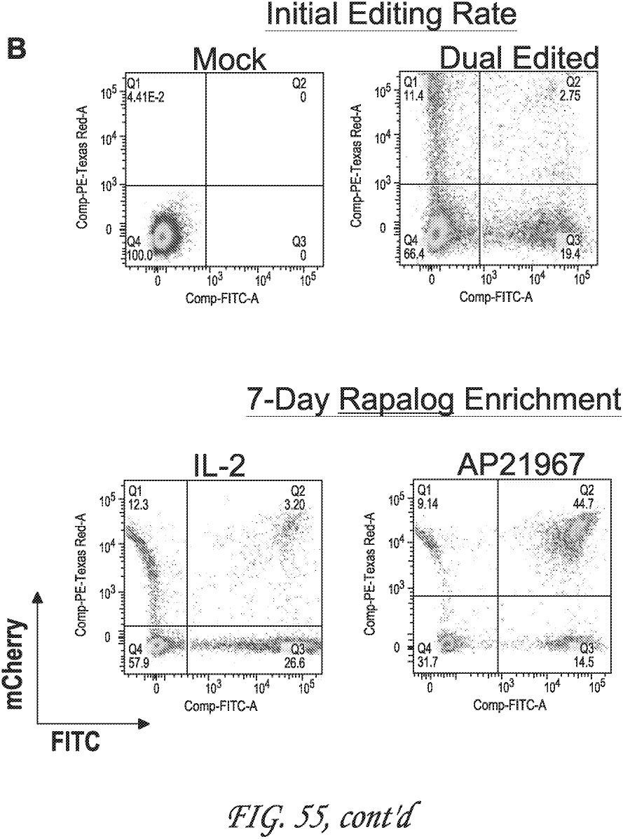

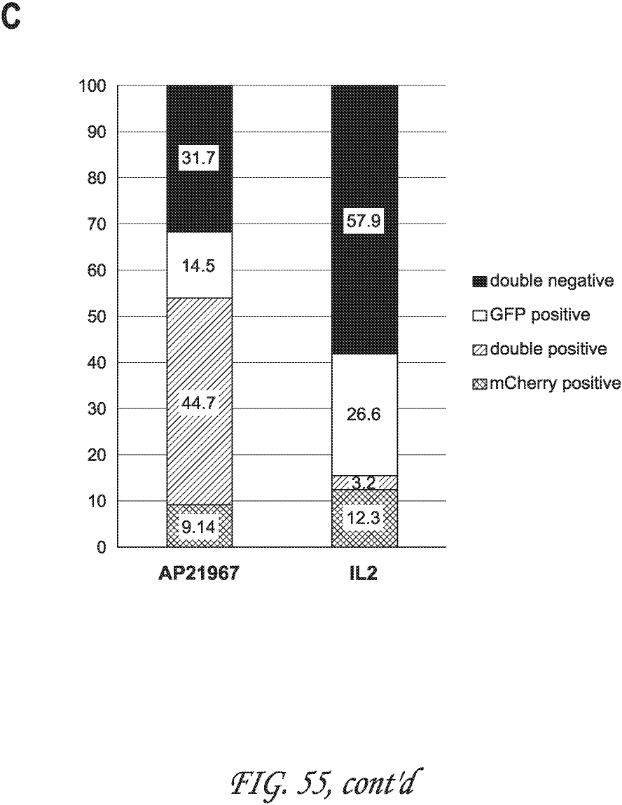

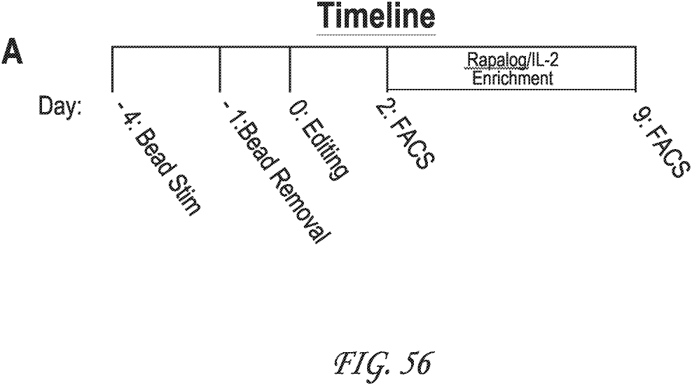

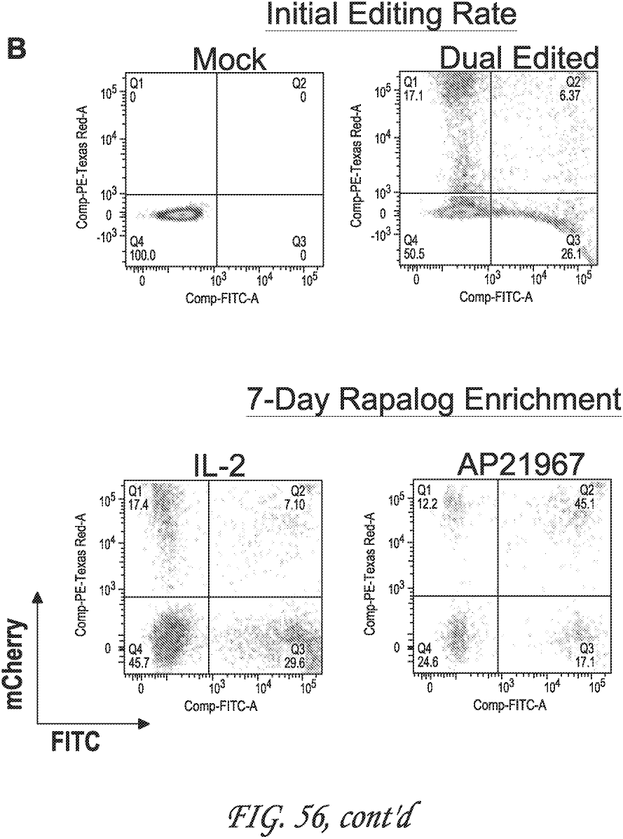

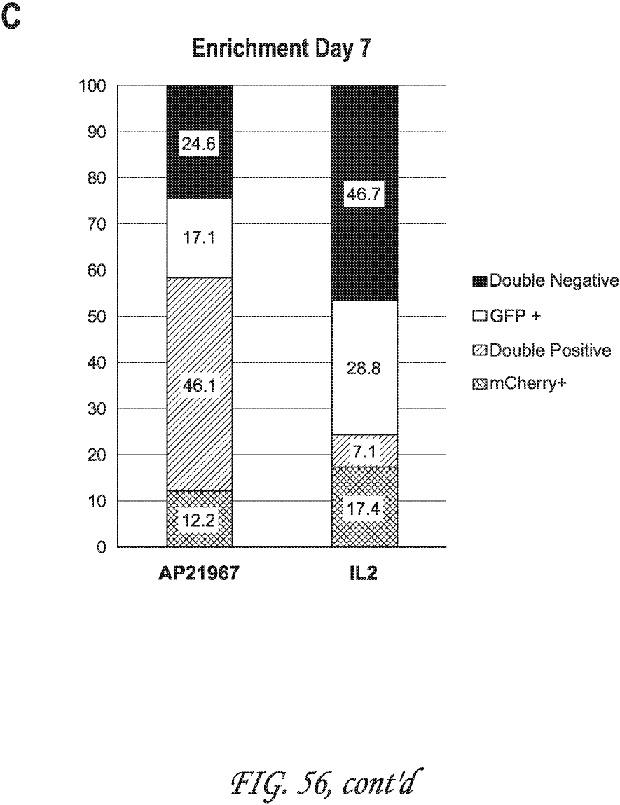

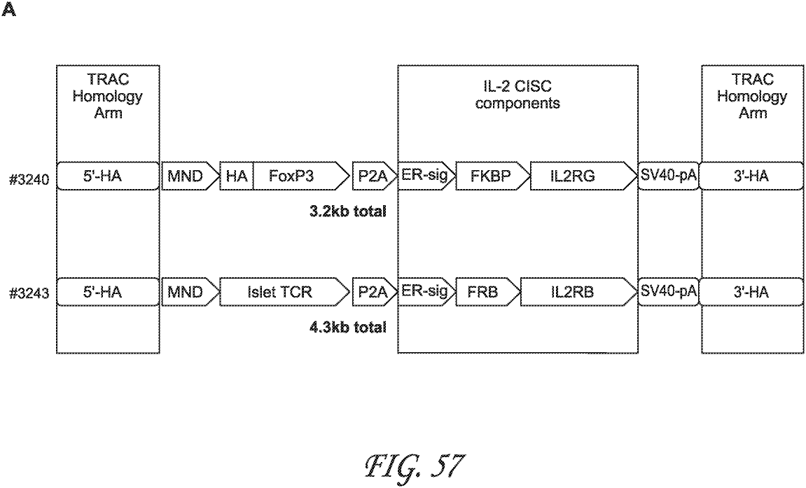

FIG. 55 and FIG. 56 relate to reproducibility between experiments and variance between donors. Two donors were edited with AAV #3207 (MND.GFP.FRB-IL2RG) and #3208 (MND.mCherry.FKBP-IL2RG), used in previous experiments and compared the repeat experiments to the original data. In these experiments, both HDR repair templates are targeted to a single sgRNA cut site within the first exon of the TRAC locus. The percent dual-editing (incorporation of both the GFP and mCherry split-CISC cassettes in a single cell) of donor R003657 was 2.75% (FIG. 55) which is similar to results observed in two prior data sets (1.44% and 1.1% for donor R003657). Percent dual-editing of the second donor (R003471) was 6.78% (FIG. 56), also similar to the 6.4% observed in the original data set. Both donors were male, Caucasian of similar age. Overall, the editing rate varied between donors, but each donor had similar editing rates between experiments, suggesting that variability is based on how well the donor can be edited and not the between different experiments of the same donor. Importantly, using edited T cells derived from both human donors, dual-edited cells were successfully enriched in the presence of a heterodimer-inducing rapamycin analog (Rapalog, AP21967) to a similar level to what was previously observed. This study yielded 44.7% and 46.1% GFP/mCherry double positive cells for donors R003657 and R003471 respectively after 7 days of Rapalog enrichment (FIG. 55, FIG. 56). As expected, there was no enrichment in the presence of IL-2.

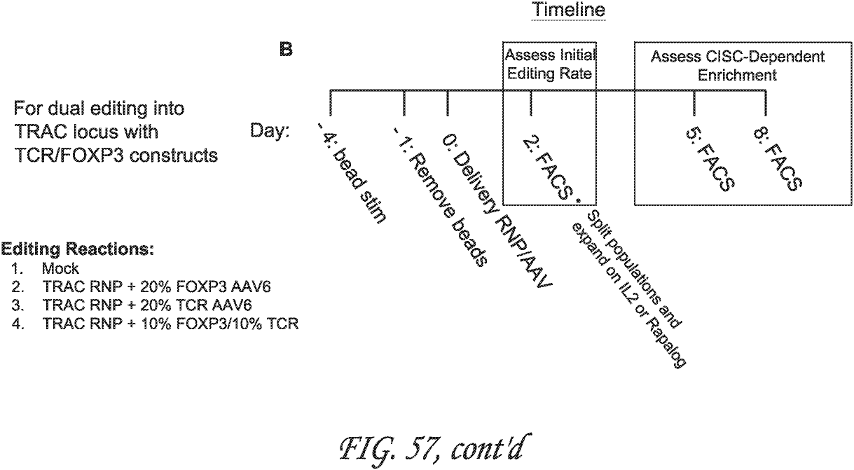

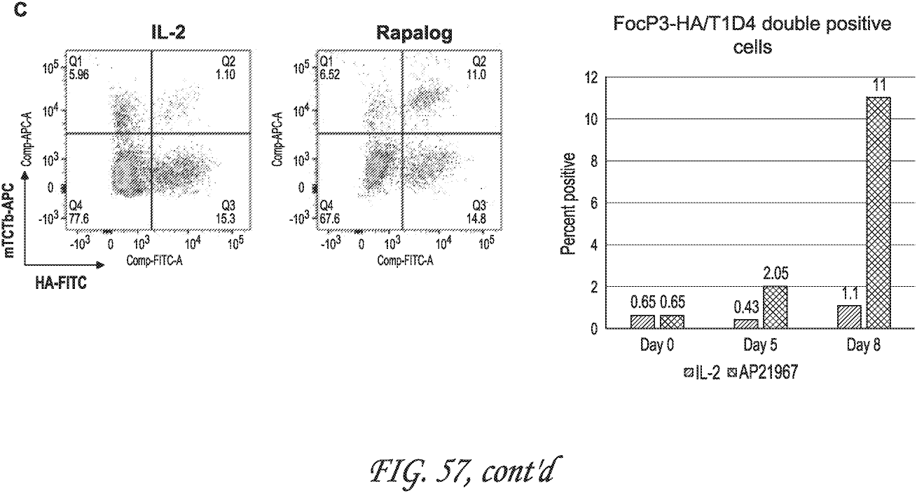

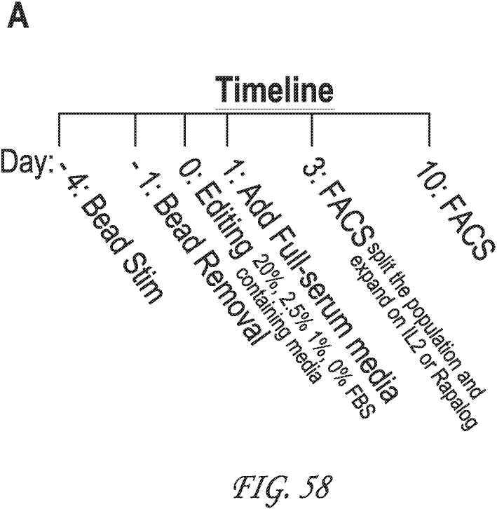

The results of these studies demonstrate that incorporation of the IL-2 split-CISC in dual HDR editing strategies provides a means of efficient selection and enrichment of dual edited cells which is reproducible between donors and repeat experiments. Following successful dual editing using the MND.GFP.FRB.IL2RB (#3207) and MND.mCherry.FKBP.IL2RG (#3208) cassettes, and enrichment of dual edited cells using Rapalog, constructs were generated that can be used in introducing HA tagged FOXP3 and a pancreatic islet antigen-specific TCR (T1D4) in combination with the IL-2 CISC components (#3240 and 3243 respectively) to generate antigen-specific FOXP3+ edTreg cells (FIG. 57).

After replacing GFP with T1D4 and mCherry with HA-tagged FOXP3, respectively, the MND.HA.FOXP3.FKBP.IL2RG (#3240) and MND.T1D4.FRB.IL2RB (#3243) constructs were used to test the initial editing rates and expansion of FOXP3 expressing T1D4 positive human edTregs. Constructs are shown in FIG. 57 (A) and the timeline and editing conditions for this experiment are shown in FIG. 57 (B). Despite lower initial editing rate compared with using the GFP/mCherry -CISC constructs (FIG. 55, FIG. 56), 2.75% and 6.37% double-positive GFP/mCherry cells versus 0.65% double-positive FOXP3/T1D4 cells, (FIG. 57), the double-positive FOXP3/T1D4 cells could be significantly enriched. In the presence of Rapalog, FOXP3/T1D4-positive cells enriched from 0.65% to 11% after 8 days of treatment compared to 1.1% with IL-2 treatment (FIG. 57(C)). The reduction of initial editing rate with these constructs compared to the Split-CISC constructs containing GFP and mCherry (#3207 and #3208) could potentially be due to the increased size of the T1D4 HDR template. MND.T1D4.FRB.IL2RB (#3243) is 4.3 kb, which was significantly larger than MND.mCherry.FKBP.IL2RB (#3208) at 2.7 kb.

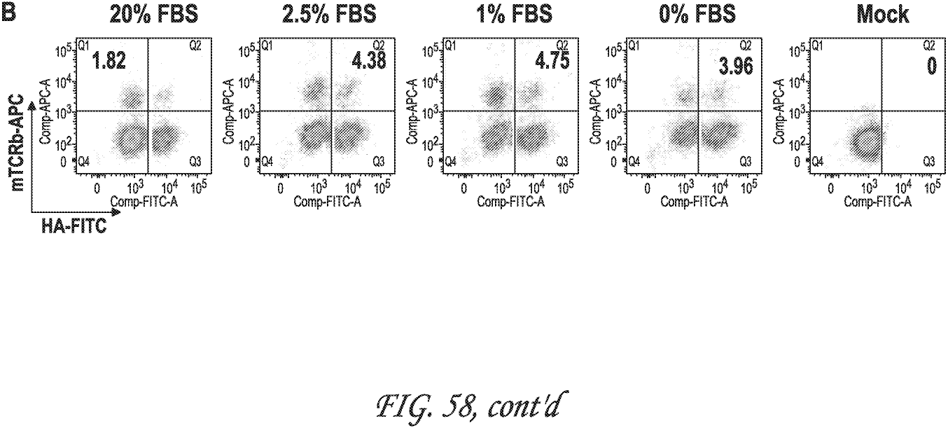

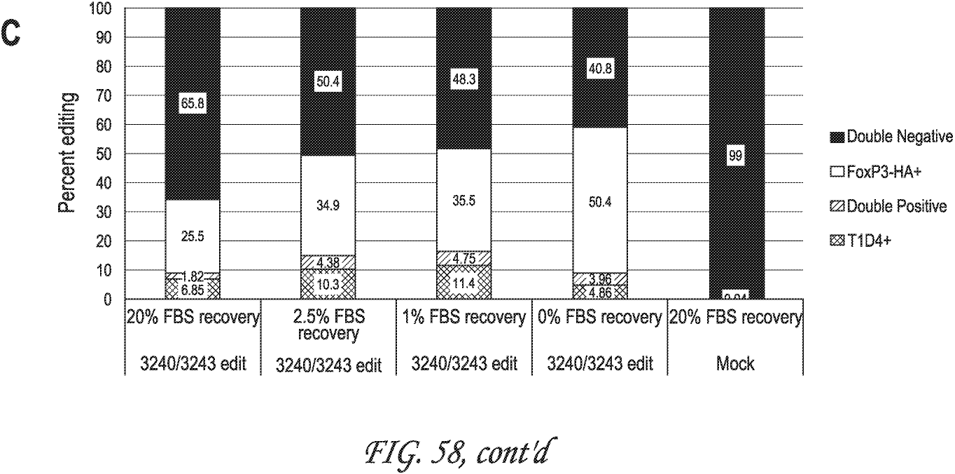

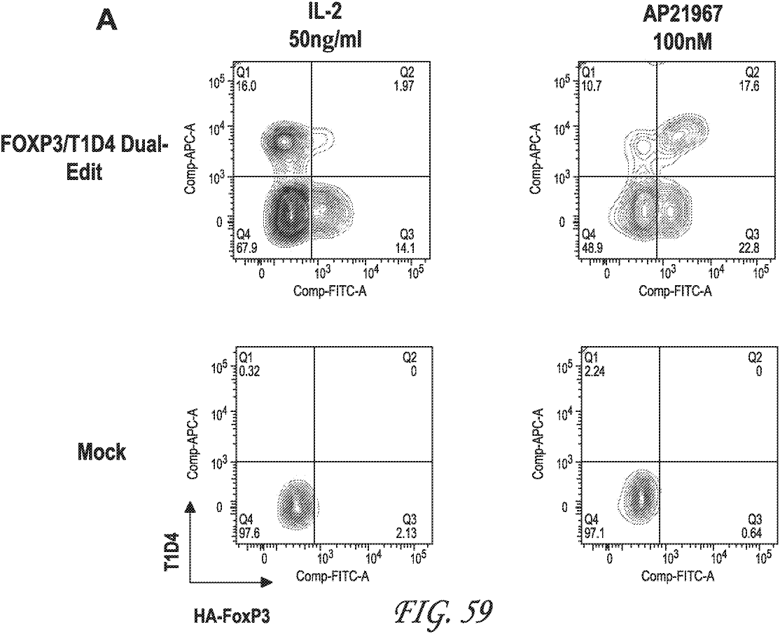

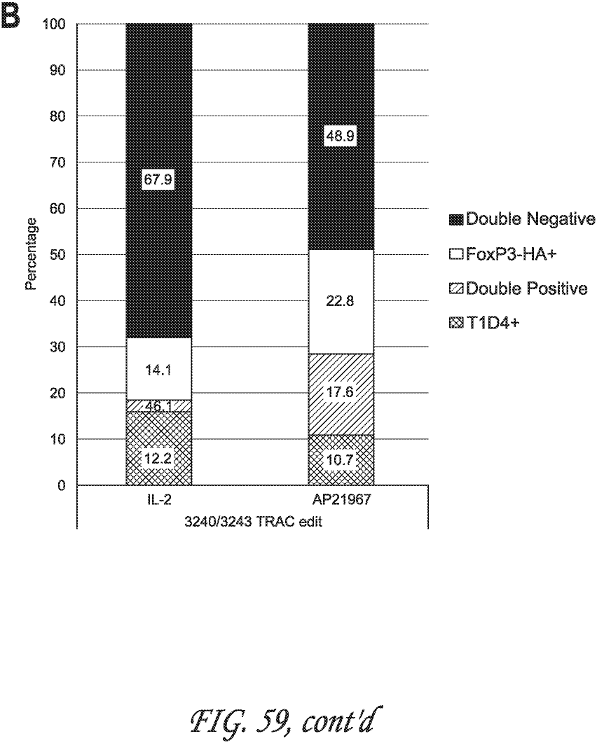

In order to obtain sufficient numbers of edited cells for therapeutic use, it may be important to increase the rate of editing and/or enrichment of FOXP3/TCR dual-positive cells. To improve upon initial editing rates, conditions were modified by varying the serum concentration during the editing phase. FIG. 58 and FIG. 59 show results from a dual-editing experiments using AAV constructs MND.HA.FOXP3.FKBP.IL2RG (#3240) and MND.T1D4.FRB.IL2RB (#3243) comparing four different concentrations of serum during the editing phase of human CD4+ T cells. The resulting FACS analysis demonstrates an improvement of initial editing rate from 1.8% with 20% FBS to 3.96%-4.75% in lower or no serum (FIG. 58). Importantly, the resulting enrichment was nearly 10-fold in cells which recovered in 2.5% FBS, increasing from 1.8% to 17.6% double-positive cells in 7 days of Rapalog treatment, compared to 1.97% with IL-2 treatment (FIG. 59).

In summary, FIGS. 55-59 demonstrate the capacity to achieve efficient levels of dual-HDR editing at the TRAC locus leading to generation of antigen-specific airT that exhibit enrichment using the IL-2 CISC platform.

### Dual Editing of Human CD4+ T Cells - Examples of Two Loci Approach

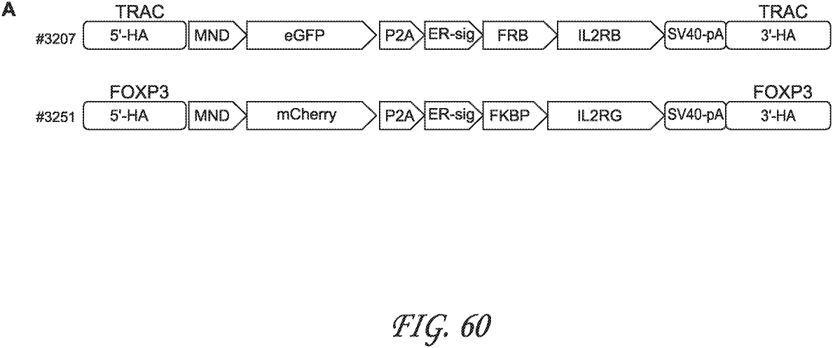

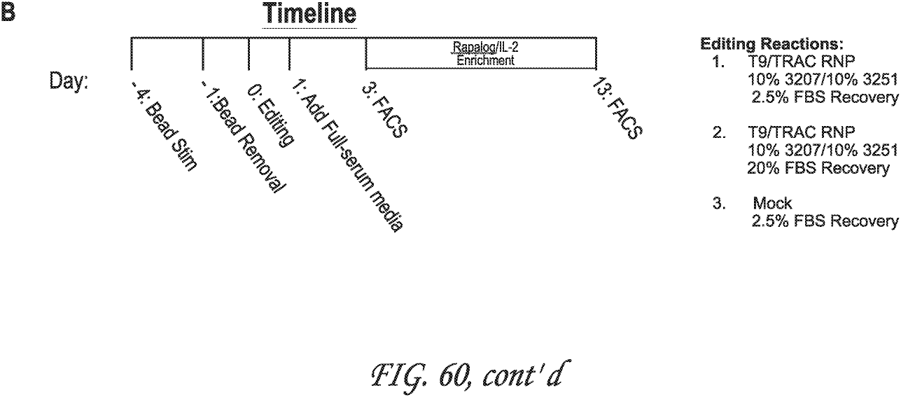

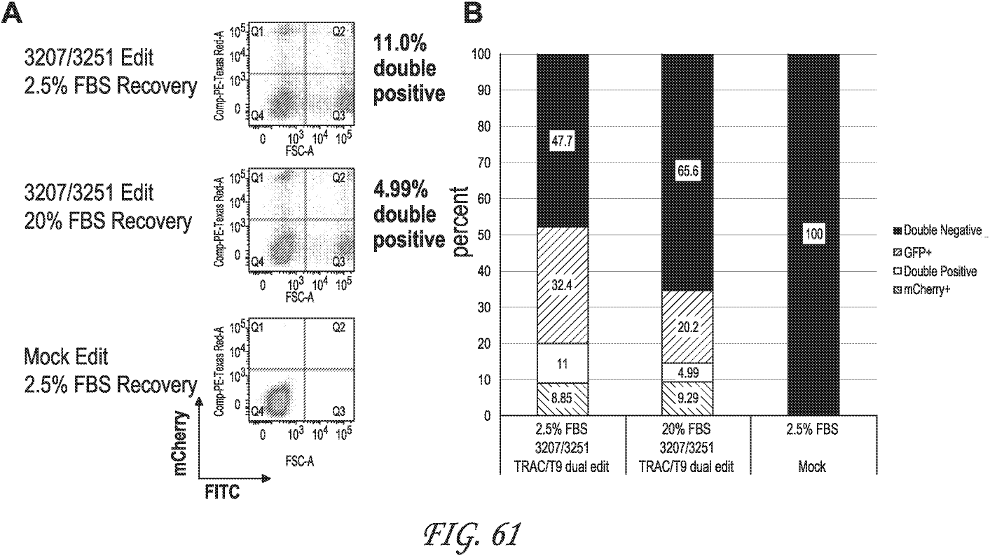

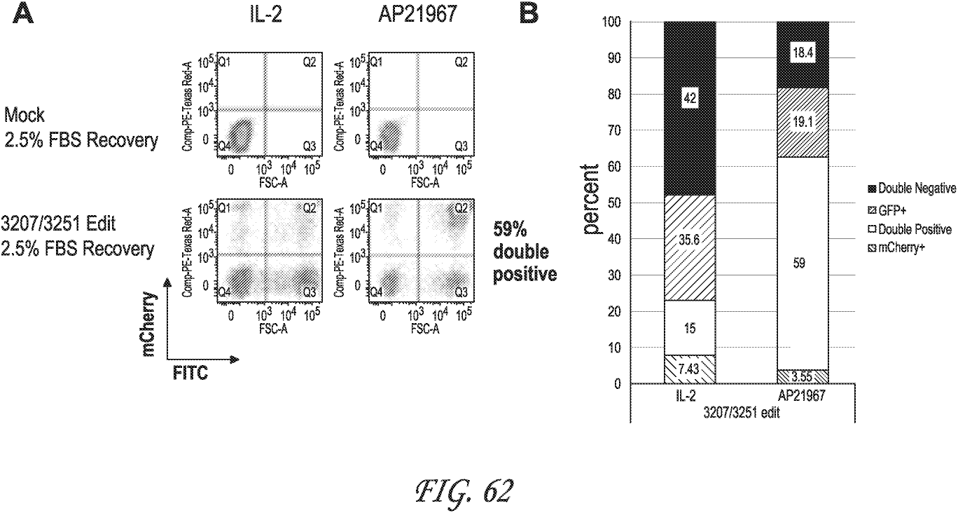

As an alternative dual-editing strategy for generating antigen-specific airT product containing IL-2 CISC components, constructs targeting to the TRAC and FOXP3 loci were developed for two-locus editing. As shown in schematic on FIG. 53, instead of having two constructs targeted to the TRAC locus, one construct is targeted to TRAC locus and the other is targeted to FOXP3 locus. This approach might lead to an improved dual-editing rate and also permit coordinated use of multiple existing strategies to mediate sustained FOXP3 expression. To test this, constructs were developed that would allow easy tracking of successfully edited cells. MND.mCherry.FKBP.IL2RG (#3251) and MND.GFP.FKB.IL2RB (#3207) cassettes with FOXP3 and TRAC homology arms, respectively, were used to test the ability to generate dual-edited cells with stable expression of both mCherry and GFP linked to the IL-2 CISC. The constructs used and time line for editing, cell expansion and analysis are shown in FIG. 60. For this experiment, high serum and low serum editing conditions were compared since the single-loci editing suggested that lower serum concentrations resulted in higher dual editing rates (FIG. 58). The resulting FACS analysis demonstrates successful editing using the two-loci strategy with both serum conditions (FIG. 61). As with the single-locus editing, 2.5% serum media during the editing phase significantly improved dual editing at day 3 (4.99% editing rate with 20% serum vs 11.0% with 2.5% serum). The cells edited in 2.5% serum containing medium were then expanded with either IL2 or Rapalog (AP21967) for 10 days. FIG. 62 shows the robust enrichment in the presence of Rapalog for a total of 59% mCherry/GFP double-positive cells.

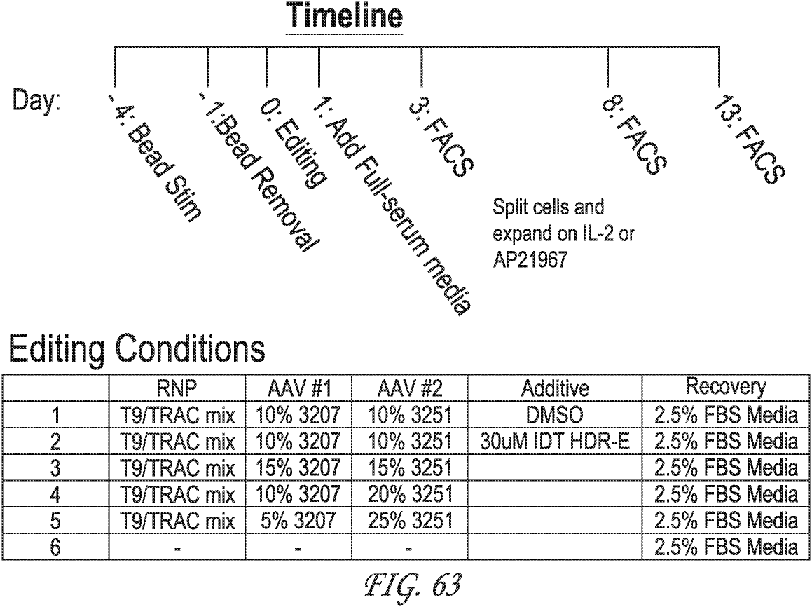

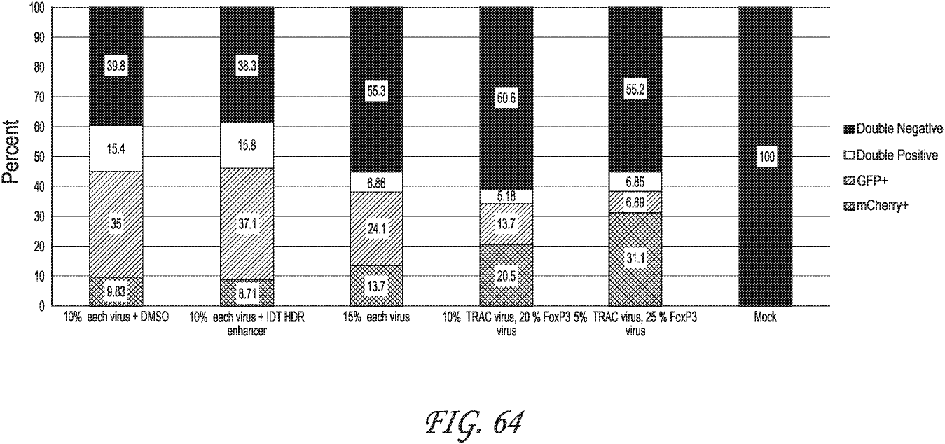

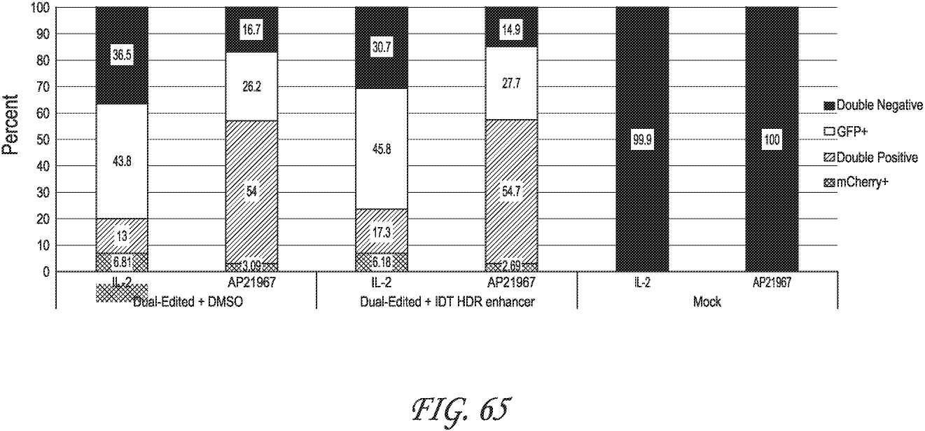

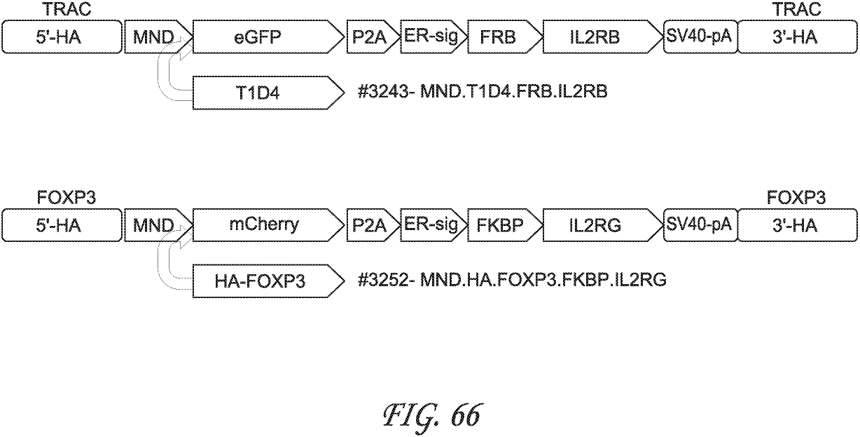

The reproducibility between experiments was tested and alternative editing conditions were explored in an attempt to further increase the initial editing rates. FIG. 63 outlines editing conditions and the time line for editing, cell expansion and analysis. For this experiment, 2.5% serum containing medium was used in the editing phase for all conditions. The percentage virus by volume was varied in the reaction and editing was compared using AAV #3207 and #3251 in the presence and absence of an HDR enhancer (HDR-E). FIG. 64 shows histograms from flow data in each condition 3 days post editing. The editing rate with 2.5% serum containing medium was similar in this study compared to the prior experiment shown in FIG. 61 (15.4% double-positive GFP/mCherry cells compared 11% double-positive cells respectively). In addition, Rapalog enrichment of the dual-edited population resulted in 54% GFP/mCherry double-positive cells (FIG. 65), similar to previous data and demonstrating reproducibility between experiments. Although the presence of HDR-E did not affect the initial editing rate, the % virus in the reaction did impact editing outcomes (FIG. 64). The results showed that 10% culture volume of each virus was optimal compared to 15% each, or any of the other combinations with a total of 30% virus.

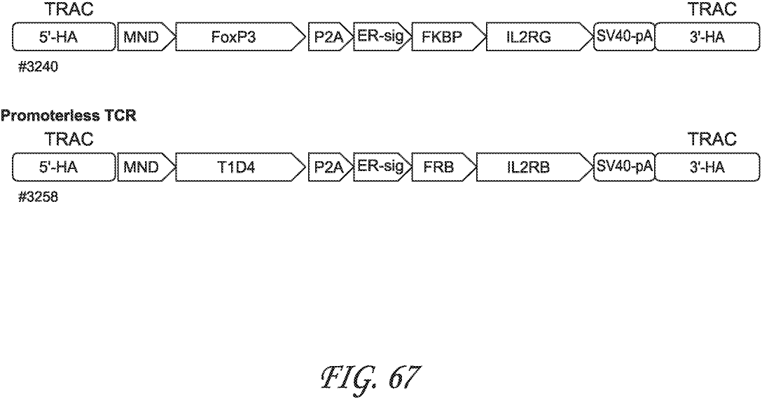

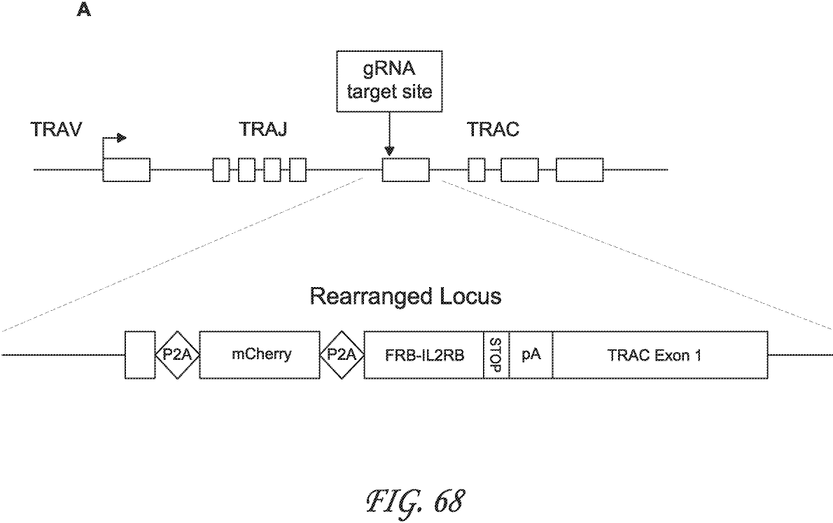

The results of these studies demonstrate that presently disclosed two-loci dual-editing strategies can be used to introduce the IL-2 split-CISC cassette and lead to efficient enrichment of dual-edited cells using Rapalog. Having been successful using this approach for generation and enrichment of dual-edited cells, constructs were designed and cloned for expression of FOXP3 and a pancreatic islet antigen-specific TCR (T1D4) in combination with IL-2 CISC components by targeting the FOXP3 and TRAC loci, respectively. These and a range of other HDR donors are used to generate antigen-specific FOXP3 airTcells (FIG. 66).

### In-Frame TRAC Knock-in as a Dual-Editing Strategy

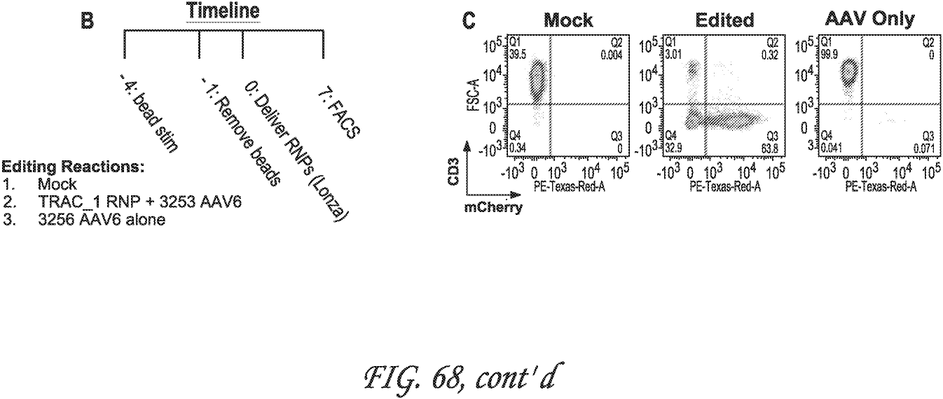

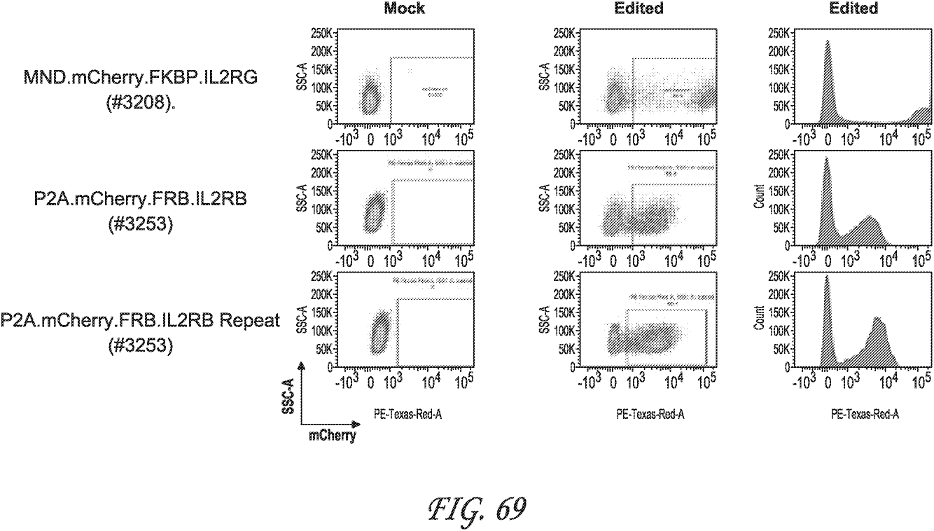

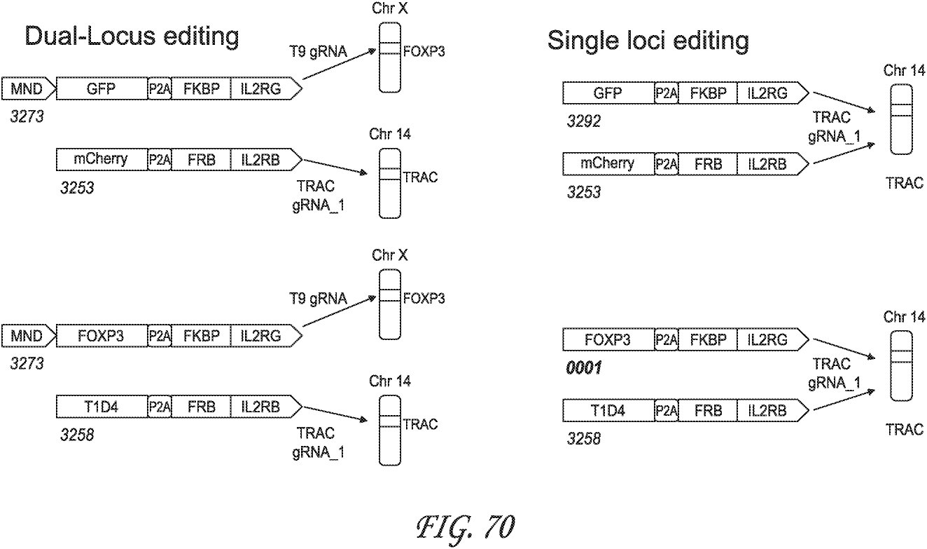

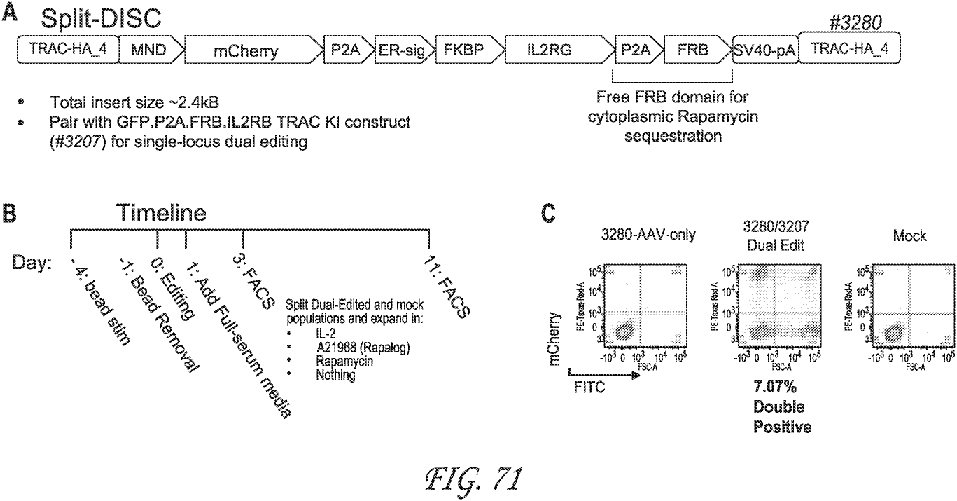

As an additional modification/improvement in our dual-editing strategies, methods were established for in-frame knock-in of a promoter-less TCR cassette including components of the IL-2 CISC, by targeting the first exon of the TRAC locus (FIG. 67). This editing strategy drives expression of the antigen-specific TCR via the promoter/enhancer activity of the endogenous TRAC locus. Advantages of this approach include elimination of endogenous TCR expression (and the potential for improper pairing with delivered TCR components) and concomitant near-endogenous levels of autoantigen-specific TCR expression. To establish this approach, gene editing and exogenous gene expression were tested with a proof-of-concept mCherry IL-2 CISC-containing construct (#3253) (FIG. 68). 63.8% of cells were mCherry-positive with concomitant loss of CD3 (down from 99.9% in AAV-only to 3.01% in P2A.mCherry.FRB.IL2RB (#3253) edited cells). Following gene-editing, mCherry expression was easily detected by flow cytometry. As expected, the MFI of mCherry expression in P2A.mCherry.FRB.IL2RB (#3253) edited cells was lower compared to mCherry expression in T cells edited in the same locus using MND promoter expression cassette (MND.mCherry.FKBP.IL2RG (#3208) FIG. 69). Thus, this HDR approach successfully permitted transgene expression via the promoter/enhancer activity of the endogenous TRAC locus.

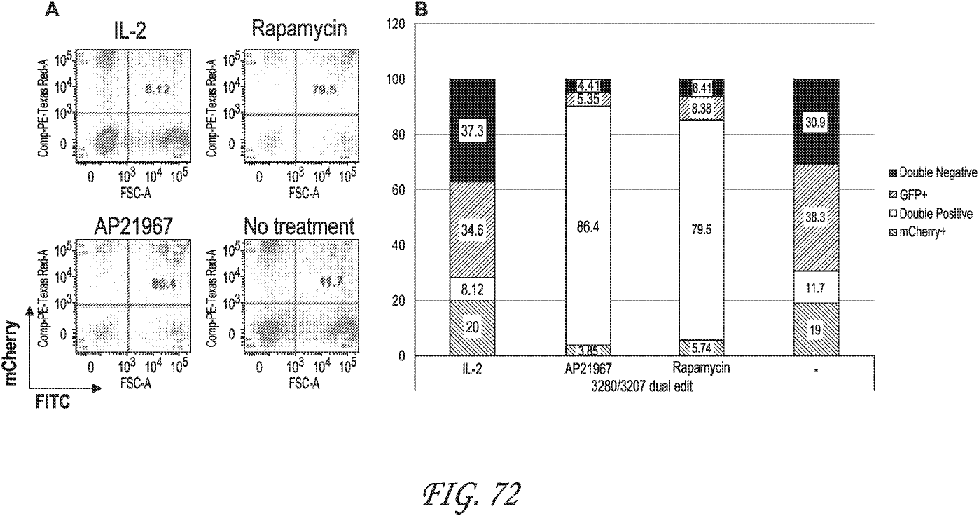

In follow-up studies, two distinct HDR donor cassettes are used to achieve dual-editing of TRAC (and capture of the endogenous promoter) and/or editing of both the TRAC and FOXP3 loci. HDR donors introduce split components of the IL-2 CISC to permit enrichment of dual-edited cells in parallel with delivery of the Ag-specific TCR (under TRAC promoter) and FOXP3 expression via cDNA expression or via locking on expression of endogenous FOXP3. Two-loci dual editing is tested using mCherry Split CISC with endogenous TRAC promoter (P2A.mCherry.FRB.IL2RB (#3253)) paired with MND.GFP.FKBP.IL2RG (#3273) for editing into the FOXP3 locus. Expression of the two components of the IL-2 split-CISC from two distinct promoters may effect overall CISC function, so single-locus dual editing is also tested using P2A.mCherry.FRB.IL2RB (#3253) and P2A.GFP.FKBP.IL2RG (#3292) to drive both components of the IL-2 CISC off the endogenous TRAC promoter (FIG. 70).

Following successful bi-allelic editing (using one or both promoter-less mCherry/GFP constructs and enrichment of dual edited cells using Rapalog), constructs are generated that can be used to introduce FOXP3 and antigen-specific TCR (T1D4) in combination with the IL-2 CISC components as an alternative approach to generate antigen-specific FOXP3+ airT cells. (See FIG. 70). airT generated using these strategies are compared to cells wherein the antigen-specific TCR is driven by the exogenous MND promoter.

### Dual Editing Using Decoy-CISC (Split-DISC) Constructs

Although CISC-expressing cells expand efficiently in the presence of a heterodimerizing Rapalog (AP21967), the FDA-approved drug, Rapamycin, may be preferred for clinical application. Intracellular binding of Rapamycin to its target mTOR has well-documented inhibitory effects on T-cell proliferation. Constructs and methods to address this problem by utilizing a naked “decoy” FRB domain expressed intracellularly in CISC-expressing cells are disclosed in WO2019210057 which is incorporated by reference in its entirety. Constructs that express the naked FRB domain along with the CISC are designated as “decoy-CISC,” or “DISC.”

To determine if the DISC would work with dual editing approach, CISC containing constructs were modified to include an additional naked FRB domain located 3′ of the CISC receptor (FIG. 70, FIG. 71). Constructs with the additional FRB domain along with the CISC components are designated as “decoy-CISC or “DISC”. When utilized in a dual-editing approach, constructs are designated as split-DISC. Using the split-DISC, the naked FRB domain competed with the endogenous FRB domain of mTOR for binding to Rapamycin, thereby resulting in Rapamycin-mediated signaling through the split-CISC components without the associated inhibition of cell growth.

As shown in FIG. 71, T cells dual-edited with the mCherry CISC construct containing the added FRB domain (mCherry DISC, #3280) and the GFP CISC construct (#3207), expressed 7.07% double-positive GFP/mCherry cells. As predicted with the DISC construct, the double-positive GFP/mCherry cells enriched to a similar level following treatment with either Rapamycin or Rapalog (AP21967) (79.5% and 86.4% respectively) (FIG. 72).

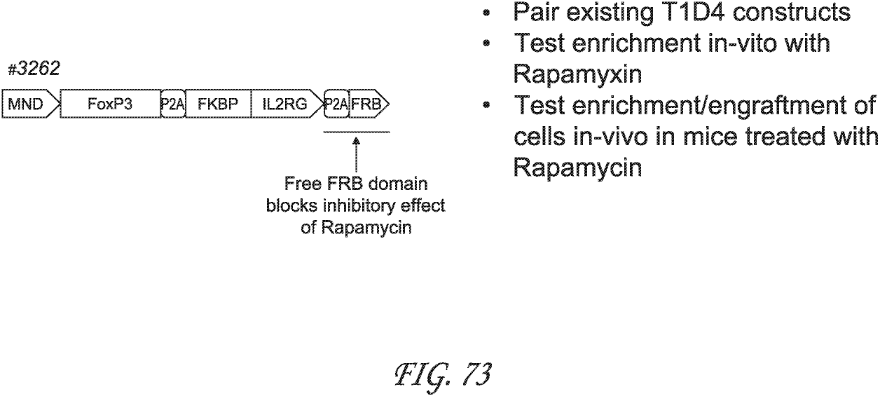

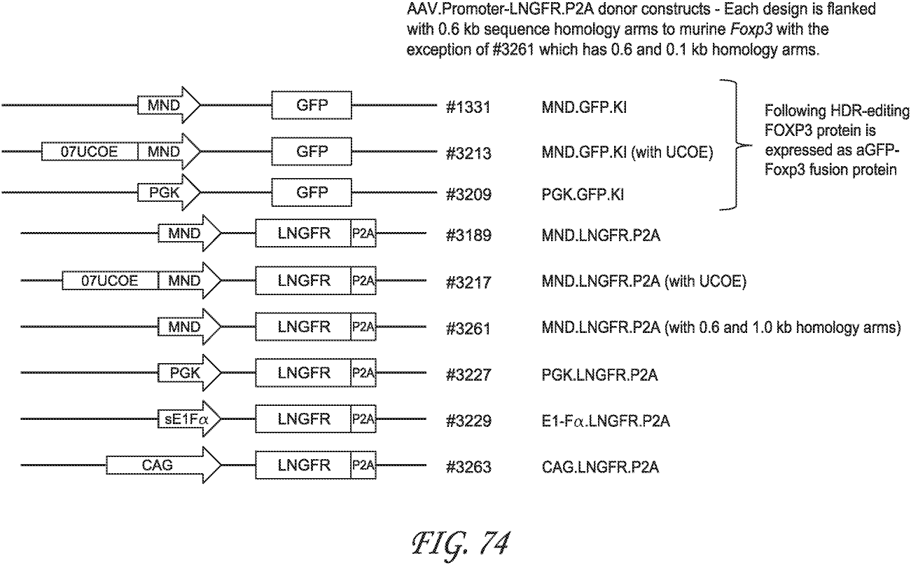

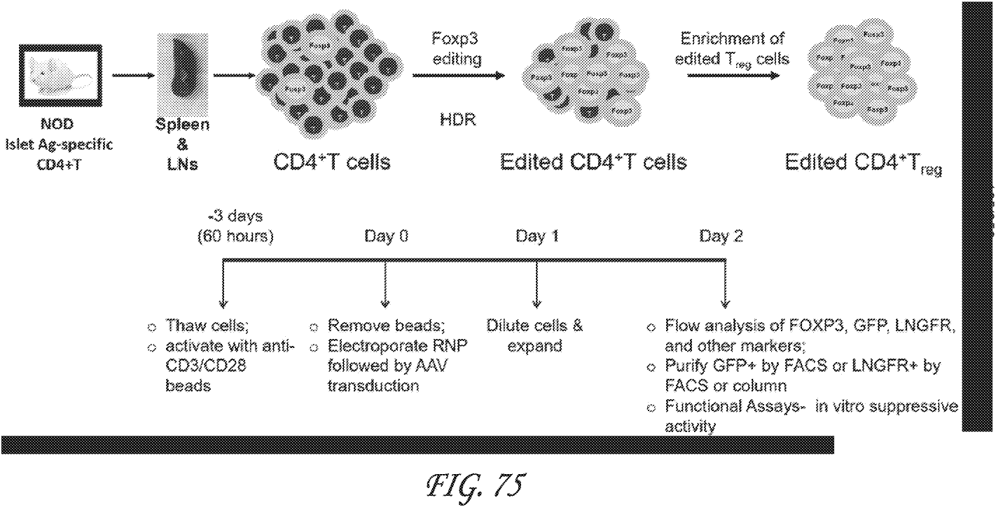

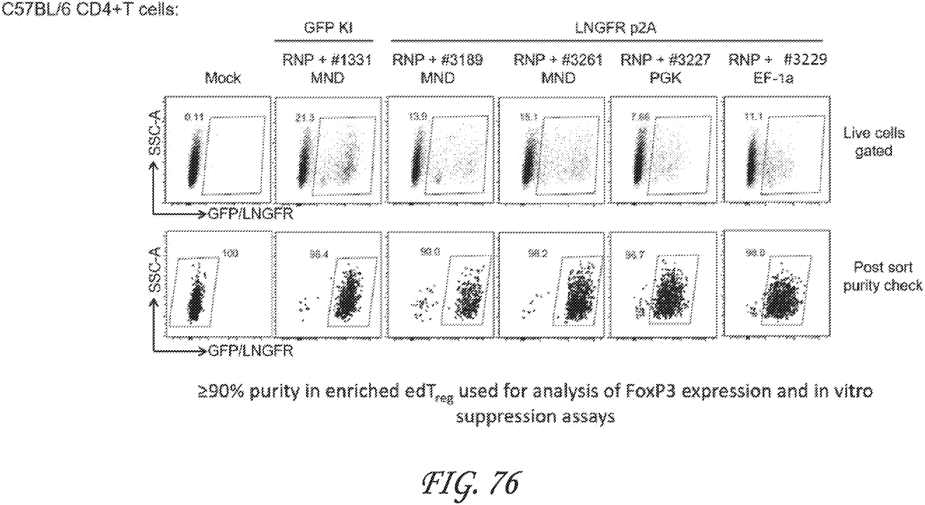

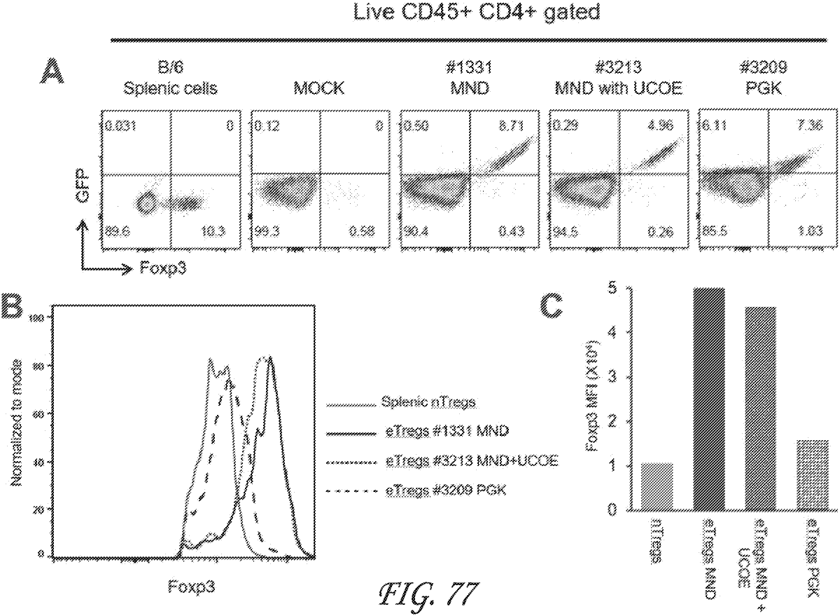

Experiments are performed to determine the in vivo enrichment/engraftment of GFP/mCherry -split-DISC edited cells in NSG mice treated with Rapamycin. Dual-edited GFP/mCherry cells exhibit increased engraftment/enrichment in vivo. These studies are expanded using FOXP3- and T1D4-containing constructs to evaluate engraftment and expansion of islet-specific airT in vivo. The DISC construct (MND.FOXP3.FKBP.IL2RG.FRB, #3262) shown in FIG. 73 is paired with existing #3243 MND.T1D4.FRB.IL2RB for dual-editing into the TRAC locus for development of islet-specific airT that can be enriched in vivo using Rapamycin.

### Example 11-In Vitro and In Vivo Functional Activities of Ag-Specific Murine airT In Vitro Characterization of Murine airT Products

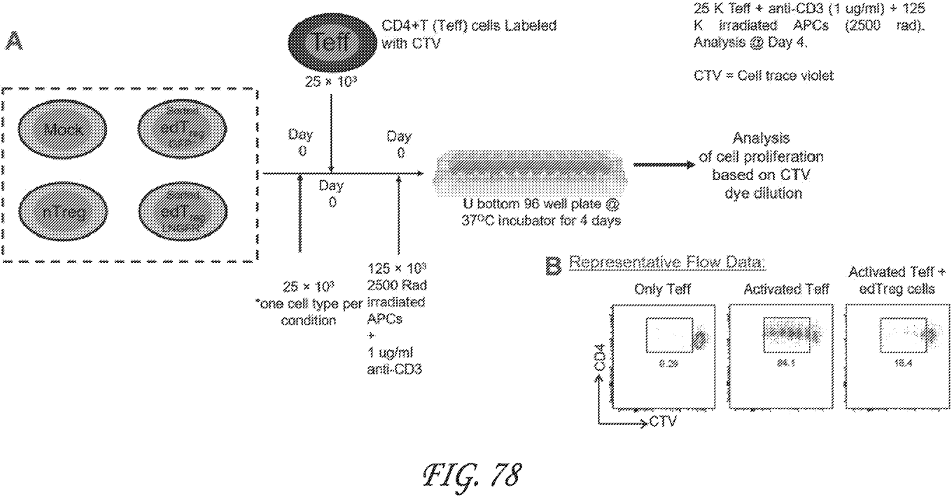

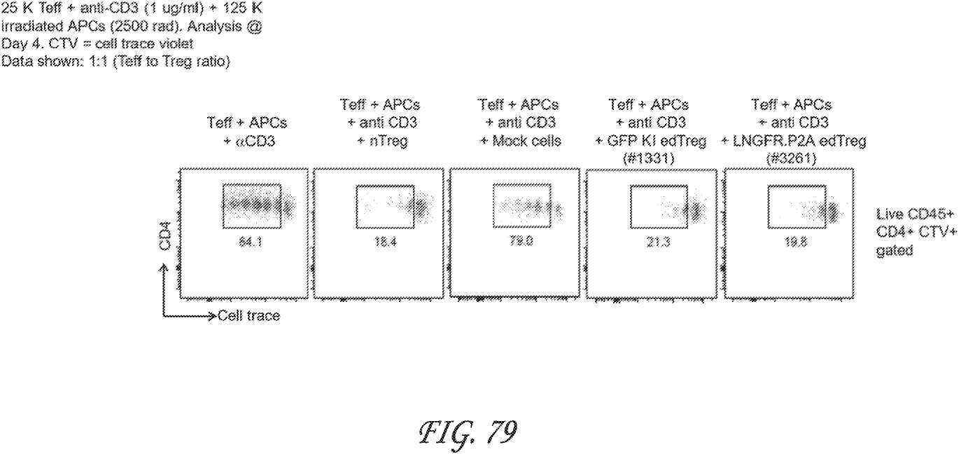

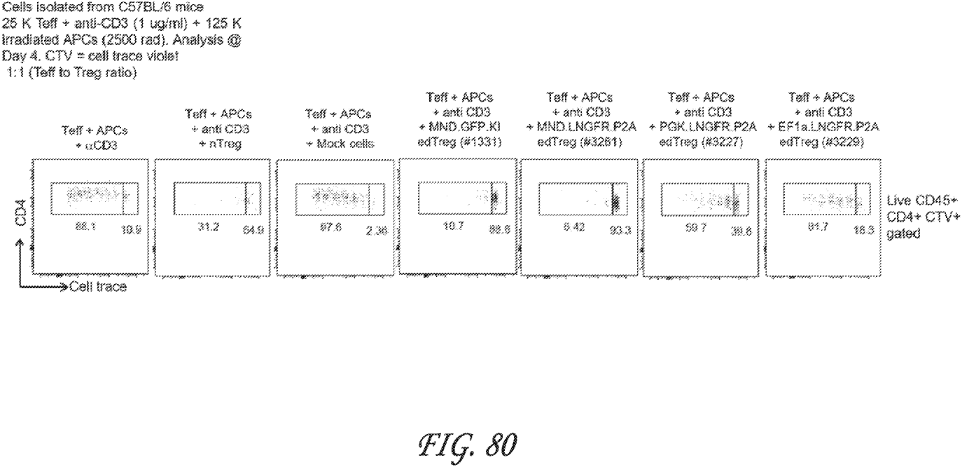

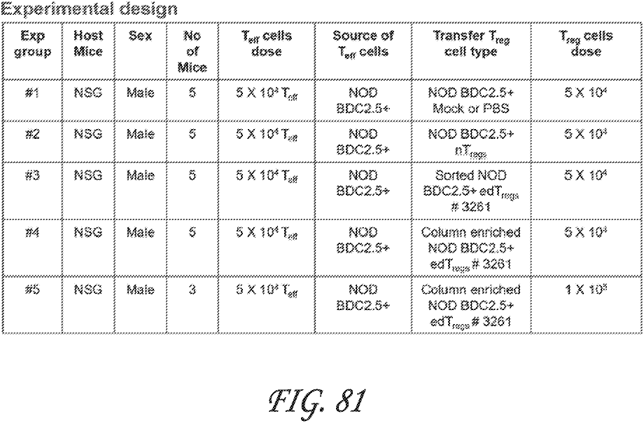

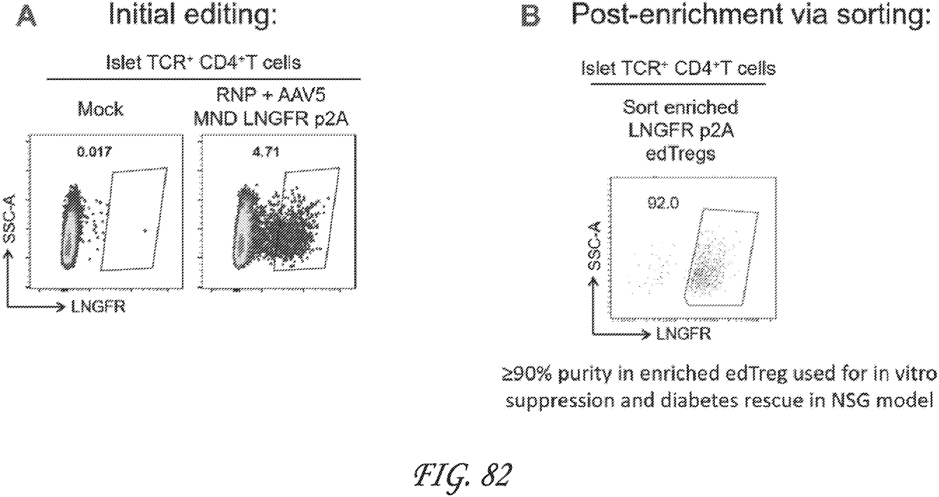

Studies designed to identify advantageous HDR donor template designs for generating airT products from murine CD4+ T cells with a suppressive Treg-like phenotype were performed. Questions addressed by these studies included: a) which of the tested promoter/enhancer constructs provided the best airT performance in vitro and, ultimately, in vivo and b) whether use of the clinically relevant cis-linked selectable marker LNGFR allowed for enrichment of airT in a manner suitable to GMP manufacturing. FIG. 74 to FIG. 80 show results from experiments that (1) evaluated the use of the clinically relevant cis-linked selectable marker LNGFR as a method to enrich murine cells following editing; (2) tested a variety of candidate promoters (in addition to MND promoter); (3) tested two Foxp3 homology arms of different size in the donor templates; and (4) compared donor templates with and without the UCOE element (as a potential means to limit silencing of an introduced promoter within the Foxp3 locus).

First, the effect of extending the homology arm of MND.LNGFR.P2A donor template from 0.6 kb to 1.0 kb of the Foxp3 gene on editing efficiency in C57BL/6 mouse CD4+ T cells was evaluated (FIG. 76). These studies indicate that MND.LNGFR.P2A #3261, encompassing a 1.0 kb arm, has a slightly higher editing efficiency compared to MND.LNGFR.P2A #3189, which contains a 0.6 kb arm. The improvement is -10%, and the increase in editing efficiency was reproducible between experiments, which enabled selection of AAV #3261 as a desirable targeted donor template for MND.LNGFR.P2A airT murine cells in the remaining studies. Also tested was the editing efficiency and purity following enrichment of C57BL/6 edTreg using AAV donor templates with alternative promoters (FIG. 76). This demonstrated that the overall editing and purity of LNGFR+ enriched cells were similar between AAV donor templates containing MND, PGK and EF-1a promoters. In addition, the purity following enrichment of LNGFR+ cells and GFP+ cells were comparable, further demonstrating that LNGFR can be used as a selection and enrichment method of murine CD4+ T cells.

Although the editing efficiency and purity of edited cells using AAV donor templates with different promoters was similar, importantly, the level of FOXP3 expression varied depending the promoter. FIG. 77 shows evaluation of GFP and FOXP3 expression in Mock-edited, MND.GFP.KI- (#1331) and PGK.GFP.KI- (#3209) in C57BL/6-edited CD4+ T cells. The results showed that FOXP3 MFI was significantly higher in cells edited using MND.GFP.KI (#1331) compared to PGK.GFP.KI (#3209). The level of FOXP3 expression in PGK.GFP.KI- (#3209) edited CD4+ cells was similar to FOXP3 expression observed in splenic nTregs. These studies demonstrated the ability to introduce alternative promoters into the endogenous Foxp3 locus to control the overall level of FOXP3 in airT products. This may provide flexibility in product generation and may lead to airT with different functional properties. The relationship between FOXP3 expression level and in vitro and in vivo function was further explored in studies described in FIG. 79 and FIG. 85 (see below). The ability of Ubiquitous Chromatin Opening Element (UCOE) to stabilize FOXP3 expression was also tested (FIG. 77). This element can function to reduce silencing and limit potential negative impacts of promoter elements. These studies showed that FOXP3 was stable with or without UCOE and that inclusion of the UCOE element did not negatively impact the relative FOXP3 expression level (MND.GFP.KI #1331 compared with MND.GFP.KI with UCOE #3213), suggesting that UCOE shielded donor works effectively and inclusion of this element may be useful in airT products as it might protect expression in vivo or over time, providing improving duration of functional activity.

Additional studies were performed to assess: (a) the functional activity of LNGFR- expressing airT cells in vitro; and (b) determine if the promoter driving endogenous FOXP3 expression manifested any impact on Treg functional activity. An in vitro suppression assay outlined in FIG. 78 was utilized to analyze T_{eff} cell proliferation in the presence and absence of sorted MND.GFP.KI- and MND.LNGFR.P2A- airTs; these effects were compared to the activity of purified murine nTreg. The results shown in FIG. 79 demonstrated that murine airT (generated with the MND.GFP.KI or MND.LNGFR.P2A HDR donors) and nTregs exhibited comparable, robust in vitro suppressive function. These findings also demonstrated that airT cells expressing the LNGFR selectable marker (MND.LNGFR.P2A #3261) can be successfully used as a selection and enhancement method of murine CD4+ cells without a loss in functional activity and are useful for modeling in vitro and in vivo functional activity of human airT products that utilize the same clinically relevant selection marker.

Using the same in vitro suppression assay, the functional activity of different strength promoters was explored to determine if FOXP3 expression levels (evaluated in FIG. 77) correlated with functional activity. FIG. 80 shows a comparison of LNGFR constructs utilizing the MND, PGK and EF-1a promoters (MND.LNGFR.P2A, PGK.LNGFR.P2A and EF-1a.LNGFR.P2A respectively) with MND.GFP.KI C57BL/6 edited CD4+ T cells and nTreg. The results showed that murine airT with MND promoter exhibited suppressive function that was comparable to nTreg. In contrast to the MND promoter constructs, airT cells utilizing the PGK promoter exhibited only partial in vitro suppressive function and airT utilizing the EF-1a promoter failed to suppress. Interestingly, although the PGK.GFP.KI-edited cells expressed FOXP3 levels similar to nTregs (FIG. 77), the in vitro suppressive activity of nTreg was significantly greater than PGK.LNGFR.P2A edited cells. These findings suggested the surprising result that a threshold level of FOXP3 expression in edited CD4+ T cells may be necessary to provide proper reprogramming and effective in vitro functional activity. The results described below also clearly demonstrate that the MND promoter was effective for reducing diabetes in vivo.

### In Vivo Functional Characterization of edTreg Products

The experiments summarized above suggest that murine airT cells containing a clinically relevant cis-linked LNGFR selectable marker retained functional activity in vitro and that the MND promotor had superior in vitro suppressive activity compared to alternative promoters. These studies were expanded to evaluate islet-specific airT product in an NSG adoptive transfer diabetes model where transfer of islet-specific NOD (murine) CD4+T cells into adult recipient NSG mice triggers rapid onset of diabetes.

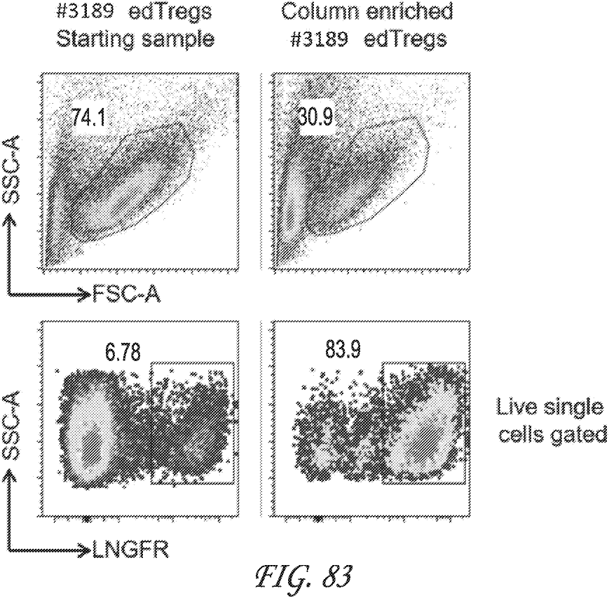

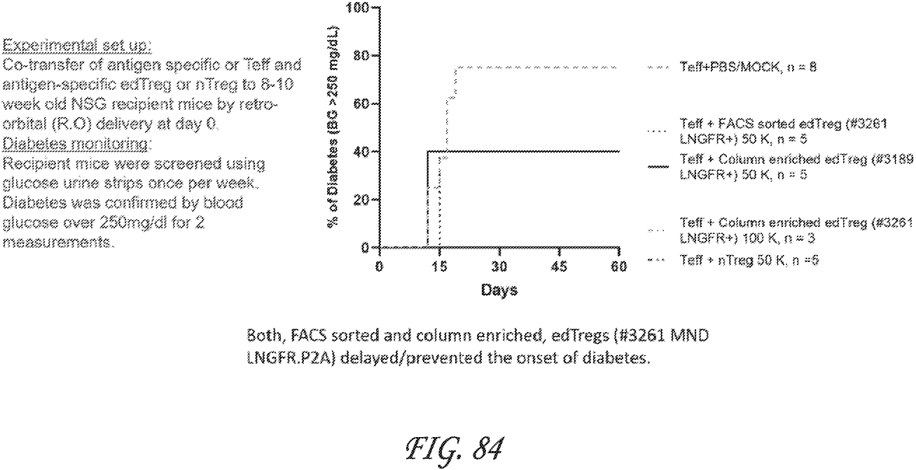

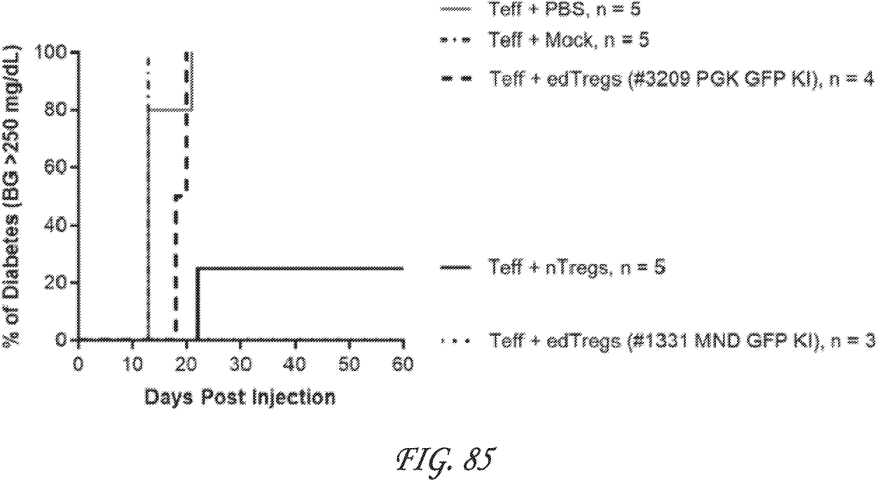

Using this model, islet-specific MND.LNGFR.P2A airT derived from NOD BDC2.5 mice were evaluated for the ability to delay or prevent diabetes development. Although the in vitro experiments described in FIGS. 76-80 utilize cells enriched via cell sorting, the sorting process is both time-consuming and costly. In addition, sorting may well impact the engraftment and/or survival of cells post-adoptive transfer in vivo. To enable efficient enrichment of murine gene-edited airT that can be used in vivo, the purification and functional activity of airT purified using alternative methods was compared: (1) LNGFR+ cell enrichment through cell sorting by flow cytometer and (2) LNGFR+ cell enrichment using LNGFR column separation. FIGS. 82-83 show the flow plots prior to and post-purification using sorting and column enrichment. Although purification using the sorting method yielded a somewhat more pure population of LNGFR+ cells, the column enrichment produced -84% pure cell population with a significant savings in time and resources. Importantly, both the column-enriched LNGFR+ airT and sorted cells delayed or prevented the onset of diabetes (FIG. 84).

Together, the data in FIG. 82 to FIG. 84 demonstrated the capacity to generate highly purified islet-specific edited cells using LNGFR column separation and by FACS sorting. Both products expressed high levels of LNGFR/FOXP3 and showed Treg-like phenotypes by reducing or preventing diabetes in vivo.

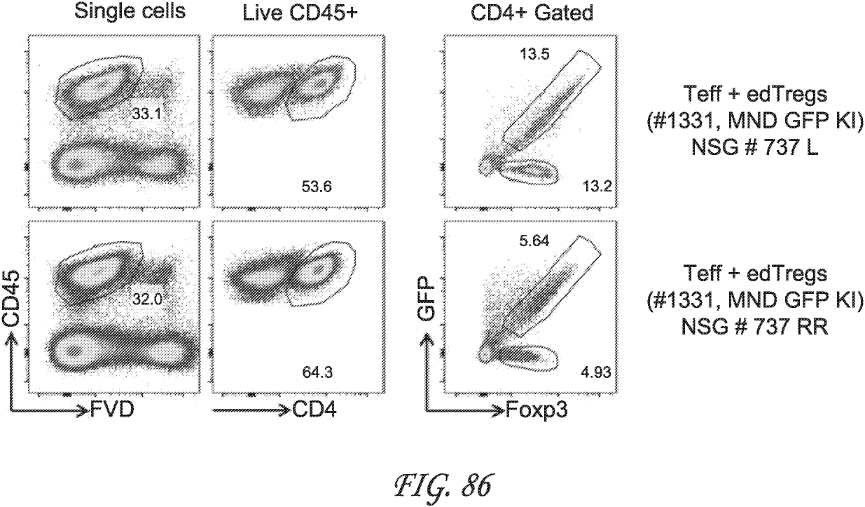

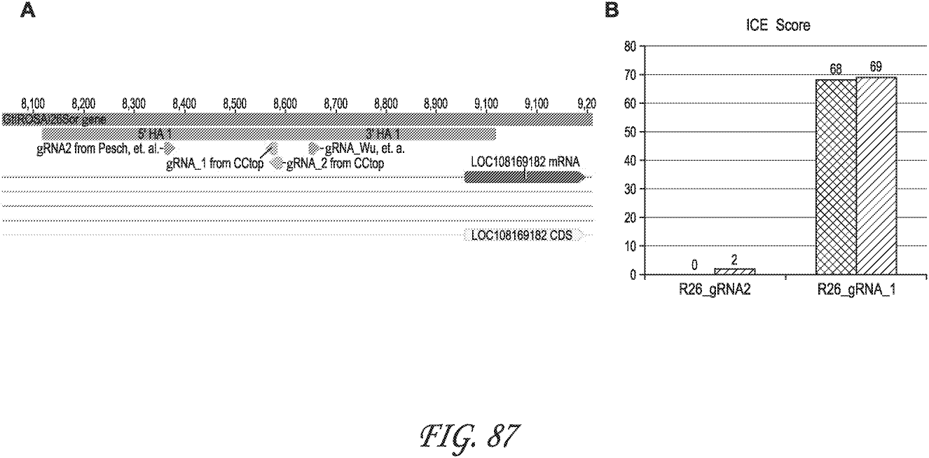

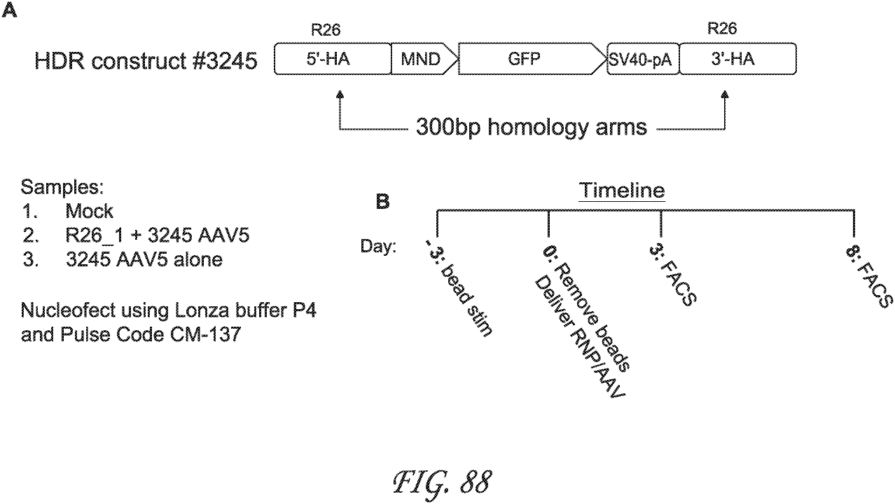

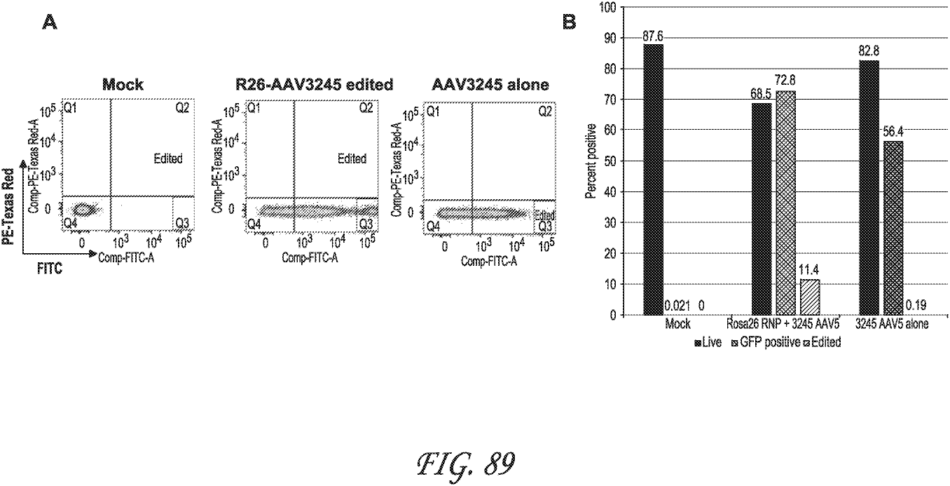

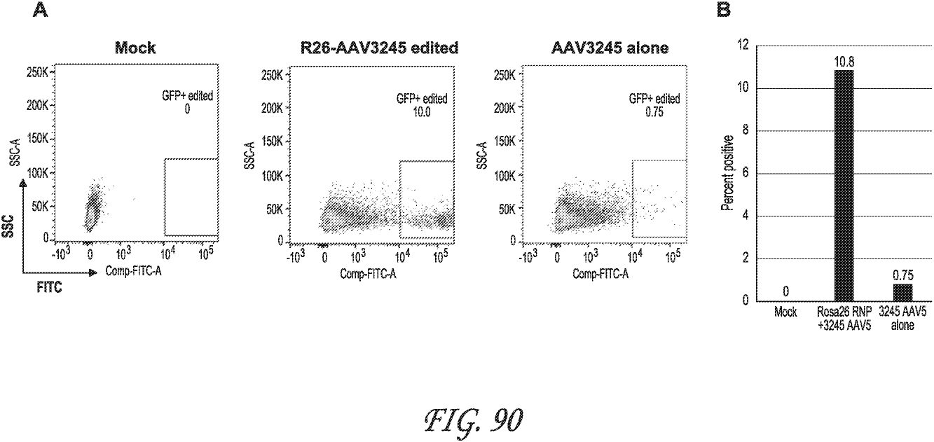

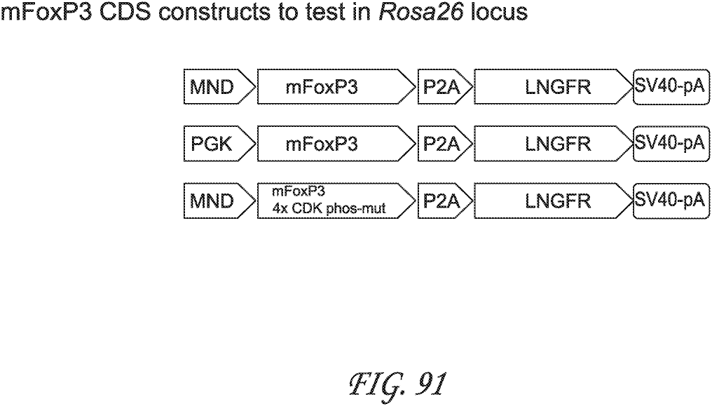

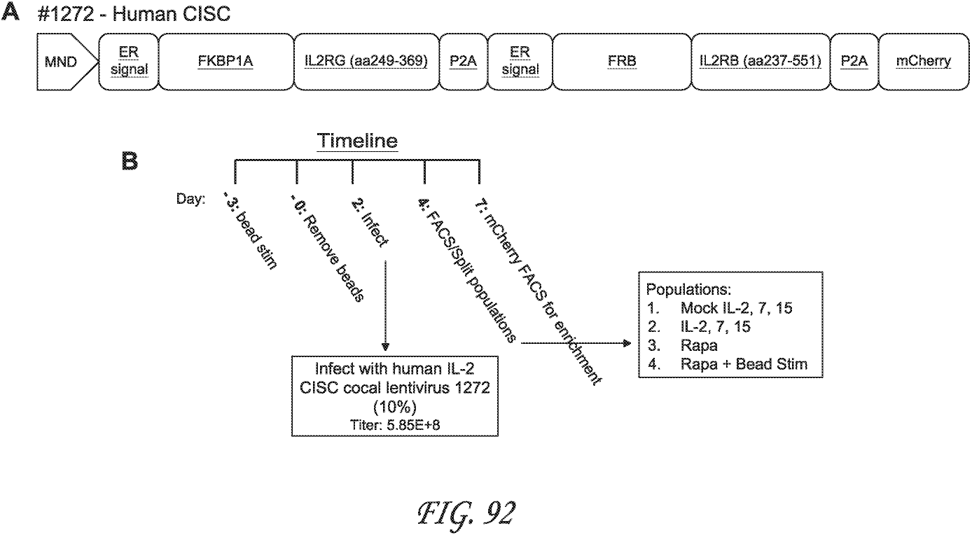

Finally, FIG. 85 shows that functional activity of islet-specific airT products in the NSG adoptive transfer model varied depending on the promoter used to drive endogenous FOXP3. Cells edited with MND.GFP.KI and nTregs both delayed the onset or prevented diabetes, but PGK.GFP.KI airT did not. This result was consistent with the in vitro suppression data shown in FIG. 80 suggesting that selection of the promoter played a role in optimal function. Importantly, consistent with protection from diabetes, islet-specific airT cells homed to the pancreas and persisted in the NSG model with stable FOXP3 expression (FIG. 86).

These data demonstrated the capacity to engineer mouse airT from T_{eff} cells for in vitro and in vivo studies. Consistent with the findings in human T cells, the MND promoter effectively converted mouse T_{eff} into airT cells with high levels of FOXP3 expression and robust in vitro suppressive activity comparable with nTreg. Importantly, murine islet-specific MND airT and nTreg: (1) exhibited comparable, robust in vitro suppressive function; (2) blocked diabetes triggered by islet-specific T_{eff} in recipient mice. Moreover, the data showed that (3) airT cells expressing the LNGFR selectable marker can be enriched in vitro without a loss in functional activity and can function in vivo and (4) MND airT outperformed airT generated with alternative promoters, including PGK and EF1A, demonstrating that choice of the promoter played a role in improved function.

The NSG adoptive transfer diabetes model described permitted rapid assessment of key functional features of murine airT including: LN trafficking, expansion, activation status, and the capacity to limit initial T_{eff} activation. These approaches were used to compare the functional activity of antigen-specific, LNGFR enriched airT in an immunocompetent NOD mouse model of T1D.

### Editing at the Rosa26 Locus for Generating Murine T Cells Edited Cells

To expand the tool set for assessing the efficacy of Ag-specific FOXP3 airT in animal models of diabetes or other autoimmune conditions, gRNA targeting the murine Rosa26 locus were designed and tested. This well-characterized safe harbor locus has historically been used for stable expression of integrated transgenes in mouse models. Two novel gRNA target sequences within an intronic region of Rosa26, proximal to published gRNA target sites, were selected and on-target site-specific activity measured by ICE (Inference of CRISPR Edits) after RNP delivery to primary mouse CD4+ T-cells. ICE confirmed specific indel induction for R26_gRNA_1 in Rosa26 (FIG. 87).

Next, the ability to edit murine T cells using constructs that would allow easy tracking of successfully edited cells was tested. A MND-GFP cassette was generated flanked by identical 300 base pair Rosa26 homology arms matched to R26_gRNA_1 (#3245) and was used to generate Rosa26 edited T cells with stable expression of GFP. The timeline for cell expansion, editing and analysis is shown in FIG. 88. The resulting FACS analysis demonstrates 11.4% GFP high cells with AAV #3245 plus RNP compared to 0.02% with AAV #3245 alone 3 days post-edit, confirming successful integration of MND-GFP repair cassette into the Rosa26 locus (FIG. 89). FACS analysis carried at 8 days post-editing showed a similar percent of GFP+ cells (10.8%), indicating that the GFP expression is stable (FIG. 90).

Having achieved editing murine T cells at the Rosa26 locus using the MND-GFP cassette, repair templates containing mFoxp3 CDS with LNGFR marker (for purification) and alternative candidate promoters (in addition to MND promoter) are developed to generate further constructs with stable expression of FOXP3 in this safe harbor locus in mouse cells (FIG. 91). These constructs are used to explore dual editing in mouse cells for the generation of murine antigen-specific FOXP3-expressing airT for use in mouse autoimmune models. Mutant FOXP3 variants are tested that are predicted to have increased stability. This includes the 4x CDK phosphorylation mutant, where a set of 4 target residues for cyclin-dependent kinase phosphorylation have been replaced with alanine, blocking phosphorylation events that have been linked to protein degradation.

These data demonstrate that the Rosa26 safe harbor locus can be used for HDR editing in mouse T cells. This advance permits dual-editing studies of mouse T cells paralleling work in human T cells, facilitating nonclinical animal modeling of Ag-specific airT.

### Developing Tools for Expansion of Murine Cells Using CISC Elements

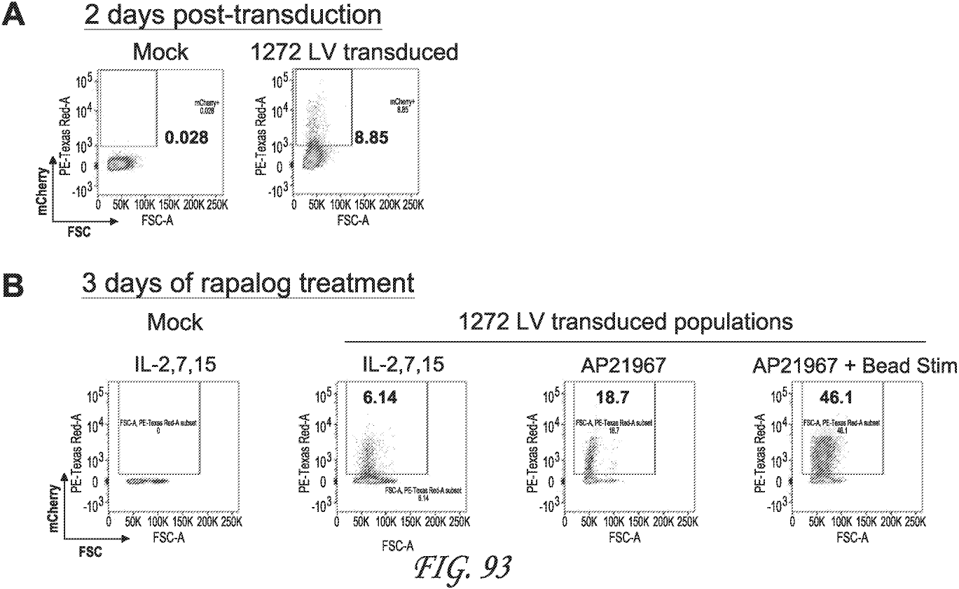

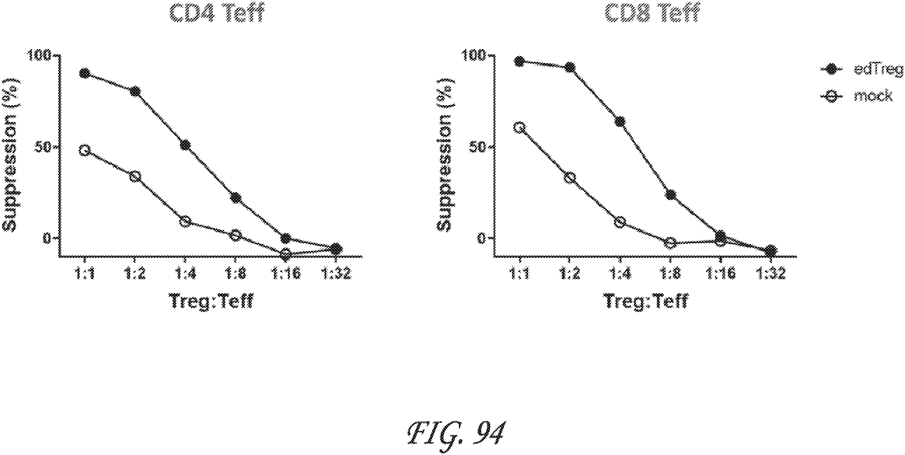

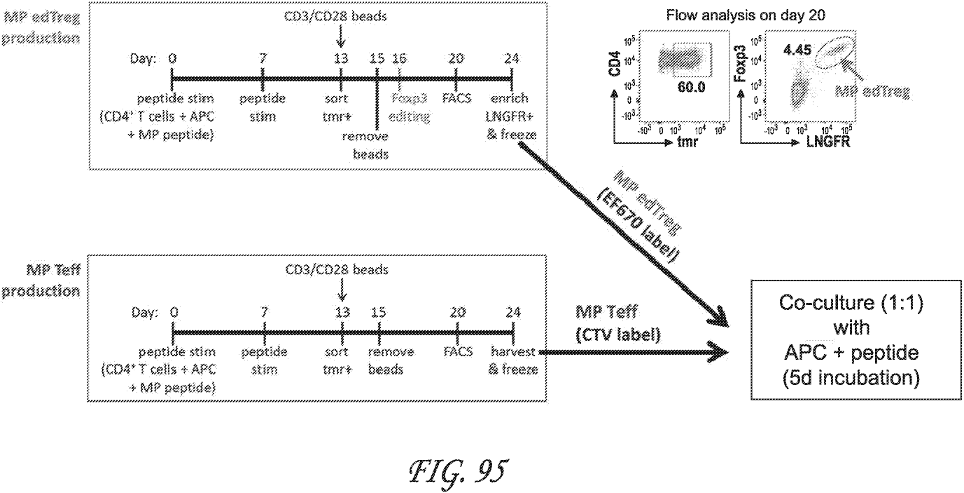

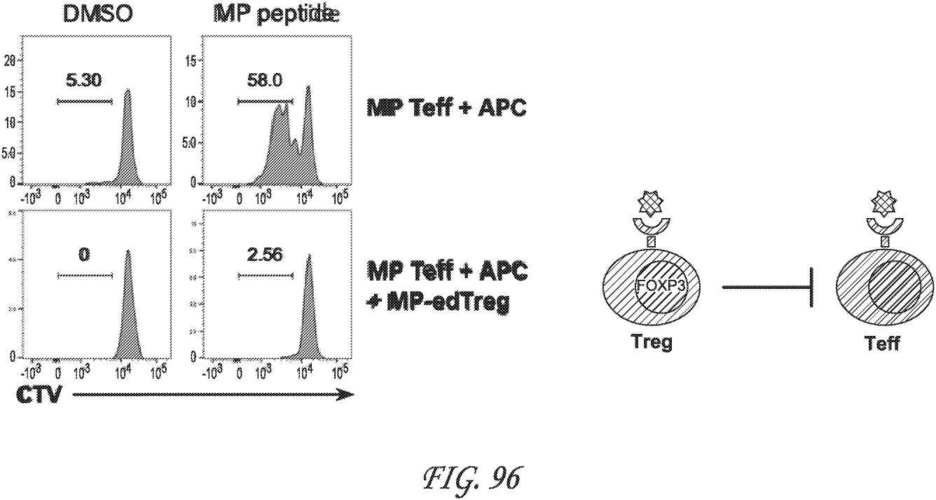

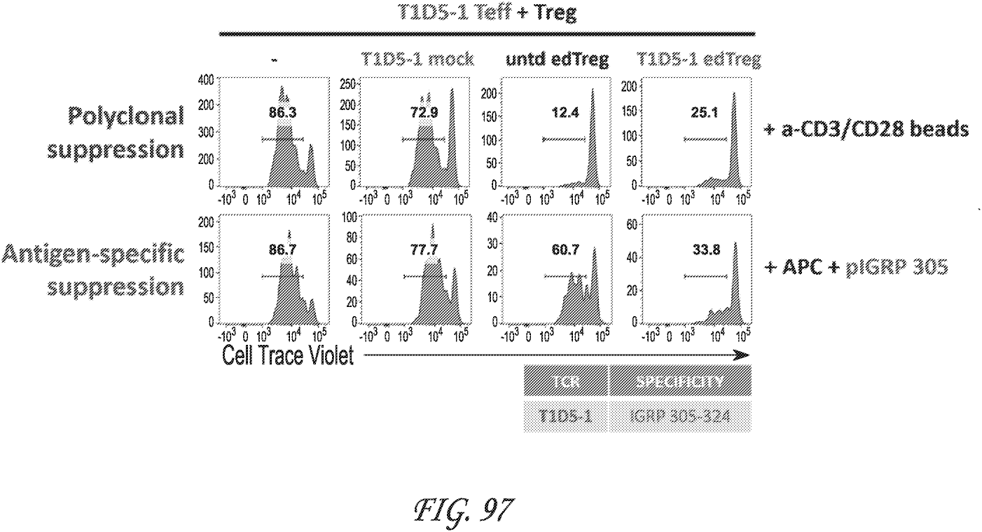

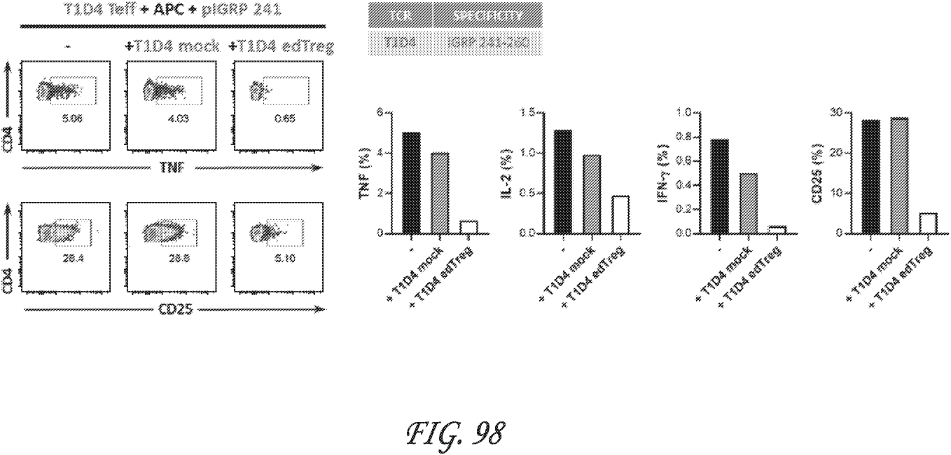

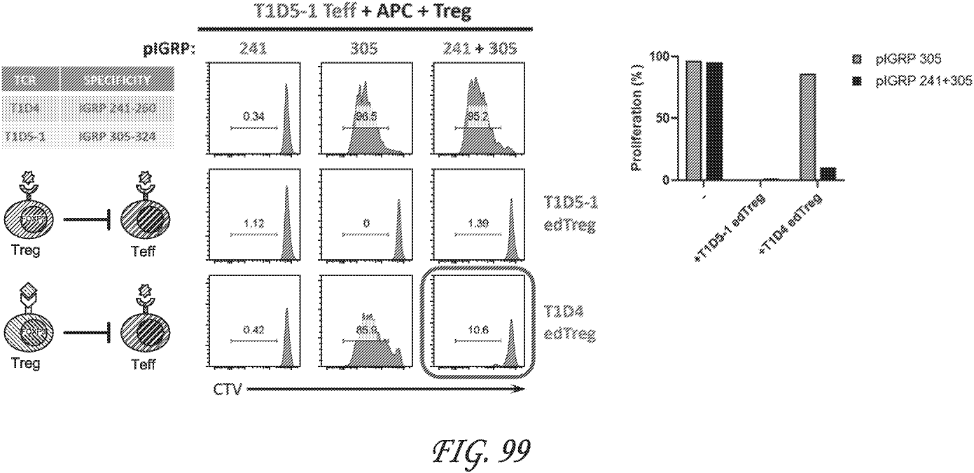

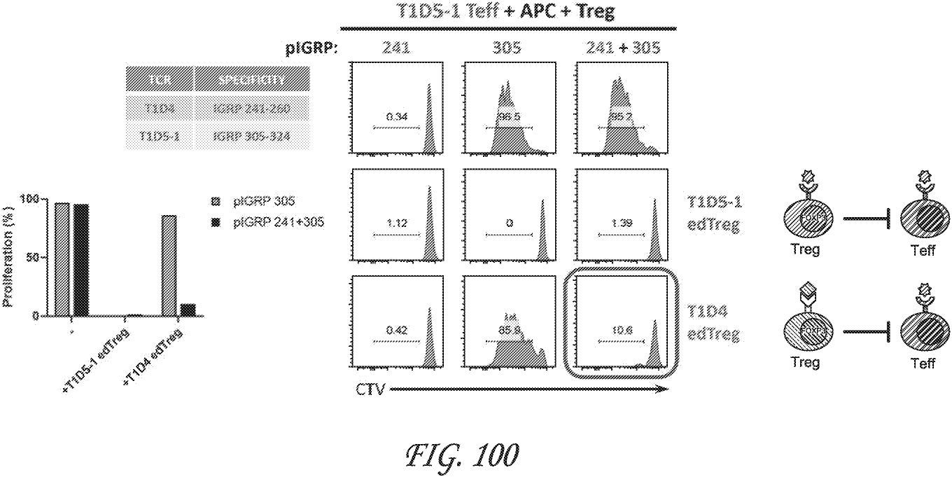

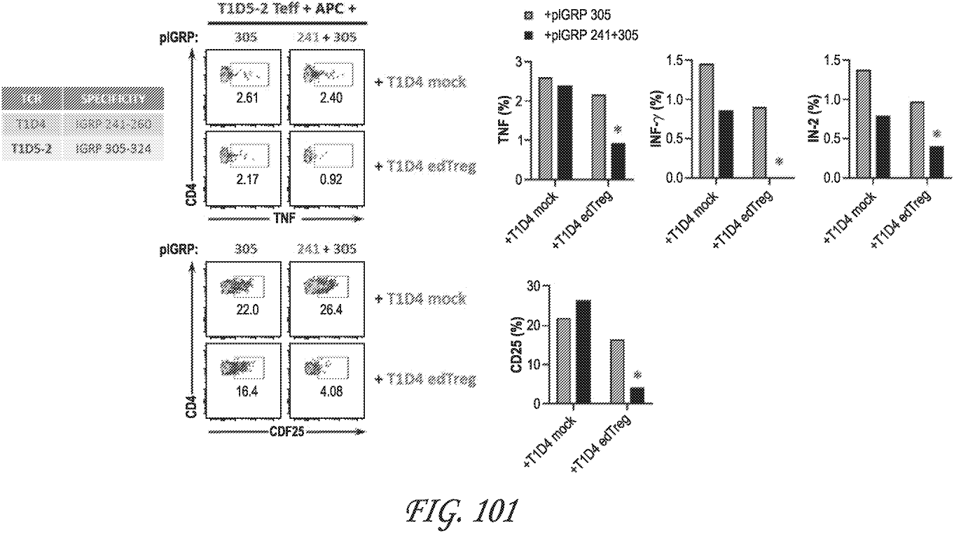

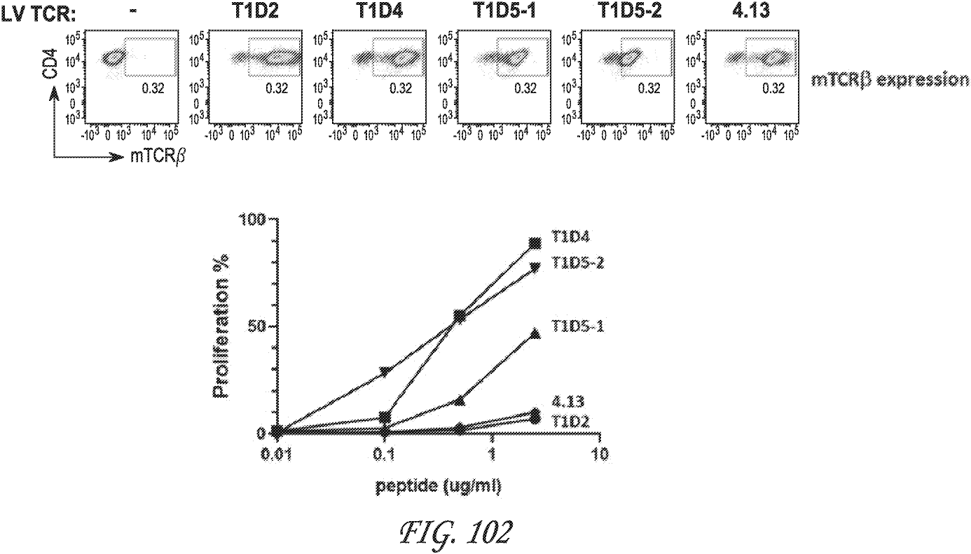

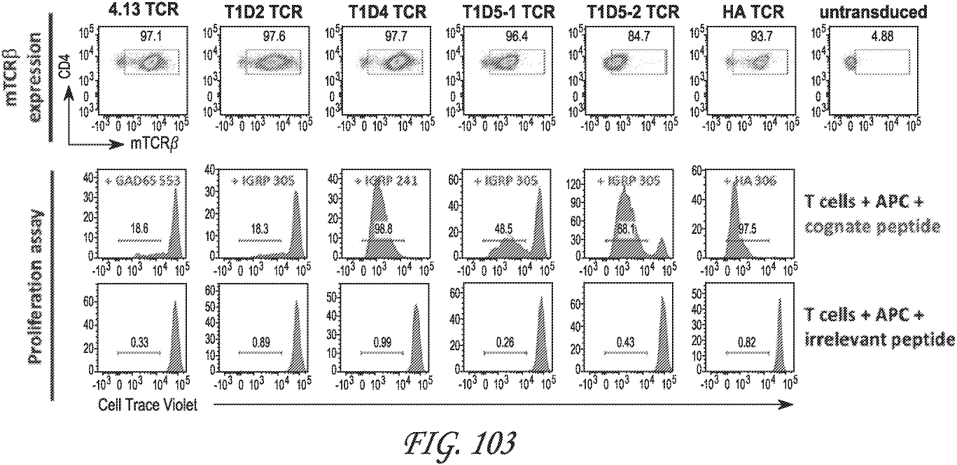

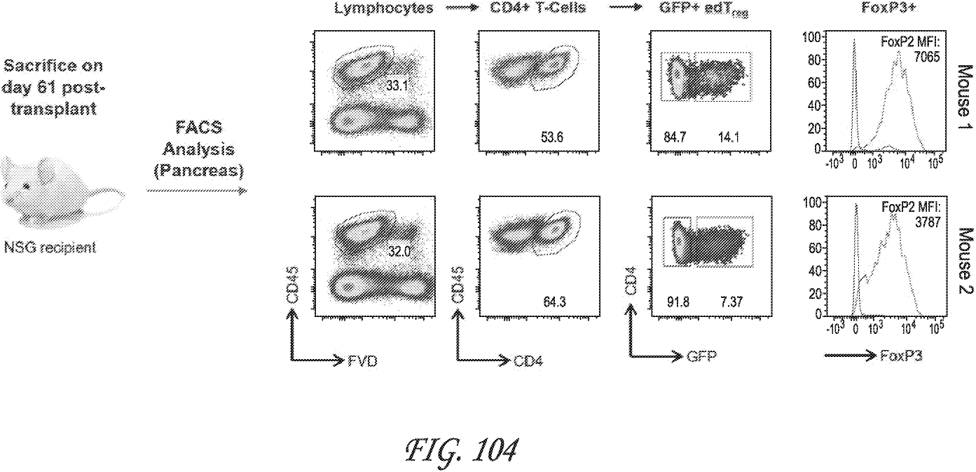

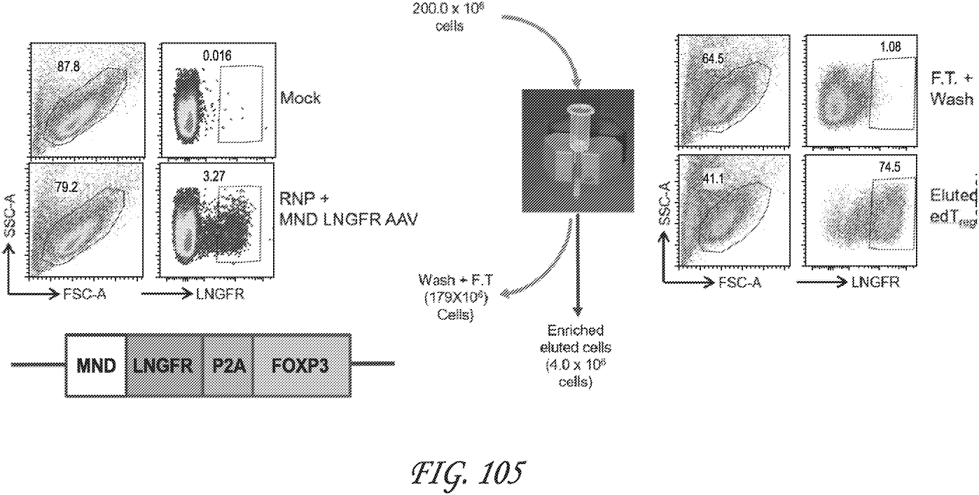

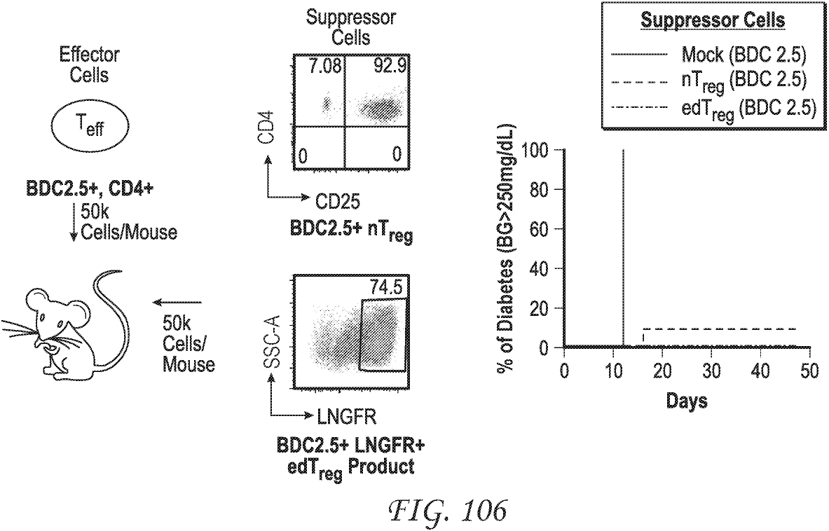

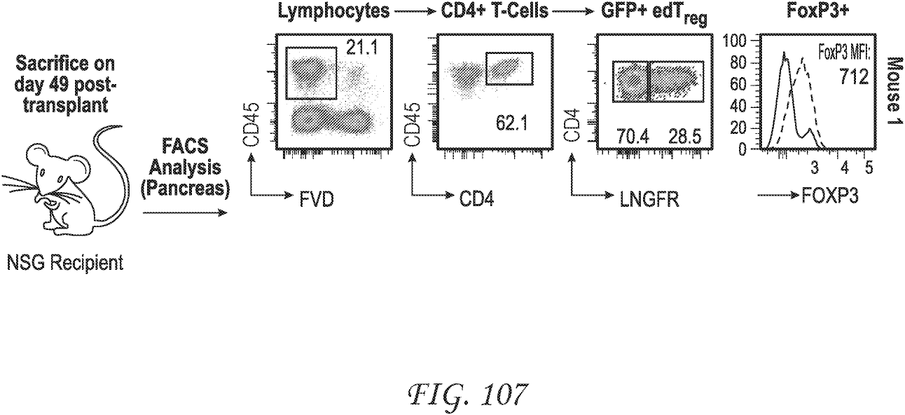

An important feature of a human antigen-specific airT platform is the potential to expand airT in vitro and in vivo using, as an example, the IL-2-CISC system. To assess the function of airT containing the IL-2-CISC cassette in immune competent animal disease models, experiments were performed to test whether the human IL-2R sequence containing CISC/DISC cassette can promote selective expansion of murine cells in vitro and in vivo with Rapalog/Rapamycin. These data demonstrated proof-of-concept using a lentiviral construct, #1272, that contained a MND promoter-driven mCherry reporter and cis-linked human IL-2 CISC elements. FIG. 92 shows the schematic of the lentiviral cassette and the timeline of T cell transduction, expansion and analysis. In this study, transduced cells were placed in either: (a) IL-2, IL-7 and IL-15; (b) Rapalog alone; or (c) Rapalog plus an additional CD3/CD28 bead stimulation 2 days after transduction. FIG. 93 demonstrates mCherry expression in 8.85% of the transduced cells and further enrichment after 3 days of Rapalog treatment. Enrichment was greatest (46.1%) when transduced T cells that were concurrently treated with both Rapalog and an additional CD3/CD28 bead stimulation.

These data show that murine CD4+ T cells can be enriched using the IL-2 CISC technology and that human CISC is functional in the mouse system. These findings demonstrate the feasibility of studies to examine enrichment and function of murine Ag-specific airT using the split-CISC/Split-DISC approach in non-clinical animal models.

### Example 12—Generation and Testing of Antigen-Specific edTreg

### RA Antigen-Specific TCRs Identified From RA Patients

RA antigen-specific TCRs were identified from T cells clones isolated from RA patients. Based on these sequences, lentiviral TCR constructs for TCR gene transfer were generated. TABLE 1 lists generated lentiviral constructs encoding RA antigen-specific TCRs, their epitope specificity, and HLA-restriction. Target T cell epitope sequences included citrulline modifications. TCRs recognizing citrullinated -vimentin, -aggrecan, -CILP, and enolase were identified from T cell clones that were previously isolated from RA patients

TABLE 1

| TCR name | Antigen | Epitope | Target sequence | SEQ ID NO | HLA-DR |
|---|---|---|---|---|---|
| Vim418 | vimentin | 418-431 | SSLNL(Cit)ETNLDSL | SEQ ID NO: 1408 | ∗0404 |
| Agg153 | aggrecan | 153-168 | IVFHY(Cit)AIST(Cit)YTLDF | SEQ ID NO: 1409 | ∗0404 |
| Agg520 | aggrecan | 520-539 | GYEQCDAGWL(Cit)DQTV(Cit)YPIV | SEQ ID NO:1410 | ∗0401 |
| Agg553 | aggrecan | 553-570 | PGV(Cit)TYGV(Cit)PSTETYDVY | SEQ ID NO:1411 | ∗0401 |
| Agg621 | aggrecan | 621-637 | KCYAGWLADGSL(Cit)YPIV | SEQ ID NO:1412 | ∗0401 |
| CILP297-1 | CILP | 297-311 | ATIKAEFV(Cit)AETPYM | SEQ ID NO:1413 | ∗0401 |
| CILP297-2 | CILP | 297-311 | ATIKAEFV(Cit)AETPYM | SEQ ID NO:1414 | ∗0401 |
| Enol326 | enolase | 326-340 | K(Cit)IAKAVNEKSCNCL | SEQ ID NO:1415 | ∗0401 |

CD4+ T cells were isolated, activated with CD3/CD28 beads, and transduced with lentiviral RA Ag-specific TCRs. Flow plots show mTCRb expression gated on CD4+ cells day 9 post-transduction (FIG. 117A). CD4+ T cells transduced with RA Ag-specific TCRs were labeled with CTV and co-cultured with APC (irradiated PBMC) and their cognate peptide or DMSO for 3 days. Flow plots show cell proliferation as CTV dilution (FIG. 117B). RA-specific TCR expression was validated by T cell proliferation assays using peptides cognate with the TCRs and antigen presenting cells (APCs). T cells transduced with RA-specific TCRs (vimentin, aggrecan, CILP and enolase) proliferated in response to their cognate peptides and APC.

### Suppressive Activity of Enolase-Specific edTreg

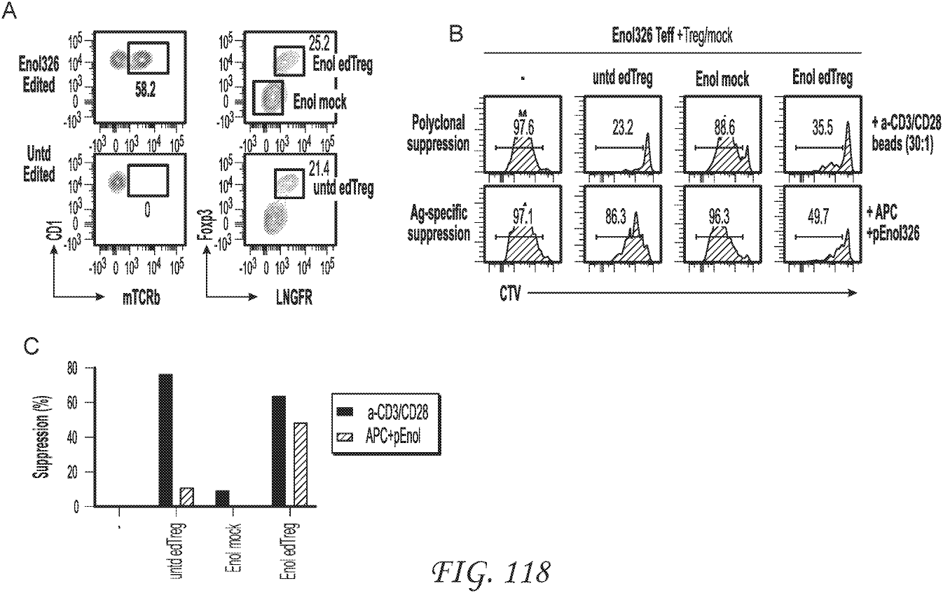

Antigen-specific Treg were generated by editing the Foxp3 locus in CD4 T cells that had been transduced with enolase TCRs. This resulted in the successful generation of enolase-specific edTreg. FIG. 118A depicts flow plots of mTCRb expression and LNGFR/Foxp3 expression in edited cells without LV transduction (Untd Edited) and edited cells expressing Enol326-TCR (Enol326 Edited) on day 7. edTreg cells were enriched by LNGFR expression on day 10 and LNGFR- cells were used as mock cells for suppression assays. The transduced Enol326-TCR had a specificity for an epitope of Enolase 326-340.

FIG. 118B depicts a polyclonal suppression assay and an antigen-specific suppressing assay using enolase-specific edTreg. Enolase-specific Teff cells were produced from LV Enol326-TCR transduction of CD4+ T cells and expanded for 15 days. For the polyclonal assay, Enol326 Teff were incubated with anti-CD3/CD28 beads at 1:30 of bead to cell ratio with no Treg, untd edTreg, Enol326 edTreg, or mock cells. For the antigen-specific suppression assay, Enol326 Teff cells were co-cultured with APCs and Enol326 peptide in the presence of no Treg, untransduced (untd) edTreg, Enol326 edTreg, or mock cells. For all the suppression assay set up, Teff and edTreg or mock cells were labeled with CTV and EF670, respectively and co-cultured at 1:1 ratio. 4 days after the co-culture, cells were stained and analyzed for Teff proliferation as dilution of CTV. FIG. 118C depicts a graph of percentage suppression of Teff proliferation by no Treg, untd edTreg, Enol edTreg, or mock in the presence of a-CD3/CD28 (black) or APC and enolase peptide (grey) calculated from percentage proliferation in FIG. 118B. The enolase-specific edTregs showed antigen-specific and polyclonal suppressive function of antigen-specific T effector cells by in vitro suppression assays.

### Suppressive Activity of CILP-Specific edTreg

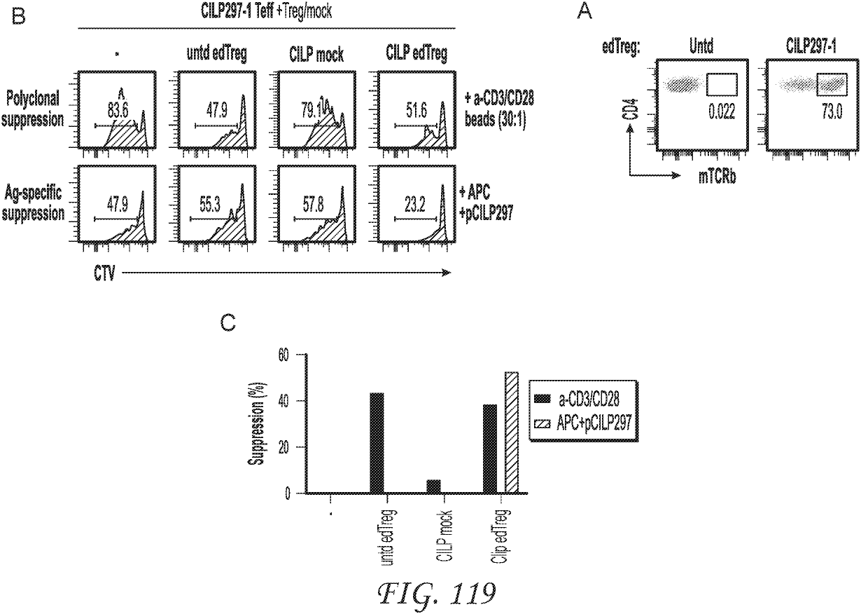

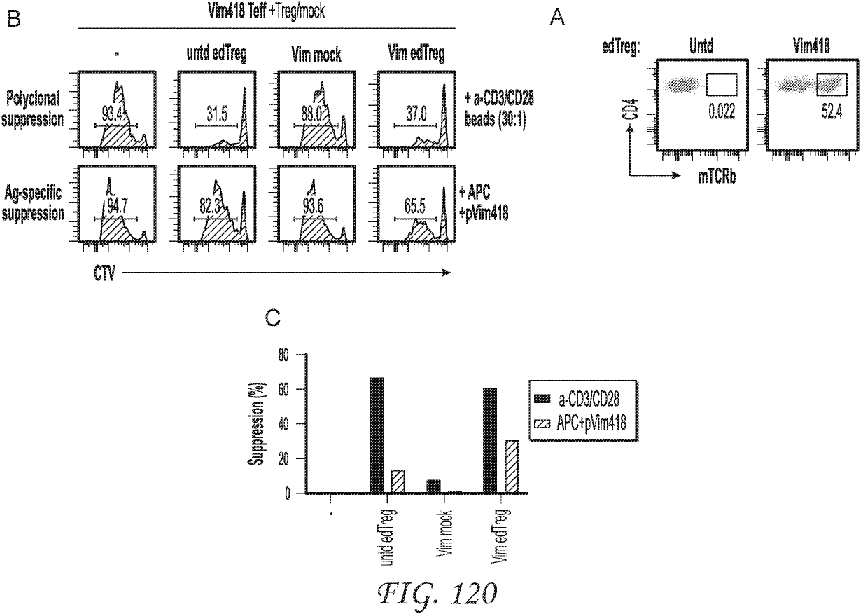

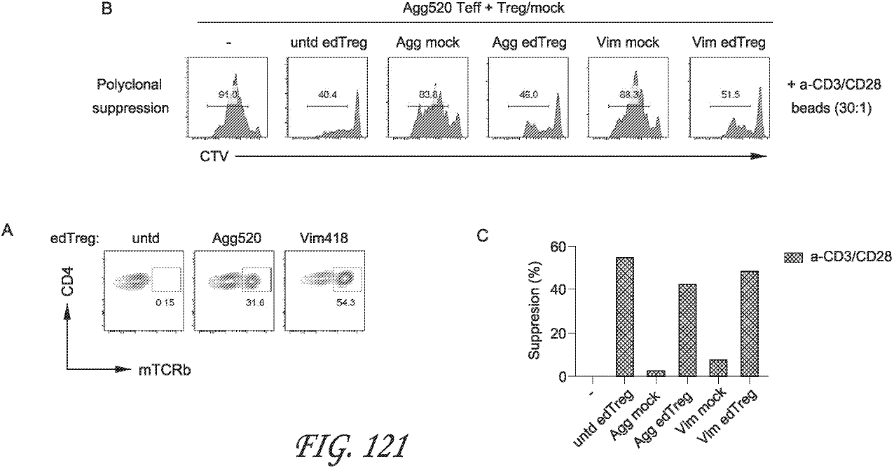

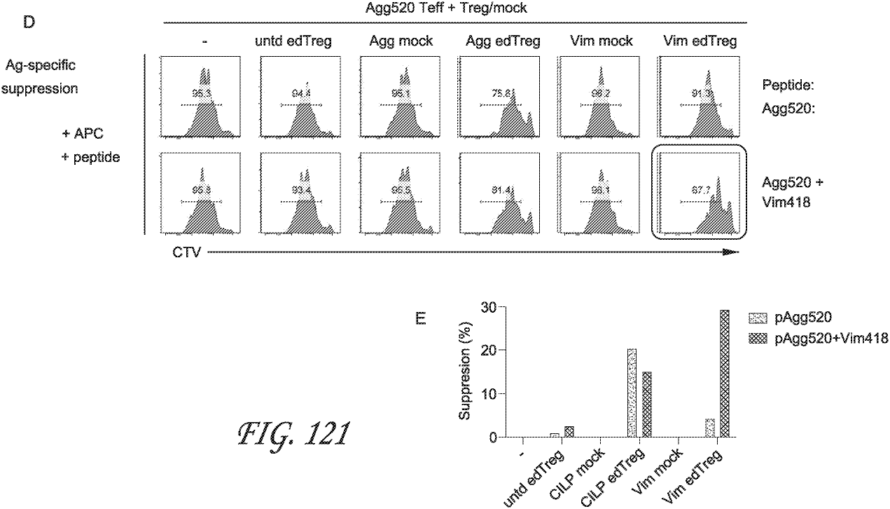

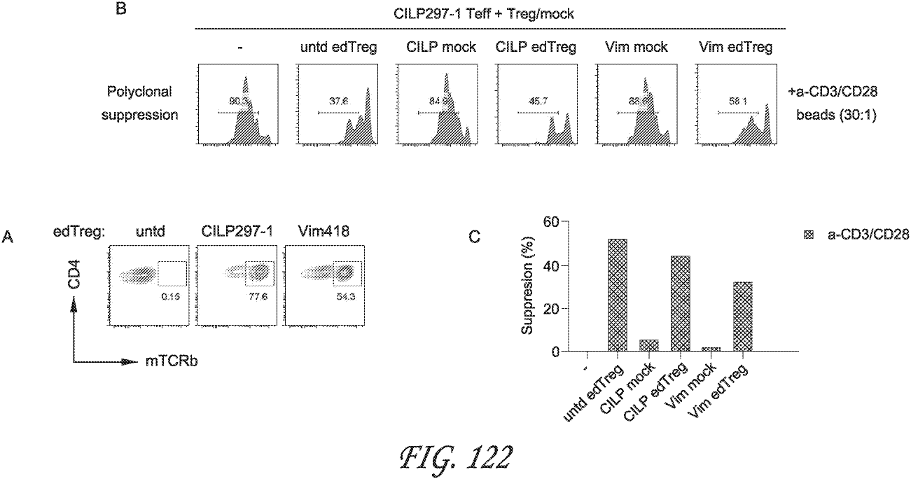

Suppressive activity of CILP-specific edTreg was determined. FIG. 119A depicts flow plots of mTCRb expression in untransduced edTreg and CILP297-1 edTreg gated on LNGFR+ Foxp3+ in edited cells transduced with no LV and LV CILP297-1-TCR, respectively. The CILP297-1 TCR had a specificity to a CILP 297-311 epitope. FIG. 119B depicts a polyclonal suppression assay and an antigen-specific suppressing assay using CILP-specific edTreg. CILP-specific Teff cells were produced from LV CILP297-1-TCR transduction of CD4+ T cells and expanded for 15 days. For the polyclonal assay, CILP Teff were incubated with anti-CD3/CD28 beads with no Treg, untd edTreg, CILP edTreg, or mock cells. For the antigen-specific suppression assay, CILP Teff cells were co-cultured with APCs and CILP297 peptide in the presence of no Treg, untd edTreg, CILP edTreg, or mock cells. FIG. 119C depicts a graph of percentage suppression of CILP Teff proliferation by no Treg, untd edTreg, CILP edTreg, or mock in the presence of a-CD3/CD28 (black) or APC and CILP peptide (grey) calculated from percentage proliferation in FIG. 119B. Similar results were seen using CILP-specific edTregs. The CILP-specific edTregs showed antigen-specific and polyclonal suppressive function of antigen-specific T effector cells by in vitro suppression assays.

### Suppressive Activity of Vimentin-Specific edTreg

Suppressive activity of vimentin-specific edTreg was determined. FIG. 120A depicts flow plots of mTCRb expression in untransduced edTreg and Vim418 edTreg gated on LNGFR+ Foxp3+ in edited cells transduced with no LV and LV Vim418-TCR, respectively. The Vim418 TCR had a specificity to the epitope vimentin 418-431.

FIG. 120B depicts a polyclonal suppression assay and an antigen-specific suppressing assay using vimentin-specific edTreg. Vimentin-specific Teff cells were produced from LV Vim418 TCR transduction of CD4+ T cells and expanded for 15 days. For the polyclonal assay, Vim Teff were incubated with anti-CD3/CD28 beads with no Treg, untd edTreg, Vim edTreg, or mock cells. For the antigen-specific suppression assay, Vim Teff cells were co-cultured with APCs and Vim418 peptide in the presence of no Treg, untd edTreg, Vim edTreg, or mock cells. FIG. 120C depicts a graph of percentage suppression of Vim Teff proliferation by no Treg, untd edTreg, Vim edTreg, or mock in the presence of a-CD3/CD28 (black) or APC and Vimentin peptide (grey) calculated from percentage proliferation in FIG. 120B. The vimentin -specific edTregs showed antigen-specific and polyclonal suppressive function of antigen-specific T effector cells by in vitro suppression assays

### Antigen-Specific and Bystander Suppression of Aggrecan-Specific Teff

Antigen-specific suppression and bystander suppression of aggrecan-specific Teff was demonstrated with aggrecan-specific edTreg and vimentin-specific edTreg, respectively.

FIG. 121A depicts flow plots show mTCRb expression in untransduced, Agg520, and Vim418 edTreg gated on LNGFR+ Foxp3+ in edited cells transduced with no LV, LV Agg520-TCR, and LV Vim418-TCR, respectively. The Agg520 TCR has specificity to the epitope Aggrecan 520-539. FIG. 121B depicts a polyclonal suppression assay using Agg520 Teff and edTreg or mock specific to Agg520 or Vim418. Aggrecan-specific Teff cells were produced from LV Agg520-TCR transduction of CD4+ T cells and expanded for 15 days. Agg520 Teff were incubated with anti-CD3/CD28 beads with no Treg, edTreg, or mock. FIG. 121C depicts a graph of percentage suppression of Agg520 Teff proliferation by no Treg, untd edTreg, Agg edTreg/mock, or Vim edTreg/mock calculated from percentage proliferation in FIG. 121B. FIG. 121D depicts an antigen-specific and a bystander suppression assay using Agg520 Teff and edTreg or mock specific to Agg520 or Vim418. Agg520 Teff cells were co-cultured with no Treg, edTreg, or mock in the presence of APCs and Agg520 peptide or Agg520+Vim418 peptide. FIG. 121E depicts a graph of percentage suppression of Agg520 Teff proliferation by no Treg, edTreg or mock calculated from percentage proliferation in FIG. 121D. Significantly, bystander suppression of aggrecan-specific Teff by vimentin-specific edTreg was demonstrated.

### Antigen-Specific and Bystander Suppression of CILP-Specific Teff

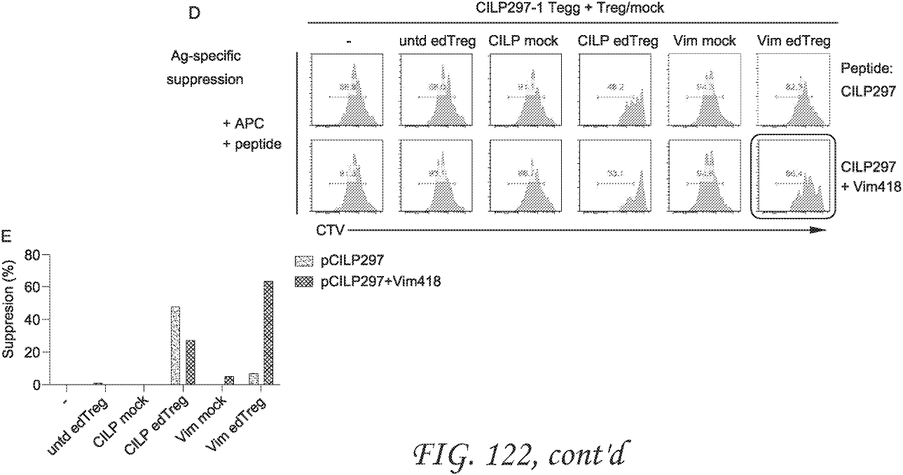

Antigen-specific suppression and bystander suppression of CILP-specific Teff was demonstrated with CILP-specific edTreg and vimentin-specific edTreg, respectively. FIG. 122A depicts flow plots of mTCRb expression in untransduced, CILP297-1, and Vim418 edTreg gated on LNGFR+ Foxp3+ in edited cells transduced with no LV, LV CILP297-1-TCR, and LV Vim418-TCR, respectively. FIG. 122B depicts a polyclonal suppression assay using CILP297-1 Teff and edTreg or mock specific to CILP297 or Vim418. CILP-specific Teff cells were produced from LV CILP297-1-TCR transduction of CD4+ T cells and expanded for 15 days. CILP297-1 Teff were incubated with anti-CD3/CD28 beads with no Treg, edTreg, or mock. FIG. 122C depicts a graph of percentage suppression of CILP Teff proliferation by no Treg, untd edTreg, CILP edTreg or mock, or Vim edTreg or mock calculated from percentage proliferation in FIG. 122B. FIG. 122D depicts an antigen-specific and bystander suppression assay using CILP297-1 Teff and edTreg specific to CILP297 and Vim418. CILP297-1 Teff cells were co-cultured with no Treg, edTreg, or mock in the presence of APCs and CILP297 peptide or CILP297+Vim418 peptide. FIG. 122E depicts a graph of percentage suppression of CILP Teff proliferation by no Treg, edTreg or mock calculated from percentage proliferation in FIG. 122D. Significantly, bystander suppression of CILP297-1 specific Teff by Vim edTregs was demonstrated.

### SLE-Specific edTreg and Their Suppressive Activity

SLE-specific edTregs were generated. CD4 T cells were transduced with a SLE3 TCR, previously identified from a lupus patient, and the Foxp3 locus was edited.

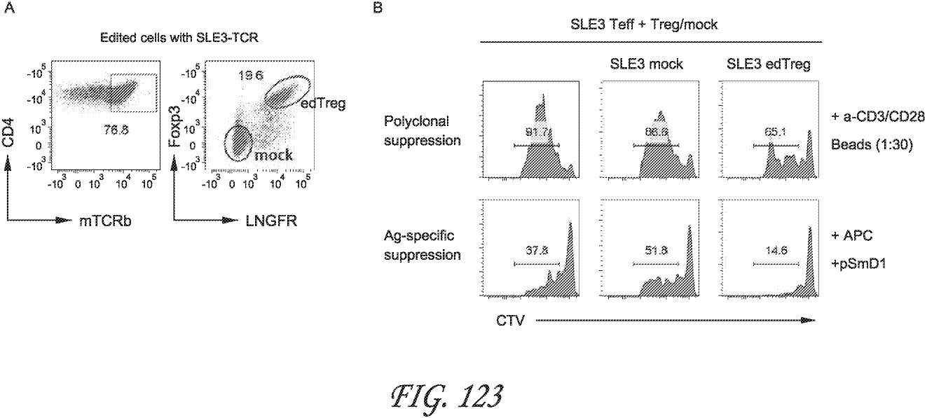

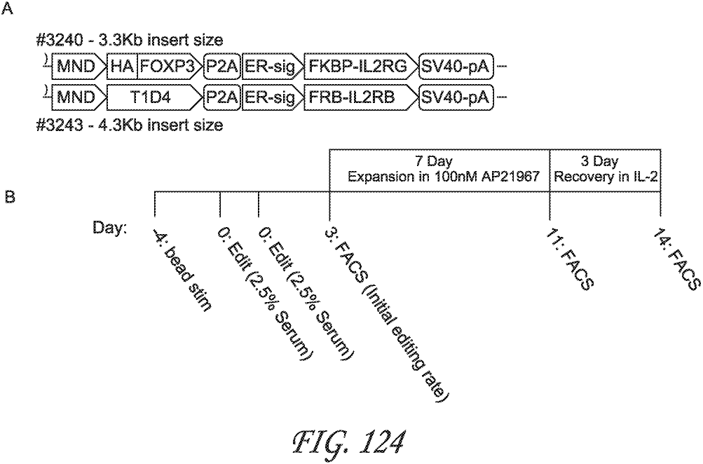

FIG. 123A depicts flow plots of mTCRb expression and LNGFR/Foxp3 expression in edited cells expressing SLE3-TCR on day 7. SLE3-TCR was previously identified from lupus patient. edTreg cells were enriched by LNGFR expression on day 10 and LNGFR- cells were used as mock cells for suppression assays. The SLE3-TCR had a specificity the epitope SmD1 65-80. FIG. 123B depicts a polyclonal suppression assay and an antigen-specific suppressing assay using SLE-specific edTreg. SLE-specific Teff cells were produced from LV SLE3-TCR transduction of CD4+ T cells and expanded for 15 days. For the polyclonal assay, SLE3 Teff were incubated with anti-CD3/CD28 beads with no Treg, SLE3 edTreg, or mock cells. For the antigen-specific suppression assay, SLE3 Teff were co-cultured with APCs and SmD1 peptide in the presence of no Treg, SLE3 edTreg, or mock cells. Edited cells expressed SLE-TCR and have polyclonal and antigen-specific suppressive activity.

At least data in this Example demonstrated the ability to generate T1D, RA and SLE antigen-specific edTdreg, with suppressive activity suggesting the potential use of antigen-specific edTreg therapies across a broad spectrum of autoimmune diseases.

### Example 13—Dual-Editing of Human CD4+ T Cells

Human CD4+ T cells were dual edited to generate edTreg to have an endogenous TCR knock-outed /inactivated, to be antigen-specific, and/or to be drug-selectable.

### Single Locus Approach

An IL-2 split-CISC system was used in a dual HDR editing strategy to provide efficient selection and enrichment of dual edited cells with endogenous TCR knockout. A challenge of the dual-editing approach is the ability to obtain sufficient numbers of edited cells for therapeutic use. This study aimed to increase cell viability during an expansion phase. The TRAC targeting AAV HDR-donor constructs used are depicted in FIG. 124A. AAV HDR-donor constructs were designed to introduce split-CISC elements into the TRAC locus using a single locus dual editing approach. CISC components were split between 2 constructs and co-expressed with either HA-FOXP3 or the T1D4 TCR (#3240 and #3243 respectively). Repair templates were flanked by homology arms matched to gRNAs targeting the TRAC locus. Only edited CD4+ T cells that incorporated both expression cassettes were predicted to selectively expand under Rapalog exposure.

A timeline for key steps for dual AAV editing of CD4+ T cells and expansion with Rapalog is depicted in FIG. 124B. The expansion protocol was adjusted from a 10-day expansion in AP21967 (a rapamycin analog) to 7 day expansion in AP21967 followed by a 3-day recovery in IL-2 containing medium. Briefly, human CD4+ T cells were edited using human TRAC gRNA_4, and #3240 (MND.HA.FOXP3.FKBP.IL2RG) and #3243 (MND.T1D4.FRB.IL2RB) AAV constructs (single-locus dual editing). Immediately following electroporation, the cells were placed in 2.5% FBS containing media (recovery media) for ~24 hours and then maintained in 20% FBS containing media throughout the rest of the experiment. FACS analysis was done on day 3 to determine editing rate and edited populations were cultured in the presence of either IL-2 or Rapalog for an additional 7 days to enrich dual FOXP3/T1D4 positive cells. Cells were allowed to recover for 3 days in media containing IL-2 prior to FACS analysis on day 14.

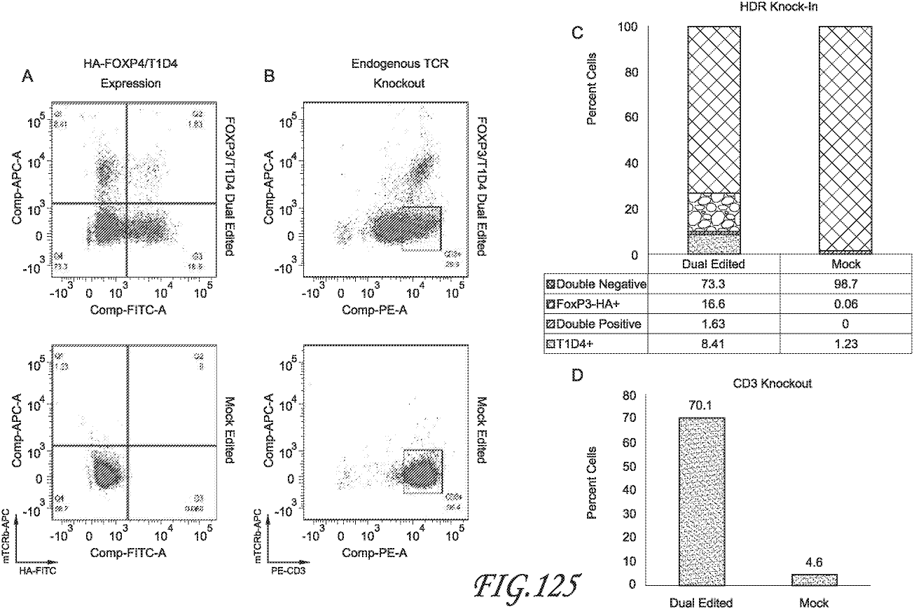

Dual editing of human CD4+ T cells using FOXP3 and T1D4 split-CISC constructs within the human TRAC locus resulted in FOXP3/T1D4 double positive cells and disrupted TCR expression. FIG. 125A depicts flow plots which show T1D4 and FOXP3 expression in mock edited, single edited and dual-edited cells (using 10% volume of both #3243 and #3240 AAV) at Day 3 post editing. Viral titers were 4.2E¹¹ and 1.3E¹² for #3243 and #3240, respectively. FIG. 125B depicts flow plots which show T1D4 and CD4 expression in mock edited, and mixed edited cells. FIG. 125C depicts histograms which show percent double negative, FOXP3-HA positive, T1D4 positive and FOXP3/T1D4 double positive cells within the dual edited cells. FIG. 125D depicts histograms which show percent CD3 knockout in FOXP3/T1D4 dual edited cells vs. mock edited cells. FACS analysis demonstrated an initial editing rate of 1.6% in T1D4/FOXP3 dual-edited cells compared to 0% in mock edited cells and CD3 knock-out (KO) of 70% in dual-edited cells.

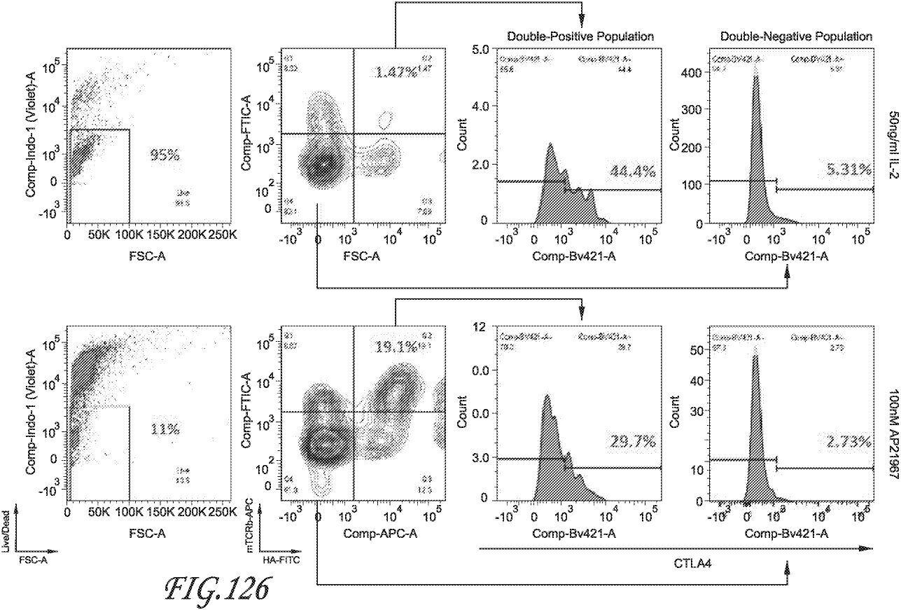

A robust enrichment of dual edited FOXP3/T1D4 expressing cells and increased CTLA4 expression was observed with AP21967 treatment of dual edited cells. TRAC locus dual-editing was performed as shown in FIG. 124A and FIG. 124B. FIG. 126A depict flow plots showing viability and T1D4 and FOXP3 expression in dual-edited cells treated with either 50 ng/mL IL-2 (upper panels) or 100 nM Rapalog (AP21967; lower panels) for 7 days. FIG. 126B depict flow plots showing CTLA4 expression of T1D4/FOXP3 double positive vs. double negative cell populations treated with either 50 ng/mL IL-2 (upper panels) or 100 nM Rapalog (AP21967; lower panels) for 7 days. FACS analysis following enrichment at day 7 showed a steady increase in FOXP3/T1D4 dual positive cells over time with 19.1% double positive cells at day 7 in AP21967 compared to 1.47% in IL-2.

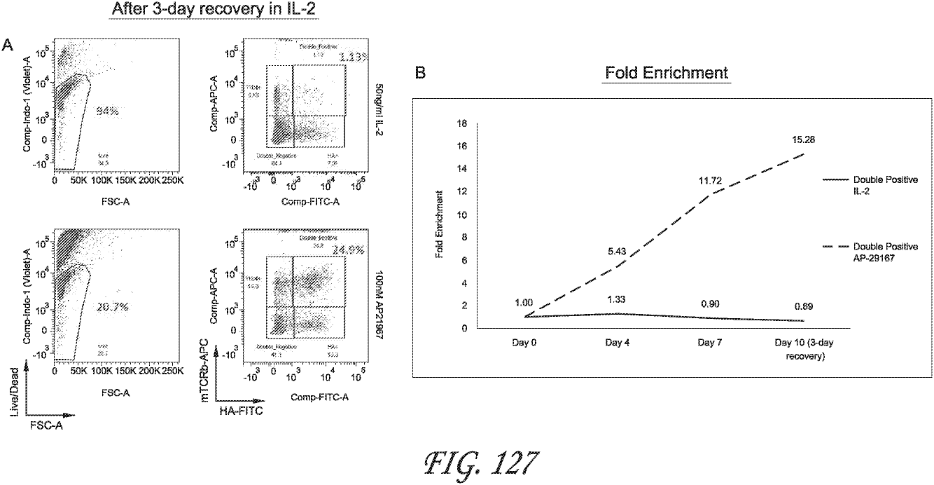

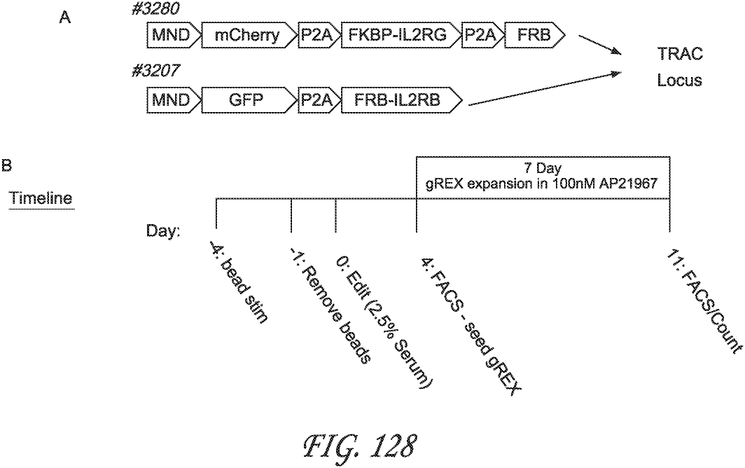

Cell viability in AP21967 declined in comparison to cells treated with 50 ng/mL IL-2 (11% viability vs. 95% viability respectively) (FIG. 126A). To improve cell viability following AP21967 treatment, cells were cultured in 50 ng/mL IL-2 containing medium following 7 days in AP21967. An improved viability and continued enrichment of dual edited FOXP3/T1D4 expressing cells was observed for cells treated with AP21967 following recovery in IL-2. TRAC locus dual-editing was performed as shown in FIG. 124A and FIG. 124B. Cells were analyzed at Day 10 following a 3-day recovery in IL-2 containing medium. FIG. 127A depicts flow plots showing viability (right plots) and T1D4 and FOXP3 expression (left plots) in dual-edited cells following treatment with 50 ng/mL IL-2 (upper plots) vs. 100 nM AP21967 (lower plots) after recovery in IL-2 medium. FIG. 127B depicts a graph showing fold enrichment of double positive T1D4/FOXP3 cells treated with either 50 ng/mL IL-2 or 100 nM Rapalog (AP21967) over a 10 day period with the last 3 days being in recovery media containing IL-2. Following recovery in IL-2, overall viability increased from 11% to 20.7% (FIG. 126A, FIG. 127A) and the percentage of double positive FOXP3/T1D4 cells continued to increase to 24.9%. Overall, the double-positive antigen-specific Treg population enriched approximately 15-fold over the course of this study (FIG. 127B), suggesting this may be an approach to improve viability and expansion.

To further characterize the T1D4/FOXP3 expressing cells, expression of CTLA4, a marker of FOXP3 expressing natural T regulatory cells (nTreg) was measured. FIG. 126B shows that double positive T1D4/FOXP3 expressing cells exhibited an increased expression of CTLA4 compared to the double-negative population consistent with a Treg-like phenotype.

This study further demonstrated that dual-editing can be used to introduce both a candidate TCR and the IL2 split-CISC cassette, and for enrichment using a Rapalog and generation of antigen-specific edTreg.

### Dual Editing Using Decoy-CISC (Split-DISC) Constructs

Rapamycin can be used in clinical studies using CISC-expressing edTreg. “Decoy-CISC” (DISC) constructs for efficient enrichment using either Rapamycin or AP21967 were tested. Split-DISC constructs were used to determine the enrichment and expansion of dual-edited T cells. The ability to scale up manufacturing to obtain cell numbers sufficient for animal studies by expanding edited CD4+ T cells in gREX flasks was assessed. In particular, dual-editing and enrichment of human CD4+ T Cells using split-DISC constructs was studied. Briefly, FIG. 128A depicts a split IL-2 DISC HDR knock-in construct (#3280), for selection of dual-edited cells in either Rapamycin or Rapalog. To generate the split decoy-CISC (split-DISC), the free FRB domain for cytoplasmic Rapamycin sequestration was added to the MND.mCherry.FKBP.IL2RG construct to generate (MND.mCherry.FKBP.IL2RG.FRB (#328)). Each repair template (#3280 and #3207) was flanked by identical homology arms matched to a gRNA targeting the TRAC locus. Edited CD4+ T cells incorporating one copy of each construct were predicted to selectively expand under Rapalog or Rapamycin treatment. FIG. 128B depicts a timeline of steps for dual AAV editing of CD4+ T cell using AAV #3280 and #3207, expansion with Rapalog/Rapamycin and analysis of enriched cells. Cells were bead stimulated (CD3/CD28) for 3 days prior to editing. Immediately following electroporation, the cells were placed in 2.5% FBS containing media (recovery media) for ~24 hours and then maintained in 20% FBS containing media throughout the rest of the experiment. Three days post editing, cells were analyzed by flow for GFP and mCherry expression, and then expanded in media containing 50 ng/ml human IL-2 or 100 nM Rapalog.

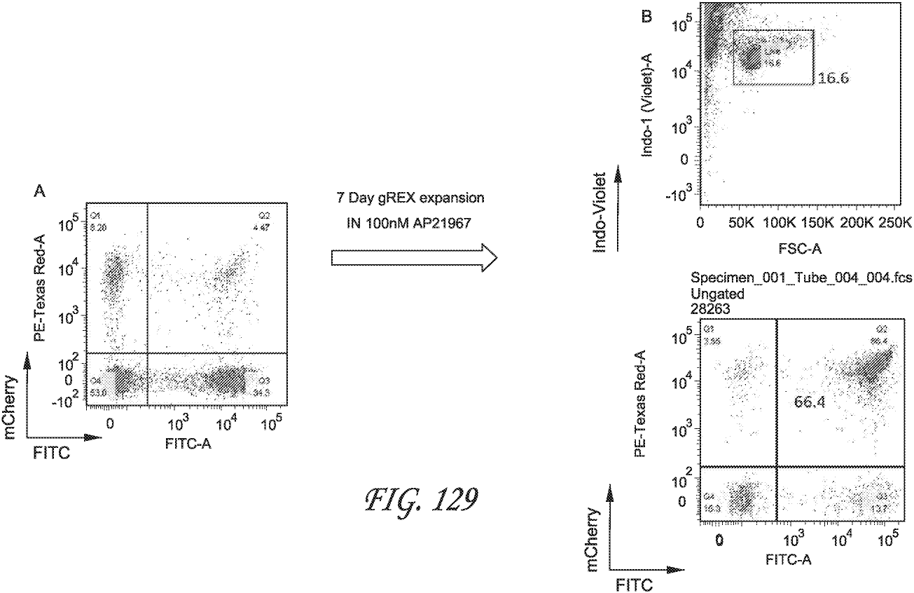

Dual editing of human CD4+ T cells using decoy-CISC (split-DISC) constructs and enrichment with AP21967 resulted in robust expansion of double positive cells. FIG. 129A depicts flow plots showing mCherry and GFP expression in dual edited cells (10% culture volume of #3280 and #3207 AAV donors, respectively) four days post editing. Viral titers were 3.30E+12 and 3.1E+10 for #3280 and #3207 respectively. FIG. 129B depicts flow plots showing viability (upper panel) and GFP and mCherry expression (lower panel) following the seeding of 7.6 million edited cells in gREX and 7 day expansion in the presence of AP21967 leading to 32-fold expansion of double-positive cells. The FACS analysis confirmed an initial editing rate of 4.47% mCherry/GFP double positive cells and enrichment to 66% mCherry/GFP double positive cells after 7 day expansion in gREX in the presence of AP21967. The results demonstrated a 32-fold expansion of double positive cells during the 7-day treatment in AP21967 resulting in a total of 11.1 million double positive cells from the original 340,000 cells seeded into gREX.

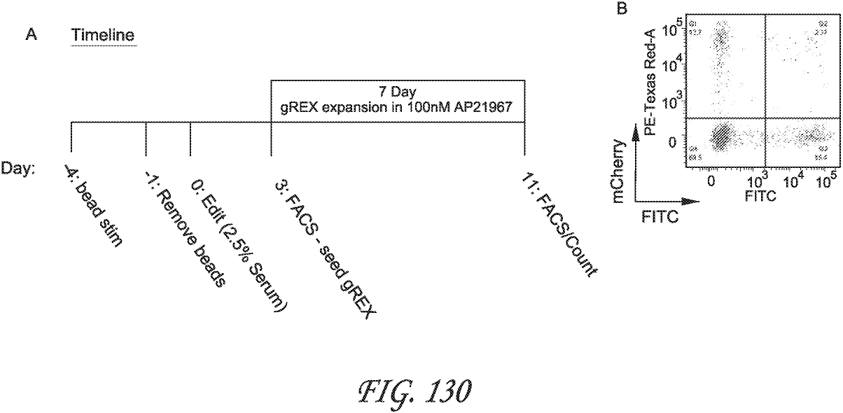

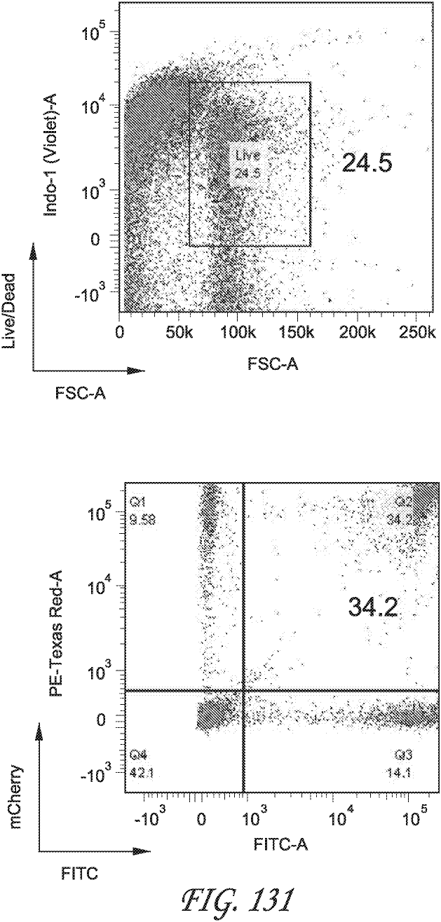

As second study was performed with a substantially similar protocol as immediately above. Dual editing of human CD4+ T cells using decoy-CISC (split-DISC) constructs and enrichment with AP21967 resulted in robust expansion of double positive cells. FIG. 130A depicts flow plots showing mCherry and GFP expression in dual edited cells (10% culture volume of #3280 and #3207 AAV donors, respectively) four days post editing. Viral titers were 3.30E+12 and 3.1E+10 for #3280 and #3207 respectively. FIG. 130B depicts flow plots show viability (upper panel) and GFP and mCherry expression (lower panel) following the seeding of 7.6 million edited cells in gREX and 7 day expansion in the presence of AP21967 leading to 32-fold expansion of double-positive cells.

Robust expansion of dual edited human CD4+ T cells using decoy-CISC (split-DISC) constructs was reproducible. FIG. 130A depicts a timeline of key steps for dual AAV editing of CD4+ T cell using AAV #3280 and #3207, expansion with Rapalog and analysis of enriched cells. Cells were bead stimulated (CD3/CD28) for 3 days prior to editing. Three days post editing, cells were analyzed by flow for GFP and mCherry expression, and then expanded in media containing 100 nM Rapalog for an additional 7 days. FIG. 130B depicts a flow plot showing mCherry and GFP expression in dual edited cells (10% #3280 and 10% #3207 AAV). Viral titers were 3.30E+12 and 3.1E+10 for #3280 MND.mCherry.FKBP.IL2RG.FRB and #3207 pAAV.MND.GFP.FRB.IL2RB respectively.

Expansion of dual edited human CD4+ T cells using decoy-CISC (split-DISC) constructs with AP21967 resulted in 45-fold increase in enriched cells. Cells were dual-edited as depicted in FIG. 130A. FIG. 131 depicts flow plots show viability and GFP and mCherry expression following the seeding of edited cells in gREX and 7 day expansion in the presence of AP21967. The total number of double positive cells in gREX at day 7 was 9.7 million, a ~45-fold increase from the initial seeding of 216,000 double positive cells.

Importantly, these studies demonstrated that dual-editing strategy into the TRAC locus using the split DISC constructs provided at least a ~45-fold expansion of dual edited cells. This level of expansion was similar to that observed using an all-in-one DISC constructs in a single editing event. Thus, this approach provides an efficient enrichment of dual-edited cells for in-vivo transplantation studies and ultimately clinical application.

### Ag-Specific Treg Mouse Studies

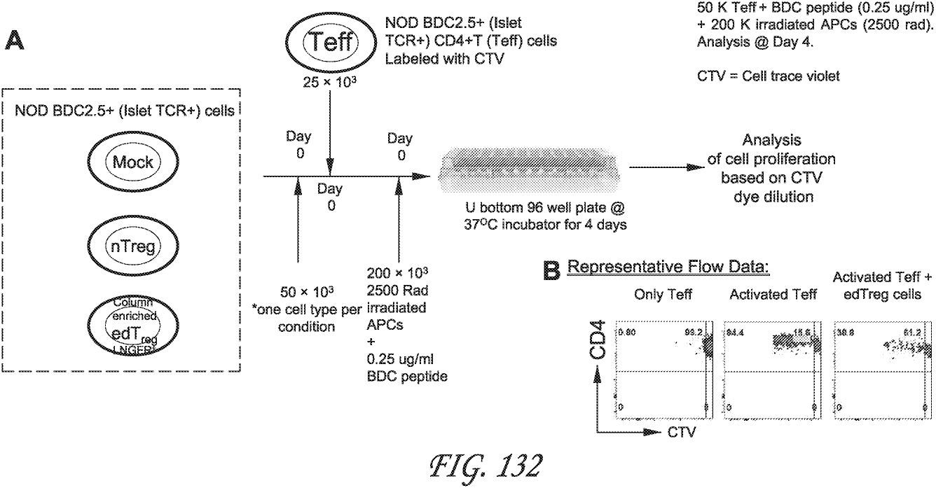

To assess functional activity of mouse Ag-specific edTreg cells, an antigen-specific in vitro suppression assay was established. The proliferation of BCD2.5 (islet-antigen specific) Teff was assessed. The BCD2.5 (islet-antigen specific) Teff were activated by a BDC peptide in the presence and absence of BCD2.5-expressing MND.LNGFR.P2A edTregs or purified BCD2.5 TCR expressing nTreg. Briefly, FIG. 132A depicts an in vitro suppression assay using mouse edTreg or nTreg. MND.LNGFR.p2A (#3261) edited Treg were enriched by anti-LNGFR column at day 2 post editing and resuspended into RPMI media containing 10% FBS. nTreg (CD4+CD25+), Teff (CD4+CD25+) and antigen presenting cells (CD4+CD25+) were isolated from the spleen and lymph nodes cells of 8 to 10 weeks old NOD BDC2.5+ mice by column enrichment. Enriched 5×10⁶ Teff were resuspended in 2 ml of PBS and labeled with cell trace violet for 15 minutes at 37° C. and then washed and resuspended in media before their addition in suppression assay. To setup this assay, 2.0 × 10⁵ irradiated APCs (2500 rad) were loaded with 0.25 µg/ml BDC peptide together with 0.5 × 10⁵ Teff and titrated numbers of BDC2.5+ nTreg or edTreg in a U bottom 96 well tissue culture plate with total volume of 250 µl media. Cells were incubated at 37° C. in CO₂ incubator of four days. At day 4 cells were washed twice with PBS and stained with live/dead, anti-CD4, anti-CD45 and CD25, and analyzed by FACS (LSRII) for the suppression of Teff proliferation by Treg. FIG. 132B depicts representative flow data obtained showing a reduction of BDC2.5+ Teff proliferation in the presence of BDC2.5+ edTreg cells. This demonstrated suppression of peptide-activated Teff cells in the presence of edTregs.

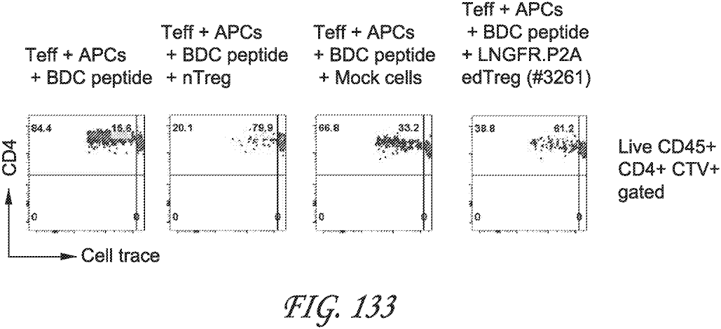

An in vitro suppressive function of murine BDC2.5+ nTreg and edTreg was observed. FIG. 133 depicts flow cytometry plots showing cell trace violet labeled CD4+ T cells in the presence and absence of mock, MND.LNGFR.p2A (#3261) edited Treg or nTregs from NOD BDC2.5+ mice. Numbers in each flow plots indicated the proportion of proliferating vs non-proliferating cells, respectively. Murine edTreg and nTregs exhibited robust in vitro suppressive function. These data demonstrated that edTreg cells expressing the LNGFR selectable marker (MND.LNGFR.P2A #3261) exhibited antigen-specific suppressive activity in vitro.

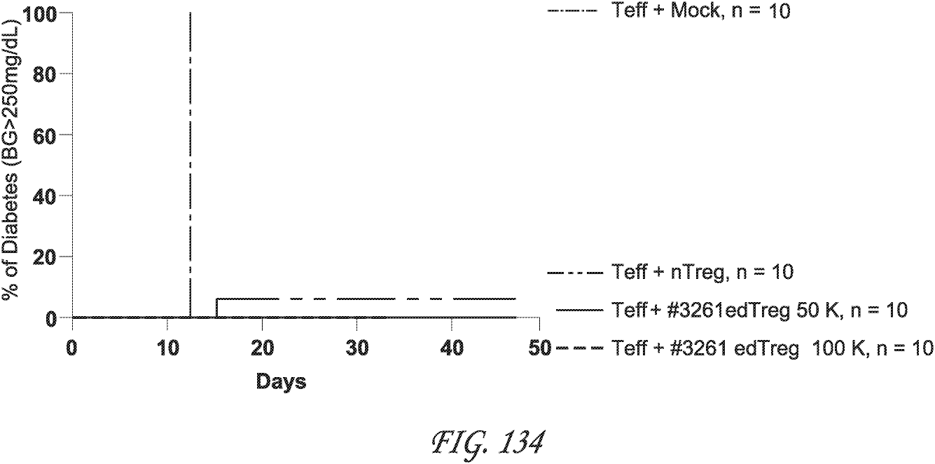

In vivo activities of edTreg were examined with methods substantially similar to those in Examples 6 and 11. In particular, antigen specific T cell function was examined in an NSG adoptive transfer model in which nTregs and column enriched edTregs were compared. Engineered BDC2.5+ antigen-specific (BDC) edTregs, or antigen-specific nTregs were infused into the mice followed by infusion of antigen-specific Teff cells. Mice were monitored for diabetes up to 49 days. FIG. 134 depicts a graph showing the percent of diabetic mice after receiving effector cells plus the designated mock edited, MND.LNGFR.P2A edited or nTreg cells from NOD BDC2.5 mice. Column enriched Ag-specific MND.LNGFR.P2A edTregs completely prevented diabetes in NSG mice and exhibited comparable function to nTregs.

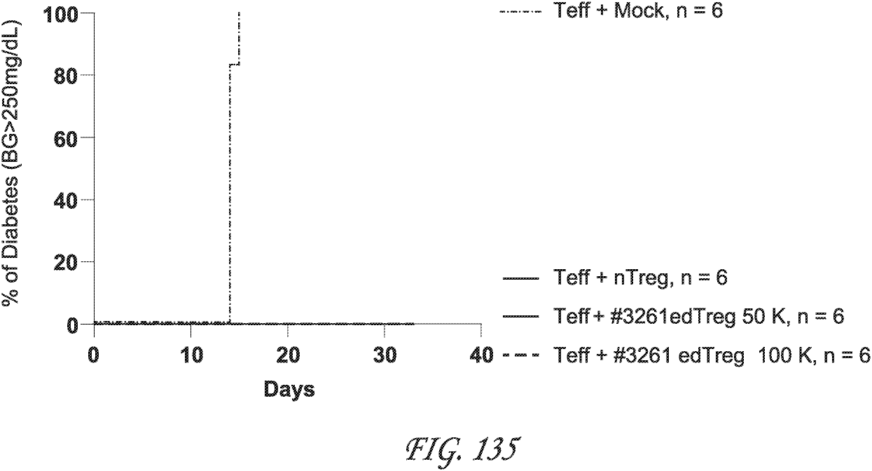

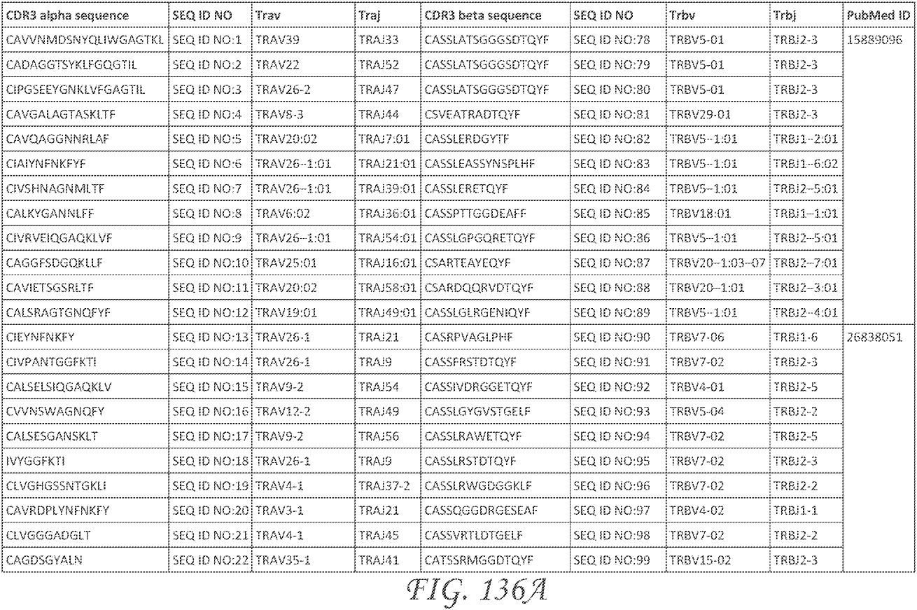

In another study, engineered BDC2.5+ antigen-specific (BDC) edTregs, or antigen-specific nTregs were infused into the mice followed by infusion of antigen-specific Teff cells. Mice were monitored for diabetes up to 33 days. FIG. 135 depicts a graph showing the percent of diabetic mice after receiving effector cells plus the designated mock edited, MND.LNGFR.P2A edited or nTreg cells from NOD BDC2.5 mice. Column enriched Ag-specific MND.LNGFR.P2A edTregs completely prevented diabetes in NSG mice and exhibited comparable function to nTregs. Strikingly, column-enriched LNGFR+ BDC2.5 edTregs completely prevented diabetes in NSG mice and exhibited comparable function to BDC2.5 nTreg in two separate experiments (FIG. 134 and FIG. 135).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1-70. (canceled)

71. A method for producing an engineered cell, comprising introducing into a cell:
(a) a first insertion donor template comprising a nucleic acid sequence encoding a first chemically inducible signaling complex (CISC) component comprising a first extracellular CISC inducer molecule-binding domain that is capable of specifically binding to a CISC inducer molecule, a first transmembrane domain, and a first intracellular activation signal transduction domain; and
(b) a second insertion donor template comprising a nucleic acid sequence encoding a second CISC component comprising a second extracellular CISC inducer molecule-binding domain that is capable of specifically binding to the CISC inducer molecule, a second transmembrane domain, and a second intracellular activation signal transduction domain,
wherein dimerization of the first and second CISC components induces a signal transduction event and expansion of the cell.

72. The method of claim 71, wherein:
(a) at least one of the first and second insertion donor templates further comprises a nucleic acid sequence encoding a third CISC component that is different from the first and second CISC components and is capable of specifically binding to the CISC inducer molecule, optionally wherein the third CISC component comprises an FKBP-rapamycin-binding (FRB) domain of mTOR;
(b) (1) the first CISC component comprises a transmembrane and intracellular domain of IL-2Rβ and the second CISC component comprises a transmembrane and intracellular domain of IL-2Rγ, or (2) the first CISC component comprises a transmembrane and intracellular domain of IL-2Rγ and the second CISC component comprises a transmembrane and intracellular domain of IL-2Rβ;
(c) (1) the first CISC component comprises an extracellular FKBP domain, a transmembrane domain of IL-2Rγ, and an intracellular domain of IL-2Rγ, and the second CISC component comprises an extracellular FRB domain, a transmembrane domain of IL-2Rβ, and an intracellular domain of IL-2Rβ, or (2) the first CISC component comprises an extracellular FRB domain, a transmembrane domain of IL-2Rβ, and an intracellular domain of IL-2Rβ, and the second CISC component comprises an extracellular FKBP domain, a transmembrane domain of IL-2Rγ, and an intracellular domain of IL-2Rγ; and/or
(d) the CISC inducer molecule is rapamycin or a rapalog, optionally wherein the rapalog is AP21967.

73. The method of claim 71, wherein:
(a) the first CISC component comprises an extracellular FKBP domain, a transmembrane domain of IL-2Rγ, and an intracellular domain of IL-2Rγ, and the second CISC component comprises an extracellular FRB domain, a transmembrane domain of IL-2Rβ, and an intracellular domain of IL-2Rβ;
(b) one of the first and second donor templates comprises a nucleic acid sequence encoding an antigen-specific T cell receptor (TCR) polypeptide; and
(c) the other of the first and second donor templates comprises:
(i) a nucleic acid sequence comprising a constitutively active promoter capable of promoting transcription of an endogenous FoxP3-encoding nucleotide sequence of the FOXP3 gene; or
(ii) a nucleic acid sequence comprising a constitutively active promoter operably linked to a nucleotide sequence encoding a FoxP3 protein or a functional derivative thereof, optionally wherein the constitutively active promoter of (i) or (ii) is an MND promoter.

74. The method of claim 73, wherein the donor template comprising the nucleic acid sequence encoding the TCR polypeptide is inserted into a native T cell receptor α (TRAC) locus by homology-directed repair to knock out the native TRAC locus.

75. The method of claim 73, wherein the antigen-specific TCR polypeptide specifically recognizes an antigen associated with pathogenesis of an autoimmune condition, an allergic condition, or an inflammatory condition, optionally wherein:
(a) the autoimmune condition is selected from type 1 diabetes mellitus, multiple sclerosis, systemic lupus erythematosus, myasthenia gravis, rheumatoid arthritis, Crohn’s disease, bullous pemphigoid, pemphigus vulgaris, autoimmune hepatitis, psoriasis, Sjogren’s syndrome, or celiac disease;
(b) the allergic condition is selected from allergic asthma, pollen allergy, food allergy, drug hypersensitivity, or contact dermatitis; and/or
(c) the inflammatory condition is selected from pancreatic islet cell transplantation, asthma, hepatitis, inflammatory bowel disease (IBD), ulcerative colitis, graft-versus-host disease (GvHD), tolerance induction for transplantation, transplant rejection, or sepsis.

76. The method of claim 73, wherein the TCR polypeptide binds to an antigen associated with a disorder selected from type 1 diabetes mellitus, multiple sclerosis, myocarditis, rheumatoid arthritis (RA), and systemic lupus erythematosus (SLE),
optionally wherein the TCR polypeptide binds to an antigen selected from the group consisting of vimentin, aggrecan, cartilage intermediate layer protein (CILP), preproinsulin, islet-specific glucose-6-phosphatase catalytic subunit-related protein (IGRP), and enolase.

77. The method of claim 73, wherein:
the TCR polypeptide comprises a CDR3α sequence having the amino acid sequence of any one of SEQ ID NOs: 1377-1390, and/or
the TCR polypeptide comprises a CDR3β sequence having the amino acid sequence of any one of SEQ ID NOs: 1391-1404.

78. The method of claim 74, wherein:
(1) the antigen associated with pathogenesis of an autoimmune condition is IGRP(305-324) peptide, wherein the autoimmune condition is type 1 diabetes, and wherein the TCR is TCR T1D2 recognizing the IGRP(305-324) peptide in an HLA DRB1*0401-restricted manner;
(2) the antigen associated with pathogenesis of an autoimmune condition is IGRP(241-270) peptide, wherein the autoimmune condition is type 1 diabetes, and wherein the TCR is TCR T1D4 recognizing the IGRP(241-270) peptide in an HLA DRB1*0401-restricted manner;
(3) the antigen associated with pathogenesis of an autoimmune condition is IGRP(305-324) peptide, wherein the autoimmune condition is type 1 diabetes, and wherein the TCR is TCR T1D5-1 recognizing the IGRP(305-324) peptide in an HLA DRB1*0401-restricted manner; or
(4) the antigen associated with pathogenesis of an autoimmune condition is IGRP(305-324) peptide, wherein the autoimmune condition is type 1 diabetes, and wherein the TCR is TCR T1D5-2 recognizing the IGRP(305-324) peptide in an HLA DRB1*0401-restricted manner.

79. An engineered cell, comprising:
(a) an inserted nucleic acid molecule comprising a nucleic acid sequence encoding a first chemically inducible signaling complex (CISC) component comprising a first extracellular CISC inducer molecule-binding domain that is capable of specifically binding to a CISC inducer molecule, a first transmembrane domain, and a first intracellular activation signal transduction domain; and
(b) a transduced polynucleotide comprising a nucleic acid sequence encoding a second CISC component comprising a second extracellular CISC inducer molecule-binding domain that is capable of specifically binding to the CISC inducer molecule, a second transmembrane domain, and a second intracellular activation signal transduction domain,
wherein dimerization of the CISC components induces a signal transduction event and expansion of the cell.

80. The cell of claim 79, wherein:
(a) the cell further comprises an artificial modification of a FOXP3 gene, wherein the modified gene constitutively expresses a FoxP3 gene product at a FoxP3 expression level that is equal to or greater than the FoxP3 expression level of a naturally occurring regulatory T (Treg) cell, wherein the inserted nucleic acid encoding the first CISC component further comprises:
(1) a constitutively active promoter at a native FOXP3 gene locus of the cell, wherein the promoter is positioned in the FOXP3 gene so as to be capable of promoting transcription of an endogenous FoxP3-encoding nucleotide sequence of the FOXP3 gene locus, or
(2) an inserted nucleic acid molecule comprising an exogenous FoxP3-encoding polynucleotide operably linked to a constitutively active promoter at a native FOXP3 gene locus of the cell,
optionally wherein the constitutive promoter is an MND promoter; and
(b) the transduced polynucleotide encodes an antigen-specific T cell receptor (TCR) polypeptide.

81. The cell of claim 80, wherein the antigen-specific TCR polypeptide specifically recognizes an antigen associated with pathogenesis of an autoimmune condition, an allergic condition, or an inflammatory condition, optionally wherein:
(a) the autoimmune condition is selected from type 1 diabetes mellitus, multiple sclerosis, systemic lupus erythematosus, myasthenia gravis, rheumatoid arthritis, Crohn’s disease, bullous pemphigoid, pemphigus vulgaris, autoimmune hepatitis, psoriasis, Sjogren’s syndrome, or celiac disease;
(b) the allergic condition is selected from allergic asthma, pollen allergy, food allergy, drug hypersensitivity, or contact dermatitis; and/or
(c) the inflammatory condition is selected from pancreatic islet cell transplantation, asthma, hepatitis, inflammatory bowel disease (IBD), ulcerative colitis, graft-versus-host disease (GvHD), tolerance induction for transplantation, transplant rejection, or sepsis.

82. The cell of claim 80, wherein the TCR polypeptide binds to an antigen associated with a disorder selected from the group consisting of type 1 diabetes mellitus, multiple sclerosis, myocarditis, rheumatoid arthritis (RA), and systemic lupus erythematosus (SLE),
optionally wherein the TCR polypeptide binds to an antigen selected from the group consisting of vimentin, aggrecan, cartilage intermediate layer protein (CILP), preproinsulin, islet-specific glucose-6-phosphatase catalytic subunit-related protein (IGRP), and enolase.

83. The cell of claim 80, wherein:
the TCR polypeptide comprises a CDR3α sequence having the amino acid sequence of any one of SEQ ID NOs: 1377-1390, and/or
the TCR polypeptide comprises a CDR3β sequence having the amino acid sequence of any one of SEQ ID NOs: 1391-1404.

84. The cell of claim 80, wherein:
(1) the antigen associated with pathogenesis of an autoimmune condition is IGRP(305-324) peptide, wherein the autoimmune condition is type 1 diabetes, and wherein the TCR is TCR T1D2 recognizing the IGRP(305-324) peptide in an HLA DRB1*0401-restricted manner;
(2) the antigen associated with pathogenesis of an autoimmune condition is IGRP(241-270) peptide, wherein the autoimmune condition is type 1 diabetes, and wherein the TCR is TCR T1D4 recognizing the IGRP(241-270) peptide in an HLA DRB1*0401-restricted manner;
(3) the antigen associated with pathogenesis of an autoimmune condition is IGRP(305-324) peptide, wherein the autoimmune condition is type 1 diabetes, and wherein the TCR is TCR T1D5-1 recognizing the IGRP(305-324) peptide in an HLA DRB1*0401-restricted manner; or
(4) the antigen associated with pathogenesis of an autoimmune condition is IGRP(305-324) peptide, wherein the autoimmune condition is type 1 diabetes, and wherein the TCR is TCR T1D5-2 recognizing the IGRP(305-324) peptide in an HLA DRB1*0401-restricted manner.

85. The cell of claim 79, wherein:
(i) (1) the first CISC component comprises a transmembrane and intracellular domain of IL-2Rβ and the second CISC component comprises a transmembrane and intracellular domain of IL-2Rγ, or (2) the first CISC component comprises a transmembrane and intracellular domain of IL-2Rγ and the second CISC component comprises a transmembrane and intracellular domain of IL-2Rβ,
optionally wherein the CISC inducer molecule is rapamycin or a rapalog, optionally wherein the rapalog is AP21967;
(ii) (1) the first CISC component comprises an extracellular FKBP domain, a transmembrane domain of IL-2Rγ, and an intracellular domain of IL-2Rγ, and the second CISC component comprises an extracellular FRB domain, a transmembrane domain of IL-2Rβ, and an intracellular domain of IL-2Rβ, or (2) the first CISC component comprises an extracellular FRB domain, a transmembrane domain of IL-2Rβ, and an intracellular domain of IL-2Rβ, and the second CISC component comprises an extracellular FKBP domain, a transmembrane domain of IL-2Rγ, and an intracellular domain of IL-2Rβγ; and/or
(iii) the inserted nucleic acid or transduced polynucleotide encoding the first or second CISC component further comprises a nucleic acid sequence encoding a third CISC component that is different from the first and second CISC components and is capable of specifically binding to the CISC inducer molecule, optionally wherein the third CISC component comprises an FKBP-rapamycin-binding (FRB) domain of mTOR.

86. The cell of claim 80, wherein the transduced polynucleotide encoding the TCR polypeptide has been inserted into a native T cell receptor α (TRAC) locus by homology-directed repair to knock out the native TRAC locus.

87. The cell of claim 80, wherein:
(i) the FoxP3 is expressed at a level sufficient for the cell to maintain a CD4+CD25+ phenotype for at least 21 days in vitro;
(ii) the FoxP3 is expressed at a level sufficient for the cell to maintain a CD4+CD25+ phenotype for at least 60 days in vivo following adoptive transfer to an immunocompatible mammalian host in need of antigen-specific immunosuppression; and/or
(iii) the cell comprises a phenotype selected from one or more of (a) HeliosLo, (b) CD152+, (c) CD127⁻, and (c) ICOS+.

88. A pharmaceutical composition comprising the cell of claim 79 and a pharmaceutically acceptable excipient.

89. A method for treating an autoimmune condition, allergic condition, or inflammatory condition, the method comprising administering the cell of claim 80 to a subject.

90. The method of claim 89, wherein:
(i) the autoimmune condition is selected from type 1 diabetes mellitus, multiple sclerosis, systemic lupus erythematosus, myasthenia gravis, rheumatoid arthritis, Crohn’s disease, bullous pemphigoid, pemphigus vulgaris, or autoimmune hepatitis;
(ii) the allergic condition is selected from allergic asthma, pollen allergy, food allergy, drug hypersensitivity, or contact dermatitis; and/or
(iii) the inflammatory condition is selected from pancreatic islet cell transplantation, asthma, hepatitis, inflammatory bowel disease (IBD), ulcerative colitis, graft-versus-host disease (GVHD), tolerance induction for transplantation, transplant rejection, or sepsis.
